SEQUENCE LISTING (45 sequences)

SEQ ID NO: 1 - protein, 346 aa, Homo sapiens
MAMVSEFLKQAWFIENEEQEYVQTVKSSKGGPGSAVSPYPTFNPSSDVAALHKAIMVKGVDEATIIDILTKRNNAQRQQIKAAYLQETGKPLDETLKKALTGHLEEVVLALLKTPAQFDADELRAAMKGLGTDEDTLIEILASRTNKEIRDINRVYREELKRDLAKDITSDTSGDFRNALLSLAKGDRSEDFGVNEDLADSDARALYEAGERRKGTDVNVFNTILTTRSYPQLRRVFQKYTKYSKHDMNKVLDLELKGDIEKCLTAIVKCATSKPAFFAEKLHQAMKGVGTRHKALIRIMVSRSEIDMNDIKAFYQKMYGISLCQAILDETKGDYEKILVALCGGN

SEQ ID NO: 2 - protein, 357 aa, Homo sapiens
MGRQLAGCGDAGKKASFKMSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 3 - protein, 339 aa, Homo sapiens
MSTVHEILCKLSLEGDHSTPPSAYGSVKAYTNFDAERDALNIETAIKTKGVDEVTIVNILTNRSNAQRQDIAFAYQRRTKKELASALKSALSGHLETVILGLLKTPAQYDASELKASMKGLGTDEDSLIEIICSRTNQELQEINRVYKEMYKTDLEKDIISDTSGDFRKLMVALAKGRRAEDGSVIDYELIDQDARDLYDAGVKRKGTDVPKWISIMTERSVPHLQKVFDRYKSYSPYDMLESIRKEVKGDLENAFLNLVQCIQNKPLYFADRLYDSMKGKGTRDKVLIRIMVSRSEVDMLKIRSEFKRKYGKSLYYYIQQDTKGDYQKALLYLCGGDD

SEQ ID NO: 4 - protein, 323 aa, Homo sapiens
MASIWVGHRGTVRDYPDFSPSVDAEAIQKAIRGIGTDEKMLISILTERSNAQRQLIVKEYQAAYGKELKDDLKGDLSGHFEHLMVALVTPPAVFDAKQLKKSMKGAGTNEDALIEILTTRTSRQMKDISQAYYTVYKKSLGDDISSETSGDFRKALLTLADGRRDESLKVDEHLAKQDAQILYKAGENRWGTDEDKFTEILCLRSFPQLKLTFDEYRNISQKDIVDSIKGELSGHFEDLLLAIVNCVRNTPAFLAERLHRALKGIGTDEFTLNRIMVSRSEIDLLDIRTEFKKHYGYSLYSAIKSDTSGDYEITLLKICGGDD

SEQ ID NO: 5 - protein, 321 aa, Homo sapiens
MAMATKGGTVKAASGFNAMEDAQTLRKAMKGLGTDEDAIISVLAYRNTAQRQEIRTAYKSTIGRDLIDDLKSELSGNFEQVIVGMMTPTVLYDVQELRRAMKGAGTDEGCLIEILASRTPEEIRRISQTYQQQYGRSLEDDIRSDTSFMFQRVLVSLSAGGRDEGNYLDDALVRQDAQDLYEAGEKKWGTDEVKFLTVLCSRNRNHLLHVFDEYKRISQKDIEQSIKSETSGSFEDALLAIVKCMRNKSAYFAEKLYKSMKGLGTDDNTLIRVMVSRAEIDMLDIRAHFKRLYGKSLYSFIKGDTSGDYRKVLLVLCGGDD

SEQ ID NO: 6 - protein, 320 aa, Homo sapiens
MAQVLRGTVTDFPGFDERADAETLRKAMKGLGTDEESILTLLTSRSNAQRQEISAAFKTLFGRDLLDDLKSELTGKFEKLIVALMKPSRLYDAYELKHALKGAGTNEKVLTEIIASRTPEELRAIKQVYEEEYGSSLEDDVVGDTSGYYQRMLVVLLQANRDPDAGIDEAQVEQDAQALFQAGELKWGTDEEKFITIFGTRSVSHLRKVFDKYMTISGFQIEETIDRETSGNLEQLLLAVVKSIRSIPAYLAETLYYAMKGAGTDDHTLIRVMVSRSEIDLFNIRKEFRKNFATSLYSMIKGDTSGDYKKALLLLCGEDD

SEQ ID NO: 7 - protein, 673 aa, Homo sapiens
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 8 - protein, 641 aa, Homo sapiens
MKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQVAAEILEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 9 - protein, 466 aa, Homo sapiens
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 10 - protein, 488 aa, Homo sapiens
MSYPGYPPTGYPPFPGYPPAGQESSFPPSGQYPYPSGFPPMGGGAYPQVPSSGYPGAGGYPAPGGYPAPGGYPGAPQPGGAPSYPGVPPGQGFGVPPGGAGFSGYPQPPSQSYGGGPAQVPLPGGFPGGQMPSQYPGGQPTYPSQINTDSFSSYPVFSPVSLDYSSEPATVTQVTQGTIRPAANFDAIRDAEILRKAMKGFGTDEQAIVDVVANRSNDQRQKIKAAFKTSYGKDLIKDLKSELSGNMEELILALFMPPTYYDAWSLRKAMQGAGTQERVLIEILCTRTNQEIREIVRCYQSEFGRDLEKDIRSDTSGHFERLLVSMCQGNRDENQSINHQMAQEDAQRLYQAGEGRLGTDESCFNMILATRSFPQLRATMEAYSRMANRDLLSSVSREFSGYVESGLKTILQCALNRPAFFAERLYYAMKGAGTDDSTLVRIVVTRSEIDLVQIKQMFAQMYQKTLGTMIAGDTSGDYRRLLLAIVGQ

SEQ ID NO: 11 - protein, 365 aa, Homo sapiens
MAWWKSWIEQEGVTVKSSSHFNPDPDAETLYKAMKGIGVGSQLLSHQAAAFAFPSSALTSVSPWGQQGHLCCNPAGTNEQAIIDVLTKRSNTQRQQIAKSFKAQFGKDLTETLKSELSGKFERLIVALMYPPYRYEAKELHDAMKGLGTKEGVIIEILASRTKNQLREIMKAYEEDYGSSLEEDIQADTSGYLERILVCLLQGSRDDVSSFVDPGLALQDAQDLYAAGEKIRGTDEMKFITILCTRSATHLLRVFEEYEKIANKSIEDSIKSETHGSLEEAMLTVVKCTQNLHSYFAERLYYAMKGAGTRDGTLIRNIVSRSEIDLNLIKCHFKKMYGKTLSSMIMEDTSGDYKNALLSLVGSDP

SEQ ID NO: 12 - protein, 327 aa, Homo sapiens
MAWWKSWIEQEGVTVKSSSHFNPDPDAETLYKAMKGIGTNEQAIIDVLTKRSNTQRQQIAKSFKAQFGKDLTETLKSELSGKFERLIVALMYPPYRYEAKELHDAMKGLGTKEGVIIEILASRTKNQLREIMKAYEEDYGSSLEEDIQADTSGYLERILVCLLQGSRDDVSSFVDPGLALQDAQDLYAAGEKIRGTDEMKFITILCTRSATHLLRVFEEYEKIANKSIEDSIKSETHGSLEEAMLTVVKCTQNLHSYFAERLYYAMKGAGTRDGTLIRNIVSRSEIDLNLIKCHFKKMYGKTLSSMIMEDTSGDYKNALLSLVGSDP

SEQ ID NO: 13 - protein, 345 aa, Homo sapiens
MSVTGGKMAPSLTQEILSHLGLASKTAAWGTLGTLRTFLNFSVDKDAQRLLRAITGQGVDRSAIVDVLTNRSREQRQLISRNFQERTQQDLMKSLQAALSGNLERIVMALLQPTAQFDAQELRTALKASDSAVDVAIEILATRTPPQLQECLAVYKHNFQVEAVDDITSETSGILQDLLLALAKGGRDSYSGIIDYNLAEQDVQALQRAEGPSREETWVPVFTQRNPEHLIRVFDQYQRSTGQELEEAVQNRFHGDAQVALLGLASVIKNTPLYFADKLHQALQETEPNYQVLIRILISRCETDLLSIRAEFRKKFGKSLYSSLQDAVKGDCQSALLALCRAEDM

SEQ ID NO: 14 - protein, 324 aa, Homo sapiens
MFCGDYVQGTIFPAPNFNPIMDAQMLGGALQGFDCDKDMLINILTQRCNAQRMMIAEAYQSMYGRDLIGDMREQLSDHFKDVMAGLMYPPPLYDAHELWHAMKGVGTDENCLIEILASRTNGEIFQMREAYCLQYSNNLQEDIYSETSGHFRDTLMNLVQGTREEGYTDPAMAAQDAMVLWEACQQKTGEHKTMLQMILCNKSYQQLRLVFQEFQNISGQDMVDAINECYDGYFQELLVAIVLCVRDKPAYFAYRLYSAIHDFGFHNKTVIRILIARSEIDLLTIRKRYKERYGKSLFHDIRNFASGHYKKALLAICAGDAEDY

SEQ ID NO: 15 - protein, 505 aa, Homo sapiens
MSYPGYPPPPGGYPPAAPGGGPWGGAAYPPPPSMPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 16 - protein, 472 aa, Homo sapiens
MPPIGLDNVATYAGQFNQDYLSGMAANMSGTFGGANMPNLYPGAPGAGYPPVPPGGFGQPPSAQQPVPPYGMYPPPGGNPPSRMPSYPPYPGAPVPGQPMPPPGQQPPGAYPGQPPVTYPGQPPVPLPGQQQPVPSYPGYPGSGTVTPAVPPTQFGSRGTITDAPGFDPLRDAEVLRKAMKGFGTDEQAIIDCLGSRSNKQRQQILLSFKTAYGKDLIKDLKSELSGNFEKTILALMKTPVLFDIYEIKEAIKGVGTDEACLIEILASRSNEHIRELNRAYKAEFKKTLEEAIRSDTSGHFQRLLISLSQGNRDESTNVDMSLAQRDAQELYAAGENRLGTDESKFNAVLCSRSRAHLVAVFNEYQRMTGRDIEKSICREMSGDLEEGMLAVVKCLKNTPAFFAERLNKAMRGAGTKDRTLIRIMVSRSETDLLDIRSEYKRMYGKSLYHDISGDTSGDYRKILLKICGGND

SEQ ID NO: 17 - protein, 316 aa, Homo sapiens
MGNRHAKASSPQGFDVDRDAKKLNKACKGMGTNEAAIIEILSGRTSDERQQIKQKYKATYGKELEEVLKSELSGNFEKTALALLDRPSEYAARQLQKAMKGLGTDESVLIEVLCTRTNKEIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 18 - protein, 357 aa, Homo sapiens
MGNRHSQSYTLSEGSQQLPKGDSQPSTVVQPLSHPSRNGEPEAPQPAKASSPQGFDVDRDAKKLNKACKGMGTNEAAIIEILSGRTSDERQQIKQKYKATYGKELEEVLKSELSGNFEKTALALLDRPSEYAARQLQKAMKGLGTDESVLIEVLCTRTNKEIIAIKEAYQRLFDRSLESDVKGDTSGNLKKILVSLLQANRNEGDDVDKDLAGQDAKDLYDAGEGRWGTDELAFNEVLAKRSYKQLRATFQAYQILIGKDIEEAIEEETSGDLQKAYLTLVRCAQDCEDYFAERLYKSMKGAGTDEETLIRIVVTRAEVDLQGIKAKFQEKYQKSLSDMVRSDTSGDFRKLLVALLH

SEQ ID NO: 19 - DNA, 1401 bp, Homo sapiens
agtgtgaaatcttcagagaagaatttctctttagttctttgcaagaaggtagagataaagacactttttcaaaaatggcaatggtatcagaattcctcaagcaggcctggtttattgaaaatgaagagcaggaatatgttcaaactgtgaagtcatccaaaggtggtcccggatcagcggtgagcccctatcctaccttcaatccatcctcggatgtcgctgccttgcataaggccataatggttaaaggtgtggatgaagcaaccatcattgacattctaactaagcgaaacaatgcacagcgtcaacagatcaaagcagcatatctccaggaaacaggaaagcccctggatgaaacactgaagaaagcccttacaggtcaccttgaggaggttgttttagctctgctaaaaactccagcgcaatttgatgctgatgaacttcgtgctgccatgaagggccttggaactgatgaagatactctaattgagattttggcatcaagaactaacaaagaaatcagagacattaacagggtctacagagaggaactgaagagagatctggccaaagacataacctcagacacatctggagattttcggaacgctttgctttctcttgctaagggtgaccgatctgaggactttggtgtgaatgaagacttggctgattcagatgccagggccttgtatgaagcaggagaaaggagaaaggggacagacgtaaacgtgttcaataccatccttaccaccagaagctatccacaacttcgcagagtgtttcagaaatacaccaagtacagtaagcatgacatgaacaaagttctggacctggagttgaaaggtgacattgagaaatgcctcacagctatcgtgaagtgcgccacaagcaaaccagctttctttgcagagaagcttcatcaagccatgaaaggtgttggaactcgccataaggcattgatcaggattatggtttcccgttctgaaattgacatgaatgatatcaaagcattctatcagaagatgtatggtatctccctttgccaagccatcctggatgaaaccaaaggagattatgagaaaatcctggtggctctttgtggaggaaactaaacattcccttgatggtctcaagctatgatcagaagactttaattatatattttcatcctataagcttaaataggaaagtttcttcaacaggattacagtgtagctacctacatgctgaaaaatatagcctttaaatcatttttatattataactctgtataatagagataagtccattttttaaaaatgttttccccaaaccataaaaccctatacaagttgttctagtaacaatacatgagaaagatgtctatgtagctgaaaataaaatgacgtcacaagacaa

SEQ ID NO: 20 - DNA, 1632 bp, Homo sapiens
gctcagcatttggggacgctctcagctctcggcgcacggcccaggtaagcggggcgcgccctgcccgcccgcgatgggccgccagctagcggggtgtggagacgctgggaagaaggcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 21 - DNA, 1665 bp, Homo sapiens
gctcagcatttggggacgctctcagctctcggcgcacggcccagggtgaaaatgtttgccattaaactcacatgaagtaggaaatatttatatggatacaaaaggcacctgcatgggataatgtcaaatttcatagatactgctttgtgcttccttcaaaatgtctactgttcacgaaatcctgtgcaagctcagcttggagggtgatcactctacacccccaagtgcatatgggtctgtcaaagcctatactaactttgatgctgagcgggatgctttgaacattgaaacagccatcaagaccaaaggtgtggatgaggtcaccattgtcaacattttgaccaaccgcagcaatgcacagagacaggatattgccttcgcctaccagagaaggaccaaaaaggaacttgcatcagcactgaagtcagccttatctggccacctggagacggtgattttgggcctattgaagacacctgctcagtatgacgcttctgagctaaaagcttccatgaaggggctgggaaccgacgaggactctctcattgagatcatctgctccagaaccaaccaggagctgcaggaaattaacagagtctacaaggaaatgtacaagactgatctggagaaggacattatttcggacacatctggtgacttccgcaagctgatggttgccctggcaaagggtagaagagcagaggatggctctgtcattgattatgaactgattgaccaagatgctcgggatctctatgacgctggagtgaagaggaaaggaactgatgttcccaagtggatcagcatcatgaccgagcggagcgtgccccacctccagaaagtatttgataggtacaagagttacagcccttatgacatgttggaaagcatcaggaaagaggttaaaggagacctggaaaatgctttcctgaacctggttcagtgcattcagaacaagcccctgtattttgctgatcggctgtatgactccatgaagggcaaggggacgcgagataaggtcctgatcagaatcatggtctcccgcagtgaagtggacatgttgaaaattaggtctgaattcaagagaaagtacggcaagtccctgtactattatatccagcaagacactaagggcgactaccagaaagcgctgctgtacctgtgtggtggagatgactgaagcccgacacggcctgagcgtccagaaatggtgctcaccatgcttccagctaacaggtctagaaaaccagcttgcgaataacagtccccgtggccatccctgtgagggtgacgttagcattacccccaacctcattttagttgcctaagcattgcctggccttcctgtctagtctctcctgtaagccaaagaaatgaacattccaaggagttggaagtgaagtctatgatgtgaaacactttgcctcctgtgtactgtgtcataaacagatgaataaactgaatttgtactttagaaacacgtactttgtggccctgctttcaactgaattgtttgaaaattaaacgtgcttggggttcagctggtgaggctgtccctgtaggaagaaagctctgggactgagctgtacagtatggttgcccctatccaagtgtcgctatttaagttaaatttaaatgaaataaaataaaataaaatcaaaaaaa

SEQ ID NO: 22 - DNA, 1432 bp, Homo sapiens
agcgcggagcacctgcgcccgcggctgacaccttcgctcgcagtttgttcgcagtttactcgcacaccagtttcccccaccgcgctttggattagtgtgatctcagctcaaggcaaaggtgggatatcatggcatctatctgggttggacaccgaggaacagtaagagattatccagactttagcccatcagtggatgctgaagctattcagaaagcaatcagaggaattggaactgatgagaaaatgctcatcagcattctgactgagaggtcaaatgcacagcggcagctgattgttaaggaatatcaagcagcatatggaaaggagctgaaagatgacttgaagggtgatctctctggccactttgagcatctcatggtggccctagtgactccaccagcagtctttgatgcaaagcagctaaagaaatccatgaagggcgcgggaacaaacgaagatgccttgattgaaatcttaactaccaggacaagcaggcaaatgaaggatatctctcaagcctattatacagtatacaagaagagtcttggagatgacattagttccgaaacatctggtgacttccggaaagctctgttgactttggcagatggcagaagagatgaaagtctgaaagtggatgagcatctggccaaacaagatgcccagattctctataaagctggtgagaacagatggggcacggatgaagacaaattcactgagatcctgtgtttaaggagctttcctcaattaaaactaacatttgatgaatacagaaatatcagccaaaaggacattgtggacagcataaaaggagaattatctgggcattttgaagacttactgttggccatagttaattgtgtgaggaacacgccggcctttttagccgaaagactgcatcgagccttgaagggtattggaactgatgagtttactctgaaccgaataatggtgtccagatcagaaattgaccttttggacattcgaacagagttcaagaagcattatggctattccctatattcagcaattaaatcggatacttctggagactatgaaatcacactcttaaaaatctgtggtggagatgactgaaccaagaagataatctccaaaggtccacgatgggctttcccaacagctccaccttacttcttctcatactatttaagagaacaagcaaatataaacagcaacttgtgttcctaacaggaattttcattgttctataacaacaacaacaaaagcgattattattttagagcatctcatttataatgtagcagctcataaatgaaattgaaaatggtattaaagatctgcaactactatccaacttatatttctgctttcaaagttaagaatctttatagttctactccattaaatataaagcaagataataaaaattgttgcttttgttaaaa

SEQ ID NO: 23 - DNA, 2651 bp, Homo sapiens
gtgacctccgcagccgcagaggaggagcgcagcccggcctcgaagaacttctgcttgggtggctgaactctgatcttgacctagagtcatggccatggcaaccaaaggaggtactgtcaaagctgcttcaggattcaatgccatggaagatgcccagaccctgaggaaggccatgaaagggctcggcaccgatgaagacgccattattagcgtccttgcctaccgcaacaccgcccagcgccaggagatcaggacagcctacaagagcaccatcggcagggacttgatagacgacctgaagtcagaactgagtggcaacttcgagcaggtgattgtggggatgatgacgcccacggtgctgtatgacgtgcaagagctgcgaagggccatgaagggagccggcactgatgagggctgcctaattgagatcctggcctcccggacccctgaggagatccggcgcataagccaaacctaccagcagcaatatggacggagccttgaagatgacattcgctctgacacatcgttcatgttccagcgagtgctggtgtctctgtcagctggtgggagggatgaaggaaattatctggacgatgctctcgtgagacaggatgcccaggacctgtatgaggctggagagaagaaatgggggacagatgaggtgaaatttctaactgttctctgttcccggaaccgaaatcacctgttgcatgtgtttgatgaatacaaaaggatatcacagaaggatattgaacagagtattaaatctgaaacatctggtagctttgaagatgctctgctggctatagtaaagtgcatgaggaacaaatctgcatattttgctgaaaagctctataaatcgatgaagggcttgggcaccgatgataacaccctcatcagagtgatggtttctcgagcagaaattgacatgttggatatccgggcacacttcaagagactctatggaaagtctctgtactcgttcatcaagggtgacacatctggagactacaggaaagtactgcttgttctctgtggaggagatgattaaaataaaaatcccagaaggacaggaggattctcaacactttgaatttttttaacttcatttttctacactgctattatcattatctcagaatgcttatttccaattaaaacgcctacagctgcctcctagaatatagactgtctgtattattattcacctataattagtcattatgatgctttaaagctgtacttgcatttcaaagcttataagatataaatggagattttaaagtagaaataaatatgtattccatgtttttaaaagattactttctactttgtgtttcacagacattgaatatattaaattattccatattttcttttcagtgaaaaattttttaaatggaagactgttctaaaatcacttttttccctaatccaatttttagagtggctagtagtttcttcatttgaaattgtaagcatccggtcagtaagaatgcccatccagttttctatatttcatagtcaaagccttgaaagcatctacaaatctctttttttaggttttgtccatagcatcagttgatccttactaagtttttcatgggagacttccttcatcacatcttatgttgaaatcactttctgtagtcaaagtataccaaaaccaatttatctgaactaaattctaaagtatggttatacaaaccatatacatctggttaccaaacataaatgctgaacattccatattattatagttaatgtcttaatccagcttgcaagtgaatggaaaaaaaaataagcttcaaactaggtattctgggaatgatgtaatgctctgaatttagtatgatataaagaaaacttttttgtgctaaaaatactttttaaaatcaattttgttgattgtagtaatttctatttgcactgtgcctttcaactccagaaacattctgaagatgtacttggatttaattaaaaagttcactttgtaagaacgtggaaaaataattttaatttaaaaatggtgtttttaggccgggggcgggggctcacgccagtaatcccaacactttgggaggccaaggcgggtggatcacctaaggtcaggagttcaagactagcctggccaacatggagaaactgcatctctactaaaaatataaaaattagccgggtgtggtggctggtgcctgtaatcccagccactcggaggctgagtcagggagaactgcttgaacccaggaggcaggaggcaaaggttgcagtgagccgagatcacgccagcctgggcgacagagcgagaatccatctaaaaaaaaaaaaaaaaaaagtgtctttaaagtgaggtatagtctttctctgatccacttttcaccttctgaggtttttcatcttggcccctgaaaggagctatttttgaaggacttgtgttactcagtttctacaggaattacaagataagaaaaaaaaaatcatatttagtcttatgcgtgcctactggctaatgttcacatatgccaaacactactcaataacataaaataatgtatgaacttattctctggaaatgagtgatgccctctgctctaagtagaccatttatattaaatatcataaatgtataaaggacattcatattctta

SEQ ID NO: 24 - DNA, 1638 bp, Homo sapiens
agtctaggtgcagctgccggatccttcagcgtctgcatctcggcgtcgccccgcgtaccgtcgcccggctctccgccgctctcccggggtttcggggcacttgggtcccacagtctggtcctgcttcaccttcccctgacctgagtagtcgccatggcacaggttctcagaggcactgtgactgacttccctggatttgatgagcgggctgatgcagaaactcttcggaaggctatgaaaggcttgggcacagatgaggagagcatcctgactctgttgacatcccgaagtaatgctcagcgccaggaaatctctgcagcttttaagactctgtttggcagggatcttctggatgacctgaaatcagaactaactggaaaatttgaaaaattaattgtggctctgatgaaaccctctcggctttatgatgcttatgaactgaaacatgccttgaagggagctggaacaaatgaaaaagtactgacagaaattattgcttcaaggacacctgaagaactgagagccatcaaacaagtttatgaagaagaatatggctcaagcctggaagatgacgtggtgggggacacttcagggtactaccagcggatgttggtggttctccttcaggctaacagagaccctgatgctggaattgatgaagctcaagttgaacaagatgctcaggctttatttcaggctggagaacttaaatgggggacagatgaagaaaagtttatcaccatctttggaacacgaagtgtgtctcatttgagaaaggtgtttgacaagtacatgactatatcaggatttcaaattgaggaaaccattgaccgcgagacttctggcaatttagagcaactactccttgctgttgtgaaatctattcgaagtatacctgcctaccttgcagagaccctctattatgctatgaagggagctgggacagatgatcataccctcatcagagtcatggtttccaggagtgagattgatctgtttaacatcaggaaggagtttaggaagaattttgccacctctctttattccatgattaagggagatacatctggggactataagaaagctcttctgctgctctgtggagaagatgactaacgtgtcacggggaagagctccctgctgtgtgcctgcaccaccccactgccttccttcagcacctttagctgcatttgtatgccagtgcttaacacattgccttattcatactagcatgctcatgaccaacacatacacgtcatagaagaaaatagtggtgcttctttctgatctctagtggagatctctttgactgctgtagtactaaagtgtacttaatgttactaagtttaatgcctggccattttccatttatatatattttttaagaggctagagtgcttttagccttttttaaaaactccatttatattacatttgtaaccatgatactttaatcagaagcttagccttgaaattgtgaactcttggaaatgttattagtgaagttcgcaactaaactaaacctgtaaaattatgatgattgtattcaaaagattaatgaaaaataaacatttctgtccccctgaattatgtgtacatgtgtgtttagatttattattaaatttatttaacaatgtt

SEQ ID NO: 25 - DNA, 2889 bp, Homo sapiens
gcggttgctgctgggctaacgggctccgatccagcgagcgctgcgtcctcgagtccctgcgcccgtgcgtccgtctgcgacccgaggcctccgctgcgcgtggattctgctgcgaaccggagaccatggccaaaccagcacagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggtggctgctgagatcttggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacaca

SEQ ID NO: 26 - DNA, 2879 bp, Homo sapiens
agagaccagagagcatccagaggcctggccggggtcctgcagtgcagacgttgggaggcacggagacggggagagggggaggcggtccaggactcactctgctccacctctgactccttgaagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggtggctgctgagatcttggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacacaaaaaaaaaaa

SEQ ID NO: 27 - DNA, 2443 bp, Homo sapiens
atcttgcgggagaccgggttgggctgtgacgctgctgctggggtcagaatgtcatacccaggctatcccccaacaggctacccacctttccctggatatcctcctgcaggtcaggagtcatcttttcccccttctggtcagtatccttatcctagtggctttcctccaatgggaggaggtgcctacccacaagtgccaagtagtggctacccaggagctggaggctaccctgcgcctggaggttatccagcccctggaggctatcctggtgccccacagccagggggagctccatcctatcccggagttcctccaggccaaggatttggagtcccaccaggtggagcaggcttttctgggtatccacagccaccttcacagtcttatggaggtggtccagcacaggttccactacctggtggctttcctggaggacagatgccttctcagtatcctggaggacaacctacttaccctagtcagcctgccacagtgactcaggtcactcaaggaactatccgaccagctgccaacttcgatgctataagagatgcagaaattcttcgtaaggcaatgaagggttttgggacagatgagcaggcaattgtggatgtggtggccaaccgttccaatgatcagaggcaaaaaattaaagcagcatttaagacctcctatggcaaggatttaatcaaagatctcaaatcagagttaagtggaaatatggaagaactgatcctggccctcttcatgcctcctacgtattacgatgcctggagcttacggaaagcaatgcagggagcaggaactcaggaacgtgtattgattgagattttgtgcacaagaacaaatcaggaaatccgagaaattgtcagatgttatcagtcagaatttggacgagaccttgaaaaggacattaggtcagatacatcaggacattttgaacgtttacttgtgtccatgtgccagggaaatcgtgatgagaaccagagtataaaccaccaaatggctcaggaagatgctcagcgtctctatcaagctggtgaggggagactagggaccgatgaatcttgctttaacatgatccttgccacaagaagctttcctcagctgagagctaccatggaggcttattctaggatggctaatcgagacttgttaagcagtgtgagccgtgagttttccggatatgtagaaagtggtttgaagaccatcttgcagtgtgccctgaaccgccctgccttctttgctgagaggctctactatgctatgaaaggtgctggcacagatgactccaccctggtccggattgtggtcactcgaagtgagattgaccttgtacaaataaaacagatgttcgctcagatgtatcagaagactctgggcacaatgattgcaggtgacacgagtggagattaccgaagacttcttctggctattgtgggccagtaggagggatttttttttttttaatgaaaaaaaatttctattcatagcttatccttcagagcaatgacctgcatgcagcaatatcaaacatcagctaaccgaaagagctttctgtcaaggaccgtatcagggtaatgtgcttggtttgcacatgttgttattgccttaattctaattttattttgttctctacatacaatcaatgtaaagccatatcacaatgatacagtaatattgcaatgtttgtaaaccttcattcttactagtttcattctaatcaagatgtcaaattgaataaaaatcacagcaatctctgattctgtgtaataatattgaataattttttagaaggttactgaaagctctgccttccggaatccctctaagtctgcttgatagagtggatagtgtgttaaaactgtgtactttaaaaaaaaattcaacctttacatctagaataatttgcatctcattttgcctaaattggttctgtattcataaacactttccacatagaaaatagattagtattacctgtggcaccttttaagaaagggtcaaatgtttatatgcttaagatacatagcctacttttttttcgcagttgttttctttttttaaattgagttatgacaaataaaaaattgcatatatttaaggtgtacaatatggtgttttgatatcagcattccttgtgtaatgattccacaattaaggtcaggctaattacgtatctgtcaccttgacatagttaccattttttcatgtgtggtgaaaacacttaagatctactaccttagcaaattttaagtgttcagtacattattaactatagatactgtgctctacattaaacctctagcatttattcgttttataactgaaagtttataccctttgaccaacatctccccattttccccacctctcacctggacaaccaccactgtgtttaagttcagctattttagattccacgtataaatggtatacaata

SEQ ID NO: 28 - DNA, 2550 bp, Homo sapiens
gcccaccctgggcccgcccccggctccatcttgcgggagaccgggttgggctgtgacgctgctgctggggtcagaatgtcatacccaggctatcccccaacaggctacccacctttccctggatatcctcctgcaggtcaggagtcatcttttcccccttctggtcagtatccttatcctagtggctttcctccaatgggaggaggtgcctacccacaagtgccaagtagtggctacccaggagctggaggctaccctgcgcctggaggttatccagcccctggaggctatcctggtgccccacagccagggggagctccatcctatcccggagttcctccaggccaaggatttggagtcccaccaggtggagcaggcttttctgggtatccacagccaccttcacagtcttatggaggtggtccagcacaggttccactacctggtggctttcctggaggacagatgccttctcagtatcctggaggacaacctacttaccctagtcagatcaatacagattctttttcttcctatcctgttttctctcctgtttctttggattatagcagtgaacctgccacagtgactcaggtcactcaaggaactatccgaccagctgccaacttcgatgctataagagatgcagaaattcttcgtaaggcaatgaagggttttgggacagatgagcaggcaattgtggatgtggtggccaaccgttccaatgatcagaggcaaaaaattaaagcagcatttaagacctcctatggcaaggatttaatcaaagatctcaaatcagagttaagtggaaatatggaagaactgatcctggccctcttcatgcctcctacgtattacgatgcctggagcttacggaaagcaatgcagggagcaggaactcaggaacgtgtattgattgagattttgtgcacaagaacaaatcaggaaatccgagaaattgtcagatgttatcagtcagaatttggacgagaccttgaaaaggacattaggtcagatacatcaggacattttgaacgtttacttgtgtccatgtgccagggaaatcgtgatgagaaccagagtataaaccaccaaatggctcaggaagatgctcagcgtctctatcaagctggtgaggggagactagggaccgatgaatcttgctttaacatgatccttgccacaagaagctttcctcagctgagagctaccatggaggcttattctaggatggctaatcgagacttgttaagcagtgtgagccgtgagttttccggatatgtagaaagtggtttgaagaccatcttgcagtgtgccctgaaccgccctgccttctttgctgagaggctctactatgctatgaaaggtgctggcacagatgactccaccctggtccggattgtggtcactcgaagtgagattgaccttgtacaaataaaacagatgttcgctcagatgtatcagaagactctgggcacaatgattgcaggtgacacgagtggagattaccgaagacttcttctggctattgtgggccagtaggagggatttttttttttttaatgaaaaaaaatttctattcatagcttatccttcagagcaatgacctgcatgcagcaatatcaaacatcagctaaccgaaagagctttctgtcaaggaccgtatcagggtaatgtgcttggtttgcacatgttgttattgccttaattctaattttattttgttctctacatacaatcaatgtaaagccatatcacaatgatacagtaatattgcaatgtttgtaaaccttcattcttactagtttcattctaatcaagatgtcaaattgaataaaaatcacagcaatctctgattctgtgtaataatattgaataattttttagaaggttactgaaagctctgccttccggaatccctctaagtctgcttgatagagtggatagtgtgttaaaactgtgtactttaaaaaaaaattcaacctttacatctagaataatttgcatctcattttgcctaaattggttctgtattcataaacactttccacatagaaaatagattagtattacctgtggcaccttttaagaaagggtcaaatgtttatatgcttaagatacatagcctacttttttttcgcagttgttttctttttttaaattgagttatgacaaataaaaaattgcatatatttaaggtgtacaatatggtgttttgatatcagcattccttgtgtaatgattccacaattaaggtcaggctaattacgtatctgtcaccttgacatagttaccattttttcatgtgtggtgaaaacacttaagatctactaccttagcaaattttaagtgttcagtacattattaactatagatactgtgctctacattaaacctctagcatttattcgttttataactgaaagtttataccctttgaccaacatctccccattttccccacctctcacctggacaaccaccactgtgtttaagttcagctattttagattccacgtataaatggtatacaataaaaaaaaaaaaaaa

SEQ ID NO: 29 - DNA, 2205 bp, Homo sapiens
ctgggtggggcctgggagccacaggagatgcccaaagccaggcagagcccgggggcgaggggacggcaggcaggtgtggcgctgccctgggcgggcttgcacccccacacccaagtgagcggcctgctcactcctcagctgcaggagccagacgtgtggagtcccagcagaggccaacctgtgtctcttcatctccctgggaaaggtgcccccgaggtgaaagagatggcctggtggaaatcctggattgaacaggagggtgtcacagtgaagagcagctcccacttcaacccagaccctgatgcagagaccctctacaaagccatgaaggggatcggtgtcgggtcccaactgctcagccaccaagcagctgccttcgccttcccctcctccgccctcaccagtgtgtcaccctgggggcagcagggtcacttgtgctgtaaccctgcagggaccaacgagcaggctatcatcgatgtgctcaccaagagaagcaacacgcagcggcagcagatcgccaagtccttcaaggctcagttcggcaaggacctcactgagaccttgaagtctgagctcagtggcaagtttgagaggctcattgtggcccttatgtacccgccatacagatacgaagccaaggagctgcatgacgccatgaagggcttaggaaccaaggagggtgtcatcattgagatcctggcctctcggaccaagaaccagctgcgggagataatgaaggcgtatgaggaagactatgggtccagcctggaggaggacatccaagcagacacaagtggctacctggagaggatcctggtgtgcctcctgcagggcagcagggatgatgtgagcagctttgtggacccaggactggccctccaagacgcacaggatctgtatgcggcaggcgagaagattcgtgggactgatgagatgaaattcatcaccatcctgtgcacgcgcagtgccactcacctgctgagagtgtttgaagagtatgagaaaattgccaacaagagcattgaggacagcatcaagagtgagacccatggctcactggaggaggccatgctcactgtggtgaaatgcacccaaaacctccacagctactttgcagagagactctactatgccatgaagggagcagggacgcgtgatgggaccctgataagaaacatcgtttcaaggagcgagattgacttaaatcttatcaaatgtcacttcaagaagatgtacggcaagaccctcagcagcatgatcatggaagacaccagcggtgactacaagaacgccctgctgagcctggtgggcagcgacccctgaggcacagaagaacaagagcaaagaccatgaagccagagtctccaggactcctcactcaacctcggccatggacgcaggttgggtgtgaggggggtcccagcctttcggtcttctatttccctatttccagtgctttccagccgggtttctgacccagagggtggaaccggcctggactcctcttcccaacttcctccaggtcatttcccagtgtgagcacaatgccaaccttagtgtttctccagccagacagatgcctcagcatgaagggcttggggacttgtggatcattccttcctccctgcaggagcttcccaagctggtcacagagtctcctgggcacaggttatacagaccccagccccattcccatctactgaaacagggtctccacaagaggggccagggaatatgggtttttaacaagcgtcttacaaaacacttctctatcatgcagccggagagctggctgggagcccttttgttttagaacacacatccttcagcagctgagaaacgaacacgaatccatcccaaccgagatgccattaacattcatctaaaaatgttaggctctaaatggacgaaaaattctctcgccatcttaataacaaaataaactacaaattcctgacccaaggacactgtgttataagaggcgtgggctcccctggtggctgaccaggtcagctgccctggccttgcacccctctgcatgcagcacagaagggtgtgaccatgccctcagcaccactcttgtccccactgaacggcaactgagactgggtacctggagattctgaagtgcctttgctgtggttttcaaaataataaagatttgtattcaactcaaaaaaaaaaaaaaaaaaaaaaaaaaaaaaa

SEQ ID NO: 30 - DNA, 2070 bp, Homo sapiens
ctgggtggggcctgggagccacaggagatgcccaaagccaggcagagcccgggggcgaggggacggcaggcaggtgtggcgctgccctgggcgggcttgcacccccacacccaagtgagcggcctgctcactcctcagctgcaggagccagacgtgtggagtcccagcagaggccaacctgtgtctcttcatctccctgggaaaggtgcccccgaggtgaaagagatggcctggtggaaatcctggattgaacaggagggtgtcacagtgaagagcagctcccacttcaacccagaccctgatgcagagaccctctacaaagccatgaaggggatcgggaccaacgagcaggctatcatcgatgtgctcaccaagagaagcaacacgcagcggcagcagatcgccaagtccttcaaggctcagttcggcaaggacctcactgagaccttgaagtctgagctcagtggcaagtttgagaggctcattgtggcccttatgtacccgccatacagatacgaagccaaggagctgcatgacgccatgaagggcttaggaaccaaggagggtgtcatcattgagatcctggcctctcggaccaagaaccagctgcgggagataatgaaggcgtatgaggaagactatgggtccagcctggaggaggacatccaagcagacacaagtggctacctggagaggatcctggtgtgcctcctgcagggcagcagggatgatgtgagcagctttgtggacccaggactggccctccaagacgcacaggatctgtatgcggcaggcgagaagattcgtgggactgatgagatgaaattcatcaccatcctgtgcacgcgcagtgccactcacctgctgagagtgtttgaagagtatgagaaaattgccaacaagagcattgaggacagcatcaagagtgagacccatggctcactggaggaggccatgctcactgtggtgaaatgcacccaaaacctccacagctactttgcagagagactctactatgccatgaagggagcagggacgcgtgatgggaccctgataagaaacatcgtttcaaggagcgagattgacttaaatcttatcaaatgtcacttcaagaagatgtacggcaagaccctcagcagcatgatcatggaagacaccagcggtgactacaagaacgccctgctgagcctggtgggcagcgacccctgaggcacagaagaacaagagcaaagaccatgaagccagagtctccaggactcctcactcaacctcggccatggacgcaggttgggtgtgaggggggtcccagcctttcggtcttctatttccctatttccagtgctttccagccgggtttctgacccagagggtggaaccggcctggactcctcttcccaacttcctccaggtcatttcccagtgtgagcacaatgccaaccttagtgtttctccagccagacagatgcctcagcatgaagggcttggggacttgtggatcattccttcctccctgcaggagcttcccaagctggtcacagagtctcctgggcacaggttatacagaccccagccccattcccatctactgaaacagggtctccacaagaggggccagggaatatgggtttttaacaagcgtcttacaaaacacttctctatcatgcagccggagagctggctgggagcccttttgttttagaacacacatccttcagcagctgagaaacgaacacgaatccatcccaaccgagatgccattaacattcatctaaaaatgttaggctctaaatggacgaaaaattctctcgccatcttaataacaaaataaactacaaattcctgacccaaggacactgtgttataagaggcgtgggctcccctggtggctgaccaggtcagctgccctggccttgcacccctctgcatgcagcacagaagggtgtgaccatgccctcagcaccactcttgtccccactgaacggcaactgagactgggtacctggagattctgaagtgcctttgctgtggttttcaaaataataaagatttgtattcaactcaaaaaaaaaa

SEQ ID NO: 31 - DNA, 1551 bp, Homo sapiens
ctctaccaggccacaccggaggcagtgctcacacaggcaagctaccaggccacaacaacgacacccacctcacctctggcacctctgagcatccacgtacttgcaagaactcttgctcacatcagctaagagattgcacctgctgacctagagattccggcctgtgctcctgtgctgctgagcagggcaaccagtagcaccatgtctgtgactggcgggaagatggcaccgtccctcacccaggagatcctcagccacctgggcctggccagcaagactgcagcgtgggggaccctgggcaccctcaggaccttcttgaacttcagcgtggacaaggatgcgcagaggctactgagggccattactggccaaggcgtggaccgcagtgccattgtggacgtgctgaccaaccggagcagagagcaaaggcagctcatctcacgaaacttccaggagcgcacccaacaggacctgatgaagtctctacaggcagcactttccggcaacctggagaggattgtgatggctctgctgcagcccacagcccagtttgacgcccaggaattgaggacagctctgaaggcctcagattctgctgtggacgtggccattgaaattcttgccactcgaaccccaccccagctgcaggagtgcctggcagtctacaaacacaatttccaggtggaggctgtggatgacatcacatctgagaccagtggcatcttgcaggacctgctgttggccctggccaaggggggccgtgacagctactctggaatcattgactataatctggcagaacaagatgtccaggcactgcagcgggcagaaggacctagcagagaggaaacatgggtcccagtcttcacccagcgaaatcctgaacacctcatccgagtgtttgatcagtaccagcggagcactgggcaagagctggaggaggctgtccagaaccgtttccatggagatgctcaggtggctctgctcggcctagcttcggtgatcaagaacacaccgctgtactttgctgacaaacttcatcaagccctccaggaaactgagcccaattaccaagtcctgattcgcatccttatctctcgatgtgagactgaccttctgagtatcagagctgagttcaggaagaaatttgggaagtccctctactcttctctccaggatgcagtgaaaggggattgccagtcagccctcctggccttgtgcagggctgaagacatgtgagacttccctgccccaccccacatgacatccgaggatctgagatttccgtgtttggctgaacctgggagaccagctgggcctccaagtaggataacccctcactgagcaccacattctctagcttcttgttgaggctggaactgtttctttaaaatcccttaattttcccatctcaaaattatatctgtacctgggtcatccagctccttcttgggtgtggggaaatgagttttctttgatagtttctgcctcactcatccctcctgtaccctggccagaacatctcactgatactcgaattcttttggcaaa

SEQ ID NO: 32 - DNA, 1447 bp, Homo sapiens
atccagatttgcttttacattttcttgcctgagtctgaggtgaacagtgaacatatttacatttgatttaacagtgaaccttaattctttctggcttcacagtgaaacaagtttatgcaatcgatcaaatattttcatccctgaggttaacaattaccatcaaaatgttttgtggagactatgtgcaaggaaccatcttcccagctcccaatttcaatcccataatggatgcccaaatgctaggaggagcactccaaggatttgactgtgacaaagacatgctgatcaacattctgactcagcgctgcaatgcacaaaggatgatgattgcagaggcataccagagcatgtatggccgggacctgattggggatatgagggagcagctttcggatcacttcaaagatgtgatggctggcctcatgtacccaccaccactgtatgatgctcatgagctctggcatgccatgaagggagtaggcactgatgagaattgcctcattgaaatactagcttcaagaacaaatggagaaattttccagatgcgagaagcctactgcttgcaatacagcaataacctccaagaggacatttattcagagacctcaggacacttcagagatactctcatgaacttggtccaggggaccagagaggaaggatatacagaccctgcgatggctgctcaggatgcaatggtcctatgggaagcctgtcagcagaagacgggggagcacaaaaccatgctgcaaatgatcctgtgcaacaagagctaccagcagctgcggctggttttccaggaatttcaaaatatttctgggcaagatatggtagatgccattaatgaatgttatgatggatactttcaggagctgctggttgcaattgttctctgtgttcgagacaaaccagcctattttgcttatagattatatagtgcaattcatgactttggtttccataataaaactgtaatcaggattctcattgccagaagtgaaatagacctgctgaccataaggaaacgatacaaagagcgatatggaaaatccctatttcatgatatcagaaattttgcttcagggcattataagaaagcactgcttgccatctgtgctggtgatgctgaggactactaaaatgaagaggacttggagtactgtgcactcctctttctagacacttccaaatagagattttctcacaaatttgtactgttcatggcactattaacaaaactatacaatcatattttctcttctatctttgaaattattctaagccaaagaaaactatgaatgaaagtatatgatactgaatttgcctactatcctgaatttgcctactatctaatcagcaattaaataaattgtgcatgatggaataatagaaaaattgcattggaatagattttatttaaatgtgaaccatcaacaacctacaacaa

SEQ ID NO: 33 - DNA, 6734 bp, Homo sapiens
ggagttttccgcccggcgctgacggctgctgcgcccgcggctccccagtgccccgagtgccccgcgggccccgcgagcgggagtgggacccagcccctaggcagaacccaggcgccgcgcccgggacgcccgcggagagagccactcccgcccacgtcccatttcgcccctcgcgtccggagtccccgtggccaggtgtgtgtctggggaagagacttacagaagtggagttgctgagtcaaagatctaaccatgagctaccctggctatcccccgcccccaggtggctacccaccagctgcaccaggtggtggtccctggggaggtgctgcctaccctcctccgcccagcatgccccccatcgggctggataacgtggccacctatgcggggcagttcaaccaggactatctctcgggaatggcggccaacatgtctgggacatttggaggagccaacatgcccaacctgtaccctggggcccctggggctggctacccaccagtgccccctggcggctttgggcagcccccctctgcccagcagcctgttcctccctatgggatgtatccacccccaggaggaaacccaccctccaggatgccctcatatccgccatacccaggggcccctgtgccgggccagcccatgccaccccccggacagcagcccccaggggcctaccctgggcagccaccagtgacctaccctggtcagcctccagtgccactccctgggcagcagcagccagtgccgagctacccaggatacccggggtctgggactgtcacccccgctgtgcccccaacccagtttggaagccgaggcaccatcactgatgctcccggctttgaccccctgcgagatgccgaggtcctgcggaaggccatgaaaggcttcgggacggatgagcaggccatcattgactgcctggggagtcgctccaacaagcagcggcagcagatcctactttccttcaagacggcttacggcaaggatttgatcaaagatctgaaatctgaactgtcaggaaactttgagaagacaatcttggctctgatgaagaccccagtcctctttgacatttatgagataaaggaagccatcaagggggttggcactgatgaagcctgcctgattgagatcctcgcttcccgcagcaatgagcacatccgagaattaaacagagcctacaaagcagaattcaaaaagaccctggaagaggccattcgaagcgacacatcagggcacttccagcggctcctcatctctctctctcagggaaaccgtgatgaaagcacaaacgtggacatgtcactcgcccagagagatgcccaggagctgtatgcggccggggagaaccgcctgggaacagacgagtccaagttcaatgcggttctgtgctcccggagccgggcccacctggtagcagttttcaatgagtaccagagaatgacaggccgggacattgagaagagcatctgccgggagatgtccggggacctggaggagggcatgctggccgtggtgaaatgtctcaagaataccccagccttctttgcggagaggctcaacaaggccatgaggggggcaggaacaaaggaccggaccctgattcgcatcatggtgtctcgcagcgagaccgacctcctggacatcagatcagagtataagcggatgtacggcaagtcgctgtaccacgacatctcgggagatacttcaggggattaccggaagattctgctgaagatctgtggtggcaatgactgaacagtgactggtggctcacttctgcccacctgccggcaacaccagtgccaggaaaaggccaaaagaatgtctgtttctaacaaatccacaaatagccccgagattcaccgtcctagagcttaggcctgtcttccacccctcctgacccgtatagtgtgccacaggacctgggtcggtctagaactctctcaggatgccttttctaccccatccctcacagcctcttgctgctaaaatagatgtttcatttttctgactcatgcaatcattcccctttgcctgtggctaagacttggcttcatttcgtcatgtaattgtatatttttatttggaggcatattttcttttcttacagtcattgccagacagaggcatacaagtctgtttgctgcatacacatttctggtgagggcgactgggtgggtgaagcaccgtgtcctcgctgaggagagaaagggaggcgtgcctgagaaggtagcctgtgcatctggtgagtgtgtcacgagctttgttactgccaaactcactcctttttagaaaaaacaaaaaaaaagggccagaaagtcattccttccatcttccttgcagaaaccacgagaacaaagccagttccctgtcagtgacagggcttcttgtaatttgtggtatgtgccttaaacctgaatgtctgtagccaaaacttgtttccacattaagagtcagccagctctggaatggtctggaaatgtcttcctggtaccaacttgttttcttctgcttgattctgccctgtggctcagaggtctggccttatcagccagtgaaagttcatgtaaccttacgtagagatttgtgtgcaggaaaccctgagcatacactagtttgcagggactcgtaaggacatgggaagggaggttcccgaaatccaggcaggaggcccagacacctgaaaggcaaagggatcttggttggttgcaggtgcagtgaagtccactgaaggtgtggtgcgaagaatgcagtccttcacccaggtcccaggagggaagaagggtgtgtgctaattcctggtgcccctcggcgggggccagagagaaggatggggacaacccagagagtcacaagaccagtgcctcccctcagggtgcctccaggctgaaaggggctcctggctctggtctctggggaccctgtgcccgttggttggtggtgtgagggaagagaatccataagagagtttctgagaattatggtgtcatgtccagaagctagagcttaccttgcatcaggggtctccacccactccttttccaacctcctgcgttgaggtttagaaaagagagaatcgactaggcactatggctcacgcctgtaatccaaggactttgggaagctgaggtgagaggatcacttgagctcaggagttcaagactagcctagccaacagcgagacccctgtctctactaaaaaatttggccaggcgtggtggctcacggctgtaatcccagcactttgggaggtgaggcgggcagatcacctgaggtcaggagttcgagacccagcctggccaacatggtgaaaccccatctctactaaaaatacaaaaattagccaggcatggtggcacattcctgtaatcccagctacacaggatgctgaggcaggagaatcacttgaacccaggaggcagaggttgtagtgagctgagatcacaccattgcacttcaacctgggtggacagagtgagactctgtctcaaaaaaaaaaaaaaatttacctggcattgtagtgcattccctatagtcggctactctggaggctgaggcaggaagatccttagagcccaagaaattgaggccgtagtaagctgtgattacaccactgcactccagcctggacaacagagcgagaccttgtctcaaatgagaaaaaaacaaaaagaaatgggagaatccagagagactaggctagatcaagcctgctgggtcctggcaggagccccagggagtagctcatctgcagacatttgcttgaggactaccccctaaacataaaggaagaatgacatccgaagggtgtggagcagccatgagctgagaactagcctggtctacctgagattgatggcaggtcctggtcaacacgtcagctctgcgtcagagtccatgcctcaagcccaagctgaagccccatccctgctgctctcccaagaactcctctgctagggcaggccccttgcccttgggtgccaggtgggacctgcctgatgggatggggtgcttggcatatacaacttgccatgaactcaaggtgaccctgggggcctcctgaattgtgatggggcctagaaccaatgtgctctgatgtgaccatattctgtgacattaccttgccctgtttactccaaagttcccagcctggtgcccagcaggcaatattgcacctacagacacatttactttggtttccaaagtgtttttagacatttgaatttgttgccaacatttaaacattgagagatttcatatttttaaaaatctggaattctggcttctcttgaaaactcagaaattctggcactatggggcttgcattcctgcatggctggagctgagttgcagctgcccctttaggcctgtactccttatttgctataggctccgtcttgtattacactaagcccatgtcacccatttggctcctgcaggcctttgggtttgagaccctggtctacacacttggagaccacctgttgtaaagtacatggatgtgctttggtcaaggaatagaccaaggtggatatccaggccagagtgactcagcgagtttaggtcacaggcgtatactccacttgttatataacctgcttgtgtaagttcatacttggctcaaagccactattgtttggaaaaggtataactgccctgctgacgctgtacagatgttcttgggctcggatgggcatggctccacgtggtgtgcactagcacccagagagagtgaagctattgacccctgtaagggagagtgaccatctggcagatagatagaggggagccaggacatggctcagcttgtgcccagagggagagttaagccgctgaccctgtagccagggagtgcacctgcaagcatgggggtggcaggagccacagagctggctgctgagaggagctgcagatctggagaagacagcctaggtaaaggtggacagtgtgagagctgctgatgagatagctgctgaataaaactacattttacctgcctatggcccgccaggttttctttcagctatcgcccatccacccagtcccctcgaacctcagcatgggctggaacctgaccctgggcatgacatttggcatagttgtggacctgacacctgtgtttgtcctagtcctgtttctccctgccttcctgttcctctcgctgccctcatggtcactcccaagagatccaacccatgttaagtatgggctggaggactgcatgaatgcctcatgatcttcccagaggcaaaggcacctactgccttccaaggtcagtgggaggttgggatcaacactgtttattatgcttaggacaaaaaagatagggagaaagatgtgcaaccttacaggtcatctttctgggatagaacacaatgggtcttctcctgcctcctggatatgttagtcaaggccagtccatgctacacatctagtctgacttctaaaatagaagcaccagatgaattcagccctgagagaattttcagcagctgtgggggcgctggaggaaacactattaaatagttttgcacctgagacagatagcctcactcgcctcaccctagtcctggtggcatttgtctcaggtgcaaaatttaagaaagaaaccttggagtgctcaccctgtggctgggtagatggtcctaaagtggtggttttcaagcctgagtgtgtatcaggatcatcaggggagcttgctaaagagcagttcctgcggtcagaccctcatgcattttgagcaggtgtggggactgggaaactgcatctgtaacctgctgtaatctaacgcttatctaaatactactgtgctcacacagagaacaccgcaaaagtagaggtgttcctccagagggcaggtgagcagatggcacagtctgcttggaattcagtcaggtgatgagagatgagatgaggcactcctagctttgggaagagggagctgaaagatgaacctttgcaggtgcccacggtcaaagtggtggtttaatgccatgccatgcccattttctgttggccttggcagggagttacagccctaccttaggacctggctccttatttctgctgtaggctctttcctgccctggccgagatggagtggaatgagacctagaaacatcaagctaaatacatgtcctcagaaagataaaggtttacattttcacccccatcaaatctgaaagctctctgcctgtgtttttctaagggatagggacatcattactcagtccacaacctggactcatgtagggtcccctgtcagtaaaggagtcagtcaagcccaccaggtataccaaggactcttaccctcagcccctactccttggaaagctgccccttggcctaatattggtgtttagcttgagcctgactccttctcaacactaagagctgatgaagtcctgaagcagaaagagctctgacctgagagtcaaacatccttattctgatctcagctcagcccctgatttgttgtgtgaccctggatatgtcacttcctgtctttttgactttttaaaatgaagggtagactagaggagagcttctaaaactttaatgtggtcaacgaaatggaataggaaattccacaagtctgtccttccacaaaagcagcaaataaggtggcaaaaactcaaatttatgggaactctggaaacgaattgaaagtttacagcaatcaggtgaatacctaagaataaaagctggatttagtaaga

SEQ ID NO: 34 - DNA, 2804 bp, Homo sapiens
gcactgcctctggcacctggggcagccgcgcccgcggagttttccgcccggcgctgacggctgctgcgcccgcggctccccagtgccccgagtgccccgcgggccccgcgagcgggagtgggacccagcccctaggcagaacccaggcgccgcgcccgggacgcccgcggagagagccactcccgcccacgtcccatttcgcccctcgcgtccggagtccccgtggccaggtgtgtgtctggggaagagacttacagaagtggagttgctgagtcaaagatctaaccatgagctaccctggctatcccccgcccccaggtggctacccaccagctgcaccaggttggctggcactggcctgggttctctctctatagtagaaatcctgccatccagatcctgccactgccacctttgctagcacagctgagcagcctctgagcagcaagagaggaggaggcaggaaatttagggaaggttcttcctggagggtctggagccctggagatgaagagccgatccgaagctgccatgtagaggaaagcatctaacaggccagaggccccatgatgatgtcgaatgcccatcgggcacccagctgagccctgcaggtggtggtccctggggaggtgctgcctaccctcctccgcccagcatgccccccatcgggctggataacgtggccacctatgcggggcagttcaaccaggactatctctcgggaatggcggccaacatgtctgggacatttggaggagccaacatgcccaacctgtaccctggggcccctggggctggctacccaccagtgccccctggcggctttgggcagcccccctctgcccagcagcctgttcctccctatgggatgtatccacccccaggaggaaacccaccctccaggatgccctcatatccgccatacccaggggcccctgtgccgggccagcccatgccaccccccggacagcagcccccaggggcctaccctgggcagccaccagtgacctaccctggtcagcctccagtgccactccctgggcagcagcagccagtgccgagctacccaggatacccggggtctgggactgtcacccccgctgtgcccccaacccagtttggaagccgaggcaccatcactgatgctcccggctttgaccccctgcgagatgccgaggtcctgcggaaggccatgaaaggcttcgggacggatgagcaggccatcattgactgcctggggagtcgctccaacaagcagcggcagcagatcctactttccttcaagacggcttacggcaaggatttgatcaaagatctgaaatctgaactgtcaggaaactttgagaagacaatcttggctctgatgaagaccccagtcctctttgacatttatgagataaaggaagccatcaagggggttggcactgatgaagcctgcctgattgagatcctcgcttcccgcagcaatgagcacatccgagaattaaacagagcctacaaagcagaattcaaaaagaccctggaagaggccattcgaagcgacacatcagggcacttccagcggctcctcatctctctctctcagggaaaccgtgatgaaagcacaaacgtggacatgtcactcgcccagagagatgcccaggagctgtatgcggccggggagaaccgcctgggaacagacgagtccaagttcaatgcggttctgtgctcccggagccgggcccacctggtagcagttttcaatgagtaccagagaatgacaggccgggacattgagaagagcatctgccgggagatgtccggggacctggaggagggcatgctggccgtggtgaaatgtctcaagaataccccagccttctttgcggagaggctcaacaaggccatgaggggggcaggaacaaaggaccggaccctgattcgcatcatggtgtctcgcagcgagaccgacctcctggacatcagatcagagtataagcggatgtacggcaagtcgctgtaccacgacatctcgggagatacttcaggggattaccggaagattctgctgaagatctgtggtggcaatgactgaacagtgactggtggctcacttctgcccacctgccggcaacaccagtgccaggaaaaggccaaaagaatgtctgtttctaacaaatccacaaatagccccgagattcaccgtcctagagcttaggcctgtcttccacccctcctgacccgtatagtgtgccacaggacctgggtcggtctagaactctctcaggatgccttttctaccccatccctcacagcctcttgctgctaaaatagatgtttcatttttctgactcatgcaatcattcccctttgcctgtggctaagacttggcttcatttcgtcatgtaattgtatatttttatttggaggcatattttcttttcttacagtcattgccagacagaggcatacaagtctgtttgctgcatacacatttctggtgagggcgactgggtgggtgaagcaccgtgtcctcgctgaggagagaaagggaggcgtgcctgagaaggtagcctgtgcatctggtgagtgtgtcacgagctttgttactgccaaactcactcctttttagaaaaaacaaaaaaaaagggccagaaagtcattccttccatcttccttgcagaaaccacgagaacaaagccagttccctgtcagtgacagggcttcttgtaatttgtggtatgtgccttaaacctgaatgtctgtagccaaaacttgtttccacattaagagtcagccagctctggaatggtctggaaatgtca

SEQ ID NO: 35 - DNA, 1456 bp, Homo sapiens
gcctgtaggaggactgatctcttgatgaaatacagaaaaaccatctcagaaaaaggaaaatgggcaatcgtcatgctaaagcgagcagtcctcagggttttgatgtggatcgagatgccaaaaagctgaacaaagcctgcaaaggaatggggaccaatgaagcagccatcattgaaatcttatcgggcaggacatcagatgagaggcaacaaatcaagcaaaagtacaaggcaacgtacggcaaggagctggaggaagtactcaagagtgagctgagtggaaacttcgagaagacagcgttggcccttctggaccgtcccagcgagtacgccgcccggcagctgcagaaggctatgaagggtctgggcacagatgagtccgtcctcattgaggtcctgtgcacgaggaccaataaggaaatcatcgccattaaagaggcctaccaaaggctatttgataggagcctcgaatcagatgtcaaaggtgatacaagtggaaacctaaaaaaaatcctggtgtctctgctgcaggctaatcgcaatgaaggagatgacgtggacaaagatctagctggtcaggatgccaaagatctgtatgatgcaggggaaggccgctggggcactgatgagcttgcgttcaatgaagtcctggccaagaggagctacaagcagttacgagccacctttcaagcctatcaaattctcattggcaaagacatagaagaagccattgaagaagaaacatcaggcgacttgcagaaggcctatttaactctcgtgagatgtgcccaggattgtgaggactattttgctgaacgtctgtacaagtcgatgaagggtgcggggaccgatgaggagacgttgattcgcatagtcgtgaccagggccgaggtggaccttcaggggatcaaagcaaagttccaagagaagtatcagaagtctctctctgacatggttcgctcagatacctccggggacttccggaaactgctagtagccctcttgcactgagccaagccagggcaataggaacacagggtggaaccgcctttgtcaagagcacattccaaatcaaacttgcaaatgagactcccgcacgaaaacccttaagagtcccggattactttcttggcagcttaagtggcgcagccaggccaagctgtgtaagttaagggcagtaacgttaagatgcgtgggcagggcaccttgaactctggcttagcaagcatctaggctgcctcttcactttcttttagcatggtaactggatgttttctaaacactaatgaaatcagcagttgatgaaaaaactatgcatttgtaatggcacatttagaaggatatgcatcacacaagtaaggtacaggaaagacaaaattaaacaatttattaattttccttctgtgtgttcaatttgaaagcctcattgttaattaaagttgtggattatgcctcta

SEQ ID NO: 36 - DNA, 1633 bp, Homo sapiens
attatgtccggggggaaaactgttgtaaactttgcctgtaggaggactgatctcttaatgaaatacagaaaaaccatctcagaaaaaggaaaatgggcaatcgtcatagccagtcgtacaccctctcagaaggcagtcaacagttgcctaaaggggactcccaaccctcgacagtcgtgcagcctctcagccacccatcacggaatggagagccagaggccccacagcctgctaaagcgagcagtcctcagggttttgatgtggatcgagatgccaaaaagctgaacaaagcctgcaaaggaatggggaccaatgaagcagccatcattgaaatcttatcgggcaggacatcagatgagaggcaacaaatcaagcaaaagtacaaggcaacgtacggcaaggagctggaggaagtactcaagagtgagctgagtggaaacttcgagaagacagcgttggcccttctggaccgtcccagcgagtacgccgcccggcagctgcagaaggctatgaagggtctgggcacagatgagtccgtcctcattgaggtcctgtgcacgaggaccaataaggaaatcatcgccattaaagaggcctaccaaaggctatttgataggagcctcgaatcagatgtcaaaggtgatacaagtggaaacctaaaaaaaatcctggtgtctctgctgcaggctaatcgcaatgaaggagatgacgtggacaaagatctagctggtcaggatgccaaagatctgtatgatgcaggggaaggccgctggggcactgatgagcttgcgttcaatgaagtcctggccaagaggagctacaagcagttacgagccacctttcaagcctatcaaattctcattggcaaagacatagaagaagccattgaagaagaaacatcaggcgacttgcagaaggcctatttaactctcgtgagatgtgcccaggattgtgaggactattttgctgaacgtctgtacaagtcgatgaagggtgcggggaccgatgaggagacgttgattcgcatagtcgtgaccagggccgaggtggaccttcaggggatcaaagcaaagttccaagagaagtatcagaagtctctctctgacatggttcgctcagatacctccggggacttccggaaactgctagtagccctcttgcactgagccaagccagggcaataggaacacagggtggaaccgcctttgtcaagagcacattccaaatcaaacttgcaaatgagactcccgcacgaaaacccttaagagtcccggattactttcttggcagcttaagtggcgcagccaggccaagctgtgtaagttaagggcagtaacgttaagatgcgtgggcagggcaccttgaactctggcttagcaagcatctaggctgcctcttcactttcttttagcatggtaactggatgttttctaaacactaatgaaatcagcagttgatgaaaaaactatgcatttgtaatggcacatttagaaggatatgcatcacacaagtaaggtacaggaaagacaaaattaaacaatttattaattttccttctgtgtgttcaatttgaaagcctcattgttaattaaagttgtggattatgcctctaaaaaaaaaaaaaaaaaaaaaa

SEQ ID NO: 37 - DNA, 276 bp, Artificial Sequence
  Synthetic Polynucleotide
aactgtgcttcacagcatttctctacacattgttgtattatagcaaattgaaaacatttatttaagcaaggaagcagctcaaagctagggactatacatagcaaacatatgaaaccattttaataagtaaattccatattcacaagcaacatgggctaatgaatgtaaaagacacaacggcatacattgatcaagaatgctataaattattatgcattaaaatcaattttctgggctgtggggggtaggattggtacttaagaagagaaaagcttc

SEQ ID NO: 38 - DNA, 407 bp, Artificial Sequence
  Synthetic Polynucleotide
tgagctatggtgtccaagcaggacacactgtcaggggacctgatgcaaccattcagatacccaggtggacttcacatactggagcaggcacagaccatgttctccagtcccctctttccaaagggctgcctttacccccatgaagtcactgtgctaattcagtgagttccaaaactggtcaataatgacactggatgctggattatagaatgggcaataaaatacctacagaggctgggcagtggtagtgtacaactttaatcccagcgcttgggaggcagaggcaggcggatctctgtgggtttgaggccagcctggtatgcaaagtgagttccaggtcagccagggtgacagagaaaccctcccccacccaatcctacatgtggtcatataccttctctgtagga

SEQ ID NO: 39 - DNA, 300 bp, Artificial Sequence
  Synthetic Polynucleotide
tttgcctcggcagtttcttaagccactgttctaagatgggactgcttgtactaccaggaaatgggtttgtgggaagcagctgccagagctgttcagacagcagcgggcacagcaggggtgaaggtatctctgctcctcaagactgaacttgaggtgtctgtctcagagtaagcttcccccccccccccccccgccttagtacattcccctctgacagatgagtaaactctcacaatacagcctgttctctaagttgaggactggtttaaccattttttggaaacacttggctcatgcctt

SEQ ID NO: 40 - DNA, 492 bp, Artificial Sequence
  Synthetic Polynucleotide
aggccggatctttgttttcttgttattgcccccccccaccccccaagacaggatttcactttgtagccctggctgtcctggaactctccctgtagaccaggctgtcctcaaaactcacagagatctgcttgcctctgcttcctgagtgctgggatcgaaggtgtgttccaccactgccctcccccatttttttgtttttagggatgaaattctgagctggagagatggacagtggttaagagcattgactgctcttctagaggacctgggttcaattcccagcacccatatggccggtcacaactgtctgcaactccaggatctaacaccctcacagagatgtacatgcaggcaaaacatcagtaagcctaaaattaaaaaatgaattatttaataaaggagtgaaattcacacaacacgaatgaaccatttaaagatgcacagtttagtggcttgggtacatcatgtctaaccacccctcttcctagttcc

SEQ ID NO: 41 - DNA, 212 bp, Artificial Sequence
  Synthetic Polynucleotide
cttgcgacaaagacgcataaatgagtaaggtggcaatttttagtctctaaaattgctccggtcgtctgcttctagttgctcctaattcaggcaactaaaaggacaacttaacttgaaccttcagggttcaggacccggagccctgagcaaaatgggccctctccaagtccctccccctgttccctgttgtccaatggctatgccagaattgg

SEQ ID NO: 42 - DNA, 211 bp, Artificial Sequence
  Synthetic Polynucleotide
gagaagatgtgggcagaaccctcctttgaaaagcttagaaagattagaagaggaggtgaggatgtgtttaaagagttctctagtaaagggaggattttccccctaagatagaagagagttgagcccctttatgagctaagaggagagagcaggaaagatgaagacacatcagagaatggtcacagtagacagaggacatggacaagaggga

SEQ ID NO: 43 - DNA, 154 bp, Artificial Sequence
  Synthetic Polynucleotide
agtcagttccaaaccgtgctgaccatggctatgatccaaaggcctgccccttacgtcagaggcgagcctccgggtccagctgaggggcagggctgtcctcccttctatatagtatttaaagcaaggagggcgggctaccaagcacagttggcct

SEQ ID NO: 44 - protein, 667 aa, Homo Sapiens
MAKPAQGAKYRGSIHDFPGFDPNQDAEALYTAMKGFGSDKEAILDIITSRSNRQRQEVCQSYKSLYGKDLIADLKYELTGKFERLIVGLMRPPAYCDAKEIKDAISGIGTDEKCLIEILASRTNEQMHQLVAAYKDAYERDLEADIIGDTSGHFQKMLVVLLQGTREEDDVVSEDLVQQDVQDLYEAGELKWGTDEAQFIYILGNRSKQHLRLVFDEYLKTTGKPIEASIRGELSGDFEKLMLAVVKCIRSTPEYFAERLFKAMKGLGTRDNTLIRIMVSRSELDMLDIREIFRTKYEKSLYSMIKNDTSGEYKKTLLKLSGGDDDAAGQFFPEAAQVAYQMWELSAVARVELKGTVRPANDFNPDADAKALRKAMKGLGTDEDTIIDIITHRSNVQRQQIRQTFKSHFGRDLMTDLKSEISGDLARLILGLMMPPAHYDAKQLKKAMEGAGTDEKALIEILATRTNAEIRAINEAYKEDYHKSLEDALSSDTSGHFRRILISLATGHREEGGENLDQAREDAQEIADTPSGDKTSLETRFMTILCTRSYPHLRRVFQEFIKMTNYDVEHTIKKEMSGDVRDAFVAIVQSVKNKPLFFADKLYKSMKGAGTDEKTLTRIMVSRSEIDLLNIRREFIEKYDKSLHQAIEGDTSGDFLKALLALCGGED

SEQ ID NO: 45 - DNA, 2871 bp, Homo Sapiens
gcggttgctgctgggctaacgggctccgatccagcgagcgctgcgtcctcgagtccctgcgcccgtgcgtccgtctgcgacccgaggcctccgctgcgcgtggattctgctgcgaaccggagaccatggccaaaccagcacagggtgccaagtaccggggctccatccatgacttcccaggctttgaccccaaccaggatgccgaggctctgtacactgccatgaagggctttggcagtgacaaggaggccatactggacataatcacctcacggagcaacaggcagaggcaggaggtctgccagagctacaagtccctctacggcaaggacctcattgctgatttaaagtatgaattgacgggcaagtttgaacggttgattgtgggcctgatgaggccacctgcctattgtgatgccaaagaaattaaagatgccatctcgggcattggcactgatgagaagtgcctcattgagatcttggcttcccggaccaatgagcagatgcaccagctggtggcagcatacaaagatgcctacgagcgggacctggaggctgacatcatcggcgacacctctggccacttccagaagatgcttgtggtcctgctccagggaaccagggaggaggatgacgtagtgagcgaggacctggtacaacaggatgtccaggacctatacgaggcaggggaactgaaatggggaacagatgaagcccagttcatttacatcttgggaaatcgcagcaagcagcatcttcggttggtgttcgatgagtatctgaagaccacagggaagccgattgaagccagcatccgaggggagctgtctggggactttgagaagctaatgctggccgtagtgaagtgtatccggagcaccccggaatattttgctgaaaggctcttcaaggctatgaagggcctggggactcgggacaacaccctgatccgcatcatggtctcccgtagtgagttggacatgctcgacattcgggagatcttccggaccaagtatgagaagtccctctacagcatgatcaagaatgacacctctggcgagtacaagaagactctgctgaagctgtctgggggagatgatgatgctgctggccagttcttcccggaggcagcgcaggtggcctatcagatgtgggaacttagtgcagtggcccgagtagagctgaagggaactgtgcgcccagccaatgacttcaaccctgacgcagatgccaaagcgctgcggaaagccatgaagggactcgggactgacgaagacacaatcatcgatatcatcacgcaccgcagcaatgtccagcggcagcagatccggcagaccttcaagtctcactttggccgggacttaatgactgacctgaagtctgagatctctggagacctggcaaggctgattctggggctcatgatgccaccggcccattacgatgccaagcagttgaagaaggccatggagggagccggcacagatgaaaaggctcttattgaaatcctggccactcggaccaatgctgaaatccgggccatcaatgaggcctataaggaggactatcacaagtccctggaggatgctctgagctcagacacatctggccacttcaggaggatcctcatttctctggccacggggcatcgtgaggagggaggagaaaacctggaccaggcacgggaagatgcccaggaaatagcagacacacctagtggagacaaaacttccttggagacacgtttcatgacgatcctgtgtacccggagctatccgcacctccggagagtcttccaggagttcatcaagatgaccaactatgacgtggagcacaccatcaagaaggagatgtctggggatgtcagggatgcatttgtggccattgttcaaagtgtcaagaacaagcctctcttctttgccgacaaactttacaaatccatgaagggtgctggcacagatgagaagactctgaccaggatcatggtatcccgcagtgagattgacctgctcaacatccggagggaattcattgagaaatatgacaagtctctccaccaagccattgagggtgacacctccggagacttcctgaaggccttgctggctctctgtggtggtgaggactagggccacagctttggcgggcacttctgccaagaaatggttatcagcaccagccgccatggccaagcctgattgttccagctccagagactaaggaaggggcaggggtggggggaggggttgggttgggctcttatcttcagtggagcttaggaaacgctcccactcccacgggccatcgagggcccagcacggctgagcggctgaaaaaccgtagccatagatcctgtccacctccactcccctctgaccctcaggctttcccagcttcctccccttgctacagcctctgccctggtttgggctatgtcagatccaaaaacatcctgaacctctgtctgtaaaatgagtagtgtctgtactttgaatgagggggttggtggcaggggccagttgaatgtgctgggcggggtggtgggaaggatagtaaatgtgctggggcaaactgacaaatcttcccatccatttcaccacccatctccatccaggccgcgctagagtactggaccaggaatttggatgcctgggttcaaatctgcatctgccatgcacttgtttctgaccttaggccagcccctttccctccctgagtctctattttcttatctacaatgagacagttggacaaaaaaatcttggcttcccttctaacattaacttcctaaagtatgcctccgattcattcccttgacactttttatttctaaggaagaaataaaaagagatacacaaacacataaacaca

(19) United States
(12) Patent Application Publication
(10) Pub. No.: US 2022/0062299 A1
(43) Pub. Date: Mar. 3, 2022

(54) USE OF GLUCOCORTICOID STEROIDS IN PREVENTING AND TREATING CONDITIONS OF MUSCLE WASTING, AGING AND METABOLIC DISORDER

(71) Applicant: NORTHWESTERN UNIVERSITY, Evanston, IL (US)

(72) Inventors: Mattia Quattrocelli, Chicago, IL (US); Alexis R. Demonbreun, Mokena, IL (US); Elizabeth M. McNally, Oak Park, IL (US)

(21) Appl. No.: 17/416,792

(22) PCT Filed: Dec. 26, 2019
(86) PCT No.: PCT/US19/68618

§ 371 Date: Jun. 21, 2021

Related U.S. Application Data
(60) Provisional application No. 62/876,238, filed on Jul. 19, 2019.
(60) Provisional application No. 62/785,029, filed on Dec. 26, 2018.

(51) Int. Cl. A61K 31/573; A61K 38/17; A61P 21/06

(52) U.S. Cl. CPC A61K 31/573; A61P 21/06; A61K 38/1709

(57) ABSTRACT

Chronic glucocorticoid steroids produce muscle atrophy, but intermittent steroid exposure can promote muscle growth and function. It is disclosed herein that, in contrast to daily administration of a steroid, once-weekly steroid administration improved muscle mass and exercise tolerance in normal subjects as well as multiple models of muscle disease.

### CROSS-REFERENCE TO RELATED APPLICATIONS

This application claims the priority benefit under 35 U.S.C. § 119(e) of U.S. Provisional Patent Application No. 62/785,029, filed Dec. 26, 2018 and U.S. Provisional Patent Application No. 62/876,238, filed Jul. 19, 2019, which are incorporated herein by reference in their entirety.

### STATEMENT OF GOVERNMENT INTEREST

This invention was made with government support under grant numbers U54 AR052646, R01 NS047726, and K01 DK121875 awarded by the National Institutes of Health. The government has certain rights in the invention.

### INCORPORATION BY REFERENCE OF MATERIAL SUBMITTED ELECTRONICALLY

The Sequence Listing, which is a part of the present disclosure, is submitted concurrently with the specification as a text file. The name of the text file containing the Sequence Listing is “2018-192R_Seqlisting.txt”, which was created on Dec. 23, 2019 and is 132,364 bytes in size. The subject matter of the Sequence Listing is incorporated herein in its entirety by reference.

### BACKGROUND

Muscle metabolism is fundamental for ergogenic performance and whole-body homeostasis (Ahn et al., 2016; Bentzinger et al., 2008; Shintaku et al., 2016). Catabolism of branched-chain amino acids (BCAA) improves muscle metabolism and glucose handling (D'Antona et al., 2010; White et al., 2018). In the mdx model of Duchenne muscular dystrophy (DMD) and in mouse models of aging and obesity, muscle mitochondrial function and NAD⁺ levels are impaired (Ryu et al., 2016; Zhang et al., 2016), and mechanisms to offset these deficiencies are useful to improve muscle function.

Glucocorticoid (GC) steroids have broad metabolic effects, mainly through interaction of the activated glucocorticoid receptor (GR) with co-factors to regulate gene expression (Vockley et al., 2016). Glucocorticoids prolong ambulation in DMD (McDonald et al., 2018). However, chronic daily intake of glucocorticoids has adverse consequences like metabolic dysfunction and obesity (Nadal et al., 2017). GC steroids have not been recommended for other genetic forms of muscular dystrophies and in dysferlin-deficient muscular dystrophy are harmful (Walter et al., 2013). Alternative GC dosing strategies may limit side effects (Connolly et al., 2002), but the mechanisms and clinical benefit of these strategies are debated.

### SUMMARY

Impaired metabolic homeostasis drives many conditions including diabetes, obesity, and deconditioning, and burdens the population by manifesting as muscle wasting/weakness, exercise intolerance and unhealthy aging. Novel strategies are needed to restore metabolic homeostasis and thereby improve quality of life. Glucocorticoids are widely prescribed drugs for chronic inflammatory conditions, but their daily administration causes adverse side effects including muscle atrophy, obesity, and osteoporosis, often overshadowing primary drug benefits. It is disclosed herein that, in contrast to daily regimen, once-weekly steroids improved muscle mass and exercise tolerance in normal mice and multiple mouse models of muscle disease (Quattrocelli et al JCI 2017, Quattrocelli et al AJP 2017; Quattrocelli et al., JCI Insight. 2019 Dec. 19; 4(24). pii: 132402. doi: 10.1172fjci.insight.132402). These benefits were achieved without eliciting the negative metabolic or endocrine side effects associated with daily dosing (Quattrocelli et al JCI 2017, Quattrocelli et al AJP 2017, Quattrocelli et al., JCI Insight. 2019 Dec. 19; 4(24). pii: 132402. doi: 10.1172rjci.insight.132402).

It is further contemplated that the methods of the disclosure are useful in treating or ameliorating additional indications, and the molecular and metabolic mechanisms associated with the favorable reprogramming induced by once-weekly glucocorticoids is described herein.

Once-weekly glucocorticoids increased glucose uptake, nutrient metabolism and energy production in muscle, blunting fat accrual and insulin resistance. This glucocorticoid-induced program correlated with increased production of the anti-adiposity molecule adiponectin, and with a corresponding profile of circulating metabolic biomarkers. These trends are clinically relevant, as similar biomarker profiles were observed in patients with Duchenne Muscular Dystrophy receiving intermittent versus daily glucocorticoid steroids. Additionally, favorable muscle metabolic remodeling was observed in experimental conditions of mice with aging-related muscle wasting. Furthermore, in mouse models of obesity, once-weekly glucocorticoids reduced fat accrual while increasing lean mass, exercise tolerance and adiponectin levels. The data provided herein indicate that once-weekly glucocorticoids remodel muscle metabolism and body-wide homeostasis, counteracting insulin resistance and wasting associated with aging and metabolic disorders.

The present disclosure provides, in some aspects, methods for preventing and treating aging, obesity, and dysmetabolism.

Applications for the methods and compositions provided herein include, but are not limited to, treatment or prevention of muscle wasting, treatment or prevention of unhealthy aging, treatment or prevention of metabolic disorders, treatment or prevention of sarcopenia, treatment or prevention of obesity, enhancement of nutrient metabolism, enhancement of energy production, enhancement of energy expenditure, enhancement of exercise tolerance, enhancement of insulin sensitivity, enhancement of adiponectin production, reduced osteoporosis, reduced muscle wasting, reduced insulin resistance, and reduced fat accrual.

Advantages provided by the disclosure include, but are not limited to, once-weekly dosing of an FDA approved drug for new therapeutic indications targeting a potentially large patient population, favorable metabolic reprogramming induced by once-weekly glucocorticoids is applicable to a range of conditions, from muscle wasting and sarcopenia to diabetes and obesity, multiple dosing routes elicit this same beneficial effect (in mice both oral and intraperitoneal injection yield the same effect), once-weekly glucocorticoids promotes production and sensitivity to the anti-adiposity molecule adiponectin, glucocorticoid steroids can be administered independent of sex, age, concomitant medical conditions, glucocorticoid steroids can be administered independent of genetic mutation, weekly glucocorticoid steroids promotes exercise tolerance and performance, and clinically-relevant biomarkers to follow favorable metabolic reprogramming in humans.

It is shown herein that:
Once-weekly glucocorticoid steroids increase nutrient metabolism, including glucose, amino acids, fatty acids and ketone bodies in muscle,
Once-weekly glucocorticoid steroids enhance nutrient flux and improves energy balance in muscle.
Once-weekly glucocorticoid steroids increase production and circulating levels of adiponectin.
Once-weekly glucocorticoid steroids decrease tissue and circulating levels of free fatty acids and ketone bodies.
Once-weekly glucocorticoid steroids reduce weight and fat accrual in obesity.
Once-weekly glucocorticoid steroids preserve or increase lean mass in obesity.
Once-weekly glucocorticoid steroids enhance muscle function including grip strength, running capacity and force generation.
Once-weekly glucocorticoid steroids increase muscle mass in aging, dysmetabolic and wasting conditions.
Once-weekly glucocorticoid steroids enhance cardiac functional output parameters.
Once-weekly glucocorticoid steroids enhance breathing parameters measured by whole-body plethysmography.
Once-weekly glucocorticoid steroids do not induce osteoporosis.
Once-weekly glucocorticoid steroids reduce negative effects normally associated with daily GCs, including bone loss, atrophy, and adrenal dysfunction.

Glucocorticoid steroids are widely prescribed drugs for chronic inflammatory conditions, and their daily intake generally correlates with muscle wasting and weakness, osteoporosis, obesity and metabolic disorders. However, it is disclosed herein that changing the dosing frequency of glucocorticoids (e.g., prednisone, deflazacort; 1 mg/kg) to once-weekly improved muscle force and mass in three murine models of muscle disease (mdx; Dysf-null; Sgcg-null), contrary to daily dosing that induced the known adverse side effects. (Quattrocelli et al, J Clin Invest 2017; Quattrocelli et al, Am J Pathol 2017; Quattrocelli et al., JCI Insight. 2019 Dec. 19; 4(24). pii: 132402. doi: 10.1172/jci.insight.132402).

As disclosed herein, multiple profiling approaches were integrated to define the molecular pathways enabled by weekly glucocorticoid dosing. Combining epigenomics (H3K27ac ChIP-seq), transcriptomics (RNA-seq) and metabolomics (untargeted mass spectroscopy), showed that once-weekly prednisone stimulates muscle metabolism of amino acids, glucose and fatty acids, which associates with increased muscle performance and metabolic function (Quattrocelli et al., JCI Insight. 2019 Dec. 19; 4(24). pii: 132402. doi: 10.1172/jci.insight.132402).

In some aspects, the present disclosure provides a method of administering a glucocorticoid steroid to a patient, wherein the patient has a serum or plasma level of one or more of the following biomarkers that is:
(a) less than about 18 μg/dL morning fasting cortisol;
(b) at least about 90 mg/dL fasting morning glucose;
(c) at least about 160 pmol/L insulin;
(d) at least about 40 μmol/L isoleucine;
(e) at least about 100 μmol/L leucine;
(f) at least about 120 μmol/L valine;
(g) at least about 700 μmol/L combined branched chain amino acids;
(h) at least about 110 mg/dL triglycerides;
(i) at least about 300 μmol/L non-esterified fatty acids; and/or
(j) at least about 100 μmol/L combined ketones;

wherein the administering of the glucocorticoid steroid comprises once-weekly administration of the glucocorticoid steroid. In some embodiments, the patient suffers from muscle wasting, obesity, a metabolic disorder, sarcopenia, an inflammatory disorder, a muscle injury, or a combination thereof. In further embodiments, the once-weekly administration of glucocorticoid steroid comprises a single dose of about 0.1 to about 5 mg/kg. In some embodiments, the once-weekly administration of glucocorticoid steroid comprises a single dose of about 1 mg/kg. In further embodiments, the once-weekly administration of glucocorticoid steroid comprises a single dose of about 0.75 mg/kg.

In some embodiments, the muscle wasting is aging-related muscle wasting, disease-related muscle wasting, diabetes-associated muscle wasting, muscle atrophy, sarcopenia, cardiomyopathy, a chronic myopathy, an inflammatory myopathy, a muscular dystrophy, or a combination thereof. In further embodiments, the cardiomyopathy is hypertrophic, dilated, congenital, arrhythmogenic, restrictive, ischemic, or heart failure. In some embodiments, the heart failure includes reduced ejection fraction. In further embodiments, the heart failure includes preserved ejection fraction.

In some embodiments, the metabolic disorder is metabolic syndrome, insulin resistance, a nutrition disorder, exercise intolerance, or a combination thereof.

In some embodiments, the administering results in one or more of decreased insulin resistance, increased glucose tolerance, increased muscle mass, decreased hyperinsulinemia, increased lean mass, increased force, increased systolic function, increased diastolic function, decreased muscle fibrosis, increased exercise tolerance, increased nutrient catabolism, increased energy production, increased serum adiponectin, decreased serum branched chain amino acids (BCAA), decreased serum lipid level, decreased serum ketone level, decreased hyperglycemia, increased serum cortisol level, increased serum corticosterone, and decreased adipocyte size compared to administering the glucocorticoid steroid in a dosing regimen that is not once-weekly or to not administering the glucocorticoid steroid.

In any of the aspects or embodiments of the disclosure, a method as disclosed herein further comprises administering an effective amount of (i) an agent that increases the activity of an annexin protein, (ii) mitsugumin 53 (MG53), (iii) a modulator of latent TGF-β binding protein 4 (LTBP4), (iv) a modulator of transforming growth factor β (TGF-β) activity, (v) a modulator of androgen response, (vi) a modulator of an inflammatory response, (vii) a promoter of muscle growth, (viii) a chemotherapeutic agent, (ix) a modulator of fibrosis, (x) a modulator of glucose homeostasis, (xi) a modulator of metabolic function, or a combination thereof. In some embodiments, the agent that increases the activity of an annexin protein is selected from the group consisting of a recombinant protein, a steroid, and a polynucleotide capable of expressing an annexin protein. In further embodiments, the polynucleotide is associated with a nanoparticle. In some embodiments, the polynucleotide is contained in a vector. In further embodiments, the vector is within a chloroplast. In still further embodiments, the vector is a viral vector. In yet additional embodiments, the viral vector is selected from the group consisting of a herpes virus vector, an adeno-associated virus (AAV) vector, an adeno virus vector, and a lentiviral vector. In some embodiments, the AAV vector is recombinant AAV5, AAV6, AAV8, AAV9, or AAV74. In further embodiments, the AAV74 vector is AAVrh74. In some embodiments, gene editing mediated by CRISPR (clustered regularly interspaced short palindromic repeats), Cas9, or a functional equivalent thereof, is used to induce genetic changes within heart or muscle for treatment (See, e.g., Pickar-Oliver & Gersbach, Nat Rev Mol Cell Biol 2019, incorporated herein by reference in its entirety). In further embodiments, the CRISPR-mediated genetic changes include, but are not limited to, gene replacement, gene reintroduction, gene correction and gene re-framing in order to restore defective protein function or to treat an underlying condition (See, e.g., Maeder M L, Gersbach C A, MOL THER, 2016 24(3); 430-46, incorporated herein by reference in its entirety).

In some embodiments, the agent increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 44, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof. In some embodiments, the agent increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 44, or a combination thereof). In some embodiments, the agent increases the activity of annexin A2 (SEQ ID NO: 2 or SEQ ID NO: 3) and annexin A6 (SEQ ID NO: 7, SEQ ID NO: 8, SEQ ID NO: 44, or a combination thereof). In further embodiments, the agent increases the activity of annexin A1 (SEQ ID NO: 1) and annexin A6 (SEQ ID NO: 7, SEQ ID NO: 8, SEQ ID NO: 44, or a combination thereof). In some embodiments, the agent increases the activity of annexin A6 (SEQ ID NO: 7, SEQ ID NO: 8, SEQ ID NO: 44, or a combination thereof).

Other features and advantages of the disclosure will be better understood by reference to the following further description, including the figures and the examples.

### BRIEF DESCRIPTION OF THE FIGURES

FIG. 1 shows that pulsatile (weekly) glucocorticoid exposure enhanced mitochondrial respiration in dystrophic muscle through BCAA. mdx mice were treated with weekly (pulsatile) or daily 1 mg/kg intraperitoneal prednisone administration, the most commonly used glucocorticoid steroid. (A) Principal Component Analysis (PCA) of 171 metabolites showed treatment-specific clustering of muscle tissues. (B) Heatmaps of metabolite levels showed that pulsatile prednisone increased BCAA and glutamine catabolism to TCA cycle, increasing ATP and phosphocreatine levels. Weekly prednisone enhanced glycolysis and NAD levels. (C) Muscle respirometry showed that weekly prednisone led to higher basal oxygen consumption in the presence of valine and higher basal lactate production in the presence of glucose. (D) Weekly prednisone increased BCKDHA levels and reduced its phosphorylation in muscle. (E-F) Weekly treatment increased amino acid sensing, mTOR pathway activation, protein translation, marked by puromycin integration in proteins, and mass in quadriceps muscle. (G)¹⁸FDG-PET of live animals showed increased glucose uptake in striated muscles (arrows) after weekly prednisone (bl., bladder). Curves depict mean±s.e.m.; histograms depict single values and mean±s.e.m.; box plots, Tukey distribution; n=3 mice/group (A-B; J-L), n=6 mice/group (C-D). *, P<0.05 vs vehicle, 2-way ANOVA test with Tukey's multiple comparison (C,G), 1-way ANOVA test with Tukey's multiple comparison (D), Welch's unpaired t-test (two-tailed) (E-F). For additional data, see also FIG. 5.

FIG. 2 shows epigenetic programs in steroid-treated dystrophic muscles. Myofiber-specific H3K27 acetylation profiles were integrated with RNAseq data from treated mdx muscle. (A) PCA analysis of H3K27ac profiles from quadriceps myofibers separates the prednisone regimens from each other and from vehicle treated controls. (B) Gene Ontology (GO) analysis of concordant genes with both increased RNAseq expression and H3K27 acetylation revealed that weekly prednisone enriched for nutrient metabolism and muscle function pathways, while daily prednisone enriched for atrophy-related terms. (C) KIf15 and Mef2C were among top concordant in upregulation and acetylation after weekly prednisone, while Foxo3 and other atrophy agonists were concordant after daily prednisone. (D) Representative H3K27ac markings across gene loci had divergent acetylation enrichment with respect to weekly or daily prednisone (blue arrows, gain; red arrow, loss of H3K27ac signal). (E) Glucocorticoid Response Elements (GRE), Klf response elements (KRE) and MEF2 binding sites were among top acetylation-enriched motifs after weekly prednisone, while the FOXO3 binding motif was among the top enriched motifs after daily prednisone. N=3 mice/group for K27ac ChIP-seq, n=5 mice/group for RNAseq; q-value, Benjamini-Hochberg test. For additional data, see also FIG. 6.

FIG. 3 shows that KLF15 and MEF2C mediate a genomewide program to support BCAA utilization, glucose metabolism, and NAD biogenesis in dystrophic muscle. (A) Pathway analysis showed that pulsatile prednisone increased transcription of genes regulating BCAA, glucose and NAD synthesis. H3K27ac ChIP-seq showed GRE enrichment after both weekly and daily steroids, but increased enrichment of KRE and MEF2 sites only after weekly prednisone. (B) Molecular model of the pro-ergogenic transcriptional program driven by pulsatile glucocorticoids. (C) Luciferase reporter plasmids were electroporated into mdx muscle and native or mutant regulatory regions from Mef2c, Bckdha, Pck1 and Nmnat3 were evaluated. Prednisone pulse and Klf15 overexpression had additive effects in increasing GRE-KRE activation ex vivo. (D) Prednisone pulse, KIf15 and Mef2C overexpression had additive effects on MEF2 sites from Bckdha, Nmnat3, Pck1 loci. Changes were blunted after specific deletion of target sites (A). N=4 mice/group (C). Histograms, single values and mean±s.e.m.; *, P<0.05 vs vehicle; $, P<0.05 vs single-factor treatment; 1-way ANOVA test with Tukey's multiple comparison. For additional data, see also FIG. 6.

FIG. 4 shows that pulsatile glucocorticoids reduce BCAA accumulation and improve insulin sensitivity in dystrophic mice and humans with DMD. (A) Long-term pulsatile prednisone improved morbidity of mdx mice. Metabolic cage analysis showed increased VO₂ and energy expenditure during the nocturnal activity phase. Treatment increased force (tibialis) and muscle mass (gastrocnemius), and reduced circulating levels of BCAA, free fatty acids and ketones, indicating higher nutrient disposal. (B) Serum biomarkers comparing DMD patients receiving either daily GC steroids or weekend GC steroids. Weekend glucocorticoids in DMD patients correlated with reduced obesity and decreased levels of circulating BCAAs and insulin resistance. (C) Long-term weekly prednisone treatment of Dysf-null mice, a model of limb girdle muscular dystrophy. Weekly prednisone improved BCAA utilization and increased ATP, NAD+ and glycogen content in striated muscles. Curves, mean±s.e.m.; histograms depict single values and mean±s.e.m.; box plots, Tukey distribution; n=10 mice/group (A,C); n=12 patients/group (C). *, Welch's unpaired t-test (two-tailed) (C-G). For additional data, see also FIGS. 7-8 and Tables 2-4.

FIG. 5 shows that pulsatile steroid treatment improves energy production and function in dystrophic mdx mice. (A-C) Weekly prednisone increased ATP and NAD⁺ levels in quadriceps muscle of mdx mice, as shown by HPLC measurements. Weekly prednisone also increased blood lactate and glycogen levels. Daily prednisone had opposing effects. (D) Weekly prednisone increased insulin sensitivity, while daily regimen induced insulin resistance. (E) Glycemia progressively increased with daily prednisone but not weekly prednisone. (F) Unlike weekly treatment, daily treatment induced adipocyte hypertrophy. (G) Endpoint tolerance tests showed that daily prednisone induced glucose intolerance and pyruvate intolerance. Conversely, weekly prednisone did not increase glucose intolerance and had modest effects on pyruvate intolerance. (H) Steroid regimens comparably increased liver gluconeogenesis, as assessed though glycogen levels. (I) Weekly prednisone increased glucose uptake in muscle, as shown by 2-NBDG uptake in live dystrophic myofibers. (J-L) Multi-modal imaging in live animals showed that weekly prednisone reduced glucose uptake in fat tissue, did not increase fat mass and did not induce osteoporosis. Curves depict mean±s.e.m.; histograms depict single values and mean±s.e.m.; box plots, Tukey distribution; n=6 mice/group (A-4); n=3 mice/group (J-L); *, P<0.05 vs vehicle, 1-way ANOVA test with Tukey's multiple comparison (A-4), 2-way ANOVA test with Tukey's multiple comparison (J-L); #, P<0.05 vs vehicle, 2-way ANOVA test with Tukey's multiple comparison.

FIG. 6 shows gene expression and acetylation profiles elicited by weekly or daily prednisone in dystrophic mouse muscle. (A) After daily prednisone, KIf15 and Mef2C showed reduced expression and K27 acetylation in treated mdx myofibers. (B) FOXO3 sites of upregulated wasting agonists were enriched in K27ac mark after daily prednisone, but not weekly prednisone. (C) Pathway-centered analysis showed that weekly prednisone increased transcription/acetylation levels of genes involved in fatty acid and ketone metabolism, whereas atrophy agonists were activated after daily prednisone. N=3 mice/group for K27ac ChIP-seq, n=5 mice/group for RNAseq.

FIG. 7 shows that weekly and daily prednisone have opposing effects on insulin resistance in treated mdx mice. (A) At endpoint, treatment increased levels of ATP, NAD and glycogen in muscle. (B) Weekly prednisone maintained glycemia unchanged while increasing blood lactate levels at endpoint. (C) Long-term weekly prednisone improved striated muscle function, as shown by grip strength, whole-body plethysmography and echocardiography. Curves, mean±s.e.m.; box plots, histograms depict single values and mean±s.e.m.; *, P<0.05 vs vehicle, Welch's unpaired t-test (two-tailed); #, P<0.05 vs vehicle, 2-way ANOVA test.

FIG. 8 shows that metabolic reprogramming improves muscle performance in Dysf-null mice, a model of limb girdle muscular dystrophy. Dysf-null mice (n=10/cohort) were treated for 32 weeks with either prednisone (i.p. 1 mg/kg once weekly), or vehicle from the age of approximately 9 months. (A) Weekly prednisone did not induce significant changes in body weight trend in treated Dysf-null mice. (B) CSA of myofibers, but not adipocytes, was increased after treatment. (C) Grip strength and endpoint tibialis anterior tetanic and specific forces were increased after weekly prednisone. (D-E) Respiratory muscle and systolic functions were enhanced by treatment. Curves depict mean±s.e.m.; histograms depict single values and mean±s.e.m.; n=10 mice/cohort; *, P<0.05 vs vehicle; Welch's unpaired t-test (two-tailed); #, P<0.05 vs vehicle, 2-way ANOVA test.

FIG. 9 shows that pulsatile (weekly) glucocorticoid exposure curbed metabolic dysfunction in mice under diet-induced obesity. Wildtype (WT) mice were fed high-fat chow and treated with either vehicle or weekly (pulsatile) 1 mg/kg intraperitoneal prednisone administration for 8 weeks. (A) As compared to vehicle treatment, weekly prednisone slightly but significantly reduced gain of body weight and fat mass, while improved lean mass retention. (B) Weekly prednisone reduced the gain of hyperglycemia, as shown by fasting blood glucose levels over time. At diet exposure endpoint, obese mice treated with weekly prednisone showed improved body-wide glucose homeostasis, as shown by glucose and insulin tolerance tests. (C) Weekly prednisone improved grip strength (forelimbs, bilateral), tetanic force production (tibialis anterior, in situ) and aerobic exercise capacity (run-to-exhaustion, treadmill) at the end of high-fat diet regimen. Curves depict mean±s.e.m.; histograms depict single values and mean±s.e.m.; n=5 mice/group. *, P<0.05 vs vehicle, Welch's unpaired t-test (two-tailed); #, P<0.05 vs vehicle, 2-way ANOVA test.

FIG. 10 shows that pulsatile (weekly) glucocorticoid treatment improved energy production and muscle function in aging mice. Wildtype (WT) mice were treated with either vehicle or weekly (pulsatile) 1 mg/kg intraperitoneal prednisone administration for 40 weeks from the age of 6 weeks. (A) As compared to vehicle treatment, weekly prednisone increased levels of ATP, NAD+ and glycogen in muscle and heart tissues. (B) In aged mice, weekly prednisone improved grip strength, tetanic and specific force, and muscle mass, seen as myofiber cross-sectional area (CSA). (C) Weekly prednisone improved parameters of respiratory function over time, as measured by whole-body plethysmography. (D) Weekly prednisone improved parameters of cardiac contractile function overtime, as measured by echocardiography. Curves depict mean±s.e.m.; histograms depict single values and mean±s.e.m.; n=10 mice/group. *, P<0.05 vs vehicle, Welch's unpaired t-test (two-tailed); #, P<0.05 vs vehicle, 2-way ANOVA test.

FIG. 11 shows that pulsatile glucocorticoid treatment increased circulating adiponectin levels in mice and humans, including dystrophic mdx mice (A), in dystrophic DMD patients (B), in mice under diet-induced obesity (C), and in aging mice (D). Dosing was weekly 1 mg/kg in mice, while weekend (two consecutive days per week) 1-4 mg/kg in humans. Histograms depict single values and mean±s.e.m.; (A) n=6 mice/group; (B) n=12 patients/group; (C) n=5 mice/group; (D) n=10 mice/group. *, P<0.05 vs vehicle, Welch's unpaired t-test (two-tailed).

FIG. 12 shows that pulsatile (weekly) glucocorticoid exposure curbed metabolic dysfunction in wildtype mice with high fat diet-induced obesity. Wildtype (WT) mice were fed high-fat chow and treated with either vehicle or once weekly (pulsatile) 1 mg/kg intraperitoneal prednisone administration for 12 weeks. (A-B) As compared to vehicle treatment, weekly prednisone reduced gain of body weight, while improving retention of lean mass, myofiber mass and specific force (measured in tibialis anterior). (C) As compared to vehicle treatment, weekly prednisone reduced accrual of whole-body fat mass and adipocyte mass in the ventral fat pad. (D) These changes correlated with significant improvement of endpoint grip strength and running endurance in mice treated with weekly prednisone as compared to mice treated with vehicle. (E) Weekly prednisone reduced the gain of hyperglycemia, as shown by fasting blood glucose levels over time. (F) At diet exposure endpoint, obese mice treated with weekly prednisone showed improved body-wide glucose homeostasis, as shown by glucose and insulin tolerance tests (left and center panels), and these findings are relevant to the use of intermittent steroids to treat diabetes mellitus. This once weekly glucocorticoid exposure also improved ex vivo uptake of the fluorescent analog 2-NBDG in freshly isolated myofibers, in both absence and presence of insulin (left panel). (G) At the end of treatment, quadriceps muscles were isolated and exposed ex vivo to either 10 mM glucose or 1 mM palmitate-BSA in the presence of 1×/min electrical stimulation. Muscles from glucocorticoid treated mice showed higher levels of ATP and phosphocreatine production as compared to vehicle-treated control muscles after both normal and high-fat diet regimens. (H) Metabolic cage assays showed that weekly prednisone increased oxidative capacity and energy expenditure in the active phase as compared to vehicle treatment, in mice fed with either normal or high-fat diet chow. Curves depict interquartile range and single values; histograms depict single values and mean±s.e.m.; n=10 mice/group in A-E; n=3 mice/group in F-H. *, P<0.05 vs same-diet vehicle control, 1-way ANOVA with Tukey's multi-comparison; #, P<0.05 vs vehicle, 2-way ANOVA test.

### DETAILED DESCRIPTION

Once-daily versus once-weekly (pulsatile) dosing of GC steroids was compared in dystrophic muscle repair (Quattrocelli et al., 2017a; Quattrocelli et al., 2017b, Quattrocelli et al., JCI Insight. 2019 Dec. 19; 4(24). pii: 132402. doi: 10.1172/jci.insight.132402). It was found that pulsatile and daily steroids both improved muscle repair. However, it was unexpectedly found that pulsatile dosing enhanced muscle performance, while daily dosing elicited muscle wasting. Moreover, in normal mice, once weekly steroids promoted lean mass in high fat diet fed animals. This was also unexpected because chronic daily glucocorticoids are associated with increased obesity and diabetes (Fardet and Feve, Drugs 2014), and once weekly glucocorticoids elicited the opposite effect.

As used in this specification and the enumerated paragraphs herein, the singular forms “a,” “an,” and “the” include plural reference unless the context clearly dictates otherwise.

As used herein, an agent that “increases the activity of an annexin protein” is one that increases a property of an annexin protein as a calcium-binding membrane associated repair protein that enhances restoration of membrane integrity. Increasing the activity of the annexin protein means that administration of the agent results in an overall increase in the activity (i.e., the increase in activity derived from administration of the agent plus any endogenous activity) of one or more annexin proteins as disclosed herein.

As used herein, the term “treating” or “treatment” refers to an intervention performed with the intention of preventing the further development of or altering the pathology of a disease or infection. Accordingly, “treatment” refers to both therapeutic treatment and prophylactic or preventative measures. “Preventing” refers to a preventative measure taken with a subject not having a condition or disease.

As used herein, an “effective amount” of a compound described herein refers to an amount sufficient to elicit the desired biological response, e.g., treating the condition. As will be appreciated by those of ordinary skill in this art, the effective amount of a compound described herein may vary depending on such factors as the desired biological endpoint, the pharmacokinetics of the compound, the condition being treated, the mode of administration, and the age and health of the subject. An effective amount encompasses therapeutic and prophylactic treatment.

### Methods of Administering a Glucocorticoid Steroid

In some aspects, the present disclosure provides methods for administering a glucocorticoid steroid to a patient, wherein the patient has a serum or plasma level of one or more of the following biomarkers that is:
(a) less than about 18 μg/dL morning fasting cortisol;
(b) at least about 90 mg/dL fasting morning glucose;
(c) at least about 160 pmol/L insulin;
(d) at least about 40 μmol/L isoleucine;
(e) at least about 100 μmol/L leucine;
(f) at least about 120 μmol/L valine;
(g) at least about 700 μmol/L combined branched chain amino acids;
(h) at least about 110 mg/dL triglycerides;
(i) at least about 300 μmol/L non-esterified fatty acids; and/or
(j) at least about 100 μmol/L combined ketones;

wherein the administering of the glucocorticoid steroid comprises once-weekly administration of the glucocorticoid steroid. In some embodiments, the once-weekly dosing comprises administering about 1 mg/kg of the glucocorticoid steroid for patients having a body weight that is up to about 70 kg. In further embodiments, the once-weekly dosing comprises administering about 0.75 mg/kg of the glucocorticoid steroid for patients having a body weight that is greater than about 70 kg. In further aspects, the disclosure also provides methods for administering a glucocorticoid steroid to a patient, wherein the patient has a serum or plasma level of one or more of the following biomarkers that is:
(a) less than about 18 μg/dL morning fasting cortisol;
(b) at least about 90 mg/dL fasting morning glucose;
(c) at least about 160 pmol/L insulin;
(d) at least about 40 μmol/L isoleucine;
(e) at least about 100 μmol/L leucine;
(f) at least about 120 μmol/L valine;
(g) at least about 700 μmol/L combined branched chain amino acids;
(h) at least about 110 mg/dL triglycerides;
(i) at least about 300 μmol/L non-esterified fatty acids; and/or
(j) at least about 100 μmol/L combined ketones;

wherein the administering of the glucocorticoid steroid comprises administration of the glucocorticoid steroid more than once per week. In some embodiments, the glucocorticoid steroid is administered once every 2-3 days, or once every 4-5 days, or once every 5-6 days. Thus, in various embodiments, administration of the glucocorticoid steroid requires one or more doses daily or weekly. Regardless of the frequency of glucocorticoid steroid administration, it is contemplated that in various embodiments each dose that is administered is from about 0.75 mg/kg to about 1 mg/kg. Patients having levels of one or more of the foregoing biomarkers according to the above levels are identified as those who would benefit from once weekly (or once every 2-3 days, or once every 4-5 days, or once every 5-6 days) administration of the glucocorticoid steroid. In some embodiments, the disclosure provides improved methods for administering a glucocorticoid steroid to a patient, wherein the patient has a serum or plasma level of one or more of the following biomarkers that is: (a) less than about 18 μg/dL morning fasting cortisol; (b) at least about 90 mg/dL fasting morning glucose; (c) at least about 160 pmol/L insulin; (d) at least about 40 μmol/L isoleucine; (e) at least about 100 μmol/L leucine; (f) at least about 120 μmol/L valine; (g) at least about 700 μmol/L combined branched chain amino acids; (h) at least about 110 mg/dL triglycerides; (i) at least about 300 μmol/L non-esterified fatty acids; and/or (j) at least about 100 μmol/L combined ketones, comprising adjusting the frequency of administration of the glucocorticoid steroid to the patient from daily administration to administration that is once-weekly, once every 2-3 days, once every 4-5 days, or once every 5-6 days. In various embodiments, the improved method of administration results in a decrease in frequency or a reduction in severity of adverse events (e.g., muscle atrophy, obesity, diabetes) that can occur with daily administration of the glucocorticoid steroid. Serum or plasma levels of the biomarkers listed above are measured via tests known in the art and described herein. These tests include, but are not limited to, standard clinical assays for molecule quantitation in blood, serum or plasma samples, such as enzymatic dosing (colorimetry), enzyme-linked immunosorbent assay (ELISA), radioimmunoassay (RIA), blood monitoring devices (glucometer).

Patients in medical need of treatment or prevention of muscle wasting, and/or treatment or prevention of unhealthy aging, and/or treatment or prevention of metabolic disorders, and/or treatment or prevention of sarcopenia, and/or treatment or prevention of obesity, and/or enhancement of nutrient metabolism, and/or enhancement of energy production, and/or enhancement of energy expenditure, and/or enhancement of exercise tolerance, and/or enhancement of insulin sensitivity, and/or enhancement of adiponectin production, and/or reduced osteoporosis, and/or reduced muscle wasting, and/or reduced insulin resistance, and/or reduced fat accrual, and who have levels of one or more of the foregoing biomarkers according to the above levels are identified as those who would benefit from once weekly administration of the glucocorticoid steroid. In addition, in those conditions where daily administration of the glucocorticoid steroid would induce any of the above conditions, once weekly administration of the glucocorticoid steroid would be used to avoid metabolic derangement. A patient “in medical need of treatment or prevention” is one who has been diagnosed by a physician as being in need of treatment or prevention.

### Annexin Proteins

In some embodiments, methods of administering a glucocorticoid steroid according to the disclosure further comprises administering an effective amount of an agent that increases the activity of an annexin protein.

The annexin protein family is characterized by the ability to bind phospholipids and actin in a Ca²⁺-dependent manner. Annexins preferentially bind phosphatidylserine, phosphatidylinositols, and cholesterol (Gerke et al., 2005). In humans, dominant or recessive mutations in annexin genes have not been associated with muscle disease. However, annexin A5 genetic variants are associated with pregnancy loss (de Laat et al., 2006). The annexin family is known to comprise over 160 distinct proteins that are present in more than 65 unique species (Gerke and Moss, 2002). Humans have 12 different annexin genes, characterized by distinct tissue expression and localization. Annexins are involved in a variety of cellular processes including membrane permeability, mobility, vesicle fusion, and membrane bending. These properties are Ca²⁺-dependent. Although annexins do not contain EF hand domains, calcium ions bind to the individual annexin repeat domains. Differential Ca²⁺ affinity allows each annexin protein to respond to changes in intracellular calcium levels under unique spatiotemporal conditions (Blackwood and Ernst, 1990).

Structurally, the annexin family of proteins contains a conserved carboxy-terminal core domain composed of multiple annexin repeats and a variable amino-terminal head. The amino-terminus differs in length and amino acid sequence amongst the annexin family members.

Additionally, post-translational modifications alter protein function and protein localization (Goulet et al., 1992; Kaetzel et al., 2001). Annexin proteins have the potential to self-oligomerize and interact with membrane surfaces and actin in the presence of Ca²⁺ (Zaks and Creutz, 1991, Hayes et al., Traffic. 5:571-576 (2004), Boye et al., Sci Rep. 8: 10309 (2018)). The amino-terminal region is thought to bind actin or one lipid membrane in a Ca²⁺-dependent manner, while the annexin core region binds an additional lipid membrane.

Annexins do not contain a predicted hydrophobic signal sequence targeting the annexins for classical secretion through the endoplasmic reticulum, yet annexins are found both on the interior and exterior of the cell (Christmas et al., 1991; Deora et al., 2004; Wallner et al., 1986). The process by which the annexins are externalized remains unknown. It is hypothesized that annexins may be released through exocytosis or cell lysis, although the method of externalization may vary by cell type. Functionally, localization both inside and outside the cell adds to the complexity of the roles annexins play within tissues and cell types. Annexin A5 is used commonly as a marker for apoptosis due to its high affinity to phosphatidylserine (PS). During cell death and injury, PS reverses membrane orientation from the inner to outer membrane, providing access for annexin binding from the cell exterior. Annexins have been shown to have anti-inflammatory, pro-fibrinolytic, and anti-thrombotic effects. The annexin A1-deleted mouse model exhibits an exacerbated inflammatory response when challenged and is resistant to the anti-inflammatory effects of glucocorticoids (Hannon et al., 2003). The annexin A2 null-mouse develops fibrin accumulation in the microvasculature and is defective in clearance of arterial thrombi (Ling et al., 2004). Although little is known about the precise function of extracellular annexins, the expression level of annexin proteins may function as a diagnostic marker for a number of diseases due to the strong correlation between high expression levels of annexins and the clinical severity of disease (Cagliani et al., 2005).

In some aspects, the disclosure contemplates methods of administering a glucocorticoid steroid to a patient, wherein the patient has a certain serum or plasma level of one or more biomarkers as disclosed herein, and in some embodiments the methods further comprise administering an effective amount of: (i) an agent that increases the activity of an annexin protein, (ii) mitsugumin 53 (MG53), (iii) a modulator of latent TGF-β binding protein 4 (LTBP4), (iv) a modulator of transforming growth factor β (TGF-β) activity, (v) a modulator of androgen response, (vi) a modulator of an inflammatory response, (vii) a promoter of muscle growth, (viii) a chemotherapeutic agent, (ix) a modulator of fibrosis, (x) a modulator of glucose homeostasis, (xi) a modulator of metabolic function, or a combination thereof.

### Proteins/Recombinant Proteins

Methods of the disclosure include those in which a recombinant protein is administered to a patient in need thereof in a therapeutically effective amount. As used herein a “protein” refers to a polymer comprised of amino acid residues. “Annexin protein” as used herein includes without limitation a wild type annexin protein, an annexin-like protein, or a fragment, analog, variant, fusion or mimetic, each as described herein. An “annexin peptide” is a shorter version (e.g., about 50 amino acids or less) of a wild type annexin protein, an annexin-like protein, or a fragment, analog, variant, fusion or mimetic that is sufficient to increase the overall activity of the annexin protein to which the annexin peptide is related.

Proteins of the present disclosure may be either naturally occurring or non-naturally occurring. Naturally occurring proteins include without limitation biologically active proteins that exist in nature or can be produced in a form that is found in nature by, for example, chemical synthesis or recombinant expression techniques. Naturally occurring proteins also include post-translationally modified proteins, such as, for example and without limitation, glycosylated proteins. Non-naturally occurring proteins contemplated by the present disclosure include but are not limited to synthetic proteins, as well as fragments, analogs and variants of naturally occurring or non-naturally occurring proteins as defined herein. Non-naturally occurring proteins also include proteins or protein substances that have D-amino acids, modified, derivatized, or non-naturally occurring amino acids in the D- or L-configuration and/or peptidomimetic units as part of their structure. The term “protein” typically refers to large polypeptides. The term “peptide” generally refers to short (e.g., about 50 amino acids or less) polypeptides.

Non-naturally occurring proteins are prepared, for example, using an automated protein synthesizer or, alternatively, using recombinant expression techniques using a modified oligonucleotide which encodes the desired protein.

As used herein a “fragment” of a protein is meant to refer to any portion of a protein smaller than the full-length protein expression product.

As used herein an “analog” refers to any of two or more proteins substantially similar in structure and having the same biological activity, but can have varying degrees of activity, to either the entire molecule, or to a fragment thereof. Analogs differ in the composition of their amino acid sequences based on one or more mutations involving substitution, deletion, insertion and/or addition of one or more amino acids for other amino acids. Substitutions can be conservative or non-conservative based on the physico-chemical or functional relatedness of the amino acid that is being replaced and the amino acid replacing it.

As used herein a “variant” refers to a protein or analog thereof that is modified to comprise additional chemical moieties not normally a part of the molecule. Such moieties may modulate, for example and without limitation, the molecule's solubility, absorption, and/or biological half-life. Moieties capable of mediating such effects are disclosed in Remington's Pharmaceutical Sciences (1980). Procedures for coupling such moieties to a molecule are well known in the art. In various aspects, polypeptides are modified by biotinylation, glycosylation, PEGylation, and/or polysialylation.

Fusion proteins, including fusion proteins wherein one fusion component is a fragment or a mimetic, are also contemplated. A “mimetic” as used herein means a peptide or protein having a biological activity that is comparable to the protein of which it is a mimetic.

In any of the aspects or embodiments of the disclosure, the recombinant protein is a recombinant wild type annexin protein, an annexin-like protein, or a fragment of a wild type annexin protein or annexin-like protein that exhibits one or more biological activities of an annexin protein. By “annexin-like protein” is meant a protein having sufficient amino acid sequence identity to a referent wild type annexin protein to exhibit the activity of an annexin protein, for example and without limitation, activity as a calcium-binding membrane associated repair protein that enhances restoration of membrane integrity through facilitating the formation of a macromolecular repair complex at the membrane lesion including proteins such as annexin A1 (SEQ ID NO: 1), annexin A2 (SEQ ID NO: 2 or SEQ ID NO: 3), EHD2, dysferlin, and MG53. In some embodiments, the annexin-like protein is a protein having about or at least about 75% amino acid sequence identity with a referent wild type human annexin protein (e.g., annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 44, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), or annexin A13 (SEQ ID NO: 17 or SEQ ID NO: 18)). In further embodiments, the annexin-like protein is a protein having about or at least about 80%, about or at least about 85%, about or at least about 90%, about or at least about 95%, or about 99% amino acid sequence identity with a wild type human annexin protein.

In some embodiments, an agent of the disclosure is an annexin protein that comprises a post-translational modification. In various embodiments, the post-translational modification increases production of an annexin or annexin-like protein, increases solubility of an annexin or annexin-like protein, decreases aggregation of an annexin or annexin-like protein, increases the half-life of an annexin or annexin-like protein, increases the stability of an annexin or annexin-like protein, enhances target membrane engagement of an annexin or annexin-like protein, or is a codon-optimized version of an annexin or annexin-like protein.

The disclosure also contemplates, in various embodiments, compositions that increase the activity of annexin A1 (SEQ ID NO: 1), annexin A2 (SEQ ID NO: 2 and/or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 44, or a combination thereof), annexin A7 (SEQ ID NO: 9 and/or SEQ ID NO: 10), annexin A8 (SEQ ID NO: 11 and/or SEQ ID NO: 12), annexin A9 (SEQ ID NO: 13), annexin A10 (SEQ ID NO: 14), annexin A11 (SEQ ID NO: 15 and/or SEQ ID NO: 16), and annexin A13 (SEQ ID NO: 17 and/or SEQ ID NO: 18) in any combination. Note that when more than one sequence identifier is used to identify an annexin protein herein (e.g., annexin A2 is identified herein by SEQ ID NO: 2 and/or SEQ ID NO: 3) it will be understood that the different sequence identifiers serve to identify isoforms of the particular annexin protein, and that the isoforms may be used interchangeably or in combination in methods and compositions of the disclosure.

Refseq Accession Number NP_000691.1
annexin A1 [Homo sapien]
(SEQ ID NO: 1):
MAMVSEFLKQAWFIENEEQEYVQTVKSSKGGPGSA
VSPYPTFNPSSDVAALHKAIMVKGVDEATIIDILT
KRNNAQRQQIKAAYLQETGKPLDETLKKALTGHLE
EVVLALLKTPAQFDADELRAAMKGLGTDEDTLIEI
LASRTNKEIRDINRVYREELKRDLAKDITSDTSGD
FRNALLSLAKGDRSEDFGVNEDLADSDARALYEAG
ERRKGTDVNVFNTILTTRSYPQLRRVFQKYTKYSK
HDMNKVLDLELKGDIEKCLTAIVKCATSKPAFFAE
KLHQAMKGVGTRHKALIRIMVSRSEIDMNDIKAFY
QKMYGISLCQAILDETKGDYEKILVALCGGN
Refseq Accession Number NP_001002858.1
annexin A2 isoform 1 [Homo sapien]
(SEQ ID NO: 2):
MGRQLAGCGDAGKKASFKMSTVHEILCKLSLEGDH
STPPSAYGSVKAYTNFDAERDALNIETAIKTKGVD
EVTIVNILTNRSNAQRQDIAFAYQRRTKKELASAL
KSALSGHLETVILGLLKTPAQYDASELKASMKGLG
TDEDSLIEIICSRTNQELQEINRVYKEMYKTDLEK
DIISDTSGDFRKLMVALAKGRRAEDGSVIDYELID
QDARDLYDAGVKRKGTDVPKWISIMTERSVPHLQK
VFDRYKSYSPYDMLESIRKEVKGDLENAFLNLVQC
IQNKPLYFADRLYDSMKGKGTRDKVLIRIMVSRSE
VDMLKIRSEFKRKYGKSLYYYIQQDTKGDYQKALL
YLCGGDD
Refseq Accession Number NP_001129487.1
annexin A2 isoform 2 [Homo sapien]
(SEQ ID NO: 3):
MSTVHEILCKLSLEGDHSTPPSAYGSVKAYTNFDA
ERDALNIETAIKTKGVDEVTIVNILTNRSNAQRQD
IAFAYQRRTKKELASALKSALSGHLETVILGLLKT
PAQYDASELKASMKGLGTDEDSLIEIICSRTNQEL
QEINRVYKEMYKTDLEKDIISDTSGDFRKLMVALA
KGRRAEDGSVIDYELIDQDARDLYDAGVKRKGTDV
PKWISIMTERSVPHLQKVFDRYKSYSPYDMLESIR
KEVKGDLENAFLNLVQCIQNKPLYFADRLYDSMKG
KGTRDKVLIRIMVSRSEVDMLKIRSEFKRKYGKSL
YYYIQQDTKGDYQKALLYLCGGDD
Refseq Accession Number NP_005130.1
annexin A3 [Homo sapien]
(SEQ ID NO: 4):
MASIWVGHRGTVRDYPDFSPSVDAEAIQKAIRGIGT
DEKMLISILTERSNAQRQLIVKEYQAAYGKELKDD
LKGDLSGHFEHLMVALVTPPAVFDAKQLKKSMKGA
GTNEDALIEILTTRTSRQMKDISQAYYTVYKKSLG
DDISSETSGDFRKALLTLADGRRDESLKVDEHLAK
QDAQILYKAGENRWGTDEDKFTEILCLRSFPQLKL
TFDEYRNISQKDIVDSIKGELSGHFEDLLLAIVNC
VRNTPAFLAERLHRALKGIGTDEFTLNRIMVSRSE
IDLLDIRTEFKKHYGYSLYSAIKSDTSGDYEITLL
KICGGDD
Refseq Accession Number NP_001144.1
annexin A4 isoform a [Homo sapien]
(SEQ ID NO: 5):
MAMATKGGTVKAASGFNAMEDAQTLRKAMKGLGTD
EDAIISVLAYRNTAQRQEIRTAYKSTIGRDLIDDL
KSELSGNFEQVIVGMMTPTVLYDVQELRRAMKGAG
TDEGCLIEILASRTPEEIRRISQTYQQQYGRSLED
DIRSDTSFMFQRVLVSLSAGGRDEGNYLDDALVRQ
DAQDLYEAGEKKWGTDEVKFLTVLCSRNRNHLLHV
FDEYKRISQKDIEQSIKSETSGSFEDALLAIVKCM
RNKSAYFAEKLYKSMKGLGTDDNTLIRVMVSRAEI
DMLDIRAHFKRLYGKSLYSFIKGDTSGDYRKVLLV
LCGGDD
Refseq Accession Number NP_001145.1
annexin A5 [Homo sapien]
(SEQ ID NO: 6):
MAQVLRGTVTDFPGFDERADAETLRKAMKGLGTDE
ESILTLLTSRSNAQRQEISAAFKTLFGRDLLDDLK
SELTGKFEKLIVALMKPSRLYDAYELKHALKGAGT
NEKVLTEIIASRTPEELRAIKQVYEEEYGSSLEDD
VVGDTSGYYQRMLVVLLQANRDPDAGIDEAQVEQD
AQALFQAGELKWGTDEEKFITIFGTRSVSHLRKVF
DKYMTISGFQIEETIDRETSGNLEQLLLAVVKSIR
SIPAYLAETLYYAMKGAGTDDHTLIRVMVSRSEID
LFNIRKEFRKNFATSLYSMIKGDTSGDYKKALLLL
CGEDD
Refseq Accession Number NP_001146.2
annexin A6 isoform 1 [Homo sapien]
(SEQ ID NO: 7):
MAKPAQGAKYRGSIHDFPGFDPNQDAEALYTAMKGF
GSDKEAILDIITSRSNRQRQEVCQSYKSLYGKDLI
ADLKYELTGKFERLIVGLMRPPAYCDAKEIKDAIS
GIGTDEKCLIEILASRTNEQMHQLVAAYKDAYERD
LEADIIGDTSGHFQKMLVVLLQGTREEDDVVSEDL
VQQDVQDLYEAGELKWGTDEAQFIYILGNRSKQHL
RLVFDEYLKTTGKPIEASIRGELSGDFEKLMLAVV
KCIRSTPEYFAERLFKAMKGLGTRDNTLIRIMVSR
SELDMLDIREIFRTKYEKSLYSMIKNDTSGEYKKT
LLKLSGGDDDAAGQFFPEAAQVAYQMWELSAVARV
ELKGTVRPANDFNPDADAKALRKAMKGLGTDEDTI
IDIITHRSNVQRQQIRQTFKSHFGRDLMTDLKSEI
SGDLARLILGLMMPPAHYDAKQLKKAMEGAGTDEK
ALIEILATRTNAEIRAINEAYKEDYHKSLEDALSS
DTSGHFRRILISLATGHREEGGENLDQAREDAQVA
AEILEIADTPSGDKTSLETRFMTILCTRSYPHLRR
VFQEFIKMTNYDVEHTIKKEMSGDVRDAFVAIVQS
VKNKPLFFADKLYKSMKGAGTDEKTLTRIMVSRSE
IDLLNIRREFIEKYDKSLHQAIEGDTSGDFLKALL
ALCGGED
Refseq Accession Number NP_001180473.1
annexin A6 isoform 2 [Homo sapien]
(SEQ ID NO: 8):
MKGFGSDKEAILDIITSRSNRQRQEVCQSYKSLYG
KDLIADLKYELTGKFERLIVGLMRPPAYCDAKEIK
DAISGIGTDEKCLIEILASRTNEQMHQLVAAYKDA
YERDLEADIIGDTSGHFQKMLVVLLQGTREEDDVV
SEDLVQQDVQDLYEAGELKWGTDEAQFIYILGNRS
KQHLRLVFDEYLKTTGKPIEASIRGELSGDFEKLM
LAVVKCIRSTPEYFAERLFKAMKGLGTRDNTLIRI
MVSRSELDMLDIREIFRTKYEKSLYSMIKNDTSGE
YKKTLLKLSGGDDDAAGQFFPEAAQVAYQMWELSA
VARVELKGTVRPANDFNPDADAKALRKAMKGLGTD
EDTIIDIITHRSNVQRQQIRQTFKSHFGRDLMTDL
KSEISGDLARLILGLMMPPAHYDAKQLKKAMEGAG
TDEKALIEILATRTNAEIRAINEAYKEDYHKSLED
ALSSDTSGHFRRILISLATGHREEGGENLDQARED
AQVAAEILEIADTPSGDKTSLETRFMTILCTRSYP
HLRRVFQEFIKMTNYDVEHTIKKEMSGDVRDAFVA
IVQSVKNKPLFFADKLYKSMKGAGTDEKTLTRIMV
SRSEIDLLNIRREFIEKYDKSLHQAIEGDTSGDFL
KALLALCGGED
Refseq Accession Number NP_001147.1
annexin A7 isoform 1 [Homo sapien]
(SEQ ID NO: 9):
MSYPGYPPTGYPPFPGYPPAGQESSFPPSGQYPYP
SGFPPMGGGAYPQVPSSGYPGAGGYPAPGGYPAPG
GYPGAPQPGGAPSYPGVPPGQGFGVPPGGAGFSGY
PQPPSQSYGGGPAQVPLPGGFPGGQMPSQYPGGQP
TYPSQPATVTQVTQGTIRPAANFDAIRDAEILRKA
MKGFGTDEQAIVDVVANRSNDQRQKIKAAFKTSYG
KDLIKDLKSELSGNMEELILALFMPPTYYDAWSLR
KAMQGAGTQERVLIEILCTRTNQEIREIVRCYQSE
FGRDLEKDIRSDTSGHFERLLVSMCQGNRDENQSI
NHQMAQEDAQRLYQAGEGRLGTDESCFNMILATRS
FPQLRATMEAYSRMANRDLLSSVSREFSGYVESGL
KTILQCALNRPAFFAERLYYAMKGAGTDDSTLVRI
VVTRSEIDLVQIKQMFAQMYQKTLGTMIAGDTSGD
YRRLLLAIVGQ
Refseq Accession Number NP_004025.1
annexin A7 isoform 2 [Homo sapien]
(SEQ ID NO: 10):
MSYPGYPPTGYPPFPGYPPAGQESSFPPSGQYPYP
SGFPPMGGGAYPQVPSSGYPGAGGYPAPGGYPAPG
GYPGAPQPGGAPSYPGVPPGQGFGVPPGGAGFSGY
PQPPSQSYGGGPAQVPLPGGFPGGQMPSQYPGGQP
TYPSQINTDSFSSYPVFSPVSLDYSSEPATVTQVT
QGTIRPAANFDAIRDAEILRKAMKGFGTDEQAIVD
VVANRSNDQRQKIKAAFKTSYGKDLIKDLKSELSG
NMEELILALFMPPTYYDAWSLRKAMQGAGTQERVL
IEILCTRTNQEIREIVRCYQSEFGRDLEKDIRSDT
SGHFERLLVSMCQGNRDENQSINHQMAQEDAQRLY
QAGEGRLGTDESCFNMILATRSFPQLRATMEAYSR
MANRDLLSSVSREFSGYVESGLKTILQCALNRPAF
FAERLYYAMKGAGTDDSTLVRIVVIRSEIDLVQIK
QMFAQMYQKTLGTMIAGDTSGDYRRLLLAIVGQ
Refseq Accession Number NP_001258631.1
annexin A8 isoform 1 [Homo sapien]
(SEQ ID NO: 11):
MAWWKSWIEQEGVTVKSSSHFNPDPDAETLYKAMK
GIGVGSQLLSHQAAAFAFPSSALTSVSPWGQQGHL
CCNPAGTNEQAIIDVLIKRSNTQRQQIAKSFKAQF
GKDLTETLKSELSGKFERLIVALMYPPYRYEAKEL
HDAMKGLGTKEGVIIEILASRTKNQLREIMKAYEE
DYGSSLEEDIQADTSGYLERILVCLLQGSRDDVSS
FVDPGLALQDAQDLYAAGEKIRGTDEMKFITILCT
RSATHLLRVFEEYEKIANKSIEDSIKSETHGSLEE
AMLTVVKCTQNLHSYFAERLYYAMKGAGTRDGTLI
RNIVSRSEIDLNLIKCHFKKMYGKTLSSMIMEDTS
GDYKNALLSLVGSDP
Refseq Accession Number NP_001035173.1
annexin A8 isoform 2 [Homo sapien]
(SEQ ID NO: 12):
MAWWKSWIEQEGVTVKSSSHFNPDPDAETLYKAMK
GIGTNEQAIIDVLTKRSNTQRQQIAKSFKAQFGKD
LTETLKSELSGKFERLIVALMYPPYRYEAKELHDA
MKGLGTKEGVIIEILASRTKNQLREIMKAYEEDYG
SSLEEDIQADTSGYLERILVCLLQGSRDDVSSFVD
PGLALQDAQDLYAAGEKIRGTDEMKFITILCTRSA
THLLRVFEEYEKIANKSIEDSIKSETHGSLEEAML
TVVKCTQNLHSYFAERLYYAMKGAGTRDGTLIRNI
VSRSEIDLNLIKCHFKKMYGKTLSSMIMEDTSGDY
KNALLSLVGSDP
Refseq Accession Number NP_003559.2
annexin A9 [Homo sapien]
(SEQ ID NO: 13):
MSVTGGKMAPSLTQEILSHLGLASKTAAWGTLGTL
RTFLNFSVDKDAQRLLRAITGQGVDRSAIVDVLTN
RSREQRQLISRNFQERTQQDLMKSLQAALSGNLER
IVMALLQPTAQFDAQELRTALKASDSAVDVAIEIL
ATRTPPQLQECLAVYKHNFQVEAVDDITSETSGIL
QDLLLALAKGGRDSYSGIIDYNLAEQDVQALQRAE
GPSREETWVPVFTQRNPEHLIRVFDQYQRSTGQEL
EEAVQNRFHGDAQVALLGLASVIKNTPLYFADKLH
QALQETEPNYQVLIRILISRCETDLLSIRAEFRKK
FGKSLYSSLQDAVKGDCQSALLALCRAEDM
Refseq Accession Number NP_009124.2
annexin A10 [Homo sapien]
(SEQ ID NO: 14):
MFCGDYVQGTIFPAPNFNPIMDAQMLGGALQGFDC
DKDMLINILTQRCNAQRMMIAEAYQSMYGRDLIGD
MREQLSDHFKDVMAGLMYPPPLYDAHELWHAMKGV
GTDENCLIEILASRTNGEIFQMREAYCLQYSNNLQ
EDIYSETSGHFRDTLMNLVQGTREEGYTDPAMAAQ
DAMVLWEACQQKTGEHKTMLQMILCNKSYQQLRLV
FQEFQNISGQDMVDAINECYDGYFQELLVAIVLCV
RDKPAYFAYRLYSAIHDFGFHNKTVIRILIARSEI
DLLTIRKRYKERYGKSLFHDIRNFASGHYKKALLA
ICAGDAEDY
Refseq Accession Number NP 665875.1
annexin A11 isoform 1 [Homo sapien]
(SEQ ID NO: 15):
MSYPGYPPPPGGYPPAAPGGGPWGGAAYPPPPSMP
PIGLDNVATYAGQFNQDYLSGMAANMSGTFGGANM
PNLYPGAPGAGYPPVPPGGFGQPPSAQQPVPPYGM
YPPPGGNPPSRMPSYPPYPGAPVPGQPMPPPGQQP
PGAYPGQPPVTYPGQPPVPLPGQQQPVPSYPGYPG
SGTVTPAVPPTQFGSRGTITDAPGFDPLRDAEVLR
KAMKGFGTDEQAIIDCLGSRSNKQRQQILLSFKTA
YGKDLIKDLKSELSGNFEKTILALMKTPVLFDIYE
IKEAIKGVGTDEACLIEILASRSNEHIRELNRAYK
AEFKKTLEEAIRSDTSGHFQRLLISLSQGNRDEST
NVDMSLAQRDAQELYAAGENRLGTDESKFNAVLCS
RSRAHLVAVFNEYQRMTGRDIEKSICREMSGDLEE
GMLAVVKCLKNTPAFFAERLNKAMRGAGTKDRTLI
RIMVSRSETDLLDIRSEYKRMYGKSLYHDISGDTS
GDYRKILLKICGGND
Refseq Accession Number NP_001265338.1
annexin A11 isoform 2 [Homo sapien]
(SEQ ID NO: 16):
MPPIGLDNVATYAGQFNQDYLSGMAANMSGTFGGA
NMPNLYPGAPGAGYPPVPPGGFGQPPSAQQPVPPY
GMYPPPGGNPPSRMPSYPPYPGAPVPGQPMPPPGQ
QPPGAYPGQPPVTYPGQPPVPLPGQQQPVPSYPGY
PGSGTVTPAVPPTQFGSRGTITDAPGFDPLRDAEV
LRKAMKGFGTDEQAIIDCLGSRSNKQRQQILLSFK
TAYGKDLIKDLKSELSGNFEKTILALMKTPVLFDI
YEIKEAIKGVGTDEACLIEILASRSNEHIRELNRA
YKAEFKKTLEEAIRSDTSGHFQRLLISLSQGNRDE
STNVDMSLAQRDAQELYAAGENRLGTDESKFNAVL
CSRSRAHLVAVFNEYQRMTGRDIEKSICREMSGDL
EEGMLAVVKCLKNTPAFFAERLNKAMRGAGTKDRT
LIRIMVSRSETDLLDIRSEYKRMYGKSLYHDISGD
TSGDYRKILLKICGGND
Refseq Accession Number NP_004297.2
annexin A13 isoform a [Homo sapien]
(SEQ ID NO: 17):
MGNRHAKASSPQGFDVDRDAKKLNKACKG
MGTNEAAIIEILSGRTSDERQQIKQKYKAT
YGKELEEVLKSELSGNFEKTALALLDRP
SEYAARQLQKAMKGLGTDESVLIEVLCTRTNKEII
AIKEAYQRLFDRSLESDVKGDTSGNLKKILVSLLQ
ANRNEGDDVDKDLAGQDAKDLYDAGEGRWGTDELA
FNEVLAKRSYKQLRATFQAYQILIGKDIEEAIEEE
TSGDLQKAYLTLVRCAQDCEDYFAERLYKSMKGAG
TDEETLIRIVVTRAEVDLQGIKAKFQEKYQKSLSD
MVRSDTSGDFRKLLVALLH
Refseq Accession Number NP_001003954.1
annexin A13 isoform b [Homo sapien]
(SEQ ID NO: 18):
MGNRHSQSYTLSEGSQQLPKGDSQPSTVVQPLSHP
SRNGEPEAPQPAKASSPQGFDVDRDAKKLNKACKG
MGTNEAAIIEILSGRTSDERQQI
KQKYKATYGKELEEVLKSELSGNFEKTALALLDRP
SEYAARQLQKAMKGLGTDESVLIEVLCTRTNKEII
AIKEAYQRLFDRSLESDVKGDTSGNLKKILVSLLQ
ANRNEGDDVDKDLAGQDAKDLYDAGEGRWGTDELA
FNEVLAKRSYKQLRATFQAYQILIGKDIEEAIEEE
TSGDLQKAYLTLVRCAQDCEDYFAERLYKSMKGAG
TDEETLIRIVVTRAEVDLQGIKAKFQEKYQKSLSD
MVRSDTSGDFRKLLVALLH
Refseq Accession Number NP_001350043.1
annexin A6 isoform 3 [Homo sapien]
(SEQ ID NO: 44):
MAKPAQGAKYRGSIHDFPGFDPNQDAEALYTAMKG
FGSDKEAILDIITSRSNRQRQEVCQSYKSLYGKDL
IADLKYELTGKFERLIVGLMRPPAYCDAKEIKDAI
SGIGTDEKCLIEILASRTNEQMHQLVAAYKDAYER
DLEADIIGDTSGHFQKMLVVLLQGTREEDDVVSED
LVQQDVQDLYEAGELKWGTDEAQFIYILGNRSKQH
LRLVFDEYLKTTGKPIEASIRGELSGDFEKLMLAV
VKCIRSTPEYFAERLFKAMKGLGTRDNTLIRIMVS
RSELDMLDIREIFRTKYEKSLYSMIKNDTSGEYKK
TLLKLSGGDDDAAGQFFPEAAQVAYQMWELSAVAR
VELKGTVRPANDFNPDADAKALRKAMKGLGTDEDT
IIDIITHRSNVQRQQIRQTFKSHFGRDLMTDLKSE
ISGDLARLILGLMMPPAHYDAKQLKKAMEGAGTDE
KALIEILATRTNAEIRAINEAYKEDYHKSLEDALS
SDTSGHFRRILISLATGHREEGGENLDQAREDAQE
IADTPSGDKTSLETRFMTILCTRSYPHLRRVFQEF
IKMTNYDVEHTIKKEMSGDVRDAFVAIVQSVKNKP
LFFADKLYKSMKGAGTDEKTLTRIMVSRSEIDLLN
IRREFIEKYDKSLHQAIEGDTSGDFLKALLALCGG
ED

The disclosure also contemplates corresponding polynucleotides that encode each of the foregoing annexin proteins. The following polynucleotides are contemplated for use according to the disclosure. Specifically, the following polynucleotides are messenger RNA (mRNA) sequences contemplated for use with a vector of the disclosure to increase activity of an annexin protein. As discussed above, when more than one sequence identifier is used to identify an mRNA sequence in relation to the same annexin species herein (e.g., mRNA sequences relating to annexin A2 are identified herein by SEQ ID NO: 20 and SEQ ID NO: 21) it will be understood that the different sequence identifiers serve to identify transcript variants that may be utilized with a vector of the disclosure to be translated into the particular annexin protein, and that the transcript variants may be used interchangeably or in combination in the methods and compositions of the disclosure.

NM_000700.3 Homo sapiens annexin A1
(ANXA1), mRNA (SEQ ID NO: 19):
AGTGTGAAATCTTCAGAGAAGAATTTCTCTTTAGT
TCTTTGCAAGAAGGTAGAGATAAAGACACTTTTTC
AAAAATGGCAATGGTATCAGAATTCCTCAAGCAGG
CCTGGTTTATTGAAAATGAAGAGCAGGAATATGTT
CAAACTGTGAAGTCATCCAAAGGTGGTCCCGGATC
AGCGGTGAGCCCCTATCCTACCTTCAATCCATCCT
CGGATGTCGCTGCCTTGCATAAGGCCATAATGGTT
AAAGGTGTGGATGAAGCAACCATCATTGACATTCT
AACTAAGCGAAACAATGCACAGCGTCAACAGATCA
AAGCAGCATATCTCCAGGAAACAGGAAAGCCCCTG
GATGAAACACTGAAGAAAGCCCTTACAGGTCACCT
TGAGGAGGTTGTTTTAGCTCTGCTAAAAACTCCAG
CGCAATTTGATGCTGATGAACTTCGTGCTGCCATG
AAGGGCCTTGGAACTGATGAAGATACTCTAATTGA
GATTTTGGCATCAAGAACTAACAAAGAAATCAGAG
ACATTAACAGGGTCTACAGAGAGGAACTGAAGAGA
GATCTGGCCAAAGACATAACCTCAGACACATCTGG
AGATTTTCGGAACGCTTTGCTTTCTCTTGCTAAGG
GTGACCGATCTGAGGACTTTGGTGTGAATGAAGAC
TTGGCTGATTCAGATGCCAGGGCCTTGTATGAAGC
AGGAGAAAGGAGAAAGGGGACAGACGTAAACGTGT
TCAATACCATCCTTACCACCAGAAGCTATCCACAA
CTTCGCAGAGTGTTTCAGAAATACACCAAGTACAG
TAAGCATGACATGAACAAAGTTCTGGACCTGGAGT
TGAAAGGTGACATTGAGAAATGCCTCACAGCTATC
GTGAAGTGCGCCACAAGCAAACCAGCTTTCTTTGC
AGAGAAGCTTCATCAAGCCATGAAAGGTGTTGGAA
CTCGCCATAAGGCATTGATCAGGATTATGGTTTCC
CGTTCTGAAATTGACATGAATGATATCAAAGCATT
CTATCAGAAGATGTATGGTATCTCCCTTTGCCAAG
CCATCCTGGATGAAACCAAAGGAGATTATGAGAAA
ATCCTGGTGGCTCTTTGTGGAGGAAACTAAACATT
CCCTTGATGGTCTCAAGCTATGATCAGAAGACTTT
AATTATATATTTTCATCCTATAAGCTTAAATAGGA
AAGTTTCTTCAACAGGATTACAGTGTAGCTACCTA
CATGCTGAAAAATATAGCCTTTAAATCATTTTTAT
ATTATAACTCTGTATAATAGAGATAAGTCCATTTT
TTAAAAATGTTTTCCCCAAACCATAAAACCCTATA
CAAGTTGTTCTAGTAACAATACATGAGAAAGATGT
CTATGTAGCTGAAAATAAAATGACGTCACAAGACAA
NM_001002858.2 Homo sapiens annexin
A2 (ANXA2), transcript variant 1, mRNA
(SEQ ID NO: 20):
GCTCAGCATTTGGGGACGCTCTCAGCTCTCGGCGC
ACGGCCCAGGTAAGCGGGGCGCGCCCTGCCCGCCC
GCGATGGGCCGCCAGCTAGCGGGGTGTGGAGACGC
TGGGAAGAAGGCTTCCTTCAAAATGTCTACTGTTC
ACGAAATCCTGTGCAAGCTCAGCTTGGAGGGTGAT
CACTCTACACCCCCAAGTGCATATGGGTCTGTCAA
AGCCTATACTAACTTTGATGCTGAGCGGGATGCTT
TGAACATTGAAACAGCCATCAAGACCAAAGGTGTG
GATGAGGTCACCATTGTCAACATTTTGACCAACCG
CAGCAATGCACAGAGACAGGATATTGCCTTCGCCT
ACCAGAGAAGGACCAAAAAGGAACTTGCATCAGCA
CTGAAGTCAGCCTTATCTGGCCACCTGGAGACGGT
GATTTTGGGCCTATTGAAGACACCTGCTCAGTATG
ACGCTTCTGAGCTAAAAGCTTCCATGAAGGGGCTG
GGAACCGACGAGGACTCTCTCATTGAGATCATCTG
CTCCAGAACCAACCAGGAGCTGCAGGAAATTAACA
GAGTCTACAAGGAAATGTACAAGACTGATCTGGAG
AAGGACATTATTTCGGACACATCTGGTGACTTCCG
CAAGCTGATGGTTGCCCTGGCAAAGGGTAGAAGAG
CAGAGGATGGCTCTGTCATTGATTATGAACTGATT
GACCAAGATGCTCGGGATCTCTATGACGCTGGAGT
GAAGAGGAAAGGAACTGATGTTCCCAAGTGGATCA
GCATCATGACCGAGCGGAGCGTGCCCCACCTCCAG
AAAGTATTTGATAGGTACAAGAGTTACAGCCCTTA
TGACATGTTGGAAAGCATCAGGAAAGAGGTTAAAG
GAGACCTGGAAAATGCTTTCCTGAACCTGGTTCAG
TGCATTCAGAACAAGCCCCTGTATTTTGCTGATCG
GCTGTATGACTCCATGAAGGGCAAGGGGACGCGAG
ATAAGGTCCTGATCAGAATCATGGTCTCCCGCAGT
GAAGTGGACATGTTGAAAATTAGGTCTGAATTCAA
GAGAAAGTACGGCAAGTCCCTGTACTATTATATCC
AGCAAGACACTAAGGGCGACTACCAGAAAGCGCTG
CTGTACCTGTGTGGTGGAGATGACTGAAGCCCGAC
ACGGCCTGAGCGTCCAGAAATGGTGCTCACCATGC
TTCCAGCTAACAGGTCTAGAAAACCAGCTTGCGAA
TAACAGTCCCCGTGGCCATCCCTGTGAGGGTGACG
TTAGCATTACCCCCAACCTCATTTTAGTTGCCTAA
GCATTGCCTGGCCTTCCTGTCTAGTCTCTCCTGTA
AGCCAAAGAAATGAACATTCCAAGGAGTTGGAAGT
GAAGTCTATGATGTGAAACACTTTGCCTCCTGTGT
ACTGTGTCATAAACAGATGAATAAACTGAATTTGT
ACTTTAGAAACACGTACTTTGTGGCCCTGCTTTCA
ACTGAATTGTTTGAAAATTAAACGTGCTTGGGGTT
CAGCTGGTGAGGCTGTCCCTGTAGGAAGAAAGCTC
TGGGACTGAGCTGTACAGTATGGTTGCCCCTATCC
AAGTGTCGCTATTTAAGTTAAATTTAAATGAAATA
AAATAAAATAAAATCAAAAAAA
NM_001136015.2 Homo sapiens annexin A2
(ANXA2), transcript variant 4, mRNA
(SEQ ID NO: 21):
GCTCAGCATTTGGGGACGCTCTCAGCTCTCGGCGC
ACGGCCCAGGGTGAAAATGTTTGCCATTAAACTCA
CATGAAGTAGGAAATATTTATATGGATACAAAAGG
CACCTGCATGGGATAATGTCAAATTTCATAGATAC
TGCTTTGTGCTTCCTTCAAAATGTCTACTGTTCAC
GAAATCCTGTGCAAGCTCAGCTTGGAGGGTGATCA
CTCTACACCCCCAAGTGCATATGGGTCTGTCAAAG
CCTATACTAACTTTGATGCTGAGCGGGATGCTTTG
AACATTGAAACAGCCATCAAGACCAAAGGTGTGGA
TGAGGTCACCATTGTCAACATTTTGACCAACCGCA
GCAATGCACAGAGACAGGATATTGCCTTCGCCTAC
CAGAGAAGGACCAAAAAGGAACTTGCATCAGCACT
GAAGTCAGCCTTATCTGGCCACCTGGAGACGGTGA
TTTTGGGCCTATTGAAGACACCTGCTCAGTATGAC
GCTTCTGAGCTAAAAGCTTCCATGAAGGGGCTGGG
AACCGACGAGGACTCTCTCATTGAGATCATCTGCT
CCAGAACCAACCAGGAGCTGCAGGAAATTAACAGA
GTCTACAAGGAAATGTACAAGACTGATCTGGAGAA
GGACATTATTTCGGACACATCTGGTGACTTCCGCA
AGCTGATGGTTGCCCTGGCAAAGGGTAGAAGAGCA
GAGGATGGCTCTGTCATTGATTATGAACTGATTGA
CCAAGATGCTCGGGATCTCTATGACGCTGGAGTGA
AGAGGAAAGGAACTGATGTTCCCAAGTGGATCAGC
ATCATGACCGAGCGGAGCGTGCCCCACCTCCAGAA
AGTATTTGATAGGTACAAGAGTTACAGCCCTTATG
ACATGTTGGAAAGCATCAGGAAAGAGGTTAAAGGA
GACCTGGAAAATGCTTTCCTGAACCTGGTTCAGTG
CATTCAGAACAAGCCCCTGTATTTTGCTGATCGGC
TGTATGACTCCATGAAGGGCAAGGGGACGCGAGAT
AAGGTCCTGATCAGAATCATGGTCTCCCGCAGTGA
AGTGGACATGTTGAAAATTAGGTCTGAATTCAAGA
GAAAGTACGGCAAGTCCCTGTACTATTATATCCAG
CAAGACACTAAGGGCGACTACCAGAAAGCGCTGCT
GTACCTGTGTGGTGGAGATGACTGAAGCCCGACAC
GGCCTGAGCGTCCAGAAATGGTGCTCACCATGCTT
CCAGCTAACAGGTCTAGAAAACCAGCTTGCGAATA
ACAGTCCCCGTGGCCATCCCTGTGAGGGTGACGTT
AGCATTACCCCCAACCTCATTTTAGTTGCCTAAGC
ATTGCCTGGCCTTCCTGTCTAGTCTCTCCTGTAAG
CCAAAGAAATGAACATTCCAAGGAGTTGGAAGTGA
AGTCTATGATGTGAAACACTTTGCCTCCTGTGTAC
TGTGTCATAAACAGATGAATAAACTGAATTTGTAC
TTTAGAAACACGTACTTTGTGGCCCTGCTTTCAAC
TGAATTGTTTGAAAATTAAACGTGCTTGGGGTTCA
GCTGGTGAGGCTGTCCCTGTAGGAAGAAAGCTCTG
GGACTGAGCTGTACAGTATGGTTGCCCCTATCCAA
GTGTCGCTATTTAAGTTAAATTTAAATGAAATAAA
ATAAAATAAAATCAAAAAAA
NM_005139.3 Homo sapiens annexin A3
(ANXA3), mRNA (SEQ ID NO: 22):
AGCGCGGAGCACCTGCGCCCGCGGCTGACACCTTC
GCTCGCAGTTTGTTCGCAGTTTACTCGCACACCAG
TTTCCCCCACCGCGCTTTGGATTAGTGTGATCTCA
GCTCAAGGCAAAGGTGGGATATCATGGCATCTATC
TGGGTTGGACACCGAGGAACAGTAAGAGATTATCC
AGACTTTAGCCCATCAGTGGATGCTGAAGCTATTC
AGAAAGCAATCAGAGGAATTGGAACTGATGAGAAA
ATGCTCATCAGCATTCTGACTGAGAGGTCAAATGC
ACAGCGGCAGCTGATTGTTAAGGAATATCAAGCAG
CATATGGAAAGGAGCTGAAAGATGACTTGAAGGGT
GATCTCTCTGGCCACTTTGAGCATCTCATGGTGGC
CCTAGTGACTCCACCAGCAGTCTTTGATGCAAAGC
AGCTAAAGAAATCCATGAAGGGCGCGGGAACAAAC
GAAGATGCCTTGATTGAAATCTTAACTACCAGGAC
AAGCAGGCAAATGAAGGATATCTCTCAAGCCTATT
ATACAGTATACAAGAAGAGTCTTGGAGATGACATT
AGTTCCGAAACATCTGGTGACTTCCGGAAAGCTCT
GTTGACTTTGGCAGATGGCAGAAGAGATGAAAGTC
TGAAAGTGGATGAGCATCTGGCCAAACAAGATGCC
CAGATTCTCTATAAAGCTGGTGAGAACAGATGGGG
CACGGATGAAGACAAATTCACTGAGATCCTGTGTT
TAAGGAGCTTTCCTCAATTAAAACTAACATTTGAT
GAATACAGAAATATCAGCCAAAAGGACATTGTGGA
CAGCATAAAAGGAGAATTATCTGGGCATTTTGAAG
ACTTACTGTTGGCCATAGTTAATTGTGTGAGGAAC
ACGCCGGCCTTTTTAGCCGAAAGACTGCATCGAGC
CTTGAAGGGTATTGGAACTGATGAGTTTACTCTGA
ACCGAATAATGGTGTCCAGATCAGAAATTGACCTT
TTGGACATTCGAACAGAGTTCAAGAAGCATTATGG
CTATTCCCTATATTCAGCAATTAAATCGGATACTT
CTGGAGACTATGAAATCACACTCTTAAAAATCTGT
GGTGGAGATGACTGAACCAAGAAGATAATCTCCAA
AGGTCCACGATGGGCTTTCCCAACAGCTCCACCTT
ACTTCTTCTCATACTATTTAAGAGAACAAGCAAAT
ATAAACAGCAACTTGTGTTCCTAACAGGAATTTTC
ATTGTTCTATAACAACAACAACAAAAGCGATTATT
ATTTTAGAGCATCTCATTTATAATGTAGCAGCTCA
TAAATGAAATTGAAAATGGTATTAAAGATCTGCAA
CTACTATCCAACTTATATTTCTGCTTTCAAAGTTA
AGAATCTTTATAGTTCTACTCCATTAAATATAAAGC
AAGATAATAAAAATTGTTGCTTTTGTTAAAA
NM_001153.5 Homo sapiens annexin A4
(ANXA4), transcript variant 2, mRNA
(SEQ ID NO: 23):
GTGACCTCCGCAGCCGCAGAGGAGGAGCGCAGCCC
GGCCTCGAAGAACTTCTGCTTGGGTGGCTGAACTC
TGATCTTGACCTAGAGTCATGGCCATGGCAACCAA
AGGAGGTACTGTCAAAGCTGCTTCAGGATTCAATG
CCATGGAAGATGCCCAGACCCTGAGGAAGGCCATG
AAAGGGCTCGGCACCGATGAAGACGCCATTATTAG
CGTCCTTGCCTACCGCAACACCGCCCAGCGCCAGG
AGATCAGGACAGCCTACAAGAGCACCATCGGCAGG
GACTTGATAGACGACCTGAAGTCAGAACTGAGTGG
CAACTTCGAGCAGGTGATTGTGGGGATGATGACGC
CCACGGTGCTGTATGACGTGCAAGAGCTGCGAAGG
GCCATGAAGGGAGCCGGCACTGATGAGGGCTGCCT
AATTGAGATCCTGGCCTCCCGGACCCCTGAGGAGA
TCCGGCGCATAAGCCAAACCTACCAGCAGCAATAT
GGACGGAGCCTTGAAGATGACATTCGCTCTGACAC
ATCGTTCATGTTCCAGCGAGTGCTGGTGTCTCTGT
CAGCTGGTGGGAGGGATGAAGGAAATTATCTGGAC
GATGCTCTCGTGAGACAGGATGCCCAGGACCTGTA
TGAGGCTGGAGAGAAGAAATGGGGGACAGATGAGG
TGAAATTTCTAACTGTTCTCTGTTCCCGGAACCGA
AATCACCTGTTGCATGTGTTTGATGAATACAAAAG
GATATCACAGAAGGATATTGAACAGAGTATTAAAT
CTGAAACATCTGGTAGCTTTGAAGATGCTCTGCTG
GCTATAGTAAAGTGCATGAGGAACAAATCTGCATA
TTTTGCTGAAAAGCTCTATAAATCGATGAAGGGCT
TGGGCACCGATGATAACACCCTCATCAGAGTGATG
GTTTCTCGAGCAGAAATTGACATGTTGGATATCCG
GGCACACTTCAAGAGACTCTATGGAAAGTCTCTGT
ACTCGTTCATCAAGGGTGACACATCTGGAGACTAC
AGGAAAGTACTGCTTGTTCTCTGTGGAGGAGATGA
TTAAAATAAAAATCCCAGAAGGACAGGAGGATTCT
CAACACTTTGAATTTTTTTAACTTCATTTTTCTAC
ACTGCTATTATCATTATCTCAGAATGCTTATTTCC
AATTAAAACGCCTACAGCTGCCTCCTAGAATATAG
ACTGTCTGTATTATTATTCACCTATAATTAGTCAT
TATGATGCTTTAAAGCTGTACTTGCATTTCAAAGC
TTATAAGATATAAATGGAGATTTTAAAGTAGAAAT
AAATATGTATTCCATGTTTTTAAAAGATTACTTTC
TACTTTGTGTTTCACAGACATTGAATATATTAAAT
TATTCCATATTTTCTTTTCAGTGAAAAATTTTTTA
AATGGAAGACTGTTCTAAAATCACTTTTTTCCCTA
ATCCAATTTTTAGAGTGGCTAGTAGTTTCTTCATT
TGAAATTGTAAGCATCCGGTCAGTAAGAATGCCCA
TCCAGTTTTCTATATTTCATAGTCAAAGCCTTGAA
AGCATCTACAAATCTCTTTTTTTAGGTTTTGTCCA
TAGCATCAGTTGATCCTTACTAAGTTTTTCATGGG
AGACTTCCTTCATCACATCTTATGTTGAAATCACT
TTCTGTAGTCAAAGTATACCAAAACCAATTTATCT
GAACTAAATTCTAAAGTATGGTTATACAAACCATA
TACATCTGGTTACCAAACATAAATGCTGAACATTC
CATATTATTATAGTTAATGTCTTAATCCAGCTTGC
AAGTGAATGGAAAAAAAAATAAGCTTCAAACTAGG
TATTCTGGGAATGATGTAATGCTCTGAATTTAGTA
TGATATAAAGAAAACTTTTTTGTGCTAAAAATACT
TTTTAAAATCAATTTTGTTGATTGTAGTAATTTCT
ATTTGCACTGTGCCTTTCAACTCCAGAAACATTCT
GAAGATGTACTTGGATTTAATTAAAAAGTTCACTT
TGTAAGAACGTGGAAAAATAATTTTAATTTAAAAA
TGGTGTTTTTAGGCCGGGGGCGGGGGCTCACGCCA
GTAATCCCAACACTTTGGGAGGCCAAGGCGGGTGG
ATCACCTAAGGTCAGGAGTTCAAGACTAGCCTGGC
CAACATGGAGAAACTGCATCTCTACTAAAAATATA
AAAATTAGCCGGGTGTGGTGGCTGGTGCCTGTAAT
CCCAGCCACTCGGAGGCTGAGTCAGGGAGAACTGC
TTGAACCCAGGAGGCAGGAGGCAAAGGTTGCAGTG
AGCCGAGATCACGCCAGCCTGGGCGACAGAGCGAG
AATCCATCTAAAAAAAAAAAAAAAAAAAGTGTCTT
TAAAGTGAGGTATAGTCTTTCTCTGATCCACTTTT
CACCTTCTGAGGTTTTTCATCTTGGCCCCTGAAAG
GAGCTATTTTTGAAGGACTTGTGTTACTCAGTTTC
TACAGGAATTACAAGATAAGAAAAAAAAAATCATA
TTTAGTCTTATGCGTGCCTACTGGCTAATGTTCAC
ATATGCCAAACACTACTCAATAACATAAAATAATG
TATGAACTTATTCTCTGGAAATGAGTGATGCCCTC
TGCTCTAAGTAGACCATTTATATTAAATATCATAA
ATGTATAAAGGACATTCATATTCTTA
NM_001154.4 Homo sapiens annexin A5
(ANXA5), mRNA (SEQ ID NO: 24):
AGTCTAGGTGCAGCTGCCGGATCCTTCAGCGTCTG
CATCTCGGCGTCGCCCCGCGTACCGTCGCCCGGCT
CTCCGCCGCTCTCCCGGGGTTTCGGGGCACTTGGG
TCCCACAGTCTGGTCCTGCTTCACCTTCCCCTGAC
CTGAGTAGTCGCCATGGCACAGGTTCTCAGAGGCA
CTGTGACTGACTTCCCTGGATTTGATGAGCGGGCT
GATGCAGAAACTCTTCGGAAGGCTATGAAAGGCTT
GGGCACAGATGAGGAGAGCATCCTGACTCTGTTGA
CATCCCGAAGTAATGCTCAGCGCCAGGAAATCTCT
GCAGCTTTTAAGACTCTGTTTGGCAGGGATCTTCT
GGATGACCTGAAATCAGAACTAACTGGAAAATTTG
AAAAATTAATTGTGGCTCTGATGAAACCCTCTCGG
CTTTATGATGCTTATGAACTGAAACATGCCTTGAA
GGGAGCTGGAACAAATGAAAAAGTACTGACAGAAA
TTATTGCTTCAAGGACACCTGAAGAACTGAGAGCC
ATCAAACAAGTTTATGAAGAAGAATATGGCTCAAG
CCTGGAAGATGACGTGGTGGGGGACACTTCAGGGT
ACTACCAGCGGATGTTGGTGGTTCTCCTTCAGGCT
AACAGAGACCCTGATGCTGGAATTGATGAAGCTCA
AGTTGAACAAGATGCTCAGGCTTTATTTCAGGCTG
GAGAACTTAAATGGGGGACAGATGAAGAAAAGTTT
ATCACCATCTTTGGAACACGAAGTGTGTCTCATTT
GAGAAAGGTGTTTGACAAGTACATGACTATATCAG
GATTTCAAATTGAGGAAACCATTGACCGCGAGACT
TCTGGCAATTTAGAGCAACTACTCCTTGCTGTTGT
GAAATCTATTCGAAGTATACCTGCCTACCTTGCAG
AGACCCTCTATTATGCTATGAAGGGAGCTGGGACA
GATGATCATACCCTCATCAGAGTCATGGTTTCCAG
GAGTGAGATTGATCTGTTTAACATCAGGAAGGAGT
TTAGGAAGAATTTTGCCACCTCTCTTTATTCCATG
ATTAAGGGAGATACATCTGGGGACTATAAGAAAGC
TCTTCTGCTGCTCTGTGGAGAAGATGACTAACGTG
TCACGGGGAAGAGCTCCCTGCTGTGTGCCTGCACC
ACCCCACTGCCTTCCTTCAGCACCTTTAGCTGCAT
TTGTATGCCAGTGCTTAACACATTGCCTTATTCAT
ACTAGCATGCTCATGACCAACACATACACGTCATA
GAAGAAAATAGTGGTGCTTCTTTCTGATCTCTAGT
GGAGATCTCTTTGACTGCTGTAGTACTAAAGTGTA
CTTAATGTTACTAAGTTTAATGCCTGGCCATTTTC
CATTTATATATATTTTTTAAGAGGCTAGAGTGCTT
TTAGCCTTTTTTAAAAACTCCATTTATATTACATT
TGTAACCATGATACTTTAATCAGAAGCTTAGCCTT
GAAATTGTGAACTCTTGGAAATGTTATTAGTGAAG
TTCGCAACTAAACTAAACCTGTAAAATTATGATGA
TTGTATTCAAAAGATTAATGAAAAATAAACATTTC
TGTCCCCCTGAATTATGTGTACATGTGTGTTTAGA
TTTATTATTAAATTTATTTAACAATGTT
NM_001155.5 Homo sapiens annexin A6
(ANXA6), transcript variant 1, mRNA
(SEQ ID NO: 25):
GCGGTTGCTGCTGGGCTAACGGGCTCCGATCCAGC
GAGCGCTGCGTCCTCGAGTCCCTGCGCCCGTGCGT
CCGTCTGCGACCCGAGGCCTCCGCTGCGCGTGGAT
TCTGCTGCGAACCGGAGACCATGGCCAAACCAGCA
CAGGGTGCCAAGTACCGGGGCTCCATCCATGACTT
CCCAGGCTTTGACCCCAACCAGGATGCCGAGGCTC
TGTACACTGCCATGAAGGGCTTTGGCAGTGACAAG
GAGGCCATACTGGACATAATCACCTCACGGAGCAA
CAGGCAGAGGCAGGAGGTCTGCCAGAGCTACAAGT
CCCTCTACGGCAAGGACCTCATTGCTGATTTAAAG
TATGAATTGACGGGCAAGTTTGAACGGTTGATTGT
GGGCCTGATGAGGCCACCTGCCTATTGTGATGCCA
AAGAAATTAAAGATGCCATCTCGGGCATTGGCACT
GATGAGAAGTGCCTCATTGAGATCTTGGCTTCCCG
GACCAATGAGCAGATGCACCAGCTGGTGGCAGCAT
ACAAAGATGCCTACGAGCGGGACCTGGAGGCTGAC
ATCATCGGCGACACCTCTGGCCACTTCCAGAAGAT
GCTTGTGGTCCTGCTCCAGGGAACCAGGGAGGAGG
ATGACGTAGTGAGCGAGGACCTGGTACAACAGGAT
GTCCAGGACCTATACGAGGCAGGGGAACTGAAATG
GGGAACAGATGAAGCCCAGTTCATTTACATCTTGG
GAAATCGCAGCAAGCAGCATCTTCGGTTGGTGTTC
GATGAGTATCTGAAGACCACAGGGAAGCCGATTGA
AGCCAGCATCCGAGGGGAGCTGTCTGGGGACTTTG
AGAAGCTAATGCTGGCCGTAGTGAAGTGTATCCGG
AGCACCCCGGAATATTTTGCTGAAAGGCTCTTCAA
GGCTATGAAGGGCCTGGGGACTCGGGACAACACCC
TGATCCGCATCATGGTCTCCCGTAGTGAGTTGGAC
ATGCTCGACATTCGGGAGATCTTCCGGACCAAGTA
TGAGAAGTCCCTCTACAGCATGATCAAGAATGACA
CCTCTGGCGAGTACAAGAAGACTCTGCTGAAGCTG
TCTGGGGGAGATGATGATGCTGCTGGCCAGTTCTT
CCCGGAGGCAGCGCAGGTGGCCTATCAGATGTGGG
AACTTAGTGCAGTGGCCCGAGTAGAGCTGAAGGGA
ACTGTGCGCCCAGCCAATGACTTCAACCCTGACGC
AGATGCCAAAGCGCTGCGGAAAGCCATGAAGGGAC
TCGGGACTGACGAAGACACAATCATCGATATCATC
ACGCACCGCAGCAATGTCCAGCGGCAGCAGATCCG
GCAGACCTTCAAGTCTCACTTTGGCCGGGACTTAA
TGACTGACCTGAAGTCTGAGATCTCTGGAGACCTG
GCAAGGCTGATTCTGGGGCTCATGATGCCACCGGC
CCATTACGATGCCAAGCAGTTGAAGAAGGCCATGG
AGGGAGCCGGCACAGATGAAAAGGCTCTTATTGAA
ATCCTGGCCACTCGGACCAATGCTGAAATCCGGGC
CATCAATGAGGCCTATAAGGAGGACTATCACAAGT
CCCTGGAGGATGCTCTGAGCTCAGACACATCTGGC
CACTTCAGGAGGATCCTCATTTCTCTGGCCACGGG
GCATCGTGAGGAGGGAGGAGAAAACCTGGACCAGG
CACGGGAAGATGCCCAGGTGGCTGCTGAGATCTTG
GAAATAGCAGACACACCTAGTGGAGACAAAACTTC
CTTGGAGACACGTTTCATGACGATCCTGTGTACCC
GGAGCTATCCGCACCTCCGGAGAGTCTTCCAGGAG
TTCATCAAGATGACCAACTATGACGTGGAGCACAC
CATCAAGAAGGAGATGTCTGGGGATGTCAGGGATG
CATTTGTGGCCATTGTTCAAAGTGTCAAGAACAAG
CCTCTCTTCTTTGCCGACAAACTTTACAAATCCAT
GAAGGGTGCTGGCACAGATGAGAAGACTCTGACCA
GGATCATGGTATCCCGCAGTGAGATTGACCTGCTC
AACATCCGGAGGGAATTCATTGAGAAATATGACAA
GTCTCTCCACCAAGCCATTGAGGGTGACACCTCCG
GAGACTTCCTGAAGGCCTTGCTGGCTCTCTGTGGT
GGTGAGGACTAGGGCCACAGCTTTGGCGGGCACTT
CTGCCAAGAAATGGTTATCAGCACCAGCCGCCATG
GCCAAGCCTGATTGTTCCAGCTCCAGAGACTAAGG
AAGGGGCAGGGGTGGGGGGAGGGGTTGGGTTGGGC
TCTTATCTTCAGTGGAGCTTAGGAAACGCTCCCAC
TCCCACGGGCCATCGAGGGCCCAGCACGGCTGAGC
GGCTGAAAAACCGTAGCCATAGATCCTGTCCACCT
CCACTCCCCTCTGACCCTCAGGCTTTCCCAGCTTC
CTCCCCTTGCTACAGCCTCTGCCCTGGTTTGGGCT
ATGTCAGATCCAAAAACATCCTGAACCTCTGTCTG
TAAAATGAGTAGTGTCTGTACTTTGAATGAGGGGG
TTGGTGGCAGGGGCCAGTTGAATGTGCTGGGCGGG
GTGGTGGGAAGGATAGTAAATGTGCTGGGGCAAAC
TGACAAATCTTCCCATCCATTTCACCACCCATCTC
CATCCAGGCCGCGCTAGAGTACTGGACCAGGAATT
TGGATGCCTGGGTTCAAATCTGCATCTGCCATGCA
CTTGTTTCTGACCTTAGGCCAGCCCCTTTCCCTCC
CTGAGTCTCTATTTTCTTATCTACAATGAGACAGT
TGGACAAAAAAATCTTGGCTTCCCTTCTAACATTA
ACTTCCTAAAGTATGCCTCCGATTCATTCCCTTGA
CACTTTTTATTTCTAAGGAAGAAATAAAAAGAGAT
ACACAAACACATAAACACA
NM_001193544.1 Homo sapiens annexin A6
(ANXA6), transcript variant 2, mRNA
(SEQ ID NO: 26):
AGAGACCAGAGAGCATCCAGAGGCCTGGCCGGGGT
CCTGCAGTGCAGACGTTGGGAGGCACGGAGACGGG
GAGAGGGGGAGGCGGTCCAGGACTCACTCTGCTCC
ACCTCTGACTCCTTGAAGGGTGCCAAGTACCGGGG
CTCCATCCATGACTTCCCAGGCTTTGACCCCAACC
AGGATGCCGAGGCTCTGTACACTGCCATGAAGGGC
TTTGGCAGTGACAAGGAGGCCATACTGGACATAAT
CACCTCACGGAGCAACAGGCAGAGGCAGGAGGTCT
GCCAGAGCTACAAGTCCCTCTACGGCAAGGACCTC
ATTGCTGATTTAAAGTATGAATTGACGGGCAAGTT
TGAACGGTTGATTGTGGGCCTGATGAGGCCACCTG
CCTATTGTGATGCCAAAGAAATTAAAGATGCCATC
TCGGGCATTGGCACTGATGAGAAGTGCCTCATTGA
GATCTTGGCTTCCCGGACCAATGAGCAGATGCACC
AGCTGGTGGCAGCATACAAAGATGCCTACGAGCGG
GACCTGGAGGCTGACATCATCGGCGACACCTCTGG
CCACTTCCAGAAGATGCTTGTGGTCCTGCTCCAGG
GAACCAGGGAGGAGGATGACGTAGTGAGCGAGGAC
CTGGTACAACAGGATGTCCAGGACCTATACGAGGC
AGGGGAACTGAAATGGGGAACAGATGAAGCCCAGT
TCATTTACATCTTGGGAAATCGCAGCAAGCAGCAT
CTTCGGTTGGTGTTCGATGAGTATCTGAAGACCAC
AGGGAAGCCGATTGAAGCCAGCATCCGAGGGGAGC
TGTCTGGGGACTTTGAGAAGCTAATGCTGGCCGTA
GTGAAGTGTATCCGGAGCACCCCGGAATATTTTGC
TGAAAGGCTCTTCAAGGCTATGAAGGGCCTGGGGA
CTCGGGACAACACCCTGATCCGCATCATGGTCTCC
CGTAGTGAGTTGGACATGCTCGACATTCGGGAGAT
CTTCCGGACCAAGTATGAGAAGTCCCTCTACAGCA
TGATCAAGAATGACACCTCTGGCGAGTACAAGAAG
ACTCTGCTGAAGCTGTCTGGGGGAGATGATGATGC
TGCTGGCCAGTTCTTCCCGGAGGCAGCGCAGGTGG
CCTATCAGATGTGGGAACTTAGTGCAGTGGCCCGA
GTAGAGCTGAAGGGAACTGTGCGCCCAGCCAATGA
CTTCAACCCTGACGCAGATGCCAAAGCGCTGCGGA
AAGCCATGAAGGGACTCGGGACTGACGAAGACACA
ATCATCGATATCATCACGCACCGCAGCAATGTCCA
GCGGCAGCAGATCCGGCAGACCTTCAAGTCTCACT
TTGGCCGGGACTTAATGACTGACCTGAAGTCTGAG
ATCTCTGGAGACCTGGCAAGGCTGATTCTGGGGCT
CATGATGCCACCGGCCCATTACGATGCCAAGCAGT
TGAAGAAGGCCATGGAGGGAGCCGGCACAGATGAA
AAGGCTCTTATTGAAATCCTGGCCACTCGGACCAA
TGCTGAAATCCGGGCCATCAATGAGGCCTATAAGG
AGGACTATCACAAGTCCCTGGAGGATGCTCTGAGC
TCAGACACATCTGGCCACTTCAGGAGGATCCTCAT
TTCTCTGGCCACGGGGCATCGTGAGGAGGGAGGAG
AAAACCTGGACCAGGCACGGGAAGATGCCCAGGTG
GCTGCTGAGATCTTGGAAATAGCAGACACACCTAG
TGGAGACAAAACTTCCTTGGAGACACGTTTCATGA
CGATCCTGTGTACCCGGAGCTATCCGCACCTCCGG
AGAGTCTTCCAGGAGTTCATCAAGATGACCAACTA
TGACGTGGAGCACACCATCAAGAAGGAGATGTCTG
GGGATGTCAGGGATGCATTTGTGGCCATTGTTCAA
AGTGTCAAGAACAAGCCTCTCTTCTTTGCCGACAA
ACTTTACAAATCCATGAAGGGTGCTGGCACAGATG
AGAAGACTCTGACCAGGATCATGGTATCCCGCAGT
GAGATTGACCTGCTCAACATCCGGAGGGAATTCAT
TGAGAAATATGACAAGTCTCTCCACCAAGCCATTG
AGGGTGACACCTCCGGAGACTTCCTGAAGGCCTTG
CTGGCTCTCTGTGGTGGTGAGGACTAGGGCCACAG
CTTTGGCGGGCACTTCTGCCAAGAAATGGTTATCA
GCACCAGCCGCCATGGCCAAGCCTGATTGTTCCAG
CTCCAGAGACTAAGGAAGGGGCAGGGGTGGGGGGA
GGGGTTGGGTTGGGCTCTTATCTTCAGTGGAGCTT
AGGAAACGCTCCCACTCCCACGGGCCATCGAGGGC
CCAGCACGGCTGAGCGGCTGAAAAACCGTAGCCAT
AGATCCTGTCCACCTCCACTCCCCTCTGACCCTCA
GGCTTTCCCAGCTTCCTCCCCTTGCTACAGCCTCT
GCCCTGGTTTGGGCTATGTCAGATCCAAAAACATC
CTGAACCTCTGTCTGTAAAATGAGTAGTGTCTGTA
CTTTGAATGAGGGGGTTGGTGGCAGGGGCCAGTTG
AATGTGCTGGGCGGGGTGGTGGGAAGGATAGTAAA
TGTGCTGGGGCAAACTGACAAATCTTCCCATCCAT
TTCACCACCCATCTCCATCCAGGCCGCGCTAGAGT
ACTGGACCAGGAATTTGGATGCCTGGGTTCAAATC
TGCATCTGCCATGCACTTGTTTCTGACCTTAGGCC
AGCCCCTTTCCCTCCCTGAGTCTCTATTTTCTTAT
CTACAATGAGACAGTTGGACAAAAAAATCTTGGCT
TCCCTTCTAACATTAACTTCCTAAAGTATGCCTCC
GATTCATTCCCTTGACACTTTTTATTTCTAAGGAA
GAAATAAAAAGAGATACACAAACACATAAACACAA
AAAAAAAAA
NM_001156.5 Homo sapiens annexin A7
(ANXA7),transcript variant 1, mRNA
(SEQ ID NO: 27):
ATCTTGCGGGAGACCGGGTTGGGCTGTGACGCTGC
TGCTGGGGTCAGAATGTCATACCCAGGCTATCCCC
CAACAGGCTACCCACCTTTCCCTGGATATCCTCCT
GCAGGTCAGGAGTCATCTTTTCCCCCTTCTGGTCA
GTATCCTTATCCTAGTGGCTTTCCTCCAATGGGAG
GAGGTGCCTACCCACAAGTGCCAAGTAGTGGCTAC
CCAGGAGCTGGAGGCTACCCTGCGCCTGGAGGTTA
TCCAGCCCCTGGAGGCTATCCTGGTGCCCCACAGC
CAGGGGGAGCTCCATCCTATCCCGGAGTTCCTCCA
GGCCAAGGATTTGGAGTCCCACCAGGTGGAGCAGG
CTTTTCTGGGTATCCACAGCCACCTTCACAGTCTT
ATGGAGGTGGTCCAGCACAGGTTCCACTACCTGGT
GGCTTTCCTGGAGGACAGATGCCTTCTCAGTATCC
TGGAGGACAACCTACTTACCCTAGTCAGCCTGCCA
CAGTGACTCAGGTCACTCAAGGAACTATCCGACCA
GCTGCCAACTTCGATGCTATAAGAGATGCAGAAAT
TCTTCGTAAGGCAATGAAGGGTTTTGGGACAGATG
AGCAGGCAATTGTGGATGTGGTGGCCAACCGTTCC
AATGATCAGAGGCAAAAAATTAAAGCAGCATTTAA
GACCTCCTATGGCAAGGATTTAATCAAAGATCTCA
AATCAGAGTTAAGTGGAAATATGGAAGAACTGATC
CTGGCCCTCTTCATGCCTCCTACGTATTACGATGC
CTGGAGCTTACGGAAAGCAATGCAGGGAGCAGGAA
CTCAGGAACGTGTATTGATTGAGATTTTGTGCACA
AGAACAAATCAGGAAATCCGAGAAATTGTCAGATG
TTATCAGTCAGAATTTGGACGAGACCTTGAAAAGG
ACATTAGGTCAGATACATCAGGACATTTTGAACGT
TTACTTGTGTCCATGTGCCAGGGAAATCGTGATGA
GAACCAGAGTATAAACCACCAAATGGCTCAGGAAG
ATGCTCAGCGTCTCTATCAAGCTGGTGAGGGGAGA
CTAGGGACCGATGAATCTTGCTTTAACATGATCCT
TGCCACAAGAAGCTTTCCTCAGCTGAGAGCTACCA
TGGAGGCTTATTCTAGGATGGCTAATCGAGACTTG
TTAAGCAGTGTGAGCCGTGAGTTTTCCGGATATGT
AGAAAGTGGTTTGAAGACCATCTTGCAGTGTGCCC
TGAACCGCCCTGCCTTCTTTGCTGAGAGGCTCTAC
TATGCTATGAAAGGTGCTGGCACAGATGACTCCAC
CCTGGTCCGGATTGTGGTCACTCGAAGTGAGATTG
ACCTTGTACAAATAAAACAGATGTTCGCTCAGATG
TATCAGAAGACTCTGGGCACAATGATTGCAGGTGA
CACGAGTGGAGATTACCGAAGACTTCTTCTGGCTA
TTGTGGGCCAGTAGGAGGGATTTTTTTTTTTTTAA
TGAAAAAAAATTTCTATTCATAGCTTATCCTTCAG
AGCAATGACCTGCATGCAGCAATATCAAACATCAG
CTAACCGAAAGAGCTTTCTGTCAAGGACCGTATCA
GGGTAATGTGCTTGGTTTGCACATGTTGTTATTGC
CTTAATTCTAATTTTATTTTGTTCTCTACATACAA
TCAATGTAAAGCCATATCACAATGATACAGTAATA
TTGCAATGTTTGTAAACCTTCATTCTTACTAGTTT
CATTCTAATCAAGATGTCAAATTGAATAAAAATCA
CAGCAATCTCTGATTCTGTGTAATAATATTGAATA
ATTTTTTAGAAGGTTACTGAAAGCTCTGCCTTCCG
GAATCCCTCTAAGTCTGCTTGATAGAGTGGATAGT
GTGTTAAAACTGTGTACTTTAAAAAAAAATTCAAC
CTTTACATCTAGAATAATTTGCATCTCATTTTGCC
TAAATTGGTTCTGTATTCATAAACACTTTCCACAT
AGAAAATAGATTAGTATTACCTGTGGCACCTTTTA
AGAAAGGGTCAAATGTTTATATGCTTAAGATACAT
AGCCTACTTTTTTTTCGCAGTTGTTTTCTTTTTTT
AAATTGAGTTATGACAAATAAAAAATTGCATATAT
TTAAGGTGTACAATATGGTGTTTTGATATCAGCAT
TCCTTGTGTAATGATTCCACAATTAAGGTCAGGCT
AATTACGTATCTGTCACCTTGACATAGTTACCATT
TTTTCATGTGTGGTGAAAACACTTAAGATCTACTA
CCTTAGCAAATTTTAAGTGTTCAGTACATTATTAA
CTATAGATACTGTGCTCTACATTAAACCTCTAGCA
TTTATTCGTTTTATAACTGAAAGTTTATACCCTTT
GACCAACATCTCCCCATTTTCCCCACCTCTCACCT
GGACAACCACCACTGTGTTTAAGTTCAGCTATTTT
AGATTCCACGTATAAATGGTATACAATA
NM_004034.3 Homo sapiens annexin A7
(ANXA7), transcript variant 2, mRNA
(SEQ ID NO: 28):
GCCCACCCTGGGCCCGCCCCCGGCTCCATCTTGCG
GGAGACCGGGTTGGGCTGTGACGCTGCTGCTGGGG
TCAGAATGTCATACCCAGGCTATCCCCCAACAGGC
TACCCACCTTTCCCTGGATATCCTCCTGCAGGTCA
GGAGTCATCTTTTCCCCCTTCTGGTCAGTATCCTT
ATCCTAGTGGCTTTCCTCCAATGGGAGGAGGTGCC
TACCCACAAGTGCCAAGTAGTGGCTACCCAGGAGC
TGGAGGCTACCCTGCGCCTGGAGGTTATCCAGCCC
CTGGAGGCTATCCTGGTGCCCCACAGCCAGGGGGA
GCTCCATCCTATCCCGGAGTTCCTCCAGGCCAAGG
ATTTGGAGTCCCACCAGGTGGAGCAGGCTTTTCTG
GGTATCCACAGCCACCTTCACAGTCTTATGGAGGT
GGTCCAGCACAGGTTCCACTACCTGGTGGCTTTCC
TGGAGGACAGATGCCTTCTCAGTATCCTGGAGGAC
AACCTACTTACCCTAGTCAGATCAATACAGATTCT
TTTTCTTCCTATCCTGTTTTCTCTCCTGTTTCTTT
GGATTATAGCAGTGAACCTGCCACAGTGACTCAGG
TCACTCAAGGAACTATCCGACCAGCTGCCAACTTC
GATGCTATAAGAGATGCAGAAATTCTTCGTAAGGC
AATGAAGGGTTTTGGGACAGATGAGCAGGCAATTG
TGGATGTGGTGGCCAACCGTTCCAATGATCAGAGG
CAAAAAATTAAAGCAGCATTTAAGACCTCCTATGG
CAAGGATTTAATCAAAGATCTCAAATCAGAGTTAA
GTGGAAATATGGAAGAACTGATCCTGGCCCTCTTC
ATGCCTCCTACGTATTACGATGCCTGGAGCTTACG
GAAAGCAATGCAGGGAGCAGGAACTCAGGAACGTG
TATTGATTGAGATTTTGTGCACAAGAACAAATCAG
GAAATCCGAGAAATTGTCAGATGTTATCAGTCAGA
ATTTGGACGAGACCTTGAAAAGGACATTAGGTCAG
ATACATCAGGACATTTTGAACGTTTACTTGTGTCC
ATGTGCCAGGGAAATCGTGATGAGAACCAGAGTAT
AAACCACCAAATGGCTCAGGAAGATGCTCAGCGTC
TCTATCAAGCTGGTGAGGGGAGACTAGGGACCGAT
GAATCTTGCTTTAACATGATCCTTGCCACAAGAAG
CTTTCCTCAGCTGAGAGCTACCATGGAGGCTTATT
CTAGGATGGCTAATCGAGACTTGTTAAGCAGTGTG
AGCCGTGAGTTTTCCGGATATGTAGAAAGTGGTTT
GAAGACCATCTTGCAGTGTGCCCTGAACCGCCCTG
CCTTCTTTGCTGAGAGGCTCTACTATGCTATGAAA
GGTGCTGGCACAGATGACTCCACCCTGGTCCGGAT
TGTGGTCACTCGAAGTGAGATTGACCTTGTACAAA
TAAAACAGATGTTCGCTCAGATGTATCAGAAGACT
CTGGGCACAATGATTGCAGGTGACACGAGTGGAGA
TTACCGAAGACTTCTTCTGGCTATTGTGGGCCAGT
AGGAGGGATTTTTTTTTTTTTAATGAAAAAAAATT
TCTATTCATAGCTTATCCTTCAGAGCAATGACCTG
CATGCAGCAATATCAAACATCAGCTAACCGAAAGA
GCTTTCTGTCAAGGACCGTATCAGGGTAATGTGCT
TGGTTTGCACATGTTGTTATTGCCTTAATTCTAAT
TTTATTTTGTTCTCTACATACAATCAATGTAAAGC
CATATCACAATGATACAGTAATATTGCAATGTTTG
TAAACCTTCATTCTTACTAGTTTCATTCTAATCAA
GATGTCAAATTGAATAAAAATCACAGCAATCTCTG
ATTCTGTGTAATAATATTGAATAATTTTTTAGAAG
GTTACTGAAAGCTCTGCCTTCCGGAATCCCTCTAA
GTCTGCTTGATAGAGTGGATAGTGTGTTAAAACTG
TGTACTTTAAAAAAAAATTCAACCTTTACATCTAG
AATAATTTGCATCTCATTTTGCCTAAATTGGTTCT
GTATTCATAAACACTTTCCACATAGAAAATAGATT
AGTATTACCTGTGGCACCTTTTAAGAAAGGGTCAA
ATGTTTATATGCTTAAGATACATAGCCTACTTTTT
TTTCGCAGTTGTTTTCTTTTTTTAAATTGAGTTAT
GACAAATAAAAAATTGCATATATTTAAGGTGTACA
ATATGGTGTTTTGATATCAGCATTCCTTGTGTAAT
GATTCCACAATTAAGGTCAGGCTAATTACGTATCT
GTCACCTTGACATAGTTACCATTTTTTCATGTGTG
GTGAAAACACTTAAGATCTACTACCTTAGCAAATT
TTAAGTGTTCAGTACATTATTAACTATAGATACTG
TGCTCTACATTAAACCTCTAGCATTTATTCGTTTT
ATAACTGAAAGTTTATACCCTTTGACCAACATCTC
CCCATTTTCCCCACCTCTCACCTGGACAACCACCA
CTGTGTTTAAGTTCAGCTATTTTAGATTCCACGTA
TAAATGGTATACAATAAAAAAAAAAAAAAA
NM_001271702.1 Homo sapiens annexin
A8 (ANXA8), transcript variant 1,
mRNA (SEQ ID NO: 29):
CTGGGTGGGGCCTGGGAGCCACAGGAGATGCCCAA
AGCCAGGCAGAGCCCGGGGGCGAGGGGACGGCAGG
CAGGTGTGGCGCTGCCCTGGGCGGGCTTGCACCCC
CACACCCAAGTGAGCGGCCTGCTCACTCCTCAGCT
GCAGGAGCCAGACGTGTGGAGTCCCAGCAGAGGCC
AACCTGTGTCTCTTCATCTCCCTGGGAAAGGTGCC
CCCGAGGTGAAAGAGATGGCCTGGTGGAAATCCTG
GATTGAACAGGAGGGTGTCACAGTGAAGAGCAGCT
CCCACTTCAACCCAGACCCTGATGCAGAGACCCTC
TACAAAGCCATGAAGGGGATCGGTGTCGGGTCCCA
ACTGCTCAGCCACCAAGCAGCTGCCTTCGCCTTCC
CCTCCTCCGCCCTCACCAGTGTGTCACCCTGGGGG
CAGCAGGGTCACTTGTGCTGTAACCCTGCAGGGAC
CAACGAGCAGGCTATCATCGATGTGCTCACCAAGA
GAAGCAACACGCAGCGGCAGCAGATCGCCAAGTCC
TTCAAGGCTCAGTTCGGCAAGGACCTCACTGAGAC
CTTGAAGTCTGAGCTCAGTGGCAAGTTTGAGAGGC
TCATTGTGGCCCTTATGTACCCGCCATACAGATAC
GAAGCCAAGGAGCTGCATGACGCCATGAAGGGCTT
AGGAACCAAGGAGGGTGTCATCATTGAGATCCTGG
CCTCTCGGACCAAGAACCAGCTGCGGGAGATAATG
AAGGCGTATGAGGAAGACTATGGGTCCAGCCTGGA
GGAGGACATCCAAGCAGACACAAGTGGCTACCTGG
AGAGGATCCTGGTGTGCCTCCTGCAGGGCAGCAGG
GATGATGTGAGCAGCTTTGTGGACCCAGGACTGGC
CCTCCAAGACGCACAGGATCTGTATGCGGCAGGCG
AGAAGATTCGTGGGACTGATGAGATGAAATTCATC
ACCATCCTGTGCACGCGCAGTGCCACTCACCTGCT
GAGAGTGTTTGAAGAGTATGAGAAAATTGCCAACA
AGAGCATTGAGGACAGCATCAAGAGTGAGACCCAT
GGCTCACTGGAGGAGGCCATGCTCACTGTGGTGAA
ATGCACCCAAAACCTCCACAGCTACTTTGCAGAGA
GACTCTACTATGCCATGAAGGGAGCAGGGACGCGT
GATGGGACCCTGATAAGAAACATCGTTTCAAGGAG
CGAGATTGACTTAAATCTTATCAAATGTCACTTCA
AGAAGATGTACGGCAAGACCCTCAGCAGCATGATC
ATGGAAGACACCAGCGGTGACTACAAGAACGCCCT
GCTGAGCCTGGTGGGCAGCGACCCCTGAGGCACAG
AAGAACAAGAGCAAAGACCATGAAGCCAGAGTCTC
CAGGACTCCTCACTCAACCTCGGCCATGGACGCAG
GTTGGGTGTGAGGGGGGTCCCAGCCTTTCGGTCTT
CTATTTCCCTATTTCCAGTGCTTTCCAGCCGGGTT
TCTGACCCAGAGGGTGGAACCGGCCTGGACTCCTC
TTCCCAACTTCCTCCAGGTCATTTCCCAGTGTGAG
CACAATGCCAACCTTAGTGTTTCTCCAGCCAGACA
GATGCCTCAGCATGAAGGGCTTGGGGACTTGTGGA
TCATTCCTTCCTCCCTGCAGGAGCTTCCCAAGCTG
GTCACAGAGTCTCCTGGGCACAGGTTATACAGACC
CCAGCCCCATTCCCATCTACTGAAACAGGGTCTCC
ACAAGAGGGGCCAGGGAATATGGGTTTTTAACAAG
CGTCTTACAAAACACTTCTCTATCATGCAGCCGGA
GAGCTGGCTGGGAGCCCTTTTGTTTTAGAACACAC
ATCCTTCAGCAGCTGAGAAACGAACACGAATCCAT
CCCAACCGAGATGCCATTAACATTCATCTAAAAAT
GTTAGGCTCTAAATGGACGAAAAATTCTCTCGCCA
TCTTAATAACAAAATAAACTACAAATTCCTGACCC
AAGGACACTGTGTTATAAGAGGCGTGGGCTCCCCT
GGTGGCTGACCAGGTCAGCTGCCCTGGCCTTGCAC
CCCTCTGCATGCAGCACAGAAGGGTGTGACCATGC
CCTCAGCACCACTCTTGTCCCCACTGAACGGCAAC
TGAGACTGGGTACCTGGAGATTCTGAAGTGCCTTT
GCTGTGGTTTTCAAAATAATAAAGATTTGTATTCA
ACTCAAAAAAAAAAAAAAAAAAAAAAAAAAAAAAA
NM_001040084.2 Homo sapiens annexin
A8 (ANXA8), transcript variant 2,
mRNA (SEQ ID NO: 30):
CTGGGTGGGGCCTGGGAGCCACAGGAGATGCCCAA
AGCCAGGCAGAGCCCGGGGGCGAGGGGACGGCAGG
CAGGTGTGGCGCTGCCCTGGGCGGGCTTGCACCCC
CACACCCAAGTGAGCGGCCTGCTCACTCCTCAGCT
GCAGGAGCCAGACGTGTGGAGTCCCAGCAGAGGCC
AACCTGTGTCTCTTCATCTCCCTGGGAAAGGTGCC
CCCGAGGTGAAAGAGATGGCCTGGTGGAAATCCTG
GATTGAACAGGAGGGTGTCACAGTGAAGAGCAGCT
CCCACTTCAACCCAGACCCTGATGCAGAGACCCTC
TACAAAGCCATGAAGGGGATCGGGACCAACGAGCA
GGCTATCATCGATGTGCTCACCAAGAGAAGCAACA
CGCAGCGGCAGCAGATCGCCAAGTCCTTCAAGGCT
CAGTTCGGCAAGGACCTCACTGAGACCTTGAAGTC
TGAGCTCAGTGGCAAGTTTGAGAGGCTCATTGTGG
CCCTTATGTACCCGCCATACAGATACGAAGCCAAG
GAGCTGCATGACGCCATGAAGGGCTTAGGAACCAA
GGAGGGTGTCATCATTGAGATCCTGGCCTCTCGGA
CCAAGAACCAGCTGCGGGAGATAATGAAGGCGTAT
GAGGAAGACTATGGGTCCAGCCTGGAGGAGGACAT
CCAAGCAGACACAAGTGGCTACCTGGAGAGGATCC
TGGTGTGCCTCCTGCAGGGCAGCAGGGATGATGTG
AGCAGCTTTGTGGACCCAGGACTGGCCCTCCAAGA
CGCACAGGATCTGTATGCGGCAGGCGAGAAGATTC
GTGGGACTGATGAGATGAAATTCATCACCATCCTG
TGCACGCGCAGTGCCACTCACCTGCTGAGAGTGTT
TGAAGAGTATGAGAAAATTGCCAACAAGAGCATTG
AGGACAGCATCAAGAGTGAGACCCATGGCTCACTG
GAGGAGGCCATGCTCACTGTGGTGAAATGCACCCA
AAACCTCCACAGCTACTTTGCAGAGAGACTCTACT
ATGCCATGAAGGGAGCAGGGACGCGTGATGGGACC
CTGATAAGAAACATCGTTTCAAGGAGCGAGATTGA
CTTAAATCTTATCAAATGTCACTTCAAGAAGATGT
ACGGCAAGACCCTCAGCAGCATGATCATGGAAGAC
ACCAGCGGTGACTACAAGAACGCCCTGCTGAGCCT
GGTGGGCAGCGACCCCTGAGGCACAGAAGAACAAG
AGCAAAGACCATGAAGCCAGAGTCTCCAGGACTCC
TCACTCAACCTCGGCCATGGACGCAGGTTGGGTGT
GAGGGGGGTCCCAGCCTTTCGGTCTTCTATTTCCC
TATTTCCAGTGCTTTCCAGCCGGGTTTCTGACCCA
GAGGGTGGAACCGGCCTGGACTCCTCTTCCCAACT
TCCTCCAGGTCATTTCCCAGTGTGAGCACAATGCC
AACCTTAGTGTTTCTCCAGCCAGACAGATGCCTCA
GCATGAAGGGCTTGGGGACTTGTGGATCATTCCTT
CCTCCCTGCAGGAGCTTCCCAAGCTGGTCACAGAG
TCTCCTGGGCACAGGTTATACAGACCCCAGCCCCA
TTCCCATCTACTGAAACAGGGTCTCCACAAGAGGG
GCCAGGGAATATGGGTTTTTAACAAGCGTCTTACA
AAACACTTCTCTATCATGCAGCCGGAGAGCTGGCT
GGGAGCCCTTTTGTTTTAGAACACACATCCTTCAG
CAGCTGAGAAACGAACACGAATCCATCCCAACCGA
GATGCCATTAACATTCATCTAAAAATGTTAGGCTC
TAAATGGACGAAAAATTCTCTCGCCATCTTAATAA
CAAAATAAACTACAAATTCCTGACCCAAGGACACT
GTGTTATAAGAGGCGTGGGCTCCCCTGGTGGCTGA
CCAGGTCAGCTGCCCTGGCCTTGCACCCCTCTGCA
TGCAGCACAGAAGGGTGTGACCATGCCCTCAGCAC
CACTCTTGTCCCCACTGAACGGCAACTGAGACTGG
GTACCTGGAGATTCTGAAGTGCCTTTGCTGTGGTT
TTCAAAATAATAAAGATTTGTATTCAACTCAAAAA
AAAAA
NM_003568.3 Homo sapiens annexin A9
(ANXA9), mRNA (SEQ ID NO: 31):
CTCTACCAGGCCACACCGGAGGCAGTGCTCACACA
GGCAAGCTACCAGGCCACAACAACGACACCCACCT
CACCTCTGGCACCTCTGAGCATCCACGTACTTGCA
AGAACTCTTGCTCACATCAGCTAAGAGATTGCACC
TGCTGACCTAGAGATTCCGGCCTGTGCTCCTGTGC
TGCTGAGCAGGGCAACCAGTAGCACCATGTCTGTG
ACTGGCGGGAAGATGGCACCGTCCCTCACCCAGGA
GATCCTCAGCCACCTGGGCCTGGCCAGCAAGACTG
CAGCGTGGGGGACCCTGGGCACCCTCAGGACCTTC
TTGAACTTCAGCGTGGACAAGGATGCGCAGAGGCT
ACTGAGGGCCATTACTGGCCAAGGCGTGGACCGCA
GTGCCATTGTGGACGTGCTGACCAACCGGAGCAGA
GAGCAAAGGCAGCTCATCTCACGAAACTTCCAGGA
GCGCACCCAACAGGACCTGATGAAGTCTCTACAGG
CAGCACTTTCCGGCAACCTGGAGAGGATTGTGATG
GCTCTGCTGCAGCCCACAGCCCAGTTTGACGCCCA
GGAATTGAGGACAGCTCTGAAGGCCTCAGATTCTG
CTGTGGACGTGGCCATTGAAATTCTTGCCACTCGA
ACCCCACCCCAGCTGCAGGAGTGCCTGGCAGTCTA
CAAACACAATTTCCAGGTGGAGGCTGTGGATGACA
TCACATCTGAGACCAGTGGCATCTTGCAGGACCTG
CTGTTGGCCCTGGCCAAGGGGGGCCGTGACAGCTA
CTCTGGAATCATTGACTATAATCTGGCAGAACAAG
ATGTCCAGGCACTGCAGCGGGCAGAAGGACCTAGC
AGAGAGGAAACATGGGTCCCAGTCTTCACCCAGCG
AAATCCTGAACACCTCATCCGAGTGTTTGATCAGT
ACCAGCGGAGCACTGGGCAAGAGCTGGAGGAGGCT
GTCCAGAACCGTTTCCATGGAGATGCTCAGGTGGC
TCTGCTCGGCCTAGCTTCGGTGATCAAGAACACAC
CGCTGTACTTTGCTGACAAACTTCATCAAGCCCTC
CAGGAAACTGAGCCCAATTACCAAGTCCTGATTCG
CATCCTTATCTCTCGATGTGAGACTGACCTTCTGA
GTATCAGAGCTGAGTTCAGGAAGAAATTTGGGAAG
TCCCTCTACTCTTCTCTCCAGGATGCAGTGAAAGG
GGATTGCCAGTCAGCCCTCCTGGCCTTGTGCAGGG
CTGAAGACATGTGAGACTTCCCTGCCCCACCCCAC
ATGACATCCGAGGATCTGAGATTTCCGTGTTTGGC
TGAACCTGGGAGACCAGCTGGGCCTCCAAGTAGGA
TAACCCCTCACTGAGCACCACATTCTCTAGCTTCT
TGTTGAGGCTGGAACTGTTTCTTTAAAATCCCTTA
ATTTTCCCATCTCAAAATTATATCTGTACCTGGGT
CATCCAGCTCCTTCTTGGGTGTGGGGAAATGAGTT
TTCTTTGATAGTTTCTGCCTCACTCATCCCTCCTG
TACCCTGGCCAGAACATCTCACTGATACTCGAATT
CTTTTGGCAAA
NM_007193.4 Homo sapiens annexin A10
(ANXA10), mRNA (SEQ ID NO: 32):
ATCCAGATTTGCTTTTACATTTTCTTGCCTGAGTC
TGAGGTGAACAGTGAACATATTTACATTTGATTTA
ACAGTGAACCTTAATTCTTTCTGGCTTCACAGTGA
AACAAGTTTATGCAATCGATCAAATATTTTCATCC
CTGAGGTTAACAATTACCATCAAAATGTTTTGTGG
AGACTATGTGCAAGGAACCATCTTCCCAGCTCCCA
ATTTCAATCCCATAATGGATGCCCAAATGCTAGGA
GGAGCACTCCAAGGATTTGACTGTGACAAAGACAT
GCTGATCAACATTCTGACTCAGCGCTGCAATGCAC
AAAGGATGATGATTGCAGAGGCATACCAGAGCATG
TATGGCCGGGACCTGATTGGGGATATGAGGGAGCA
GCTTTCGGATCACTTCAAAGATGTGATGGCTGGCC
TCATGTACCCACCACCACTGTATGATGCTCATGAG
CTCTGGCATGCCATGAAGGGAGTAGGCACTGATGA
GAATTGCCTCATTGAAATACTAGCTTCAAGAACAA
ATGGAGAAATTTTCCAGATGCGAGAAGCCTACTGC
TTGCAATACAGCAATAACCTCCAAGAGGACATTTA
TTCAGAGACCTCAGGACACTTCAGAGATACTCTCA
TGAACTTGGTCCAGGGGACCAGAGAGGAAGGATAT
ACAGACCCTGCGATGGCTGCTCAGGATGCAATGGT
CCTATGGGAAGCCTGTCAGCAGAAGACGGGGGAGC
ACAAAACCATGCTGCAAATGATCCTGTGCAACAAG
AGCTACCAGCAGCTGCGGCTGGTTTTCCAGGAATT
TCAAAATATTTCTGGGCAAGATATGGTAGATGCCA
TTAATGAATGTTATGATGGATACTTTCAGGAGCTG
CTGGTTGCAATTGTTCTCTGTGTTCGAGACAAACC
AGCCTATTTTGCTTATAGATTATATAGTGCAATTC
ATGACTTTGGTTTCCATAATAAAACTGTAATCAGG
ATTCTCATTGCCAGAAGTGAAATAGACCTGCTGAC
CATAAGGAAACGATACAAAGAGCGATATGGAAAAT
CCCTATTTCATGATATCAGAAATTTTGCTTCAGGG
CATTATAAGAAAGCACTGCTTGCCATCTGTGCTGG
TGATGCTGAGGACTACTAAAATGAAGAGGACTTGG
AGTACTGTGCACTCCTCTTTCTAGACACTTCCAAA
TAGAGATTTTCTCACAAATTTGTACTGTTCATGGC
ACTATTAACAAAACTATACAATCATATTTTCTCTT
CTATCTTTGAAATTATTCTAAGCCAAAGAAAACTA
TGAATGAAAGTATATGATACTGAATTTGCCTACTA
TCCTGAATTTGCCTACTATCTAATCAGCAATTAAA
TAAATTGTGCATGATGGAATAATAGAAAAATTGCA
TTGGAATAGATTTTATTTAAATGTGAACCATCAAC
AACCTACAACAA
NM_145868.2 Homo sapiens annexin
A11 (ANXA11), transcript variant b,
mRNA (SEQ ID NO: 33):
GGAGTTTTCCGCCCGGCGCTGACGGCTGCTGCGCC
CGCGGCTCCCCAGTGCCCCGAGTGCCCCGCGGGCC
CCGCGAGCGGGAGTGGGACCCAGCCCCTAGGCAGA
ACCCAGGCGCCGCGCCCGGGACGCCCGCGGAGAGA
GCCACTCCCGCCCACGTCCCATTTCGCCCCTCGCG
TCCGGAGTCCCCGTGGCCAGGTGTGTGTCTGGGGA
AGAGACTTACAGAAGTGGAGTTGCTGAGTCAAAGA
TCTAACCATGAGCTACCCTGGCTATCCCCCGCCCC
CAGGTGGCTACCCACCAGCTGCACCAGGTGGTGGT
CCCTGGGGAGGTGCTGCCTACCCTCCTCCGCCCAG
CATGCCCCCCATCGGGCTGGATAACGTGGCCACCT
ATGCGGGGCAGTTCAACCAGGACTATCTCTCGGGA
ATGGCGGCCAACATGTCTGGGACATTTGGAGGAGC
CAACATGCCCAACCTGTACCCTGGGGCCCCTGGGG
CTGGCTACCCACCAGTGCCCCCTGGCGGCTTTGGG
CAGCCCCCCTCTGCCCAGCAGCCTGTTCCTCCCTA
TGGGATGTATCCACCCCCAGGAGGAAACCCACCCT
CCAGGATGCCCTCATATCCGCCATACCCAGGGGCC
CCTGTGCCGGGCCAGCCCATGCCACCCCCCGGACA
GCAGCCCCCAGGGGCCTACCCTGGGCAGCCACCAG
TGACCTACCCTGGTCAGCCTCCAGTGCCACTCCCT
GGGCAGCAGCAGCCAGTGCCGAGCTACCCAGGATA
CCCGGGGTCTGGGACTGTCACCCCCGCTGTGCCCC
CAACCCAGTTTGGAAGCCGAGGCACCATCACTGAT
GCTCCCGGCTTTGACCCCCTGCGAGATGCCGAGGT
CCTGCGGAAGGCCATGAAAGGCTTCGGGACGGATG
AGCAGGCCATCATTGACTGCCTGGGGAGTCGCTCC
AACAAGCAGCGGCAGCAGATCCTACTTTCCTTCAA
GACGGCTTACGGCAAGGATTTGATCAAAGATCTGA
AATCTGAACTGTCAGGAAACTTTGAGAAGACAATC
TTGGCTCTGATGAAGACCCCAGTCCTCTTTGACAT
TTATGAGATAAAGGAAGCCATCAAGGGGGTTGGCA
CTGATGAAGCCTGCCTGATTGAGATCCTCGCTTCC
CGCAGCAATGAGCACATCCGAGAATTAAACAGAGC
CTACAAAGCAGAATTCAAAAAGACCCTGGAAGAGG
CCATTCGAAGCGACACATCAGGGCACTTCCAGCGG
CTCCTCATCTCTCTCTCTCAGGGAAACCGTGATGA
AAGCACAAACGTGGACATGTCACTCGCCCAGAGAG
ATGCCCAGGAGCTGTATGCGGCCGGGGAGAACCGC
CTGGGAACAGACGAGTCCAAGTTCAATGCGGTTCT
GTGCTCCCGGAGCCGGGCCCACCTGGTAGCAGTTT
TCAATGAGTACCAGAGAATGACAGGCCGGGACATT
GAGAAGAGCATCTGCCGGGAGATGTCCGGGGACCT
GGAGGAGGGCATGCTGGCCGTGGTGAAATGTCTCA
AGAATACCCCAGCCTTCTTTGCGGAGAGGCTCAAC
AAGGCCATGAGGGGGGCAGGAACAAAGGACCGGAC
CCTGATTCGCATCATGGTGTCTCGCAGCGAGACCG
ACCTCCTGGACATCAGATCAGAGTATAAGCGGATG
TACGGCAAGTCGCTGTACCACGACATCTCGGGAGA
TACTTCAGGGGATTACCGGAAGATTCTGCTGAAGA
TCTGTGGTGGCAATGACTGAACAGTGACTGGTGGC
TCACTTCTGCCCACCTGCCGGCAACACCAGTGCCA
GGAAAAGGCCAAAAGAATGTCTGTTTCTAACAAAT
CCACAAATAGCCCCGAGATTCACCGTCCTAGAGCT
TAGGCCTGTCTTCCACCCCTCCTGACCCGTATAGT
GTGCCACAGGACCTGGGTCGGTCTAGAACTCTCTC
AGGATGCCTTTTCTACCCCATCCCTCACAGCCTCT
TGCTGCTAAAATAGATGTTTCATTTTTCTGACTCA
TGCAATCATTCCCCTTTGCCTGTGGCTAAGACTTG
GCTTCATTTCGTCATGTAATTGTATATTTTTATTT
GGAGGCATATTTTCTTTTCTTACAGTCATTGCCAG
ACAGAGGCATACAAGTCTGTTTGCTGCATACACAT
TTCTGGTGAGGGCGACTGGGTGGGTGAAGCACCGT
GTCCTCGCTGAGGAGAGAAAGGGAGGCGTGCCTGA
GAAGGTAGCCTGTGCATCTGGTGAGTGTGTCACGA
GCTTTGTTACTGCCAAACTCACTCCTTTTTAGAAA
AAACAAAAAAAAAGGGCCAGAAAGTCATTCCTTCC
ATCTTCCTTGCAGAAACCACGAGAACAAAGCCAGT
TCCCTGTCAGTGACAGGGCTTCTTGTAATTTGTGG
TATGTGCCTTAAACCTGAATGTCTGTAGCCAAAAC
TTGTTTCCACATTAAGAGTCAGCCAGCTCTGGAAT
GGTCTGGAAATGTCTTCCTGGTACCAACTTGTTTT
CTTCTGCTTGATTCTGCCCTGTGGCTCAGAGGTCT
GGCCTTATCAGCCAGTGAAAGTTCATGTAACCTTA
CGTAGAGATTTGTGTGCAGGAAACCCTGAGCATAC
ACTAGTTTGCAGGGACTCGTAAGGACATGGGAAGG
GAGGTTCCCGAAATCCAGGCAGGAGGCCCAGACAC
CTGAAAGGCAAAGGGATCTTGGTTGGTTGCAGGTG
CAGTGAAGTCCACTGAAGGTGTGGTGCGAAGAATG
CAGTCCTTCACCCAGGTCCCAGGAGGGAAGAAGGG
TGTGTGCTAATTCCTGGTGCCCCTCGGCGGGGGCC
AGAGAGAAGGATGGGGACAACCCAGAGAGTCACAA
GACCAGTGCCTCCCCTCAGGGTGCCTCCAGGCTGA
AAGGGGCTCCTGGCTCTGGTCTCTGGGGACCCTGT
GCCCGTTGGTTGGTGGTGTGAGGGAAGAGAATCCA
TAAGAGAGTTTCTGAGAATTATGGTGTCATGTCCA
GAAGCTAGAGCTTACCTTGCATCAGGGGTCTCCAC
CCACTCCTTTTCCAACCTCCTGCGTTGAGGTTTAG
AAAAGAGAGAATCGACTAGGCACTATGGCTCACGC
CTGTAATCCAAGGACTTTGGGAAGCTGAGGTGAGA
GGATCACTTGAGCTCAGGAGTTCAAGACTAGCCTA
GCCAACAGCGAGACCCCTGTCTCTACTAAAAAATT
TGGCCAGGCGTGGTGGCTCACGGCTGTAATCCCAG
CACTTTGGGAGGTGAGGCGGGCAGATCACCTGAGG
TCAGGAGTTCGAGACCCAGCCTGGCCAACATGGTG
AAACCCCATCTCTACTAAAAATACAAAAATTAGCC
AGGCATGGTGGCACATTCCTGTAATCCCAGCTACA
CAGGATGCTGAGGCAGGAGAATCACTTGAACCCAG
GAGGCAGAGGTTGTAGTGAGCTGAGATCACACCAT
TGCACTTCAACCTGGGTGGACAGAGTGAGACTCTG
TCTCAAAAAAAAAAAAAAATTTACCTGGCATTGTA
GTGCATTCCCTATAGTCGGCTACTCTGGAGGCTGA
GGCAGGAAGATCCTTAGAGCCCAAGAAATTGAGGC
CGTAGTAAGCTGTGATTACACCACTGCACTCCAGC
CTGGACAACAGAGCGAGACCTTGTCTCAAATGAGA
AAAAAACAAAAAGAAATGGGAGAATCCAGAGAGAC
TAGGCTAGATCAAGCCTGCTGGGTCCTGGCAGGAG
CCCCAGGGAGTAGCTCATCTGCAGACATTTGCTTG
AGGACTACCCCCTAAACATAAAGGAAGAATGACAT
CCGAAGGGTGTGGAGCAGCCATGAGCTGAGAACTA
GCCTGGTCTACCTGAGATTGATGGCAGGTCCTGGT
CAACACGTCAGCTCTGCGTCAGAGTCCATGCCTCA
AGCCCAAGCTGAAGCCCCATCCCTGCTGCTCTCCC
AAGAACTCCTCTGCTAGGGCAGGCCCCTTGCCCTT
GGGTGCCAGGTGGGACCTGCCTGATGGGATGGGGT
GCTTGGCATATACAACTTGCCATGAACTCAAGGTG
ACCCTGGGGGCCTCCTGAATTGTGATGGGGCCTAG
AACCAATGTGCTCTGATGTGACCATATTCTGTGAC
ATTACCTTGCCCTGTTTACTCCAAAGTTCCCAGCC
TGGTGCCCAGCAGGCAATATTGCACCTACAGACAC
ATTTACTTTGGTTTCCAAAGTGTTTTTAGACATTT
GAATTTGTTGCCAACATTTAAACATTGAGAGATTT
CATATTTTTAAAAATCTGGAATTCTGGCTTCTCTT
GAAAACTCAGAAATTCTGGCACTATGGGGCTTGCA
TTCCTGCATGGCTGGAGCTGAGTTGCAGCTGCCCC
TTTAGGCCTGTACTCCTTATTTGCTATAGGCTCCG
TCTTGTATTACACTAAGCCCATGTCACCCATTTGG
CTCCTGCAGGCCTTTGGGTTTGAGACCCTGGTCTA
CACACTTGGAGACCACCTGTTGTAAAGTACATGGA
TGTGCTTTGGTCAAGGAATAGACCAAGGTGGATAT
CCAGGCCAGAGTGACTCAGCGAGTTTAGGTCACAG
GCGTATACTCCACTTGTTATATAACCTGCTTGTGT
AAGTTCATACTTGGCTCAAAGCCACTATTGTTTGG
AAAAGGTATAACTGCCCTGCTGACGCTGTACAGAT
GTTCTTGGGCTCGGATGGGCATGGCTCCACGTGGT
GTGCACTAGCACCCAGAGAGAGTGAAGCTATTGAC
CCCTGTAAGGGAGAGTGACCATCTGGCAGATAGAT
AGAGGGGAGCCAGGACATGGCTCAGCTTGTGCCCA
GAGGGAGAGTTAAGCCGCTGACCCTGTAGCCAGGG
AGTGCACCTGCAAGCATGGGGGTGGCAGGAGCCAC
AGAGCTGGCTGCTGAGAGGAGCTGCAGATCTGGAG
AAGACAGCCTAGGTAAAGGTGGACAGTGTGAGAGC
TGCTGATGAGATAGCTGCTGAATAAAACTACATTT
TACCTGCCTATGGCCCGCCAGGTTTTCTTTCAGCT
ATCGCCCATCCACCCAGTCCCCTCGAACCTCAGCA
TGGGCTGGAACCTGACCCTGGGCATGACATTTGGC
ATAGTTGTGGACCTGACACCTGTGTTTGTCCTAGT
CCTGTTTCTCCCTGCCTTCCTGTTCCTCTCGCTGC
CCTCATGGTCACTCCCAAGAGATCCAACCCATGTT
AAGTATGGGCTGGAGGACTGCATGAATGCCTCATG
ATCTTCCCAGAGGCAAAGGCACCTACTGCCTTCCA
AGGTCAGTGGGAGGTTGGGATCAACACTGTTTATT
ATGCTTAGGACAAAAAAGATAGGGAGAAAGATGTG
CAACCTTACAGGTCATCTTTCTGGGATAGAACACA
ATGGGTCTTCTCCTGCCTCCTGGATATGTTAGTCA
AGGCCAGTCCATGCTACACATCTAGTCTGACTTCT
AAAATAGAAGCACCAGATGAATTCAGCCCTGAGAG
AATTTTCAGCAGCTGTGGGGGCGCTGGAGGAAACA
CTATTAAATAGTTTTGCACCTGAGACAGATAGCCT
CACTCGCCTCACCCTAGTCCTGGTGGCATTTGTCT
CAGGTGCAAAATTTAAGAAAGAAACCTTGGAGTGC
TCACCCTGTGGCTGGGTAGATGGTCCTAAAGTGGT
GGTTTTCAAGCCTGAGTGTGTATCAGGATCATCAG
GGGAGCTTGCTAAAGAGCAGTTCCTGCGGTCAGAC
CCTCATGCATTTTGAGCAGGTGTGGGGACTGGGAA
ACTGCATCTGTAACCTGCTGTAATCTAACGCTTAT
CTAAATACTACTGTGCTCACACAGAGAACACCGCA
AAAGTAGAGGTGTTCCTCCAGAGGGCAGGTGAGCA
GATGGCACAGTCTGCTTGGAATTCAGTCAGGTGAT
GAGAGATGAGATGAGGCACTCCTAGCTTTGGGAAG
AGGGAGCTGAAAGATGAACCTTTGCAGGTGCCCAC
GGTCAAAGTGGTGGTTTAATGCCATGCCATGCCCA
TTTTCTGTTGGCCTTGGCAGGGAGTTACAGCCCTA
CCTTAGGACCTGGCTCCTTATTTCTGCTGTAGGCT
CTTTCCTGCCCTGGCCGAGATGGAGTGGAATGAGA
CCTAGAAACATCAAGCTAAATACATGTCCTCAGAA
AGATAAAGGTTTACATTTTCACCCCCATCAAATCT
GAAAGCTCTCTGCCTGTGTTTTTCTAAGGGATAGG
GACATCATTACTCAGTCCACAACCTGGACTCATGT
AGGGTCCCCTGTCAGTAAAGGAGTCAGTCAAGCCC
ACCAGGTATACCAAGGACTCTTACCCTCAGCCCCT
ACTCCTTGGAAAGCTGCCCCTTGGCCTAATATTGG
TGTTTAGCTTGAGCCTGACTCCTTCTCAACACTAA
GAGCTGATGAAGTCCTGAAGCAGAAAGAGCTCTGA
CCTGAGAGTCAAACATCCTTATTCTGATCTCAGCT
CAGCCCCTGATTTGTTGTGTGACCCTGGATATGTC
ACTTCCTGTCTTTTTGACTTTTTAAAATGAAGGGT
AGACTAGAGGAGAGCTTCTAAAACTTTAATGTGGT
CAACGAAATGGAATAGGAAATTCCACAAGTCTGTC
CTTCCACAAAAGCAGCAAATAAGGTGGCAAAAACT
CAAATTTATGGGAACTCTGGAAACGAATTGAAAGT
TTACAGCAATCAGGTGAATACCTAAGAATAAAAGC
TGGATTTAGTAAGA
NM_001278409.1 Homo sapiens annexin
A11 (ANXA11), transcript variant f,
mRNA (SEQ ID NO: 34):
GCACTGCCTCTGGCACCTGGGGCAGCCGCGCCCGC
GGAGTTTTCCGCCCGGCGCTGACGGCTGCTGCGCC
CGCGGCTCCCCAGTGCCCCGAGTGCCCCGCGGGCC
CCGCGAGCGGGAGTGGGACCCAGCCCCTAGGCAGA
ACCCAGGCGCCGCGCCCGGGACGCCCGCGGAGAGA
GCCACTCCCGCCCACGTCCCATTTCGCCCCTCGCG
TCCGGAGTCCCCGTGGCCAGGTGTGTGTCTGGGGA
AGAGACTTACAGAAGTGGAGTTGCTGAGTCAAAGA
TCTAACCATGAGCTACCCTGGCTATCCCCCGCCCC
CAGGTGGCTACCCACCAGCTGCACCAGGTTGGCTG
GCACTGGCCTGGGTTCTCTCTCTATAGTAGAAATC
CTGCCATCCAGATCCTGCCACTGCCACCTTTGCTA
GCACAGCTGAGCAGCCTCTGAGCAGCAAGAGAGGA
GGAGGCAGGAAATTTAGGGAAGGTTCTTCCTGGAG
GGTCTGGAGCCCTGGAGATGAAGAGCCGATCCGAA
GCTGCCATGTAGAGGAAAGCATCTAACAGGCCAGA
GGCCCCATGATGATGTCGAATGCCCATCGGGCACC
CAGCTGAGCCCTGCAGGTGGTGGTCCCTGGGGAGG
TGCTGCCTACCCTCCTCCGCCCAGCATGCCCCCCA
TCGGGCTGGATAACGTGGCCACCTATGCGGGGCAG
TTCAACCAGGACTATCTCTCGGGAATGGCGGCCAA
CATGTCTGGGACATTTGGAGGAGCCAACATGCCCA
ACCTGTACCCTGGGGCCCCTGGGGCTGGCTACCCA
CCAGTGCCCCCTGGCGGCTTTGGGCAGCCCCCCTC
TGCCCAGCAGCCTGTTCCTCCCTATGGGATGTATC
CACCCCCAGGAGGAAACCCACCCTCCAGGATGCCC
TCATATCCGCCATACCCAGGGGCCCCTGTGCCGGG
CCAGCCCATGCCACCCCCCGGACAGCAGCCCCCAG
GGGCCTACCCTGGGCAGCCACCAGTGACCTACCCT
GGTCAGCCTCCAGTGCCACTCCCTGGGCAGCAGCA
GCCAGTGCCGAGCTACCCAGGATACCCGGGGTCTG
GGACTGTCACCCCCGCTGTGCCCCCAACCCAGTTT
GGAAGCCGAGGCACCATCACTGATGCTCCCGGCTT
TGACCCCCTGCGAGATGCCGAGGTCCTGCGGAAGG
CCATGAAAGGCTTCGGGACGGATGAGCAGGCCATC
ATTGACTGCCTGGGGAGTCGCTCCAACAAGCAGCG
GCAGCAGATCCTACTTTCCTTCAAGACGGCTTACG
GCAAGGATTTGATCAAAGATCTGAAATCTGAACTG
TCAGGAAACTTTGAGAAGACAATCTTGGCTCTGAT
GAAGACCCCAGTCCTCTTTGACATTTATGAGATAA
AGGAAGCCATCAAGGGGGTTGGCACTGATGAAGCC
TGCCTGATTGAGATCCTCGCTTCCCGCAGCAATGA
GCACATCCGAGAATTAAACAGAGCCTACAAAGCAG
AATTCAAAAAGACCCTGGAAGAGGCCATTCGAAGC
GACACATCAGGGCACTTCCAGCGGCTCCTCATCTC
TCTCTCTCAGGGAAACCGTGATGAAAGCACAAACG
TGGACATGTCACTCGCCCAGAGAGATGCCCAGGAG
CTGTATGCGGCCGGGGAGAACCGCCTGGGAACAGA
CGAGTCCAAGTTCAATGCGGTTCTGTGCTCCCGGA
GCCGGGCCCACCTGGTAGCAGTTTTCAATGAGTAC
CAGAGAATGACAGGCCGGGACATTGAGAAGAGCAT
CTGCCGGGAGATGTCCGGGGACCTGGAGGAGGGCA
TGCTGGCCGTGGTGAAATGTCTCAAGAATACCCCA
GCCTTCTTTGCGGAGAGGCTCAACAAGGCCATGAG
GGGGGCAGGAACAAAGGACCGGACCCTGATTCGCA
TCATGGTGTCTCGCAGCGAGACCGACCTCCTGGAC
ATCAGATCAGAGTATAAGCGGATGTACGGCAAGTC
GCTGTACCACGACATCTCGGGAGATACTTCAGGGG
ATTACCGGAAGATTCTGCTGAAGATCTGTGGTGGC
AATGACTGAACAGTGACTGGTGGCTCACTTCTGCC
CACCTGCCGGCAACACCAGTGCCAGGAAAAGGCCA
AAAGAATGTCTGTTTCTAACAAATCCACAAATAGC
CCCGAGATTCACCGTCCTAGAGCTTAGGCCTGTCT
TCCACCCCTCCTGACCCGTATAGTGTGCCACAGGA
CCTGGGTCGGTCTAGAACTCTCTCAGGATGCCTTT
TCTACCCCATCCCTCACAGCCTCTTGCTGCTAAAA
TAGATGTTTCATTTTTCTGACTCATGCAATCATTC
CCCTTTGCCTGTGGCTAAGACTTGGCTTCATTTCG
TCATGTAATTGTATATTTTTATTTGGAGGCATATT
TTCTTTTCTTACAGTCATTGCCAGACAGAGGCATA
CAAGTCTGTTTGCTGCATACACATTTCTGGTGAGG
GCGACTGGGTGGGTGAAGCACCGTGTCCTCGCTGA
GGAGAGAAAGGGAGGCGTGCCTGAGAAGGTAGCCT
GTGCATCTGGTGAGTGTGTCACGAGCTTTGTTACT
GCCAAACTCACTCCTTTTTAGAAAAAACAAAAAAA
AAGGGCCAGAAAGTCATTCCTTCCATCTTCCTTGC
AGAAACCACGAGAACAAAGCCAGTTCCCTGTCAGT
GACAGGGCTTCTTGTAATTTGTGGTATGTGCCTTA
AACCTGAATGTCTGTAGCCAAAACTTGTTTCCACA
TTAAGAGTCAGCCAGCTCTGGAATGGTCTGGAAAT
GTCA
NM_004306.4 Homo sapiens annexin A13
(ANXA13), transcript variant 1, mRNA
(SEQ ID NO: 35):
GCCTGTAGGAGGACTGATCTCTTGATGAAATACAG
AAAAACCATCTCAGAAAAAGGAAAATGGGCAATCG
TCATGCTAAAGCGAGCAGTCCTCAGGGTTTTGATG
TGGATCGAGATGCCAAAAAGCTGAACAAAGCCTGC
AAAGGAATGGGGACCAATGAAGCAGCCATCATTGA
AATCTTATCGGGCAGGACATCAGATGAGAGGCAAC
AAATCAAGCAAAAGTACAAGGCAACGTACGGCAAG
GAGCTGGAGGAAGTACTCAAGAGTGAGCTGAGTGG
AAACTTCGAGAAGACAGCGTTGGCCCTTCTGGACC
GTCCCAGCGAGTACGCCGCCCGGCAGCTGCAGAAG
GCTATGAAGGGTCTGGGCACAGATGAGTCCGTCCT
CATTGAGGTCCTGTGCACGAGGACCAATAAGGAAA
TCATCGCCATTAAAGAGGCCTACCAAAGGCTATTT
GATAGGAGCCTCGAATCAGATGTCAAAGGTGATAC
AAGTGGAAACCTAAAAAAAATCCTGGTGTCTCTGC
TGCAGGCTAATCGCAATGAAGGAGATGACGTGGAC
AAAGATCTAGCTGGTCAGGATGCCAAAGATCTGTA
TGATGCAGGGGAAGGCCGCTGGGGCACTGATGAGC
TTGCGTTCAATGAAGTCCTGGCCAAGAGGAGCTAC
AAGCAGTTACGAGCCACCTTTCAAGCCTATCAAAT
TCTCATTGGCAAAGACATAGAAGAAGCCATTGAAG
AAGAAACATCAGGCGACTTGCAGAAGGCCTATTTA
ACTCTCGTGAGATGTGCCCAGGATTGTGAGGACTA
TTTTGCTGAACGTCTGTACAAGTCGATGAAGGGTG
CGGGGACCGATGAGGAGACGTTGATTCGCATAGTC
GTGACCAGGGCCGAGGTGGACCTTCAGGGGATCAA
AGCAAAGTTCCAAGAGAAGTATCAGAAGTCTCTCT
CTGACATGGTTCGCTCAGATACCTCCGGGGACTTC
CGGAAACTGCTAGTAGCCCTCTTGCACTGAGCCAA
GCCAGGGCAATAGGAACACAGGGTGGAACCGCCTT
TGTCAAGAGCACATTCCAAATCAAACTTGCAAATG
AGACTCCCGCACGAAAACCCTTAAGAGTCCCGGAT
TACTTTCTTGGCAGCTTAAGTGGCGCAGCCAGGCC
AAGCTGTGTAAGTTAAGGGCAGTAACGTTAAGATG
CGTGGGCAGGGCACCTTGAACTCTGGCTTAGCAAG
CATCTAGGCTGCCTCTTCACTTTCTTTTAGCATGG
TAACTGGATGTTTTCTAAACACTAATGAAATCAGC
AGTTGATGAAAAAACTATGCATTTGTAATGGCACA
TTTAGAAGGATATGCATCACACAAGTAAGGTACAG
GAAAGACAAAATTAAACAATTTATTAATTTTCCTT
CTGTGTGTTCAATTTGAAAGCCTCATTGTTAATTA
AAGTTGTGGATTATGCCTCTA
NM_001003954.2 Homo sapiens annexin A13
(ANXA13), transcript variant 2, mRNA
(SEQ ID NO: 36):
ATTATGTCCGGGGGGAAAACTGTTGTAAACTTTGC
CTGTAGGAGGACTGATCTCTTAATGAAATACAGAA
AAACCATCTCAGAAAAAGGAAAATGGGCAATCGTC
ATAGCCAGTCGTACACCCTCTCAGAAGGCAGTCAA
CAGTTGCCTAAAGGGGACTCCCAACCCTCGACAGT
CGTGCAGCCTCTCAGCCACCCATCACGGAATGGAG
AGCCAGAGGCCCCACAGCCTGCTAAAGCGAGCAGT
CCTCAGGGTTTTGATGTGGATCGAGATGCCAAAAA
GCTGAACAAAGCCTGCAAAGGAATGGGGACCAATG
AAGCAGCCATCATTGAAATCTTATCGGGCAGGACA
TCAGATGAGAGGCAACAAATCAAGCAAAAGTACAA
GGCAACGTACGGCAAGGAGCTGGAGGAAGTACTCA
AGAGTGAGCTGAGTGGAAACTTCGAGAAGACAGCG
TTGGCCCTTCTGGACCGTCCCAGCGAGTACGCCGC
CCGGCAGCTGCAGAAGGCTATGAAGGGTCTGGGCA
CAGATGAGTCCGTCCTCATTGAGGTCCTGTGCACG
AGGACCAATAAGGAAATCATCGCCATTAAAGAGGC
CTACCAAAGGCTATTTGATAGGAGCCTCGAATCAG
ATGTCAAAGGTGATACAAGTGGAAACCTAAAAAAA
ATCCTGGTGTCTCTGCTGCAGGCTAATCGCAATGA
AGGAGATGACGTGGACAAAGATCTAGCTGGTCAGG
ATGCCAAAGATCTGTATGATGCAGGGGAAGGCCGC
TGGGGCACTGATGAGCTTGCGTTCAATGAAGTCCT
GGCCAAGAGGAGCTACAAGCAGTTACGAGCCACCT
TTCAAGCCTATCAAATTCTCATTGGCAAAGACATA
GAAGAAGCCATTGAAGAAGAAACATCAGGCGACTT
GCAGAAGGCCTATTTAACTCTCGTGAGATGTGCCC
AGGATTGTGAGGACTATTTTGCTGAACGTCTGTAC
AAGTCGATGAAGGGTGCGGGGACCGATGAGGAGAC
GTTGATTCGCATAGTCGTGACCAGGGCCGAGGTGG
ACCTTCAGGGGATCAAAGCAAAGTTCCAAGAGAAG
TATCAGAAGTCTCTCTCTGACATGGTTCGCTCAGA
TACCTCCGGGGACTTCCGGAAACTGCTAGTAGCCC
TCTTGCACTGAGCCAAGCCAGGGCAATAGGAACAC
AGGGTGGAACCGCCTTTGTCAAGAGCACATTCCAA
ATCAAACTTGCAAATGAGACTCCCGCACGAAAACC
CTTAAGAGTCCCGGATTACTTTCTTGGCAGCTTAA
GTGGCGCAGCCAGGCCAAGCTGTGTAAGTTAAGGG
CAGTAACGTTAAGATGCGTGGGCAGGGCACCTTGA
ACTCTGGCTTAGCAAGCATCTAGGCTGCCTCTTCA
CTTTCTTTTAGCATGGTAACTGGATGTTTTCTAAA
CACTAATGAAATCAGCAGTTGATGAAAAAACTATG
CATTTGTAATGGCACATTTAGAAGGATATGCATCA
CACAAGTAAGGTACAGGAAAGACAAAATTAAACAA
TTTATTAATTTTCCTTCTGTGTGTTCAATTTGAAA
GCCTCATTGTTAATTAAAGTTGTGGATTATGCCTC
TAAAAAAAAAAAAAAAAAAAAAA
NM_001363114.2 Homo sapiens annexin A6
(ANXA6), transcript variant 3, mRNA
(SEQ ID NO: 45):
GCGGTTGCTGCTGGGCTAACGGGCTCCGATCCAGC
GAGCGCTGCGTCCTCGAGTCCCTGCGCCCGTGCGT
CCGTCTGCGACCCGAGGCCTCCGCTGCGCGTGGAT
TCTGCTGCGAACCGGAGACCATGGCCAAACCAGCA
CAGGGTGCCAAGTACCGGGGCTCCATCCATGACTT
CCCAGGCTTTGACCCCAACCAGGATGCCGAGGCTC
TGTACACTGCCATGAAGGGCTTTGGCAGTGACAAG
GAGGCCATACTGGACATAATCACCTCACGGAGCAA
CAGGCAGAGGCAGGAGGTCTGCCAGAGCTACAAGT
CCCTCTACGGCAAGGACCTCATTGCTGATTTAAAG
TATGAATTGACGGGCAAGTTTGAACGGTTGATTGT
GGGCCTGATGAGGCCACCTGCCTATTGTGATGCCA
AAGAAATTAAAGATGCCATCTCGGGCATTGGCACT
GATGAGAAGTGCCTCATTGAGATCTTGGCTTCCCG
GACCAATGAGCAGATGCACCAGCTGGTGGCAGCAT
ACAAAGATGCCTACGAGCGGGACCTGGAGGCTGAC
ATCATCGGCGACACCTCTGGCCACTTCCAGAAGAT
GCTTGTGGTCCTGCTCCAGGGAACCAGGGAGGAGG
ATGACGTAGTGAGCGAGGACCTGGTACAACAGGAT
GTCCAGGACCTATACGAGGCAGGGGAACTGAAATG
GGGAACAGATGAAGCCCAGTTCATTTACATCTTGG
GAAATCGCAGCAAGCAGCATCTTCGGTTGGTGTTC
GATGAGTATCTGAAGACCACAGGGAAGCCGATTGA
AGCCAGCATCCGAGGGGAGCTGTCTGGGGACTTTG
AGAAGCTAATGCTGGCCGTAGTGAAGTGTATCCGG
AGCACCCCGGAATATTTTGCTGAAAGGCTCTTCAA
GGCTATGAAGGGCCTGGGGACTCGGGACAACACCC
TGATCCGCATCATGGTCTCCCGTAGTGAGTTGGAC
ATGCTCGACATTCGGGAGATCTTCCGGACCAAGTA
TGAGAAGTCCCTCTACAGCATGATCAAGAATGACA
CCTCTGGCGAGTACAAGAAGACTCTGCTGAAGCTG
TCTGGGGGAGATGATGATGCTGCTGGCCAGTTCTT
CCCGGAGGCAGCGCAGGTGGCCTATCAGATGTGGG
AACTTAGTGCAGTGGCCCGAGTAGAGCTGAAGGGA
ACTGTGCGCCCAGCCAATGACTTCAACCCTGACGC
AGATGCCAAAGCGCTGCGGAAAGCCATGAAGGGAC
TCGGGACTGACGAAGACACAATCATCGATATCATC
ACGCACCGCAGCAATGTCCAGCGGCAGCAGATCCG
GCAGACCTTCAAGTCTCACTTTGGCCGGGACTTAA
TGACTGACCTGAAGTCTGAGATCTCTGGAGACCTG
GCAAGGCTGATTCTGGGGCTCATGATGCCACCGGC
CCATTACGATGCCAAGCAGTTGAAGAAGGCCATGG
AGGGAGCCGGCACAGATGAAAAGGCTCTTATTGAA
ATCCTGGCCACTCGGACCAATGCTGAAATCCGGGC
CATCAATGAGGCCTATAAGGAGGACTATCACAAGT
CCCTGGAGGATGCTCTGAGCTCAGACACATCTGGC
CACTTCAGGAGGATCCTCATTTCTCTGGCCACGGG
GCATCGTGAGGAGGGAGGAGAAAACCTGGACCAGG
CACGGGAAGATGCCCAGGAAATAGCAGACACACCT
AGTGGAGACAAAACTTCCTTGGAGACACGTTTCAT
GACGATCCTGTGTACCCGGAGCTATCCGCACCTCC
GGAGAGTCTTCCAGGAGTTCATCAAGATGACCAAC
TATGACGTGGAGCACACCATCAAGAAGGAGATGTC
TGGGGATGTCAGGGATGCATTTGTGGCCATTGTTC
AAAGTGTCAAGAACAAGCCTCTCTTCTTTGCCGAC
AAACTTTACAAATCCATGAAGGGTGCTGGCACAGA
TGAGAAGACTCTGACCAGGATCATGGTATCCCGCA
GTGAGATTGACCTGCTCAACATCCGGAGGGAATTC
ATTGAGAAATATGACAAGTCTCTCCACCAAGCCAT
TGAGGGTGACACCTCCGGAGACTTCCTGAAGGCCT
TGCTGGCTCTCTGTGGTGGTGAGGACTAGGGCCAC
AGCTTTGGCGGGCACTTCTGCCAAGAAATGGTTAT
CAGCACCAGCCGCCATGGCCAAGCCTGATTGTTCC
AGCTCCAGAGACTAAGGAAGGGGCAGGGGTGGGGG
GAGGGGTTGGGTTGGGCTCTTATCTTCAGTGGAGC
TTAGGAAACGCTCCCACTCCCACGGGCCATCGAGG
GCCCAGCACGGCTGAGCGGCTGAAAAACCGTAGCC
ATAGATCCTGTCCACCTCCACTCCCCTCTGACCCT
CAGGCTTTCCCAGCTTCCTCCCCTTGCTACAGCCT
CTGCCCTGGTTTGGGCTATGTCAGATCCAAAAACA
TCCTGAACCTCTGTCTGTAAAATGAGTAGTGTCTG
TACTTTGAATGAGGGGGTTGGTGGCAGGGGCCAGT
TGAATGTGCTGGGCGGGGTGGTGGGAAGGATAGTA
AATGTGCTGGGGCAAACTGACAAATCTTCCCATCC
ATTTCACCACCCATCTCCATCCAGGCCGCGCTAGA
GTACTGGACCAGGAATTTGGATGCCTGGGTTCAAA
TCTGCATCTGCCATGCACTTGTTTCTGACCTTAGG
CCAGCCCCTTTCCCTCCCTGAGTCTCTATTTTCTT
ATCTACAATGAGACAGTTGGACAAAAAAATCTTGG
CTTCCCTTCTAACATTAACTTCCTAAAGTATGCCT
CCGATTCATTCCCTTGACACTTTTTATTTCTAAGG
AAGAAATAAAAAGAGATACACAAACACATAAACACA

### Polynucleotides

In some embodiments, an agent of the disclosure that increases activity of an annexin protein is a polynucleotide capable of expressing an annexin protein as described herein. The term “nucleotide” or its plural as used herein is interchangeable with modified forms as discussed herein and otherwise known in the art. In certain instances, the art uses the term “nucleobase” which embraces naturally-occurring nucleotide, and non-naturally-occurring nucleotides which include modified nucleotides. Thus, nucleotide or nucleobase means the naturally occurring nucleobases A, G, C, T, and U. Non-naturally occurring nucleobases include, for example and without limitations, xanthine, diaminopurine, 8-oxo-N6-methyladenine, 7-deazaxanthine, 7-deazaguanine, N4,N4-ethanocytosin, N′,N′-ethano-2,6-diaminopurine, 5-methylcytosine (mC), 5-(C3-C6)-alkynyl-cytosine, 5-fluorouracil, 5-bromouracil, pseudoisocytosine, 2-hydroxy-5-methyl-4-tr-iazolopyridin, isocytosine, isoguanine, inosine and the “non-naturally occurring” nucleobases described in Benner et al., U.S. Pat. No. 5,432,272 and Susan M. Freier and Karl-Heinz Altmann, 1997, Nucleic Acids Research, vol. 25: pp 4429-4443. The term “nucleobase” also includes not only the known purine and pyrimidine heterocycles, but also heterocyclic analogues and tautomers thereof. Further naturally and non-naturally occurring nucleobases include those disclosed in U.S. Pat. No. 3,687,808 (Merigan, et al.), in Chapter 15 by Sanghvi, in Antisense Research and Application, Ed. S. T. Crooke and B. Lebleu, CRC Press, 1993, in Englisch et al., 1991, Angewandte Chemie, International Edition, 30: 613-722 (see especially pages 622 and 623, and in the Concise Encyclopedia of Polymer Science and Engineering, J. I. Kroschwitz Ed., John Wiley & Sons, 1990, pages 858-859, Cook, Anti-Cancer Drug Design 1991, 6, 585-607, each of which are hereby incorporated by reference in their entirety). In various aspects, polynucleotides also include one or more “nucleosidic bases” or “base units” which are a category of non-naturally-occurring nucleotides that include compounds such as heterocyclic compounds that can serve like nucleobases, including certain “universal bases” that are not nucleosidic bases in the most classical sense but serve as nucleosidic bases. Universal bases include 3-nitropyrrole, optionally substituted indoles (e.g., 5-nitroindole), and optionally substituted hypoxanthine. Other desirable universal bases include, pyrrole, diazole or triazole derivatives, including those universal bases known in the art.

Modified nucleotides are described in EP 1 072 679 and WO 97/12896, the disclosures of which are incorporated herein by reference. Modified nucleobases include without limitation, 5-methylcytosine (5-me-C), 5-hydroxymethyl cytosine, xanthine, hypoxanthine, 2-aminoadenine, 6-methyl and other alkyl derivatives of adenine and guanine, 2-propyl and other alkyl derivatives of adenine and guanine, 2-thiouracil, 2-thiothymine and 2-thiocytosine, 5-halouracil and cytosine, 5-propynyl uracil and cytosine and other alkynyl derivatives of pyrimidine bases, 6-azo uracil, cytosine and thymine, 5-uracil (pseudouracil), 4-thiouracil, 8-halo, 8-amino, 8-thiol, 8-thioalkyl, 8-hydroxyl and other 8-substituted adenines and guanines, 5-halo particularly 5-bromo, 5-trifluoromethyl and other 5-substituted uracils and cytosines, 7-methylguanine and 7-methyladenine, 2-F-adenine, 2-amino-adenine, 8-azaguanine and 8-azaadenine, 7-deazaguanine and 7-deazaadenine and 3-deazaguanine and 3-deazaadenine. Further modified bases include tricyclic pyrimidines such as phenoxazine cytidine(1H-pyrimido[5,4-b][1,4]benzoxazin-2(3H)-one), phenothiazine cytidine (1H-pyrimido[5,4-b][1,4]benzothiazin-2(3H)-one), G-clamps such as a substituted phenoxazine cytidine (e.g. 9-(2-aminoethoxy)-H-pyrimido[5,4-b][1,4]benzox-azin-2(3H)-one), carbazole cytidine (2H-pyrimido[4,5-b]indol-2-one), pyridoindole cytidine (H-pyrido[3′,2′:4,5]pyrrolo[2,3-d]pyrimidin-2-one). Modified bases may also include those in which the purine or pyrimidine base is replaced with other heterocycles, for example 7-deaza-adenine, 7-deazaguanosine, 2-aminopyridine and 2-pyridone. Additional nucleobases include those disclosed in U.S. Pat. No. 3,687,808, those disclosed in The Concise Encyclopedia Of Polymer Science And Engineering, pages 858-859, Kroschwitz, J. I., ed. John Wiley & Sons, 1990, those disclosed by Englisch et al., 1991, Angewandte Chemie, International Edition, 30: 613, and those disclosed by Sanghvi, Y. S., Chapter 15, Antisense Research and Applications, pages 289-302, Crooke, S. T. and Lebleu, B., ed., CRC Press, 1993. Certain of these bases are useful for increasing binding affinity and include 5-substituted pyrimidines, 6-azapyrimidines and N-2, N-6 and 0-6 substituted purines, including 2-aminopropyladenine, 5-propynyluracil and 5-propynylcytosine. 5-methylcytosine substitutions have been shown to increase nucleic acid duplex stability by 0.6-1.2° C. and are, in certain aspects combined with 2′-O-methoxyethyl sugar modifications. See, U.S. Pat. Nos. 3,687,808, 4,845,205; 5,130,302; 5,134,066; 5,175,273; 5,367,066; 5,432,272; 5,457,187; 5,459,255; 5,484,908; 5,502,177; 5,525,711; 5,552,540; 5,587,469; 5,594,121, 5,596,091; 5,614,617; 5,645,985; 5,830,653; 5,763,588; 6,005,096; 5,750,692 and 5,681,941, the disclosures of which are incorporated herein by reference.

Methods of making polynucleotides of a predetermined sequence are well-known. See, e.g., Sambrook et al., Molecular Cloning: A Laboratory Manual (2nd ed. 1989) and F. Eckstein (ed.) Oligonucleotides and Analogues, 1st Ed. (Oxford University Press, New York, 1991). Solid-phase synthesis methods are preferred for both polyribonucleotides and polydeoxyribonucleotides (the well-known methods of synthesizing DNA are also useful for synthesizing RNA). Polynucleotides and polyribonucleotides can also be prepared enzymatically via, e.g., polymerase chain reaction (PCR). Non-naturally occurring nucleobases can be incorporated into the polynucleotide, as well. See, e.g., U.S. Pat. No. 7,223,833; Katz, J. Am. Chem. Soc., 74:2238 (1951); Yamane, et al., J. Am. Chem. Soc., 83:2599 (1961); Kosturko, et al., Biochemistry, 13:3949 (1974); Thomas, J. Am. Chem. Soc., 76:6032 (1954); Zhang, et al., J. Am. Chem. Soc., 127:74-75 (2005); and Zimmermann, et al., J. Am. Chem. Soc., 124:13684-13685 (2002).

In various embodiments, a polynucleotide of the disclosure is associated with a nanoparticle. Nanoparticles contemplated by the disclosure are generally known in the art and include, without limitation, organic and inorganic nanoparticles. Organic nanoparticles include polymer and liposomal nanoparticles, while inorganic nanoparticles include metallic (e.g., gold, silver) nanoparticles. Nanoparticles contemplated for use may be from about 1 to about 250 nanometers (nm), or from about 10 to about 100 nm, or from about 20 to about 50 nm, in diameter.

### Steroids

In some embodiments of the disclosure, the agent that increases the activity of an annexin protein is a steroid. In further embodiments, the steroid is a corticosteroid, a glucocorticoid, or a mineralocorticoid. In still further embodiments, the corticosteroid is Betamethasone, Budesonide, Cortisone, Dexamethasone, Hydrocortisone, Methylprednisolone, Prednisolone, Prednisone, Deflazacort, or a derivative thereof. In some embodiments, the corticosteroid is salmeterol, fluticasone, or budesonide. Thus, in some embodiments, an additional steroid (i.e., a steroid in addition to the glucocorticoid steroid being administered to a patient) is administered.

In some embodiments, the steroid is an anabolic steroid. In further embodiments anabolic steroids, include, but are not limited to, testosterone or related steroid compounds with muscle growth inducing properties, such as cyclostanazol or methadrostenol, prohomones or derivatives thereof, modulators of estrogen, and selective androgen receptor modulators (SARMS).

### Vectors

An appropriate expression vector may be used to deliver exogenous nucleic acid to a recipient muscle cell in the methods of the disclosure. In order to achieve effective gene therapy, the expression vector must be designed for efficient cell uptake and gene product expression. In some embodiments, the vector is within a chloroplast. In some embodiments, the vector is a viral vector. In some embodiments, the viral vector is selected from the group consisting of a herpes virus vector, an adeno-associated virus (AAV) vector, an adeno virus vector, and a lentiviral vector.

Use of adenovirus or adeno-associated virus (AAV) based vectors for gene delivery have been described [Berkner, Current Topics in Microbiol. and Imunol. 158: 39-66 (1992); Stratford-Perricaudet et al., Hum. Gene Ther. 1: 241-256 (1990); Rosenfeld et al., Cell 8: 143-144 (1992); Stratford-Perricaudet et al., J. Clin. Invest. 90: 626-630 (1992)]. In various embodiments, the adeno-associated virus vector is AAV5, AAV6, AAV8, AAV9, or AAV74. In some embodiments, the adeno-associated virus vector is AAV9. In further embodiments, the adeno-associated virus vector is AAVrh74. In further embodiments, gene editing mediated by CRISPR (clustered regularly interspaced short palindromic repeats) is used to induce genetic changes within heart or muscle for treatment.

Specific methods for gene therapy useful in the context of the present disclosure depend largely upon the expression system employed; however, most methods involve insertion of coding sequence at an appropriate position within the expression vector, and subsequent delivery of the expression vector to the target muscle tissue for expression.

Additional delivery systems useful in the practice of the methods of the disclosure are discussed in U.S. Patent Publication Numbers 2012/0046345 and 2012/0039806, each of which is incorporated herein by reference in its entirety.

### Modulators of Ltbp4

LTBP4 is located on human chromosome 19q13.1-q13.2, and is an extracellular matrix protein that binds and sequesters TGFβ. LTBP4 modifies murine muscular dystrophy through a polymorphism in the Ltbp4 gene. See U.S. Pat. No. 9,873,739, which is incorporated by reference herein in its entirety. There are two common variants of the Ltbp4 gene in mice. Most strains of mice, including the mdx mouse, have the Ltbp4 insertion allele (Ltbp4^{I/I}). Insertion of 36 base pairs (12 amino acids) into the proline-rich region of LTBP4 encoded by Ltbp4^{I/I} leads to milder disease. Deletion of 36 bp/12aa in the proline-rich region is associated with more severe disease (Ltbp4^{D/D}). It was found that the Ltbp4 genotype correlated strongly with two different aspects of muscular dystrophy pathology, i.e., membrane leakage and fibrosis, and these features define DMD pathology.

Modulators of LTBP4 are described in U.S. Pat. No. 9,873,739, which is incorporated by reference herein in its entirety.

### Modulators of TGF-β Activity

Transforming Growth Factor-β (TGF-β) superfamily is a family of secreted proteins that is comprised of over 30 members including activins, nodals, bone morphogenic proteins (BMPs) and growth and differentiation factors (GDFs). Superfamily members are generally ubiquitously expressed and regulate numerous cellular processes including growth, development, and regeneration. Mutations in TGF-β superfamily members result in a multitude of diseases including autoimmune disease, cardiac disease, fibrosis and cancer.

TGF-β ligand family includes TGF-β1, TGF-β2, and TGF-β3. TGF-β is secreted into the extracellular matrix in an inactive form bound to latency associated peptide (LAP). Latent TGF-β proteins (LTBPs) bind the TGF-β/LAP complex and provide yet another level of regulation. Extracellular proteases cleave LTBP/LAP/TGF-β releasing TGF-β. As a result, TGF-β is free to bind its receptors TGFBRI or TGFBRII. TGF-β/receptor binding, activates downstream canonical and non-canonical SMAD pathways, including activation of SMAD factors, leading to gene transcription. TGF-β signaling has emerged as a prominent mediator of the fibrotic response and disease progression in muscle disease and its expression is upregulated in dystrophy in both mouse and human. Blockade of TGF-β signaling in mice through expression of a dominant negative receptor (TGFBRII) expression, improved the dystrophic pathology, enhanced regeneration, and reduced muscle injury of 6-sarcoglycan-null mice, a mouse model of muscular dystrophy (Accornero, McNally et al Hum Mol Genet 2014). Additionally, antibody-mediated blockade of TGF-β signaling with a pan anti-TGF-β antibody, 1d11 monocloncal antibody, improved respiratory outcome measures in a mouse model of Duchenne muscular dystrophy (Nelson, Wentworth et al Am J Pathol 2011). Thus, therapeutic approaches against TGF-β signaling are contemplated herein to improve repair and delay disease progression.

Therapeutics contemplated as effective against TGF-β signaling include galunisertib (LY2157299 monohydrate), TEW-7917, monoclonal antibodies against TGF-β ligands (TGF-β 1, 2, 3 alone or pan 1, 2, 3), Fresolimemub (GC-1008), TGF-β peptide P144, LY2382770, small molecule, SB-525334, and GW788388.

### Modulators of an Androgen Response

Selective androgen receptor modulators (SARMs) are a class of androgen receptor ligands that activate androgenic signaling and exist in nonsteroidal and steroidal forms. Studies have shown that SARMs have the potential to increase both muscle and bone mass. Testosterone is one of the most well-known SARMs, which promotes skeletal muscle growth in healthy and diseased tissue. Testosterone and dihydrotestosterone (DHT) promote myocyte differentiation and upregulate follistatin, while also downregulates TGF-β signaling, resulting in muscle growth (Singh et al 2003, Singh et al 2009, Gupta et al 2008). It is conceivable that SARM-mediated inhibition of TGF-β protects against muscle injury and improves repair. SARMS may include, testosterone, estrogen, dihydrotestosterone, estradiol, include dihydronandrolone, nandrolone, nandrolone decanoate, Ostarine, Ligandrol, LGD-3303, andarine, cardarine, 7-alpha methyl, 19-nortestosterone aryl-propionamide, bicyclic hydantoin, quinolinones, tetrahydroquinoline analog, benizimidazole, imidazolopyrazole, indole, and pyrazoline derivatives, azasteroidal derivatives, and aniline, diaryl aniline, and bezoxazepinones derivatives.

### Modulators of an Inflammatory Response

A modulator of an inflammatory response includes the following agents. In some embodiments of the disclosure, the modulator of an inflammatory response is a beta2-adrenergic receptor agonist (e.g., albuterol). The term beta2-adrenergic receptor agonist is used herein to define a class of drugs which act on the P2-adrenergic receptor, thereby causing smooth muscle relaxation resulting in dilation of bronchial passages, vasodilation in muscle and liver, relaxation of uterine muscle and release of insulin. In one embodiment, the beta2-adrenergic receptor agonist for use according to the disclosure is albuterol, an immunosuppressant drug that is widely used in inhalant form for asthmatics. Albuterol is thought to slow disease progression by suppressing the infiltration of macrophages and other immune cells that contribute to inflammatory tissue loss. Albuterol also appears to have some anabolic effects and promotes the growth of muscle tissue. Albuterol may also suppress protein degradation (possibly via calpain inhibition).

In Duchenne Muscular Dystrophy (DMD), the loss of dystrophin leads to breaks in muscle cell membrane, and destabilizes neuronal nitric oxide synthase (nNOS), a protein that normally generates nitric oxide (NO). It is thought that at least part of the muscle degeneration observed in DMD patients may result from the reduced production of muscle membrane-associated neuronal nitric oxide synthase. This reduction may lead to impaired regulation of the vasoconstrictor response and eventual muscle damage.

In one embodiment, modulators of an inflammatory response suitable for use in compositions of the disclosure are Nuclear Factor Kappa-B (NF-κB) inhibitors. NF-κB is a major transcription factor modulating cellular immune, inflammatory and proliferative responses. NF-κB functions in activated macrophages to promote inflammation and muscle necrosis and in skeletal muscle fibers to limit regeneration through the inhibition of muscle progenitor cells. The activation of this factor in DMD contributes to diseases pathology. Thus, NF-κB plays an important role in the progression of muscular dystrophy and the IKK/NF-κB signaling pathway is a potential therapeutic target for the treatment of a TGFβ-related disease. Inhibitors of NF-κB (for example and without limitation, IRFI 042, a vitamin E analog) enhance muscle function, decrease serum creatine kinase (CK) level and muscle necrosis and enhance muscle regeneration. Edasalonexent is a small molecule inhibitor NF-κB. Edasalonexent administered orally as 100 mg/kg delayed muscle disease progression in Duchenne muscular dystrophy boys. Furthermore, specific inhibition of NF-κB-mediated signaling by IKK has similar benefits.

In a further embodiment, the modulator of an inflammatory response is a tumor necrosis factor alpha antagonist. TNF-α is one of the key cytokines that triggers and sustains the inflammation response. In one specific embodiment of the disclosure, the modulator of an inflammatory response is the TNF-α antagonist infliximab.

TNF-α antagonists for use according to the disclosure include, in addition to infliximab (Remicade™), a chimeric monoclonal antibody comprising murine VK and VH domains and human constant Fc domains. The drug blocks the action of TNF-α by binding to it and preventing it from signaling the receptors for TNF-α on the surface of cells. Another TNF-α antagonist for use according to the disclosure is adalimumab (Humira™). Adalimumab is a fully human monoclonal antibody. Another TNF-α antagonist for use according to the disclosure is etanercept (Enbrel™). Etanercept is a dimeric fusion protein comprising soluble human TNF receptor linked to an Fc portion of an IgG1. It is a large molecule that binds to TNF-α and thereby blocks its action. Etanercept mimics the inhibitory effects of naturally occurring soluble TNF receptors, but as a fusion protein it has a greatly extended half-life in the bloodstream and therefore a more profound and long-lasting inhibitory effect.

Another TNF-α antagonist for use according to the disclosure is pentoxifylline (Trental™), chemical name 1-(5-oxohexyl)-3,7-dimethylxanthine. The usual dosage in controlled-release tablet form is one tablet (400 mg) three times a day with meals.

Dosing: Remicade is administered by intravenous infusion, typically at 2-month intervals. The recommended dose is 3 mg/kg given as an intravenous infusion followed with additional similar doses at 2 and 6 weeks after the first infusion, then every 8 weeks thereafter. For patients who have an incomplete response, consideration may be given to adjusting the dose up to 10 mg/kg or treating as often as every 4 weeks. Humira is marketed in both preloaded 0.8 ml (40 mg) syringes and also in preloaded pen devices, both injected subcutaneously, typically by the patient at home. Etanercept can be administered at a dose of 25 mg (twice weekly) or 50 mg (once weekly).

In another embodiment of the disclosure, the modulator of an inflammatory response is cyclosporin. Cyclosporin A, the main form of the drug, is a cyclic nonribosomal peptide of 11 amino acids produced by the fungus Tolypocladium inflatum. Cyclosporin is thought to bind to the cytosolic protein cyclophilin (immunophilin) of immunocompetent lymphocytes (especially T-lymphocytes). This complex of cyclosporin and cyclophylin inhibits calcineurin, which under normal circumstances is responsible for activating the transcription of interleukin-2. It also inhibits lymphokine production and interleukin release and therefore leads to a reduced function of effector T-cells. It does not affect cytostatic activity. It has also an effect on mitochondria, preventing the mitochondrial PT pore from opening, thus inhibiting cytochrome c release (a potent apoptotic stimulation factor). Cyclosporin may be administered at a dose of 1-10 mg/kg/day.

### Promoters of Muscle Growth

In some embodiments of the disclosure, a therapeutically effective amount of a promoter of muscle growth is administered to a patient. Promoters of muscle growth contemplated by the disclosure include, but are not limited to, insulin-like growth factor-1 (IGF-1), Akt/protein kinase B, clenbuterol, creatine, decorin (see U.S. Patent Publication Number 20120058955), a steroid (for example and without limitation, a corticosteroid or a glucocorticoid steroid), testosterone and a myostatin antagonist.

### Myostatin Antagonists

Myostatin is upregulated after exposure to chronic daily steroids but not with steroids administered less frequently (e.g., weekly (Quattrocelli JCI 2017)). Accordingly, another class of promoters of muscle growth suitable for use in the combinations of the disclosure is the class of myostatin antagonists. Myostatin, also known as growth/differentiation factor 8 (GDF-8) is a transforming growth factor-β (TGFβ) superfamily member involved in the regulation of skeletal muscle mass. Most members of the TGF-β-GDF family are widely expressed and are pleiotropic; however, myostatin is primarily expressed in skeletal muscle tissue where it negatively controls skeletal muscle growth. Myostatin is synthesized as an inactive preproprotein which is activated by proteolyic cleavage. The precursor protein is cleaved to produce an approximately 109-amino-acid COOH-terminal protein which, in the form of a homodimer of about 25 kDa, is the mature, active form. The mature dimer appears to circulate in the blood as an inactive latent complex bound to the propeptide. As used herein the term “myostatin antagonist” defines a class of agents that inhibits or blocks at least one activity of myostatin, or alternatively, blocks or reduces the expression of myostatin or its receptor (for example, by interference with the binding of myostatin to its receptor and/or blocking signal transduction resulting from the binding of myostatin to its receptor). Such agents therefore include agents which bind to myostatin itself or to its receptor.

Myostatin antagonists for use according to the disclosure include antibodies to GDF-8; antibodies to GDF-8 receptors; soluble GDF-8 receptors and fragments thereof (e.g., the ActRIIB fusion polypeptides as described in U.S. Patent Publication Number 2004/0223966, which is incorporated herein by reference in its entirety, including soluble ActRIIB receptors in which ActRIIB is joined to the Fc portion of an immunoglobulin); GDF-8 propeptide and modified forms thereof (e.g., as described in WO 2002/068650 or U.S. Pat. No. 7,202,210, including forms in which GDF-8 propeptide is joined to the Fc portion of an immunoglobulin and/or form in which GDF-8 is mutated at an aspartate (asp) residue, e.g., asp-99 in murine GDF-8 propeptide and asp-100 in human GDF-8 propeptide); a small molecule inhibitor of GDF-8; follistatin (e.g., as described in U.S. Pat. No. 6,004,937, incorporated herein by reference) or follistatin-domain-containing proteins (e.g., GASP-1 or other proteins as described in U.S. Pat. Nos. 7,192,717 and 7,572,763, each incorporated herein by reference); and modulators of metalloprotease activity that affect GDF-8 activation, as described in U.S. Patent Publication Number 2004/0138118, incorporated herein by reference.

Additional myostatin antagonists include myostatin antibodies which bind to and inhibit or neutralize myostatin (including the myostatin proprotein and/or mature protein, in monomeric or dimeric form). Myostatin antibodies are mammalian or non-mammalian derived antibodies, for example an IgNAR antibody derived from sharks, or humanized antibodies, or comprise a functional fragment derived from antibodies. Such antibodies are described, for example, in WO 2005/094446 and WO 2006/116269, the content of which is incorporated herein by reference. Myostatin antibodies also include those antibodies that bind to the myostatin proprotein and prevent cleavage into the mature active form. Additional antibody antagonists include the antibodies described in U.S. Pat. Nos. 6,096,506 and 6,468,535 (each of which is incorporated herein by reference). In some embodiments, the GDF-8 inhibitor is a monoclonal antibody or a fragment thereof that blocks GDF-8 binding to its receptor. Further embodiments include murine monoclonal antibody JA-16 (as described in U.S. Pat. No. 7,320,789 (ATCC Deposit No. PTA-4236); humanized derivatives thereof and fully human monoclonal anti-GDF-8 antibodies (e.g., Myo29, Myo28 and Myo22, ATCC Deposit Nos. PTA-4741, PTA-4740, and PTA-4739, respectively, or derivatives thereof) as described in U.S. Pat. No. 7,261,893 and incorporated herein by reference.

In still further embodiments, myostatin antagonists include soluble receptors which bind to myostatin and inhibit at least one activity thereof. The term “soluble receptor” herein includes truncated versions or fragments of the myostatin receptor that specifically bind myostatin thereby blocking or inhibiting myostatin signal transduction. Truncated versions of the myostatin receptor, for example, include the naturally occurring soluble domains, as well as variations produced by proteolysis of the N- or C-termini. The soluble domain includes all or part of the extracellular domain of the receptor, either alone or attached to additional peptides or other moieties. Because myostatin binds activin receptors (including the activin type IEB receptor (ActRHB) and activin type HA receptor (ActRHA)), activin receptors can form the basis of soluble receptor antagonists. Soluble receptor fusion proteins can also be used, including soluble receptor Fc (see U.S. Patent Publication Number 2004/0223966 and WO 2006/012627, both of which are incorporated herein by reference in their entireties).

Other myostatin antagonists based on the myostatin receptors are ALK-5 and/or ALK-7 inhibitors (see for example WO 2006/025988 and WO 2005/084699, each incorporated herein by reference). As a TGF-β cytokine, myostatin signals through a family of single transmembrane serine/threonine kinase receptors. These receptors can be divided in two classes, the type I or activin-like kinase (ALK) receptors and type II receptors. The ALK receptors are distinguished from the Type II receptors in that the ALK receptors (a) lack the serine/threonine-rich intracellular tail, (b) possess serine/threonine kinase domains that are highly homologous among Type I receptors, and (c) share a common sequence motif called the GS domain, consisting of a region rich in glycine and serine residues. The GS domain is at the amino terminal end of the intracellular kinase domain and is believed to be critical for activation by the Type II receptor. Several studies have shown that TGF-β signaling requires both the ALK (Type I) and Type II receptors. Specifically, the Type II receptor phosphorylates the GS domain of the Type 1 receptor for TGFβ ALK5, in the presence of TGFβ. The ALK5, in turn, phosphorylates the cytoplasmic proteins smad2 and smad3 at two carboxy terminal serines. Generally, it is believed that in many species, the Type II receptors regulate cell proliferation and the Type I receptors regulate matrix production. Various ALK5 receptor inhibitors have been described (see, for example, U.S. Pat. Nos. 6,465,493, 6,906,089, U.S. Patent Publication Numbers 2003/0166633, 2004/0063745 and 2004/0039198, the disclosures of which are incorporated herein by reference). Thus, the myostatin antagonists for use according to the disclosure may comprise the myostatin binding domain of an ALK5 and/or ALK7 receptor.

Other myostatin antagonists include soluble ligand antagonists that compete with myostatin for binding to myostatin receptors. The term “soluble ligand antagonist” herein refers to soluble peptides, polypeptides or peptidomimetics capable of non-productively binding the myostatin receptor(s) (e.g., the activin type HB receptor (ActRHA)) and thereby competitively blocking myostatin-receptor signal transduction. Soluble ligand antagonists include variants of myostatin, also referred to as “myostatin analogs” that have homology to, but not the activity of, myostatin. Such analogs include truncates (such as N- or C-terminal truncations, substitutions, deletions, and other alterations in the amino acid sequence, such as variants having non-amino acid substitutions).

Additional myostatin antagonists contemplated by the disclosure include inhibitory nucleic acids as described herein. These antagonists include antisense or sense polynucleotides comprising a single-stranded polynucleotide sequence (either RNA or DNA) capable of binding to target mRNA (sense) or DNA (antisense) sequences. Thus, RNA interference (RNAi) produced by the introduction of specific small interfering RNA (siRNA), may also be used to inhibit or eliminate the activity of myostatin.

In specific embodiments, myostatin antagonists include, but are not limited to, follistatin, the myostatin prodomain, growth and differentiation factor 11 (GDF-11) prodomain, prodomain fusion proteins, antagonistic antibodies or antibody fragments that bind to myostatin, antagonistic antibodies or antibody fragments that bind to the activin type IEB receptor, soluble activin type IHB receptor, soluble activin type IEB receptor fusion proteins, soluble myostatin analogs (soluble ligands), polynucleotides, small molecules, peptidomimetics, and myostatin binding agents. Other antagonists include the peptide immunogens described in U.S. Pat. No. 6,369,201 and WO 2001/05820 (each of which is incorporated herein by reference) and myostatin multimers and immunoconjugates capable of eliciting an immune response and thereby blocking myostatin activity. Other antagonists include the protein inhibitors of myostatin described in WO 2002/085306 (incorporated herein by reference), which include the truncated Activin type II receptor, the myostatin pro-domain, and follistatin. Other myostatin inhibitors include those released into culture from cells overexpressing myostatin (see WO 2000/43781), dominant negative myostatin proteins (see WO 2001/53350) including the protein encoded by the Piedmontese allele, and mature myostatin peptides having a C-terminal truncation at a position either at or between amino acid positions 335 to 375. The small peptides described in U.S. Patent Publication Number 2004/0181033 (incorporated herein by reference) that comprise the amino acid sequence WMCPP, are also suitable for use in the compositions of the disclosure.

### Chemotherapeutic Agents

Chemotherapeutic agents contemplated for use in the methods of the disclosure include, without limitation, alkylating agents including: nitrogen mustards, such as mechlor-ethamine, cyclophosphamide, ifosfamide, melphalan and chlorambucil; nitrosoureas, such as carmustine (BCNU), lomustine (CCNU), and semustine (methyl-CCNU); ethylenimines/methylmelamine such as thriethylenemelamine (TEM), triethylene, thiophosphoramide (thiotepa), hexamethylmelamine (HMM, altretamine); alkyl sulfonates such as busulfan; triazines such as dacarbazine (DTIC); antimetabolites including folic acid analogs such as methotrexate and trimetrexate, pyrimidine analogs such as 5-fluorouracil, fluorodeoxyuridine, gemcitabine, cytosine arabinoside (AraC, cytarabine), 5-azacytidine, 2,2′-difluorodeoxycytidine, purine analogs such as 6-mercaptopurine, 6-thioguanine, azathioprine, 2′-deoxycoformycin (pentostatin), erythrohydroxynonyladenine (EHNA), fludarabine phosphate, and 2-chlorodeoxyadenosine (cladribine, 2-CdA); natural products including antimitotic drugs such as paclitaxel, vinca alkaloids including vinblastine (VLB), vincristine, and vinorelbine, taxotere, estramustine, and estramustine phosphate; epipodophylotoxins such as etoposide and teniposide; antibiotics such as actimomycin D, daunomycin (rubidomycin), doxorubicin, mitoxantrone, idarubicin, bleomycins, plicamycin (mithramycin), mitomycin C, and actinomycin; enzymes such as L-asparaginase; biological response modifiers such as interferon-alpha, IL-2, G-CSF and GM-CSF; miscellaneous agents including platinum coordination complexes such as cisplatin and carboplatin, anthracenediones such as mitoxantrone, substituted urea such as hydroxyurea, methylhydrazine derivatives including N-methylhydrazine (MIH) and procarbazine, adrenocortical suppressants such as mitotane (o,p′-DDD) and aminoglutethimide; hormones and antagonists including adrenocorticosteroid antagonists such as prednisone and equivalents, dexamethasone and aminoglutethimide; progestin such as hydroxyprogesterone caproate, medroxyprogesterone acetate and megestrol acetate; estrogen such as diethylstilbestrol and ethinyl estradiol equivalents; antiestrogen such as tamoxifen; androgens including testosterone propionate and fluoxymesterone/equivalents; antiandrogens such as flutamide, gonadotropin-releasing hormone analogs and leuprolide; and non-steroidal antiandrogens such as flutamide.

### Modulators of Fibrosis

A “modulator of fibrosis” as used herein is synonymous with antifibrotic agent. The term “antifibrotic agent” refers to a chemical compound that has antifibrotic activity (i.e., prevents or reduces fibrosis) in mammals. This takes into account the abnormal formation of fibrous connective tissue, which is typically comprised of collagen. These compounds may have different mechanisms of action, some reducing the formation of collagen or another protein, others enhancing the catabolism or removal of collagen in the affected area of the body. All such compounds having activity in the reduction of the presence of fibrotic tissue are included herein, without regard to the particular mechanism of action by which each such drug functions. Antifibrotic agents useful in the methods and compositions of the disclosure include those described in U.S. Pat. No. 5,720,950, incorporated herein by reference. Additional antifibrotic agents contemplated by the disclosure include, but are not limited to, Type II interferon receptor agonists (e.g., interferon-gamma); pirfenidone and pirfenidone analogs; anti-angiogenic agents, such as VEGF antagonists, VEGF receptor antagonists, bFGF antagonists, bFGF receptor antagonists, TGFβ antagonists, TGFβ receptor antagonists; anti-inflammatory agents, IL-1 antagonists, such as IL-1Ra, angiotensin-converting-enzyme (ACE) inhibitors, angiotensin receptor blockers and aldosterone antagonists.

### Modulators of Glucose Homeostasis

In some embodiments of the disclosure, a method of administering a glucocorticoid steroid to a patient further comprises administering a modulator of glucose homeostasis.

Modulators of glucose homeostasis contemplated by the disclosure include, but are not limited to, a peptide as disclosed in U.S. Patent Application Publication No. 2019/0091282 (incorporated by reference herein in its entirety), stem cell factor (see, e.g., U.S. Patent Application Publication No. 2019/0070261), insulin and other agents that are commonly used to control blood glucose, such as but not limited to metformin, pioglitazone, repaglinide, acarbose, sitagliptin, liraglutide, sulfonylureas (e.g., acetohexamide, carbutamide, chlorpropamide, glycyclamide (tolhexamide), metahexamide, tolazamide, tolbutamide, glibenclamide (glyburide), glibomuride, gliclazide, glipizide, gliquidone, glisoxepide, glyclopyramide, glimepride), sodium-glucose cotransporter-2 inhibitors (e.g., canagliflozin, dapagliflozin, empagliflozin, ertugliflozin, ipragliflozin, luseogliflozin, remogliflozin, sergliflozin, sotagliflozin, tofogliflozin).

### Modulators of Metabolic Function

In some embodiments of the disclosure, a method of administering a glucocorticoid steroid to a patient further comprises administering a modulator of metabolic function.

Modulators of metabolic function contemplated by the disclosure include, but are not limited to, pharmacological modulators of the peroxisome proliferator-activator receptor family members (e.g., clofibrate, gemfibrozil, ciprofibrate, bezafibrate, fenofibrate, thiazolidinediones, indoles, GW-9662, GW501516, aleglitazar, muraglitazar, tesaglitazar, saroglitazar), pharmacological modulators of cholesterol and tryglyceride levels (e.g., statins, niacin, bile acid resins), amino acid supplements (e.g., leucine, isoleucine, valine), hormonal modulators of satiety and adiposity (e.g., leptin, adiponectin), performance-enhancing drugs (ergogenic aids; e.g., human growth hormone, caffeine, ephedrine, methylphenidate, amphetamine).

### Disorders/Injuries

In various aspects, the disclosure provides methods and compositions for treating, delaying onset, enhancing recovery from, or preventing a condition of muscle wasting, aging, and metabolic disorder, comprising administering a glucocorticoid steroid to a patient in need thereof.

Such a patient is one that is suffering from, for example, muscle wasting, obesity, a metabolic disorder, sarcopenia, an inflammatory disorder, a muscle injury, or a combination thereof. In some embodiments, the muscle wasting is aging-related muscle wasting, disease-related muscle wasting, diabetes-associated muscle wasting, muscle atrophy, sarcopenia, cardiomyopathy, a chronic myopathy, an inflammatory myopathy (for example and without limitation: polymyositis, dermatomyositis), a muscular dystrophy, or a combination thereof. In further embodiments, the metabolic disorder is type I diabetes, type II diabetes, metabolic syndrome, insulin resistance, a nutrition disorder, exercise intolerance, or a combination thereof. It was generally understood in the art that administration of glucocorticoid steroids can actually lead to adverse events such as diabetes, obesity, and cardiovascular events (see, e.g., Fardet et al., Drugs 74: 1731-1745 (2014)). Moreover, it has recently been shown that daily administration of glucocorticoid steroids can effectively counteract the beneficial effects of anti-myostatin therapies in myopathic muscle (Hammers et al, JCI Insight 2019 in press, https://doi.org/10.1172/jci.insight.133276. As disclosed herein, however, it was unexpectedly found that administering glucocorticoid steroids according to the methods of the disclosure can treat, delay onset, enhance recovery from, or prevent conditions such as obesity, diabetes, and cardiovascular events.

Thus, the patient may be suffering from Duchenne Muscular Dystrophy, Limb Girdle Muscular Dystrophy, Becker Muscular Dystrophy, Emery-Dreifuss Muscular Dystrophy (EDMD), Myotonic Dystrophy, Fascioscapulohumeral Dystrophy (FSHD), Oculopharyngeal Muscular Dystrophy, Distal Muscular Dystrophy, Congenital Muscular Dystrophy, cystic fibrosis, pulmonary fibrosis, muscle atrophy, spinal muscle atrophy, amyotrophic lateral sclerosis (motor neuron disease, Lou Gehrig's disease), cerebral palsy, an epithelial disorder, an epidermal disorder, a kidney disorder, a liver disorder, sarcopenia, cardiomyopathy, myopathy, cystic fibrosis, pulmonary fibrosis, cardiomyopathy (including hypertrophic, dilated, congenital, arrhythmogenic, restrictive, ischemic, or heart failure), acute lung injury, acute muscle injury, acute myocardial injury, radiation-induced injury, colon cancer, idiopathic pulmonary fibrosis, idiopathic interstitial pneumonia, autoimmune lung diseases, benign prostate hypertrophy, cerebral infarction, musculoskeletal fibrosis, post-surgical adhesions, liver cirrhosis, renal fibrotic disease, fibrotic vascular disease, neurofibromatosis, Alzheimer's disease, diabetic retinopathy, skin lesions, lymph node fibrosis associated with HIV, chronic obstructive pulmonary disease (COPD), inflammatory pulmonary fibrosis, rheumatoid arthritis; rheumatoid spondylitis; osteoarthritis; gout, other arthritic conditions; sepsis; septic shock; endotoxic shock; gram-negative sepsis; toxic shock syndrome; myofacial pain syndrome (MPS); Shigellosis; asthma; adult respiratory distress syndrome; inflammatory bowel disease; Crohn's disease; psoriasis; eczema; ulcerative colitis; glomerular nephritis; scleroderma; chronic thyroiditis; Grave's disease; Ormond's disease; autoimmune gastritis; myasthenia gravis; autoimmune hemolytic anemia; autoimmune neutropenia; thrombocytopenia; pancreatic fibrosis; chronic active hepatitis including hepatic fibrosis; renal fibrosis, irritable bowel syndrome; pyresis; restenosis; cerebral malaria; stroke and ischemic injury; neural trauma; Huntington's disease; Parkinson's disease; allergies, including allergic rhinitis and allergic conjunctivitis; cachexia; Reiter's syndrome; acute synoviitis; muscle degeneration, bursitis; tendonitis; tenosynoviitis; osteopetrosis; thrombosis; silicosis; pulmonary sarcosis; bone resorption diseases, such as osteoporosis or multiple myeloma-related bone disorders; cancer, including but not limited to metastatic breast carcinoma, colorectal carcinoma, malignant melanoma, gastric cancer, and non-small cell lung cancer; graft-versus-host reaction; and auto-immune diseases, such as multiple sclerosis, lupus and fibromyalgia; viral diseases such as Herpes Zoster, Herpes Simplex I or II, influenza virus, Severe Acute Respiratory Syndrome (SARS) and cytomegalovirus.

As used herein, “cardiomyopathy” refers to any disease or dysfunction of the myocardium (heart muscle) in which the heart is abnormally enlarged, thickened and/or stiffened. As a result, the heart muscle's ability to pump blood is usually weakened, often leading to congestive heart failure. The disease or disorder can be, for example, inflammatory, metabolic, toxic, infiltrative, fibrotic, hematological, genetic, or unknown in origin. Such cardiomyopathies may result from a lack of oxygen. Other diseases include those that result from myocardial injury which involves damage to the muscle or the myocardium in the wall of the heart as a result of disease or trauma. Myocardial injury can be attributed to many things such as, but not limited to, cardiomyopathy, myocardial infarction, or congenital heart disease. The cardiac disorder may be pediatric in origin. Cardiomyopathy includes, but is not limited to, cardiomyopathy (dilated, hypertrophic, restrictive, arrhythmogenic, ischemic, genetic, idiopathic and unclassified cardiomyopathy), sporadic dilated cardiomyopathy, X-linked Dilated Cardiomyopathy (XLDC), acute and chronic heart failure, right heart failure, left heart failure, biventricular heart failure, congenital heart defects, myocardiac fibrosis, mitral valve stenosis, mitral valve insufficiency, aortic valve stenosis, aortic valve insufficiency, tricuspidal valve stenosis, tricuspidal valve insufficiency, pulmonal valve stenosis, pulmonal valve insufficiency, combined valve defects, myocarditis, acute myocarditis, chronic myocarditis, viral myocarditis, diastolic heart failure, systolic heart failure, diabetic heart failure and accumulation diseases. In some embodiments, the heart failure includes reduced ejection fraction. In further embodiments, the heart failure includes preserved ejection fraction.

### Therapeutic Endpoints

In various aspects of the disclosure, administration of the glucocorticoid steroid and optional further agent(s)/compound(s) as described herein provide one or more benefits related to specific therapeutic endpoints relative to a patient not receiving the glucocorticoid steroid and optional further agent(s)/compound(s). For example and without limitation, the administering results in one or more of decreased insulin resistance, increased glucose tolerance, increased muscle mass, decreased hyperinsulinemia, increased lean mass, increased force, increased systolic function, increased diastolic function, decreased muscle fibrosis, increased exercise tolerance, increased nutrient catabolism, increased energy production (as measured by increased muscle nicotinamide adenine dinucleotide (NAD) and/or increased muscle adenosine triphosphate (ATP)), increased serum adiponectin, decreased serum branched chain amino acids (BCAA), decreased serum lipid level, decreased serum ketone level, decreased hyperglycemia, increased serum cortisol level, increased serum corticosterone, and decreased adipocyte size compared to administering the glucocorticoid steroid in a dosing regimen that is not once-weekly or to not administering the glucocorticoid steroid. Each of the foregoing markers is quantifiable by methods known in the art.

In addition, creatine kinase (CK) is a clinically validated serum biomarker of skeletal muscle, cardiac, kidney, and brain injury. Lactate dehydrogenase (LDH) is a clinically validated serum biomarker of skeletal muscle, cardiac, kidney, liver, lung, and brain injury. Creatine kinase and lactate dehydrogenase levels in serum are elevated with both acute and chronic tissue injury. In theoretical or verified conditions of comparable muscle mass levels, a reduction in creatine kinase and/or lactate dehydrogenase may be indicative of enhanced repair or protection against injury. Aspartate aminotransferase (AST) is yet another clinically validated serum biomarker of skeletal muscle, cardiac, kidney, liver, and brain injury. Additionally, increased serum troponin is indicative of cardiac injury, while elevated alanine transaminase (ALT) is a biomarker of liver injury. Reduction in AST, ALT, or troponin in the acute period following injury may indicate enhanced repair or protection against injury. Evan's blue due is a vital dye that binds serum albumin and is normally excluded from healthy, intact muscle. Membrane disruption due to acute or chronic injury promotes the influx of dye into the damaged cell. Evan's blue dye is commonly used to quantify cellular damage in experimental settings, measuring inherent dye fluorescence and/or through measuring radiolabeled-dye uptake. Reduction in dye uptake after acute injury or in models of chronic damage would indicate protection against injury and/or enhanced repair. Indocyanine green (ICG) is a near-infrared dye that binds plasma proteins and is used clinically to evaluate blood flow and tissue damage (ischemia; necrosis) in organs including heart, liver, kidney, skin, vasculature, lung, muscle and eye. Improved blood flow and reduction in ischemic areas indicate protection from injury and/or improved repair.

Additionally, histological benefits may be noted in the muscle of treated patients, including decreased necrosis, decreased inflammation, reduced fibrosis, reduced fatty infiltrate and reduced edema. These beneficial effects may also be visible through MR and PET imaging.

### Dosing/Administration/Kits

A particular administration regimen for a particular subject will depend, in part, upon the agent and optional additional agent used, the amount of the agent and optional additional agent administered, the route of administration, the particular ailment being treated, and the cause and extent of any side effects. The amount of glucocorticoid steroid and other agents/compounds disclosed herein administered to a subject (e.g., a mammal, such as a human) is an amount sufficient to effect the desired response. Dosage typically depends upon a variety of factors, including the particular agent and/or additional agent employed, the age and body weight of the subject, as well as the existence and severity of any disease or disorder in the subject. The size of the dose also will be determined by the route, timing, and frequency of administration. Accordingly, the clinician may titer the dosage and modify the route of administration to obtain optimal therapeutic effect, and conventional range-finding techniques are known to those of ordinary skill in the art. In various embodiments, the amount of glucocorticoid steroid that is administered as a once-weekly single dose is from about 0.1 to about 5 mg/kg. In further embodiments, the amount of glucocorticoid steroid that is administered as a once-weekly single dose is from about 0.1 to about 4 mg/kg, or about 0.1 to about 3 mg/kg, or about 0.1 to about 2 mg/kg, or about 0.1 to about 1 mg/kg, or about 0.5 to about 4 mg/kg, or about 0.5 to about 3 mg/kg, or about 0.5 to about 2 mg/kg, or about 0.5 to about 1 mg/kg, or about 0.5 to about 0.8 mg/kg, or about 1 to about 4 mg/kg, or about 1 to about 3 mg/kg, or about 1 to about 2 mg/kg. In further embodiments, the amount of glucocorticoid steroid that is administered as a once-weekly single dose is or is at least about 0.1, is or is at least about 0.2, is or is at least about 0.3, is or is at least about 0.4, is or is at least about 0.5, is or is at least about 0.6, is or is at least about 0.7, is or is at least about 0.75, is or is at least about 0.8, is or is at least about 0.9, is or is at least about 1, is or is at least about 1.5, is or is at least about 2, is or is at least about 2.5, is or is at least about 3, is or is at least about 3.5, is or is at least about 4, is or is at least about 4.5, or is or is at least about 5 mg/kg. In further embodiments, the amount of glucocorticoid steroid that is administered as a once-weekly single dose is less than about 0.2, less than about 0.3, less than about 0.4, less than about 0.5, less than about 0.6, less than about 0.7, less than about 0.8, less than about 0.9, less than about 1, less than about 1.5, less than about 2, less than about 2.5, less than about 3, less than about 3.5, less than about 4, less than about 4.5, or less than about 5 mg/kg. In some embodiments, the frequency of glucocorticoid steroid administration ranges from one dose every day to one dose every 14 days. In further embodiments, the frequency of glucocorticoid steroid administration is about one dose every 3 days, or about one dose every 4 days, or about one dose every 5 days, or about one dose every 6 days, or about one dose every 7 days, or about one dose every 8 days, or about one dose every 9 days, or about one dose every 10 days.

Regarding the other agents/compounds disclosed herein, and in various embodiments, the methods of the disclosure comprise administering an agent/compound of the disclosure (e.g., a protein), e.g., from about 0.1 μg/kg up to about 100 mg/kg or more, depending on the factors mentioned above. In other embodiments, the dosage may range from 1 μg/kg up to about 75 mg/kg; or 5 μg/kg up to about 50 mg/kg; or 10 μg/kg up to about 20 mg/kg. In certain embodiments, the dose comprises about 0.5 mg/kg to about 20 mg/kg (e.g., about 1 mg/kg, 1.5 mg/kg, 2 mg/kg, 2.3 mg/kg, 2.5 mg/kg, 3 mg/kg, 3.5 mg/kg, 4 mg/kg, 4.5 mg/kg, 5 mg/kg, 5.5 mg/kg, 6 mg/kg, 6.5 mg/kg, 7 mg/kg, 8 mg/kg, 9 mg/kg, or 10 mg/kg) of agent and optional additional agent. In embodiments in which a glucocorticoid steroid and a further agent/compound are administered, the above dosages are contemplated to represent the amount of each agent administered, or in further embodiments the dosage represents the total dosage administered. In some embodiments wherein a chronic condition is treated, it is envisioned that a subject will receive the glucocorticoid steroid and/or the further agent/compound over a treatment course lasting weeks, months, or years.

In some embodiments, administration of the further agent/compound may require one or more doses daily or weekly. Dosages are also contemplated for once daily, twice daily (BID) or three times daily (TID) dosing. A unit dose may be formulated in either capsule or tablet form. In other embodiments, the further agent/compound is administered to treat an acute condition (e.g., acute muscle injury or acute myocardial injury) for a relatively short treatment period, e.g., one to 14 days.

Suitable methods of administering a physiologically-acceptable composition (comprising, in various embodiments, the glucocorticoid steroid and/or the further agent/compound) are well known in the art. Although more than one route can be used to administer an agent and/or additional agent, a particular route can provide a more immediate and more effective avenue than another route. Depending on the circumstances, a pharmaceutical composition is applied or instilled into body cavities, absorbed through the skin or mucous membranes, ingested, inhaled, and/or introduced into circulation. In some embodiments, a composition of the disclosure is administered intravenously, intraarterially, or intraperitoneally to introduce the composition into circulation. Non-intravenous administration also is appropriate, particularly with respect to low molecular weight therapeutics. In certain circumstances, it is desirable to deliver a pharmaceutical composition orally, topically, sublingually, vaginally, rectally; through injection by intracerebral (intra-parenchymal), intracerebroventricular, intramuscular, intra-ocular, intraportal, intralesional, intramedullary, intrathecal, intraventricular, transdermal, subcutaneous, intranasal, urethral, or enteral means; by sustained release systems; or by implantation devices. If desired, the composition is administered regionally via intraarterial or intravenous administration to a region of interest, e.g., via the femoral artery for delivery to the leg. In one embodiment, the composition is administered via implantation of a membrane, sponge, or another appropriate material within or upon which the desired agent and optional additional agent has been absorbed or encapsulated. Where an implantation device is used, the device in one aspect is implanted into any suitable tissue, and delivery of the composition is, in various embodiments, effected via diffusion, time-release bolus, or continuous administration. In other embodiments, the composition is administered directly to exposed tissue during surgical procedures or treatment of injury, or is administered via transfusion of blood products. Therapeutic delivery approaches are well known to the skilled artisan, some of which are further described, for example, in U.S. Pat. No. 5,399,363.

In some embodiments facilitating administration, the composition is formulated into a physiologically acceptable composition comprising a carrier (i.e., vehicle, adjuvant, buffer, or diluent). The particular carrier employed is limited only by chemico-physical considerations, such as solubility and lack of reactivity with the agent and/or additional agent, by the route of administration, and by the requirement of compatibility with the recipient organism. Physiologically acceptable carriers are well known in the art. Illustrative pharmaceutical forms suitable for injectable use include, without limitation, sterile aqueous solutions or dispersions and sterile powders for the extemporaneous preparation of sterile injectable solutions or dispersions (for example, see U.S. Pat. No. 5,466,468). Injectable formulations are further described in, e.g., Pharmaceutics and Pharmacy Practice, J. B. Lippincott Co., Philadelphia. Pa., Banker and Chalmers. eds., pages 238-250 (1982), and ASHP Handbook on Injectable Drugs, Toissel, 4th ed., pages 622-630 (1986), incorporated herein by reference).

A pharmaceutical composition as provided herein is optionally placed within containers/kits, along with packaging material that provides instructions regarding the use of such pharmaceutical compositions. Generally, such instructions include a tangible expression describing the reagent concentration, as well as, in certain embodiments, relative amounts of excipient ingredients or diluents that may be necessary to reconstitute the pharmaceutical composition.

The disclosure thus includes embodiments for administering to a subject a glucocorticoid steroid optionally in combination with one or more further agent(s)/compound(s), each being administered according to a regimen suitable for that medicament. Administration strategies include concurrent administration (i.e., substantially simultaneous administration) and non-concurrent administration (i.e., administration at different times, in any order, whether overlapping or not). It will be appreciated that different components are optionally administered in the same or in separate compositions, and by the same or different routes of administration.

All publications, patents and patent applications cited in this specification are herein incorporated by reference as if each individual publication or patent application were specifically and individually indicated to be incorporated by reference. In addition, the entire document is intended to be related as a unified disclosure, and it should be understood that all combinations of features described herein are contemplated, even if the combination of features are not found together in the same sentence, or paragraph, or section of this document. For example, where protein therapy is described, embodiments involving polynucleotide therapy (using polynucleotides/vectors that encode the protein) are specifically contemplated, and the reverse also is true. With respect to elements described as one or more members of a set, it should be understood that all combinations within the set are contemplated.

### Compositions

Any of the glucocorticoid steroid, optionally in combination with one or more further agent(s)/compound(s) described herein (or nucleic acids encoding any of the further agent(s)/compound(s) described herein) also is provided in a composition. In this regard, glucocorticoid steroid, optionally in combination with one or more further agent(s)/compound(s) described herein is formulated with a physiologically-acceptable (i.e., pharmacologically acceptable) carrier, buffer, or diluent, as described further herein. Optionally, a protein/recombinant protein as disclosed herein is in the form of a physiologically acceptable salt, which is encompassed by the disclosure. “Physiologically acceptable salts” means any salts that are pharmaceutically acceptable. Some examples of appropriate salts include acetate, trifluoroacetate, hydrochloride, hydrobromide, sulfate, citrate, tartrate, glycolate, and oxalate.

### EXAMPLES

Chronic glucocorticoid steroids produce muscle atrophy, but intermittent steroid exposure can promote muscle growth, especially in dystrophic muscle. It is disclosed herein that intermittent prednisone treatment of two mouse models of muscular dystrophy, mdx and dysferlin-null, enhanced mitochondrial respiration through branched-chain amino acid catabolism, while increasing glycolysis and NAD⁺ levels. Integration of transcriptomic and epigenomic analyses of glucocorticoid-treated myofibers identified a glucocorticoid receptor-responsive KLF15-MEF2C axis driving a genomewide nutrient metabolic shift. Metabolic profiling and live animal imaging showed improvement of branched-chain amino acid metabolism and glucose uptake in muscle. Serum biomarkers from Duchenne Muscular Dystrophy patients supported that intermittent steroid use augmented BCAA disposal while blunting obesity and insulin resistance compared to chronic daily exposure. Together these findings showed that pulsatile administration of glucocorticoids promotes pro-ergogenic muscle remodeling, favoring enhanced branched-chain amino acid utilization and increasing insulin sensitivity.

The present disclosure demonstrates that pulsatile GC steroids induce a distinct epigenomic program in dystrophic muscle centered on the transcriptional regulators KLF15 and MEF2C. Glucocorticoid-responsive metabolic reprogramming enhanced BCAA utilization and energy production in mdx and even in dysferlin-deficient mice. Moreover, it was found that pulsatile, compared to daily GC steroids, reduced obesity and biomarkers of insulin resistance and BCAAs in DMD patients. Together, these findings define the molecular and metabolic mechanisms of pro-ergogenic glucocorticoid treatments in mice and humans with muscular dystrophies.

By means of multi-modal live imaging and serum biomarker analyses in mice and humans, it is disclosed herein that once-weekly glucocorticoids increases glucose uptake in muscle but not in fat; does not induce osteoporosis, an important adverse side effect of current glucocorticoid indications. Weekly steroids enhance production and circulation of adiponectin, an anti-adiposity peptide, while decreasing free fatty acid and ketone body levels, markers of metabolic dysfunction. Similar biomarker profiles were observed in boys with Duchenne muscular dystrophy (DMD), where metabolic biomarkers reflected weekend glucocorticoid intake reduces the metabolic and endocrinologic adverse side effects caused by daily glucocorticoid intake. Daily glucocorticoid treated DMD boys showed biomarkers of insulin resistance, osteoporosis and obesity as described herein.

Whether weekly steroid dosing was beneficial in aging mice was tested, where mice were treated for 12 months with weekly prednisone. Once-weekly prednisone was found to improve muscle mass and strength, cardiac function and respiratory function in aged mice. Moreover, once-weekly prednisone promoted muscle bioenergetics, seen as higher levels of ATP, NAD+ and glycogen. Serum levels of adiponectin, free fatty acids and ketone body showed similar profiles in aging mice treated with once-weekly prednisone as described herein.

Using a mouse model of obesity (mice fed a high-fat diet for 8 weeks), it was found that once-weekly prednisone decreased weight and fat accrual while improving lean mass. Once-weekly glucocorticoid intake was linked to increased force production and endurance, as well as improved glucose homeostasis, insulin sensitivity and adiponectin levels in obese mice as described herein.

Once-weekly glucocorticoid steroids improves energy production, metabolic function and muscle mass. Thus, in some aspects, this treatment is a candidate for a large set of new and unanticipated indications, ranging from muscle wasting to unhealthy aging and metabolic disorders.

### Example 1

### Methods

Animal handling and steroid regimens. Mice were housed in a pathogen-free dedicated vivarium in accordance with Institutional Animal Care and Use Committee (IACUC) guidelines. Euthanasia was performed through carbon dioxide inhalation followed by cervical dislocation and heart removal. All methods using living animals in this study were performed in ethical accordance with the American Veterinary Medical Association (AVMA) and under protocols fully approved by both the Institutional Animal Care and Use Committee (IACUC) at Northwestern University Feinberg School of Medicine (protocol number ISO00000761). Consistent with the ethical approvals, all efforts were made to minimize suffering. Mice were fed ad libitum with Mouse Breeder Sterilizable Diet (#7904; Harlan Teklad, Indianapolis, Ind.) and maintained on a 12-hour light/dark cycle. mdx mice from the DBA/2J background were obtained from the Jackson Laboratory (Bar Harbor, Me.; stock #013141) and interbred. Male mice were used for reported experiments. Age at start was approximately 6 months for short-term experiments, approximately 6 weeks for long-term experiments. Dysferlin-null (Dysf-null) mice from the 129T2/SvEmsJ background were previously characterized (Demonbreun et al., 2011; Demonbreun et al., 2014). Age at start was approximately 9 months for long-term experiments. For experiments with Dysf-null and wildtype mice, both females and males (approximately 1:1 ratio) were randomized in treatment groups. Prednisone (#P6254; Sigma-Aldrich; St. Louis, Mo.) was resuspended in DMSO (#D2650; Sigma-Aldrich; St. Louis, Mo.) to a stock concentration of 5 mg/ml. Dosing was based on weekly weight measurements (1 mg/kg body weight, (Sali et al., 2012)) in 100 μl total PBS volume per dose. Mice were injected daily via intraperitoneal injection at 7 AM. On injection days, stock solutions stored at −20° C. were diluted into sterile Eppendorf tubes containing sterile phosphate buffered saline (PBS) (#14190; Thermo Fisher, Waltham, Mass.). Puromycin (cat #A1113803, Thermo Scientific, Waltham, Mass.) was administered i.p. as 0.040 μmol/body g, and tissues were snap-frozen 30 minutes after injection. Sterile BD Micro-Fine IV Insulin Syringes (#14-829-1A; Fisher Scientific, Waltham, Mass.) were used to inject the intraperitoneal cavity of non-sedated animals. All animal analyses both during treatment and at endpoint were conducted blinded to treatment groups.

Human sample collection. Individuals in Muscular Dystrophy Association Clinic at the Ann & Robert H. Lurie Children's Hospital of Chicago with a confirmed genetic diagnosis of Duchenne Muscular Dystrophy (DMD) were asked for consent as part of a clinical trial (NCT03319030). Institutional approval was granted by the institution's Institutional Review Board (2017-1264). All protocols and consents were conducted in accordance with the Declaration of Helsinki and other international ethical guidelines. Blood samples were sterilely collected in a red top tube at end of individual's clinic appointment (generally late morning-early afternoon) on Thursdays. Samples were centrifuged at 2000 g for 10 minutes at 4° C. Serum was isolated, pre-aliquoted for downstream assays to avoid repeated freeze/thaw and stored at −80° C. Dual X-ray absorptiometry (DEXA) data were collected from regular measurements that individuals with DMD undergo annually as part of standard of care. All scans were performed on a GE Lunar iDXA (Boston, Mass.) during same clinic visit as blood sample collection or at most recent clinic visit, approximately 6 months prior. Z-scores were established based on age-standardized controls provided by computer program on machine. For Brooke's functional scoring, physical therapists assessed the Brooke's Functional scale score at each clinic visit and documented it as part of their clinic notes. The scale is scored on a 9-point scale: a score of 1 indicates the highest level of ambulation versus a score of 9 indicates the individual is confined to a wheelchair. Data were collected on day of blood collection. For 10-meter run tests, individuals diagnosed with DMD and who are ambulatory perform the 10-meter run test as part of their clinical assessment. Physical therapist timed individuals with a stopwatch. Individuals performed 10-meter run test as fast as safely permissible while barefoot. Data were collected on day of blood collection. For ECG data, individuals with DMD undergo 12 lead ECGs on a GE MAC5500HD (Milwaukee, Wis.) on standard ECG paper (10 mv, 25 mm/s, 150 Hz) as part of their clinical care. ECGs were collected at the same clinic visit as blood collection or at prior clinic encounter, approximately 6 months prior. ECG's were read and confirmed by a pediatric cardiologist at our institution. For heart function measurements, individuals with DMD undergo routine echocardiogram assessment annually. Echocardiographic measurements used in this study were either performed at the same clinic visit as serum collection or during most recent clinic encounter, approximately 6 months prior. Echocardiography was performed on a Philips iE33 Ultrasound machine (Philips, Andover, Mass.) and read routinely by pediatric cardiologists at our institution. All analyses related to serum samples were conducted blinded to treatment groups and to other clinical assessments.

Dosing of metabolic and endocrine biomarkers. Glycogen was quantitated using the Glycogen Assay Kit (#ab65620; Abcam, Cambridge, Mass.) from approximately 25 mg frozen-pulverized whole tissue, following manufacturer's instructions and internal standards for calculating μg/mg values. For measurement of whole-tissue ATP/NAD⁺ levels, approximately 25 mg frozen-pulverized tissue was extracted in 10% perchloric acid and neutralized in 0.75 M K₂CO₃, as previously described (Ramsey et al., 2009). NAD+ and ATP were measured by high-pressure liquid chromatography (HPLC) with Shimadzu LC-20A pump (Shimadzu Scientific Instr Inc, Addison, Ill.) and UV-VIS detector, using a Supelco LC-18-T column (15 cm×4.6 cm; #58970-U; Millipore-Sigma, St Louis, Mo.). The HPLC was run at a flow rate of 1 ml/min with 100% buffer A (0.5 M KH₂PO₄, 0.5 M K₂HPO₄) from 0 to 5 min, a linear gradient to 95% buffer A/5% buffer B (100% methanol) from 5 to 6 min, 95% buffer A/5% buffer B from 6 to 11 min, a linear gradient to 85% buffer A/15% buffer B from 11 to 13 min, 85% buffer A/15% buffer B from 13 to 23 min, and a linear gradient to 100% buffer A from 23 to 30 minutes. ATP and NAD⁺ eluted as sharp peaks at 3 and 14 minutes, respectively, and were normalized to tissue weight of frozen liver tissue for calculating pmol/mg values. Corticosterone was measured in mouse serum and cortisol was measured in human serum using dedicated ELISA kits (#ADI-900-097, Enzo Life Science, Farmingdale, N.Y.; #K7430-100, BioVision, Milpitas, Calif.) according to manufacturer's instructions and internal standards to calculate ng/ml values. Insulin levels were quantitated in mouse and human serum with species-specific ELISA kits (#10-1247-01 (mouse-specific); #10-1113-01 (human-specific); Mercodia, Uppsala, Sweden), following manufacturer's instructions and internal standards to calculate ng/ml values. Free fatty acids were quantitated using Enzychrom Free Fatty Acid Assay kit (#EFFA-100; BioAssay Systems, Hayward, Calif.), following kit's instructions and standards to calculate μM (serum) and nmol/mg (tissue) values. For ketone body dosing, beta-hydroxybutyrate was quantitated using a dedicated colorimetric assay kit (#700190; Cayman Chemical, Ann Arbor, Mich.), following manufacturer's instructions and standards to calculate μM (serum) and nmol/mg (tissue) values. For BCAA dosing, BCAA levels (not discriminating individual amino acid concentrations) were assayed using a dedicated colorimetric kit (#ab83374; Abcam, Cambridge, Mass.), following manufacturer's instructions and standards to calculate μM (serum) and nmol/mg extracted protein (tissue) values. All dosing assays relied on triplicates for each standard or sample; tests were run on either serum or approximately 25 mg frozen-pulverized whole tissue (treated according to each kit's procedure). Colorimetric reactions were quantitated using a Synergy HTX multi-mode plate reader (BioTek®, Winooski, Vt.) and averaging four reads/sample at appropriate wavelengths. All dosing assays were conducted blinded to treatment groups.

H3K27ac ChIP-seq on muscle myofibers. Freshly-isolated whole quadriceps muscles (both per mouse) were finely minced and digested in 10 ml/muscle of PBS supplemented with 1 mM CaCl₂ and 100 U/ml collagenase II (Cat #17101, Life Technologies, Grand Island, N.Y.) at 37° C. for 1 hour with shaking. The suspension was then filtered through a 40 μm strainer (Cat #22363547, Fisher Scientific, Waltham, Mass.) and the unfiltered fraction (enriched in myofibers) was kept for further steps. Separation of mononuclear fraction in the filtered fraction was confirmed at the microscope. Myofibers were fixed in 10 ml 1% PFA for 30 minutes at room temperature with gentle nutation. Fixation was quenched 1 ml of 1.375M glycine (Cat #BP381-5, Fisher Scientific, Waltham, Mass.) with gentle nutation for 5 minutes at room temperature. After centrifugation at 3000 g for 5 minutes, myofibers were lysed in 1.4 ml lysis buffer with approximately 25 μl 2.3 mm zirconia/silica beads (Cat #11079125z, BioSpec, Bartlesville, Okla.). Lysis buffer consisted of 10 mM HEPES (pH 7.3; Cat #H3375), 10 mM KCl (Cat #P9541), 5 mM MgCl₂ (Cat #M8266), 0.5 mM DTT (Cat #646563), 3 μg/ml cytochalasin B (C6762; all reagents from Sigma, St. Louis, Mo.); protease inhibitor cocktail (Cat #11852700, Roche, Mannheim, Germany)). Myofibers were them homogenized by means of Mini-BeadBeater-16 (Cat #607, Biospec, Bartlesville, Okla.) for 30 sec, then by rotating at 4° C. for 30 minutes. Samples were centrifuged at 3000 g for 5 minutes at 4° C.; supernatant was removed; pellet was resuspended in cell lysis buffer as per reported conditions (Carey et al., 2009), supplementing the cell lysis buffer with 3 μg/ml cytochalasin B and rotating for 10 minutes at 4° C. Nuclei were pelleted at 300 g for 10 minutes at 4° C., and subsequently processed following reported protocol with the adjustment of adding 3 μg/ml cytochalasin B into all solutions for chromatin preparation and sonication, antibody incubation, and wash steps. Chromatin was then sonicated for 15 cycles (30 sec, high power; 30 sec pause; 200p volume) in a water bath sonicator set at 4° C. (Bioruptor 300; Diagenode, Denville, N.J.). After centrifuging at 10000 g for 10 minutes at 4° C., sheared chromatin was checked on agarose gel for a shear band comprised between approximately 150 and approximately 600 bp. Two μg of chromatin was kept for pooled input controls, whereas leftover chromatin (approximately 50 μg) used for each pull-down reaction: Sp H3K27ac primary antibody (cat #39133, Active Motif, Carlsbad, Calif.) in 2 ml volume rotating at 4° C. overnight. Chromatin complexes were precipitated with 100 μl proteinA/G magnetic beads (cat #88803; Thermo Scientific, Waltham, Mass.). After washes and elution, samples were treated with proteinase K (cat #19131; Qiagen, Hilden, Germany) at 55° C. and cross-linking was reversed through overnight incubation at 65° C. DNA was purified using the MinElute purification kit (cat #28004; Qiagen, Hilden, Germany), quantitated using Qubit reader and reagents. Library preparation and sequencing were conducted at the NU Genomics Core, using TrueSeq ChiP-seq library prep (with size exclusion) on 5 ng chromatin per ChIP sample or pooled input, and HiSeq 50 bp single-read sequencing (approximately 60 million read coverage per sample). Peak analysis was conducted using HOMER software (v4.10, (Heinz et al., 2010)) and synthax (e.g., makeTagDirectory, makeUCSCfile, findPeaks, mergePeaks, annotatePeaks.pl, getDifferentialPeakReplicates.pl, findMotifsGenome.pl) after aligning fastq files to the mm10 mouse genome using bowtie2 (Langmead and Salzberg, 2012). Homer motifs used for peak annotation after unsupervised motif analysis were gre.motif, klf3.motif and mef2c.motif. PCA was conducted using ClustVis (Metsalu and Vilo, 2015). Gene ontology pathway enrichment was conducted (cutoff, 1.5-fold transcriptional change) using the Gene Onthology analysis tool (Ashbumer et al., 2000).

RNA-seq. RNA-seq datasets used for analyses in this work can be accessed on the NCBI GEO databse (GSE95682). Total RNA was purified from approximately 30 mg quadriceps muscle tissue of treated and control DBA/2J-mdx male 6 month-old mice with the RNeasy Protect Mini Kit (Cat #74124; Qiagen, Hilden, Germany) as per manufacturer's instructions. RNA quantity and quality were respectively analyzed with Qubit fluorometer (Cat #033216; Thermo Fisher Scientific, Waltham, Mass.) and 2100 Bioanalyzer (Cat #G2943; Agilent Technologies, Santa Clara, Calif.). Libraries were prepared from approximately 1 mg RNA/sample with TruSeq Stranded Total RNA Library Prep Kit (Cat #RS-122-2203; Illumina, San Diego, Calif.). Libraries were sequenced through the NextSeq 500 System (high-throughput, paired-end 150 bp fragment sequencing; #SY-415-1001; Illumina, San Diego, Calif.). Raw reads were aligned with TopHat v2.1.0 to the mm10 genome assembly (grcm38, version 78) (Trapnell et al., 2009). Transcripts were assessed and raw read counts per gene were quantified with HTseq (Anders et al., 2015). Reads Per Kilobase of transcript per Million mapped reads (RPKM) and fold-changes between groups were calculated using EdgeR from the Bioconductor package (Robinson et al., 2010). Differentially expressed genes were identified by adjusted P-value <0.05. Heatmaps were visualized with GiTools (Perez-Llamas and Lopez-Bigas, 2011).

Muscle metabolomics. Total hydrophilic metabolite content was extracted from quadriceps muscle tissue at treatment endpoint through methanol-water (80:20) extraction, adapting conditions described previously (Bruno et al., 2018). Briefly, total metabolite content from quadriceps muscle was obtained from approximately 100 mg (wet weight) quadriceps muscle tissue per animal. Frozen (−80° C.) muscle was pulverized in liquid nitrogen and homogenized with approximately 250 μl 2.3 mm zirconia/silica beads (Cat #11079125z, BioSpec, Bartlesville, Okla.) in 1 ml methanol/water 80:20 (vol/vol) by means of Mini-BeadBeater-16 (Cat #607, Biospec, Bartlesville, Okla.) for 1 minute. After centrifuging at 5000 rpm for 5 minutes, 200 μl of supernatant were transferred into a tube pre-added with 800 μL of ice-cold methanol/water 80% (vol/vol). Samples were vortexed for 1 minute, and then centrifuged at approximately 20,160×g for 15 minutes at 4° C. Metabolite-containing extraction solution was then dried using SpeedVac (medium power). 200 ul of 50% Acetonitrile were added to the tube for reconstitution following by overtaxing for 1 minute. Samples solution were then centrifuged for 15 minutes at 20,000 g, 4° C. Supernatant was collected for LC-MS analysis for Hydrophilic Metabolites Profiling as follows. Samples were analyzed by High-Performance Liquid Chromatography and High-Resolution Mass Spectrometry and Tandem Mass Spectrometry (HPLC-MS/MS). Specifically, system consisted of a Thermo Q-Exactive in line with an electrospray source and an Ultimate3000 (Thermo) series HPLC consisting of a binary pump, degasser, and auto-sampler outfitted with a Xbridge Amide column (Waters; dimensions of 4.6 mm×100 mm and a 3.5 μm particle size). The mobile phase A contained 95% (vol/vol) water, 5% (vol/vol) acetonitrile, 20 mM ammonium hydroxide, 20 mM ammonium acetate, pH=9.0; B was 100% Acetonitrile. The gradient was as following: 0 min, 15% A; 2.5 min, 30% A; 7 min, 43% A; 16 min, 62% A; 16.1-18 min, 75% A; 18-25 min, 15% A with a flow rate of 400 μL/min. The capillary of the ESI source was set to 275° C., with sheath gas at 45 arbitrary units, auxiliary gas at 5 arbitrary units and the spray voltage at 4.0 kV. In positive/negative polarity switching mode, an m/z scan range from 70 to 850 was chosen and MS1 data was collected at a resolution of 70,000. The automatic gain control (AGC) target was set at 1×10⁶ and the maximum injection time was 200 ms. The top 5 precursor ions were subsequently fragmented, in a data-dependent manner, using the higher energy collisional dissociation (HCD) cell set to 30% normalized collision energy in MS2 at a resolution power of 17,500. The sample volumes of 25 μl were injected. Data acquisition and analysis were carried out by Xcalibur 4.0 software and Tracefinder 2.1 software, respectively (both from Thermo Fisher Scientific). Metabolite levels were analyzed as peak area normalized to wet tissue weight and total iron content. Gene-metabolite pathway enrichment was conducted using the MetaboAnalyst platform (v4.0; Joint Pathway Analysis mode) (Chong et al., 2018).

Multi-modal Imaging (FDG-PET, microCT, MR). Mice were anesthetized in an induction chamber with 3% isoflurane in oxygen, weighed, and then transferred to a dedicated imaging bed with isoflurane delivered via nosecone at 1-2%. Mice were placed in the prone position on a plastic bed and immobilized to minimize changes in position between scans. Respiratory signals were monitored using a digital monitoring system developed by Mediso (Mediso-USA, Boston, Mass.). Mice were imaged with a preclinical microPET/CT imaging system (nanoScan PET/CT, Mediso-USA, Boston, Mass.). CT data was acquired with a 2.2× magnification, <60 μm focal spot, 2×2 binning, with 480 projection views over a full circle, using 50 kVp/520 pA, with a 300 ms exposure time. The projection data was reconstructed with a voxel size of 250 μm and using filtered (Butterworth filter) backprojection software from Mediso. A bone mineral density standard (GRM GmbH, Moehrendorf, Germany) with hydroxyapatite (HA) from 0 to 1200 mg HA/cm³ was used to convert the CT images from Hounsfield units to bone mineral density. The HA standard was imaged with the same parameters. For PET imaging, a target of 10 MBq of ¹⁸F-fluordeoxyglucose (FDG) was injected intravenously after mice had been fasted for four hours. PET acquisition parameters were as follows: 1:1 coincidence detection and 30-minute acquisition time. MLEM reconstruction was used with CT for attenuation correction and scattering. Pixel size was set to 0.3×0.3 mm. After completion of PET/CT, each mouse was transferred to the MRI scanner and a reference standard consisting of one tube of canola oil and one tube of water was positioned above its back. MRI was performed on a 9.4T Bruker Biospec MRI system with a 30 cm bore, a 12 cm gradient insert, and an AutoPac laser positioned motorized bed (Bruker Biospin Inc, Billerica, Mass.). Respiratory signals and temperature were monitored using an MR-compatible physiologic monitoring system (SA Instruments, Stonybrook, N.Y.); a warm water circulating system was used to maintain body temperature. A 72 mm quadrature volume coil (Bruker Biospin, Inc, Billerica, Mass.) was used to image each mouse's whole body in two overlapping fields of view. First, the mouse was positioned with the thorax at the magnet's isocenter and imaged using a T₁-weighted accelerated spin echo sequence (Rapid Acquisition with Relaxation Enhancement, RARE) with five pairs of interleaved axial slice stacks covering brain to mid-abdomen. TR was nominally set at 1000 ms; with respiratory gating the functional TR was approximately 1500 ms (range 1300-2000 ms). The following additional parameters were used: TE=6.25 ms, RARE factor 4, MTX=256×256, FOV 45×45 mm, 15 slices of 1 mm thick, 4 mm gap between slices, and 2 signal averages. Each image stack was acquired with and without fat saturation. Acquisition time was approximately 3 minutes per scan. After imaging the upper portion of the mouse, the imaging bed was moved deeper into the magnet and two more pairs of interleaved image stacks were acquired to cover the lower abdomen and legs. Parameters were the same as above, except for a 1 mm gap between slices and 3 signal averages. The reconstructed data was visualized in Amira 6.5 (FEI, Houston, Tex.). The interleaved MRI stacks for upper body and lower body were individually merged, then normalized to the water signal from the reference standard. Then the upper and lower body stacks were registered to each other using a combination of normalized mutual information and manual registration, and merged to create whole body fat-suppressed and non-fat-suppressed MR images. A difference (fat only) image was created by subtracting the normalized fat-suppressed image from the normalized non-fat-suppressed image and segmented by thresholding (using the water and canola oil references as a guide). A small amount of manual segmentation was necessary in regions near the testes where fat suppression pulses were less effective. CT images were registered to the MRI data using normalized mutual information. The fat region of interest (ROI) was used in both the MRI data and FDG-PET data for quantitative analysis. Additionally, each leg was segmented into its own ROI for FDG-PET analysis using the MRI images without fat saturation. A skeleton ROI was generated for each mouse by using a 750 HU threshold in the CT image. The % injected dose (% ID) of FDG in fat and muscle tissue was calculated by dividing the total PET signal found in the ROI with the total PET signal in a mouse whole-body ROI. Mass of body fat was determined by multiplying the volume of fat ROIs with the average density of adipose tissue (0.92 g/cm³) (Hill et al., 2007). The HA standard was segmented with ROIs of 0, 50, 200, 800, and 1200 mg/cm³ and used to create a linear correlation between HU and bone density with a r² of 0.99.

Metabolic cages. VO₂ (ml/h/kg) and energy expenditure to body weight (kcal/h/kg) were assessed via indirect calorimetry using the TSE Automated Phenotyping System PhenoMaster (TSE system, Chesterfield, Mo.). Mice were singly housed in their home cages in an enclosed environmental chamber (part of the TSE system) with controlled temperature and light/dark cycles (12 hours each; 6 AM-6 PM). After a three-day period of acclimation to the metabolic chamber, data collection started at 48 hours after prednisone or vehicle injection and lasted for 5 days. Measurements of CO2 production and O2 consumption occurred using the attached gas analyzer to assess energy expenditure. In addition, physical activity in three dimensions was monitored via infrared beam breaks through frames mounted on the perimeter of the metabolic cages. Enrichment items were omitted to avoid insulation from sensors and infrared light beam path obstruction. Results are expressed as 12 hour-period values (light/dark; 10 values per mouse). Metabolic cage assays were conducted blinded to treatment groups.

Luciferase experiments in live myofibers. Luciferase plasmids containing regulatory fragments were obtained cloning genomic sequences in the pGL4.23 backbone (#E8411; Promega, Madison, Wis.) using the KpnI-XhoI sites upstream of the minimal promoter site. Fragments were cloned conserving the genomic orientation with regards to transcriptional orientation, adding KpnI and XhoI tails to the appropriate extremities via Phusion PCR. Wildtype fragments with responsive site ablation were cloned from wildtype C57Bl/6J genomic DNA, while mutated fragments (Δ sites) were amplified from ad-hoc synthetized DNA oligonucleotides, using genomic sequences from the C57Bl/6J genomic background (see Table 5 for a complete list of sequences). Flexor digitorum brevis (FDB) fibers were transfected by in vivo electroporation. Methods were described previously in (DiFranco et al., 2009) with modifications described in (Demonbreun and McNally, 2015). Briefly, the hindlimb footpad was injected with 10 μl hyaluronidase (8 units) (Cat #H4272, Sigma, St. Louis, Mo.). After two hours, up to 40 μg in 20 μl of endotoxin-free plasmid (10 μl luciferase vector, 2 μl Renilla vector, 3 μl Klf15 vector (#MR206548; Origene, Rockville, Md.) or Mef2C vector (#32515; Addgene, Cambridge, Mass.; (Kozhemyakina et al., 2009)) was injected into the footpad. Electroporation was conducted by applying 20 pulses, 20 ms in duration/each, at 1 Hz, at 100 V/cm. Animals were allowed to recover for a minimum of seven days and not more than ten days after electroporation to avoid examining injured muscle and to allow sufficient time for plasmid expression (Kerr et al., 2013). GR activation was promoted with a pulse of 1 mg/kg i.p. prednisone 24 hours before luciferase analysis. Ex vivo luciferase assay was performed on whole, electroporated FDB muscles. Muscles were minced and homogenized in lysate buffer and experiments were performed according to Dual Luciferase Assay Kit (Cat #1910; Promega, Madison, Wis.) instructions. Luminescence was recorded at the Synergy HTX multi-mode 96-well plate reader (BioTek®, Winooski, Vt.). Raw values were normalized to Renilla luciferase, then to protein content (MyHC) and finally to vehicle-treated muscles with same plasmids. Results are expressed as fold change to average vehicle. All luciferase quantitation assays were conducted blinded to treatment groups.

TABLE 5
Regulatory sequences and transcription
factor binding sites for luciferase
assays in electroporated myofibers.

| transcription | sequence (GRE sites bold |
|---|---|
| factor binding | and underlined; KRE sites |
| site | bold and double underlined; |
| (position from | MEF2 sites |
| TSS) | in bold and italic) |
| Mef2C | AACTGTGCTTCACAGCATTTCTCTA |
| GRE-KRE | CACATTGTTGTATTATAGCAAATTG |
| (I intron; | AAAACATTTATTTAAGCAAGGAAGC |
| +1173 bp) | AGCTCAAAGCTAGGGACTATACATA |
| | GCAAACATATGAAACCATTTTAATA |
| | AGTAAATTCCATATTCACAAGCAAC |
| | ATGGGCTAATGAATGTAAAAGACAC |
| | AACGGCATACATTGATCAAGAATGC |
| | TATAAATTATTATGCATTAAAATGA |
| | ATTTTCTGGGCT [symbol] A |
| | TTGGTACTTAAGAAGAGAAAAGCTT |
| | C |
| | (SEQ ID NO: 37) |
| Bckdha | TGAGCTATGGTGTCCAAGCAGGACA |
| GRE-KRE | CACTGTCAGGGGACCTGATGCAACC |
| (I intron; | ATTCAGATACCCAGGTGGACTTCAC |
| +12354 bp) | ATACTGGAGCAGGCACAGACCATGT |
| | TCTCCAGTCCCCTCTTTCCAAAGGG |
| | CTGCCTTTACCCCCATGAAGTCACT |
| | GTGCTAATTCAGTGAGTTCCAAAAC |
| | TGGTCAATAATGACACTGGATGCTG |
| | GATTATAGAATGGGCAATAAAATAC |
| | CTACAGAGGCTGGGCAGTGGTAGTG |
| | TACAACTTTAATCCCAGCGCTTGGG |
| | AGGCAGAGGCAGGCGGATCTCTGTG |
| | GGTTTGAGGCCAGCCTGGTATGCAA |
| | AGTGAGTTCCAGGTCAGCCAGGGTG |
| | ACAGAGAAAC [symbol] CAA |
| | TCCTACATGTGGTCATATACCTTCT |
| | CTGTAGGA |
| | (SEQ ID NO: 38) |
| Nmnat3 | TTTGCCTCGGCAGTTTCTTAAGCCA |
| GRE-KRE | CTGTTCTAAGATGGGACTGCTTGTA |
| (I intron; | CTACCAGGAAATGGGTTTGTGGGAA |
| +10206 bp) | GCAGCTGCCAGAGCTGTTCAGACAG |
| | CAGCGGGCACAGCAGGGGTGAAGGT |
| | ATCTCTGCTCCTCAAGACTGAACTT |
| | GAGGTGTCTGTCTCAGAGTAAGCTT |
| | CCCCCCCCC [symbol] TTAGT |
| | ACATTCCCCTCTGACAGATGAGTAA |
| | ACTCTCACAATACAGCCTGTTCTCT |
| | AAGTTGAGGACTGGTTTAACCATTT |
| | TTTGGAAACACTTGGCTCATGCCTT |
| | (SEQ ID NO: 39) |
| Pck1 | AGGGCGGATCTTTGTTTTCTTGTTAT |
| GRE-KRE | TGGCC [symbol] CCCCGAAGACA |
| (promoter; | GGATTTCACTTTGTAGCCCTGGCTG |
| −6335 bp) | TCCTGGAACTCTCCCTGTAGACCAG |
| | GCTGTCCTCAAAACTCACAGAGATC |
| | TGCTTGCCTCTGCTTCCTGAGTGCT |
| | GGGATCGAAGGTGTGTTCCACCACT |
| | GCCCTCCCCCATTTTTTTGTTTTTA |
| | GGGATGAAATTCTGAGCTGGAGAGA |
| | TGGACAGTGGTTAAGAGCATTGACT |
| | GCTCTTCTAGAGGACCTGGGTTCAA |
| | TTCCCAGCACCCATATGGCCGGTCA |
| | CAACTGTCTGCAACTCCAGGATCTA |
| | ACACCCTCACAGAGATGTACATGCA |
| | GGCAAAACATCAGTAAGCCTAAAAT |
| | TAAAAAATGAATTATTTAATAAAGG |
| | AGTGAAATTCACACAACACGAATGA |
| | ACCATTTAAAGATGCACAGTTTAGT |
| | GGCTTGGGTACATCATGTCTAACCA |
| | CCCCTCTTCCTAGTTCC |
| | (SEQ ID NO: 40) |
| Bckdha | CTTGCGACAAAGACGCATAAATGAG |
| MEF2 | TAAGGTGG [symbol] CTCTA |
| (promoter; | AAATTGCTCCGGTCGTCTGCTTCTA |
| −92 bp) | GTTGCTCCTAATTCAGGCAACTAAA |
| | AGGACAACTTAACTTGAACCTTCAG |
| | GGTTCAGGACCCGGAGCCCTGAGCA |
| | AAATGGGCCCTCTCCAAGTCCCTCC |
| | CCCTGTTCCCTGTTGTCCAATGGCT |
| | ATGCCAGAATTGG |
| | (SEQ ID NO: 41) |
| Nmnat3 MEF2 | GAGAAGATGTGGGCAGAACCCTCCT |
| (promoter; | TTGAAAAGCTTAGAAAGATTAGAAG |
| −6589 bp) | AGGAGGTGAGGATGTGTTTAAAGAG |
| | TTCTCTAGTAAAGGGAGGATTTTCC |
| | C [symbol] AAGAGACTTGAGCC |
| | CCTTTATGAGCTAAGAGGAGAGAGC |
| | AGGAAAGATGAAGACACATCAGAGA |
| | ATGGTCACAGTAGACAGAGGACATG |
| | GACAAGAGGGA |
| | (SEQ ID NO: 42) |
| Pck1 MEF2 | AGTCAGTTCCAAACCGTGCTGACCA |
| (promoter; | TGGCTATGATCCAAAGGCCTGCCCC |
| −22 bp) | TTACGTCAGAGGCGAGCCTCCGGGT |
| | CCAGCTGAGGGGCAGGGCTGTCCTC |
| | CCTTCTATATA [symbol] AAGG |
| | AGGGCGGGCTACCAAGCACAGTTGG |
| | CCT (SEQ ID NO: 43) |

Tissue respirometry. Whole-tissue analysis of basal rates of oxygen consumption (OCR) and extracellular acidification (ECAR) was conducted adapting reported conditions for intact muscle tissue analysis (Shintaku and Guttridge, 2016) to the XF96 Extracellular Flux Analyzer platform (Agilent, Santa Clara, Calif.). Immediately after mouse sacrification, target muscle (quadriceps) tissues were quickly collected, rinsed in clean PBS buffer and dissected into approximately 2×2×2 mm pieces. At least three biopsies were sampled for each tissue. Each biopsy was placed at the bottom of a dedicated 96-microplate well (#101085; Agilent, Santa Clara, Calif.), covered with 225 μl of basal respirometry medium and equilibrated at 37° C. in a CO₂-free incubator for 1 hour. Respirometry medium was based on XF Base Medium without Phenol Red (#103335-100; Agilent, Santa Clara, Calif.) supplemented with either 10 mM glucose, 2 mM glutamine, or 2 mM valine. pH was adjusted to 7.4 for all media. Nutrients (#G7021, #V0500, Millipore-Sigma, St Louis, Mo.; #25030-081, Thermo Fisher, Waltham, Mass.) were diluted from 100× stock solutions in XF Base Medium. During biopsy equilibration, a Seahorse XFe96 FluxPak cartridge (#102601-100; Agilent, Santa Clara, Calif.), previously hydrated overnight with 300 μg/well XF calibrant (#100840; Agilent, Santa Clara, Calif.) at 37° C. in a CO₂-free incubator, was loaded with 25 μl appropriate chemical compounds in designated ports and calibrated in the Analyzer. Respirometry analysis was then performed on equilibrated tissue biopsies using the following protocol for each basal or post-injection read: 3 min mix, 5 min delay, 2 min measure. Basal rate reads were collected for 6 consecutive times, then drugs were injected and control reads gathered for additional 3 consecutive times. Drugs to validate basal metabolic rates (catalogue number, referenced inhibitory activity and final concentration are reported after each compound; all compounds from Millipore-Sigma, St Louis, Mo.): to control OCR values, R162 (#538098; inhibitor of glutamate dehydrogenase (Choi and Park, 2018)), 100 μm; DE-NONOate (#D184-50; inhibitor of methylmalonyl-CoA mutase (Kambo et al., 2005)), 5 mM; to control ECAR values, Fx11 (#427218-10 mg; inhibitor of lactate dehydrogenase (Xian et al., 2015)). Compound concentrations were determined on literature and/or preliminary test assays on wildtype muscle biopsies, and the concentration of the compound when loaded in the cartridge port was 10× in appropriate solvent (typically DMSO or ddH₂O). OCR/ECAR reads were averaged for same tissue replicates and subtracted of background noise values (empty wells with only medium and appropriate compound). OCR/ECAR reads were then normalized to biopsy dry weight, measured after overnight incubation of biopsy plate after respirometry analysis at 55° C., hence obtaining pmol O₂/min/mg values for OCR and mph/min/mg values for ECAR. All respirometry analyses were conducted blinded to treatment groups.

2-NBDG uptake assay and glycemia/lactate monitoring. 2-NBDG uptake assay in live myofibers was conducted adapting previously reported conditions (Zou et al., 2005). FDB muscles were collected and carefully treated with collagenase type II and hand pipetting to liberate single myofibers, following reported procedures (Demonbreun and McNally, 2015). Myofibers from two FDB muscles were collected in 1 ml Ringer's solution (for 1 l, 7.2 g NaCl, 0.17 g CaCl₂, 0.37 g KCl; pH, 7.4). 200 μl of myofiber suspension were dispensed per well of chambered coverglass (#155382; Thermo Fisher, Waltham, Mass.) and imaged as baseline condition for both transmitted light (1 ms integration) and green fluorescent channels (100 ms integration) at the Zeiss Axio Observer A1 microscope, using 20× short-range objective and the ZEN 2 software (version 2011; Zeiss, Jena, Germany). Immediately after baseline imaging, myofibers were supplemented with 2 mM glucose (#D8375-1 g; Millipore Sigma, St Louis, Mo.) and 50 μM 2-NBDG (#11046; Cayman Chemical, Ann Arbor, Mich.). For insulin-dependent uptake, insulin (#12585014; Thermo Fisher, Waltham, Mass.) was added to a final 85 μM concentration. To control Glut1-/Glut4-dependent uptake, negative control wells were further supplemented with 10 μM cytochalasin B (#C6762; Millipore Sigma, St Louis, Mo.). Myofibers were incubated for 30 minutes in a 37° C./10% CO₂ incubator, then washed twice in Ringer's solution and immediately imaged in fresh Ringers' solution, using the same integration and objective settings used for pre-incubation pictures. 2-NBDG uptake was quantitated as relative fluorescent units, calculated as intra-myofiber fluorescence after incubation subtracted of average baseline fluorescence. Fluorescence intensity was quantitated through serial analysis of acquired images (3 areas of approximately 85 μm² were analyzed for average fluorescence value per myofiber; >10 myofibers were analyzed per mouse) with ImageJ software (Schneider et al., 2012). All glucose uptake assays were conducted blinded to treatment groups.

Glucose was measured in blood (first drop from tail venipuncture) or serum (5 μl of 1:2 dilution) with an AimStrip Plus glucometer system (Germaine Laboratories, San Antonio, Tex.) and expressed as mg/dl values. Lactate was measured in blood (second drop from tail venipuncture) or serum (5 μl of 1:2 dilution) with a Lactate Plus reader (Nova Biomedical, Waltham, Mass.) and expressed as mM values. Fasting glycemia was measured in mice after 4 hours fasting (7 AM-11 AM). Glucose, insulin and pyruvate tolerance tests were conducted after 4 hours fasting in individual cages immediately after baseline fasting glucose monitoring. Mice were injected with either 1 g/kg glucose (#D8375-1 g; Millipore Sigma, St Louis, Mo.), or 0.5 U/kg insulin (#12585014; Thermo Fisher, Waltham, Mass.), or 2.5 g/kg pyruvate (#P5280-25 g; Millipore Sigma, St Louis, Mo.) in 200 μl intraperitoneal injections, and glucose was then monitored by tail venipuncture at 10 min, 20 min, 30 min, 60 min, 120 min after injection. All glucose and pyruvate tolerance tests were conducted blinded to treatment groups.

MRI scan. Magnetic resonance imaging (MRI) scans to determine fat and lean mass ratios (% of total body weight) were conducted in non-anesthetized, non-fasted mice at 2 PM using the EchoMRI-100H Whole Body Composition analyzer (EchoMRI, Houston, Tex.). Mice were weighed immediately prior to MRI scan. Before each measurement session, system was calibrated using the standard internal calibrator tube (canola oil). Mice were typically scanned in sample tubes dedicated to mice comprised between 20 g and 40 g body mass. Data were collected through built-in software EchoMRI version 140320. Data were analyzed when hydration ratio >85%. MRI scans were conducted blinded to treatment groups.

Histology. Excised tissues (muscles, omental fat, heart) were placed in 10% formaldehyde (Cat #245-684; Fisher Scientific, Waltham, Mass.) for histologic processing. Seven μm sections from the center of paraffin-embedded muscles were stained with hematoxylin and eosin (H&E; cat #12013B, 1070C; Newcomer Supply, Middleton, Wis.) and Masson's trichrome (Cat #HT-15; Sigma-Aldrich; St. Louis, Mo.). Myofiber/adipocyte CSA quantitation was conducted on 400 myofibers/adipocytes per tissue per mouse. Imaging was performed using a Zeiss Axio Observer A1 microscope, using 10× and 20× (short-range) objectives. Brightfield pictures were acquired via Gryphax software (version 1.0.6.598; Jenoptik, Jena, Germany). Area quantitation was performed by means of ImageJ (Schneider et al., 2012). Sample processing, imaging and CSA quantitation were conducted blinded to treatment groups.

CK dosing. Serum creatine kinase (CK) was analyzed in triplicate for each mouse using the EnzyChrom Creatine Kinase Assay (Cat #ECPK-100; BioAssay Systems, Hayward, Calif.) following manufacturer's instructions. Results were acquired with the Synergy HTX multi-mode plate reader (BioTek®, Winooski, Vt.) and expressed as U/ml for murine and U/i for human samples. Both HOP and CK dosing assays were conducted blinded to treatment groups.

Muscle function, whole-body plethysmography, echocardiography. Forelimb grip strength was monitored using a meter (Cat #1027SM; Columbus Instruments, Columbus, Ohio) blinded to treatment groups. Animals performed ten pulls with 5 seconds rest on a flat surface between pulls. Immediately before sacrifice, in situ tetanic force from tibialis anterior muscle was measured using a Whole Mouse Test System (Cat #1300A; Aurora Scientific, Aurora, ON, Canada) with a 1N dual-action lever arm force transducer (3000-LR, Aurora Scientific, Aurora, ON, Canada) in anesthetized animals (0.8 I/min of 1.5% isoflurane in 100% O₂). Tetanic isometric contraction was induced with following specifications: initial delay, 0.1 sec; frequency, 200 Hz; pulse width, 0.5 msec; duration, 0.5 sec; using 100 mA stimulation (Quattrocelli et al., 2015). Length was adjusted to a fixed baseline of 50 mN resting tension for all muscles/conditions. Fatigue analysis was conducted by repeating tetanic contractions every 10 seconds until complete exhaustion of the muscle (50 cycles). Time of contraction was assessed as time to max tetanic value within the 0.0-0.5 sec range of each tetanic contraction, while time of relaxation was assessed as time to 90% min tetanic value within the 0.5-0.8 sec range of every tetanus. Unanesthetized whole-body plethysmography (WBP) was used to measure respiratory function using a Buxco Finepointe 4-site apparatus (Data Sciences International, New Brighton, Minn.). Individual mice were placed in a calibrated cylindrical chamber at room temperature. Each mouse was allowed to acclimate to the plethysmography chamber for 120 minutes before recording was initiated. Data was recorded for a total of 15 minutes broken into 3 consecutive 5-minute periods. All physiological studies were conducted blinded to treatment groups. Cardiac function was assessed by echocardiography, which was conducted under anesthesia (0.8 L/min of 1.5% vaporized isoflurane in 100% O₂) on mice between 2 and 5 days before sacrifice. Echocardiography was performed using a Visual Sonics Vevo 2100 imaging system with an MS550D 22-55 MHz solid-state transducer (FujiFilm, Toronto, ON, Canada). Longitudinal and circumferential strain measurements were calculated using parasternal long-axis and short-axis B-mode recordings of three consecutive cardiac cycles, analyzed by the Vevo Strain software (FujiFilm, Toronto, ON, Canada). Recording and analysis were conducted blinded to treatment group.

Protein analysis. Protein lysates from approximately 50 mg muscle tissue were obtained with homogenization at the TissueLyser II (cat #85300; Qiagen, Hilden, Germany) for two rounds of 2 minutes each with 2 minutes pause in between, using sample plates chilled at −20° C. o/n and one stainless 5 mm bead per sample (cat #69989; Qiagen, Hilden, Germany). Each tissue was homogenized in 250 μl RIPA buffer (cat #89900, Thermo Scientific, Waltham, Mass.) supplemented with protease and phosphatase inhibitors (cat #04693232001 and #04906837001, Roche, Basel, Switzerland). Homogenized samples were then sonicated for 15 cycles (30 sec, high power; 30 sec pause; 200 μl volume) in a water bath sonicator set at 4° C. (Bioruptor 300; Diagenode, Denville, N.J.) and approximately 10 μg protein lysate was mixed with 1:1 volume of 2× Laemmli buffer (cat #161-0737; Bio-Rad, Hercules, Calif.) and incubated at 95° C. for 15 minutes. Protein electrophoresis was performed in 4-15% gradient gels (cat #456-1086; Bio-Rad, Hercules, Calif.) in running buffer containing 25 mM TRIS, 192 mM glycine, 0.1% SDS, pH 8.3. Proteins were then blotted on 0.2 μm PVDF membranes (cat #16220177; Bio-Rad, Hercules, Calif.), previously activated for 3 minutes in 100% methanol, in transfer buffer containing 25 mM TRIS, 192 mM glycine, 20% methanol at 300 mA for approximately 3.5 hours at 4° C. Membranes were washed with TBS-T buffer containing 20 mM TRIS, 150 mM NaCl, 0.1% Tween-20, pH 7.6, and then blocked with StartingBlock (cat #37543, Thermo Scientific, Waltham, Mass.). Primary antibody incubation was performed overnight at 4° C. with the following antibodies: rabbit anti-phospho BCKDHA (ser293; cat #A304-672A-T), anti-total BCKDHA (cat #A303-790A-T), rabbit anti-mTOR (cat #A301-143A-T), rabbit anti-RagC (cat #A304-299A-T), rabbit anti-S6K (cat #A300-510A-T), rabbit anti-4EBP1 (cat #A300-501A-T; Bethyl Laboratories, Montgomery, Tex.); rabbit anti-phopsho-S6K (Thr389; cat #AP0564), rabbit anti-phosho-4EBP1 (Ser65; cat #AP0032; ABclonal, Woburn, Mass.); mouse anti-myosin heavy chain (cat #MF20), mouse anti-puromycin (cat #PMY-2A4; DSHB, Iowa City, Iowa). Secondary antibody incubation was performed at room temperature for 1 hour with the following antibodies: donkey anti-rabbit and anti-mouse (cat #sc-2313 and #2314; Santa-Cruz Biotechnology; Dallas, Tex.). Blots were developed with Super Signal Femto (cat #34096; Thermo Scientific, Waltham, Mass.) using the iBrightCL1000 developer system (cat #A32749; Thermo Scientific, Waltham, Mass.) with automatic exposure settings. Protein density was analyzed using the Gel Analysis tool in ImageJ software (Schneider et al., 2012). Only bands from samples run and blotted in parallel on the same gels/membranes were analyzed for ratios. Phosphorylation levels were quantitated as ratio versus total protein; co-IP levels were quantitated as ratio versus bait protein; total protein levels were quantitated as ratio to housekeeping/structural protein control. Image acquisition and densitometric analysis were conducted blinded to treatment group.

Statistical analysis. Statistical analyses were performed using Prism software v7.0a (Graphpad, La Jolla, Calif.). Normality of data pools was tested with the Pearson-D'Agostino normality test. When comparing two groups, two-tailed Student's t-test with Welch's correction (unequal variances) was used. When comparing three groups of data for one variable, one-way ANOVA with Tukey multi-comparison was used. When comparing data groups for more than one related variable, two-way ANOVA was used and the Tukey multi-comparison additionally used when comparing more than two data groups. For ANOVA and t-test analyses, a P value less than 0.05 was considered significant. Stacks of p-value were analyzed with Benjamini-Hochberg test to calculate a q-value (metabolomics, epigenomics). Data were presented as single values (dot plots, histograms) when the number of data points was less than 15. In analyses pooling larger data point sets per group (typically >50 data points), Tukey distribution bars were used to emphasize data range distribution. Analyses pooling data points over time were presented as marked line plots. Tables, dot plots, histograms and marked line plots depict mean±SEM. Box plots depict the Tukey distribution of the data pool.

### Example 2

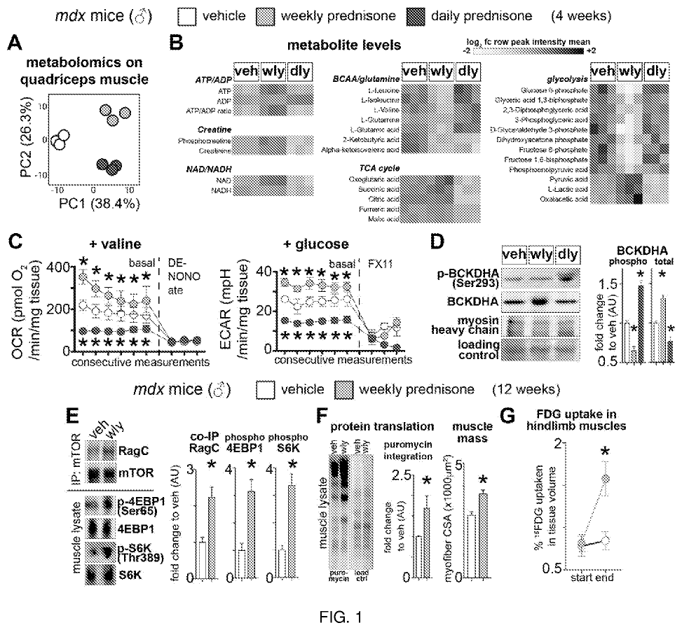

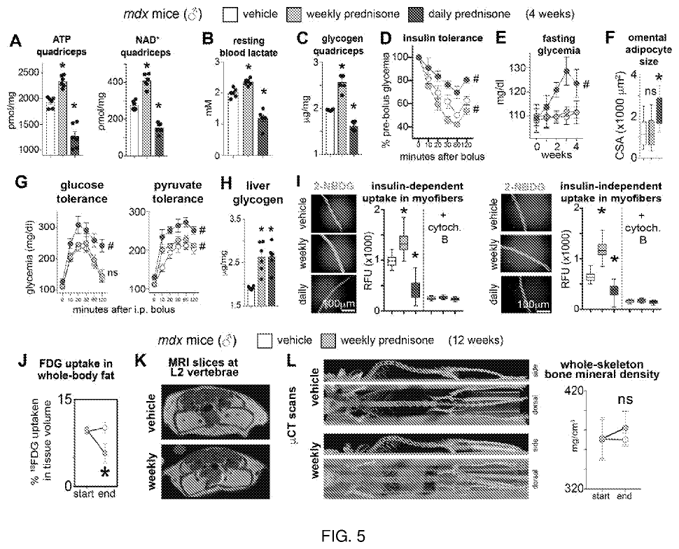

Pulsatile glucocorticoid exposure enhanced mitochondrial respiration in dystrophic muscle through BCAA. Weekly prednisone promotes dystrophic muscle growth and force, while daily dosing evokes wasting and weakness (Quattrocelli et al., 2017a; Quattrocelli et al., 2017b). To pinpoint the metabolic pathways altered in muscle by these prednisone regimens, unbiased metabolomics was performed on mdx muscles (n=3, 4 wk exposure). Principal component analyses (PCA) showed clustering of metabolite profiles according to steroid regimen across 171 hydrophilic metabolites (FIG. 1A). Weekly prednisone increased ATP, phosphocreatine, and NAD⁺ (FIG. 1B, left). This correlated with increased catabolism of BCAAs, seen as reduced levels of precursors and intermediates and increased levels of oxoglutarate and succinate in the TCA cycle (FIG. 1B, center). Glycolysis was also increased with increased levels of pyruvate and lactate (FIG. 1B, right). Conversely, daily prednisone correlated with loss of both NAD⁺ and NADH, and substantial impairment of BCAA and glucose catabolism (FIG. 1B). Opposing shifts were confirmed by HPLC analyses of muscle ATP and NAD⁺and by resting blood lactate (FIGS. 5A and 5B), and these changes correlated with parallel shifts in muscle glycogen content (FIG. 5C).

Respirometry assays on quadriceps muscle (n=6 mice/group) showed that, opposite to daily dosing, weekly prednisone improved valine-fueled oxygen consumption and glucose-fueled lactate production (FIG. 1C). In quadriceps muscle, weekly prednisone also increased total levels and reduced phosphorylation of branched chain keto acid dehydrogenase (BCKDHA), which commits BCAA to oxidative metabolism and is inhibited by phosphorylation (White et al., 2018) (FIG. 1D). Thus, pulsatile prednisone improved BCAA utilization to mitochondrial respiration and energy production, while increasing glucose catabolism and NAD⁺ levels.

Daily prednisone impaired glucose homeostasis in mdx mice (FIG. 5D-I; Table 1). In contrast, weekly prednisone-treated mice showed higher insulin sensitivity (FIG. 5D; Table 1), thereby offsetting glucocorticoid-driven gluconeogenesis and normalizing glycemia (FIG. 5E-H). Weekly prednisone enhanced myogenic glucose uptake, as quantitated through 2-NBDG (fluorescent glucose analog) in isolated myofibers (FIG. 5I. Protein analysis for mechanistic target of rapamycin (mTOR) pathway members in mdx muscle showed that, after a 12-week-long treatment, weekly prednisone increased levels of mTOR-bound RagC and phosphorylation of S6K and 4EBP1 (FIG. 1E), indicating increased amino acid sensing and mTOR activation (Sancak et al., 2008). Accordingly, weekly treatment increased protein translation and muscle mass, as shown by increased puromycin incorporation and myofiber size (FIG. 1F). With multi-modal imaging of live animals, weekly prednisone was found to increase muscle uptake of ¹⁸FDG, a glucose analog, while decreasing uptake in fat (FIG. 1G; FIG. 5J). Daily dosing induces obesity and osteoporosis. However, magnetic resonance and tomography showed that weekly prednisone did not increase fat mass accumulation or reduce bone mineral density (FIG. 5K-L). Thus, compared to daily intake, a weekly GC steroids improved amino acid sensing, insulin sensitivity, and muscle growth in mdx mice.

TABLE 1
Weekly and daily prednisone regimens exert opposing effects on BCAA
disposal and insulin sensitivity in mdx mice (4-week treatment).

| | vehicle | daily prednisone | | weekly prednisone | |
|---|---|---|---|---|---|
| | mean ± | mean ± | P value | mean ± | P value |
| | s.e.m | s.e.m | vs vehicle | s.e.m | vs vehicle |
| SERUM | | | | | |
| insulin (ng/ml) | 1.64 ± 0.09 | 3.09 ± 0.14 | <0.0001 | 1.73 ± 0.07 | 0.820 |
| BCAA (μM) | 572 ± 25.3 | 657 ± 24.2 | 0.037 | 457 ± 14.1 | 0.005 |
| free fatty acids (μM) | 462 ± 11 | 546 ± 13.5 | 0.002 | 386 ± 17.3 | 0.005 |
| β-hydroxybutyrate (μM) | 451 ± 3.05 | 476 ± 4.81 | 0.002 | 425 ± 4.92 | 0.002 |
| corticosterone (ng/ml) | 181 ± 1.01 | 127 ± 5.34 | <0.0001 | 194 ± 5.05 | 0.118 |
| TISSUE BCAA (nmol/mg) | | | | | |
| quadriceps | 105 ± 3.37 | 118 ± 2.74 | 0.019 | 89.6 ± 3.06 | 0.008 |
| diaphragm | 51.6 ± 1.51 | 64 ± 3.09 | 0.002 | 43.8 ± 0.93 | 0.045 |
| heart | 2.03 ± 0.08 | 2.38 ± 0.09 | 0.017 | 1.65 ± 0.07 | 0.012 |
| omental fat | 23.9 ± 1.32 | 28.2 ± 0.99 | 0.035 | 18.1 ± 0.94 | 0.005 |
| TISSUE FREE FATTY | | | | | |
| ACIDS (nmol/mg) | | | | | |
| quadriceps | 2.26 ± 0.1 | 4.11 ± 0.11 | <0.0001 | 1.25 ± 0.07 | <0.0001 |
| diaphragm | 1.78 ± 0.07 | 2.07 ± 0.09 | 0.021 | 1.11 ± 0.07 | <0.0001 |
| heart | 0.776 ± 0.06 | 0.86 ± 0.03 | <0.0001 | 0.44 ± 0.02 | <0.0001 |
| omental fat | 5.08 ± 0.1 | 6.01 ± 0.3 | 0.008 | 4.35 ± 0.07 | 0.038 |
| TISSUE β- | | | | | |
| HYDROXYBUTYRATE | | | | | |
| (nmol/mg) | | | | | |
| quadriceps | 23.4 ± 0.34 | 31.8 ± 0.48 | <0.0001 | 16.4 ± 1.48 | 0.001 |
| diaphragm | 24.7 ± 0.75 | 28.4 ± 0.93 | 0.017 | 17.1 ± 0.77 | <0.0001 |
| heart | 21.1 ± 0.66 | 24.6 ± 0.88 | 0.001 | 16.5 ± 0.56 | 0.001 |
| omental fat | 24.1 ± 0.66 | 58.8 ± 2.29 | <0.0001 | 19 ± 0.39 | 0.041 |

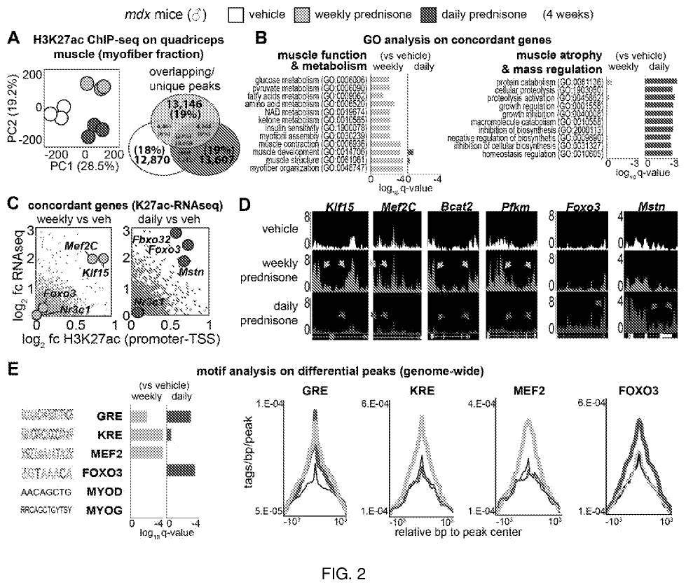

Epigenetic programs in steroid-treated dystrophic muscles. To explore the epigenetic and transcriptional programs elicited by steroid treatment of dystrophic muscle, the genomewide distribution of histone 3 lysine 27 acetylation (H3K27ac), a marker of transcriptional activation at enhancers and promoters (Rivera and Ren, 2013), was analyzed. H3K27ac analysis of the myofiber fraction of mdx muscle (n=3 mice/group) was integrated with the muscle-matched RNAseq transcriptome (GSE95682; n=5 mice/group). PCA analysis of global H3K27ac data clustered the profiles according to prednisone regimen (FIG. 2A). Gene ontology (GO) analysis was conducted on concordant genes, i.e. genes with concordant gain in promoter acetylation and transcriptional activation or vice versa. For weekly prednisone, the GO terms for nutrient metabolism and muscle function were highly enriched, while GO terms for muscle atrophy were enriched for daily prednisone (FIG. 2B). Notably, the glucocorticoid receptor (GR) gene, Nr3c1, was not significantly changed in H3K27ac marking or expression, suggesting GR activity and/or downstream cascades as mediators (FIG. 2C).

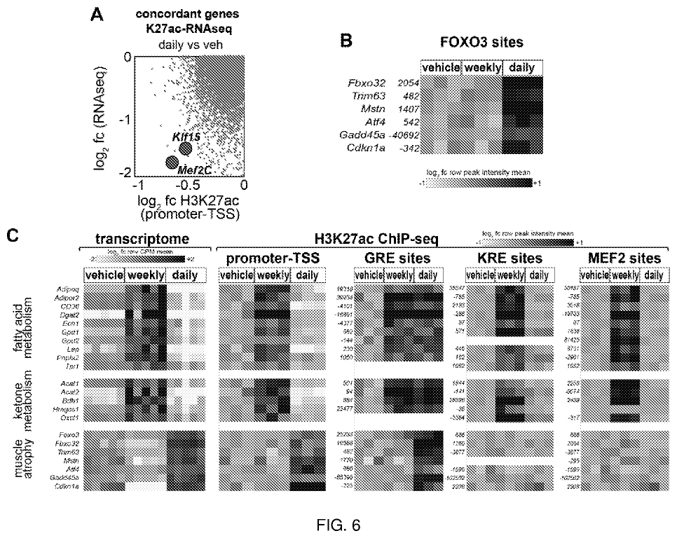

Weekly prednisone increased H3K27ac marks and transcription of KIf15, a GR-activated KLF factor (Morrison-Nozik et al., 2015), and Mef2C, a regulator of muscle growth (Lin et al., 1997), along with BCAA and glucose pathway genes (FIG. 2C-D). These changes were regimen-sensitive, as daily prednisone correlated with reduced levels of KIf15 and Mef2C (FIG. 6A) and upregulation of Foxo3 and other muscle atrophy factors (FIG. 2C-D). Unbiased motif analysis on differential H3K27ac peaks showed that weekly prednisone induced higher H3K27ac marking at GR elements (GRE), KLF-responsive elements (KRE) and MEF2 binding sites, consistent with increased activities of GR, KLF15 and MEF2C, but not at binding sites for canonical myogenic factors like MyoD and myogenin (FIG. 2E). In contrast, daily prednisone induced H3K27ac enrichment at GRE and FOXO3 sites, but not KRE or MEF2 sites, of atrogenes such as Fbxo32, Trim63, Mstn, Atf4, Gadd45a and Cdkn1a (p21) (FIG. 2E; FIG. 6B), consistent with a muscle wasting profile (Bodine et al., 2001; Bullard et al., 2016; Sandri et al., 2004).

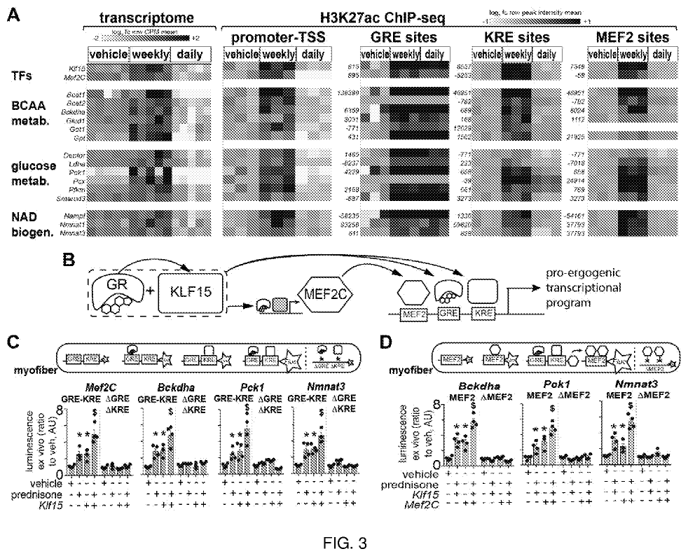

KLF15 and MEF2C mediate genomewide program supporting BCAA utilization, glucose metabolism and NAD biogenesis in dystrophic muscle. To determine the epigenomic impact of glucocorticoids on metabolic networks, pathways of BCAA utilization, glucose metabolism and NAD biogenesis were interrogated. Pathway-centered heat-maps show that weekly prednisone led to a concerted upregulation in expression and H3K27ac marking at promoters and enhancers containing GRE, KRE and MEF2 sites in loci of key genes involved in these metabolic cascades, along with the transcription factors KIf15 and Mef2C (FIG. 3A). Daily prednisone induced similar enrichment in H3K27ac at GRE sites, but had opposing effects on H3K27ac marking at KRE and MEF2 sites and expression levels, highlighting the importance of KLF15 and MEF2C in discriminating between GR-responsive pro-ergogenic and pro-atrophic programs (FIG. 3A). Gene pathways involved in fatty acid and ketone body metabolism were also upregulated by weekly prednisone, reflecting activated muscle metabolism (FIG. 6C). In aggregate with motif density analyses, these data depict an epigenomic program of functional cooperation between activated GR, KLF15 and MEF2C in driving a pro-ergogenic reprogramming of muscle metabolism (FIG. 3B).

This hypothesis was tested in myofibers by expressing reporter constructs carrying GRE-KRE and MEF2 genomic sites upstream from key downstream regulators including Mef2C, Bckdha (BCAA utilization), Pck1 (glucose metabolism) and Nmnat3 (NAD biogenesis). Reporter activation was monitored by measuring firefly luciferase (Fluc) activation in electroporated mdx myofibers (n=4 mice/group) in the presence of either a prednisone pulse (1 mg/kg), or a Klf15 overexpression pulse, or the combination thereof. Experiments were controlled with similar vectors specifically deleted for the GRE and KRE sequences (AGRE-KRE). Prednisone and Klf15 pulses had an additive effect on Fluc reporter activity, whereas Fluc upregulation was blunted in the absence of GRE-KRE sites (FIG. 3C). Moreover, MEF2 site-containing regulatory regions of Bckdha, Pck1 and Nmnat3 demonstrated the same pattern. Prednisone, Klf15 and Mef2C pulses had an additive effect on Fluc activation, while Fluc activity remained unchanged with ΔMEF2 reporter vectors (FIG. 3D). Together KLF15 and MEF2C cooperate with activated GR to enhance BCAA utilization, glucose metabolism and NAD biogenesis.

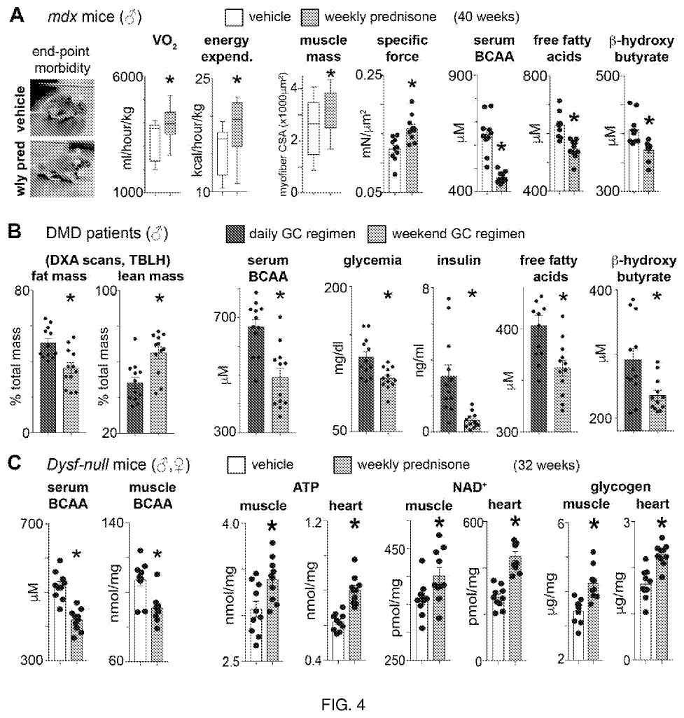

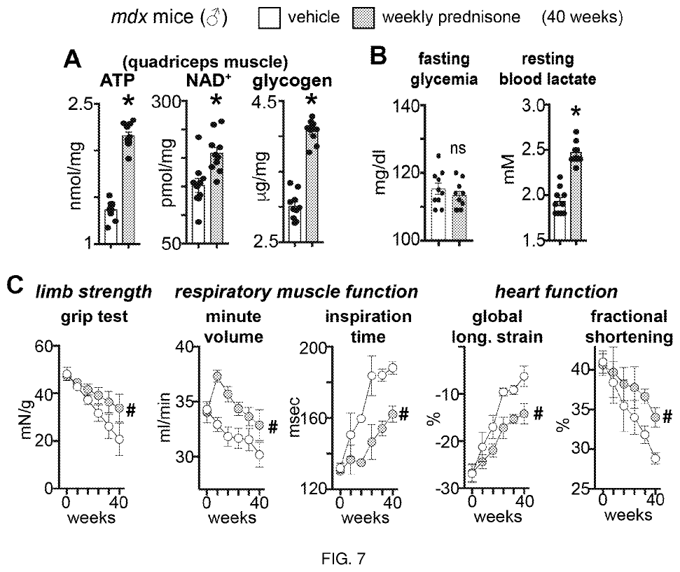

Pulsatile glucocorticoids reduce BCAA accumulation and improve insulin sensitivity in dystrophic mice and humans with Duchenne Muscular Dystrophy. To test the durability of favorable muscle reprogramming, mdx male mice were treated with weekly prednisone for 40 weeks beginning at 6 weeks of age (n=10 mice/group). Prednisone treatment improved morbidity and increased oxygen consumption (VO₂) and energy expenditure during nocturnal activity (FIG. 4A). The same effects were seen after 40 weeks of weekly prednisone with an increase in ATP, NAD+, and glycogen in muscle and blood lactate with no change in blood glucose (FIG. 7A-B). Furthermore, 40 wk-treated mice showed increased muscle mass and force, and reduced levels of BCAA, free fatty acids and ketones in circulation and peripheral tissues, indicating higher levels of BCAA utilization and nutrient sensitivity (FIG. 4A; Table 2). Favorable muscle reprogramming correlated with improved performance of limb muscles, respiratory muscles and heart (FIG. 7C). Therefore, BCAA utilization and pro-ergogenic reprogramming were durable in long-term weekly prednisone treated mdx mice.

TABLE 2
Long-term weekly prednisone boosts BCAA disposal and utilization in
peripheral tissues, along with free fatty acids and ketone bodies.

| | vehicle | weekly prednisone | |
|---|---|---|---|
| | mean ± s.e.m | mean ± s.e.m | P value |
| BLOOD and SERUM | | | |
| creatine kinase (U/ml) | 5.42 ± 0.4 | 3.1 ± 0.16 | 0.001 |
| insulin (ng/ml) | 1.3 ± 0.1 | 1.51 ± 0.14 | 0.219 |
| corticosterone (ng/ml) | 150 ± 10.6 | 133 ± 8.36 | 0.228 |
| BCAA (μM) | 647 ± 26 | 462 ± 7.32 | <0.0001 |
| free fatty acids (μM) | 629 ± 13.5 | 547 ± 10.4 | 0.001 |
| β-hydroxybutyrate (μM) | 407 ± 8.04 | 372 ± 5 | 0.002 |
| TISSUE BCAA (nmol/mg) | | | |
| quadriceps | 118 ± 2.3 | 95.3 ± 3.21 | <0.0001 |
| diaphragm | 57.5 ± 2.9 | 49.4 ± 2.14 | 0.037 |
| heart | 2.46 ± 0.05 | 1.74 ± 0.08 | <0.0001 |
| omental fat | 31.3 ± 1.46 | 23.5 ± 0.43 | 0.001 |
| TISSUE FREE FATTY | | | |
| ACIDS (nmol/mg) | | | |
| quadriceps | 2.05 ± 0.08 | 0.752 ± 0.05 | <0.0001 |
| diaphragm | 2.4 ± 0.07 | 1.46 ± 0.05 | <0.0001 |
| heart | 0.739 ± 0.03 | 0.211 ± 0.01 | <0.0001 |
| omental fat | 4.54 ± 0.21 | 3.91 ± 0.12 | 0.019 |
| TISSUE β- | | | |
| HYDROXYBUTYRATE | | | |
| (nmol/mg) | | | |
| quadriceps | 55.9 ± 4.83 | 24.7 ± 1.58 | <0.0001 |
| diaphragm | 28.6 ± 0.96 | 12.9 ± 0.89 | <0.0001 |
| heart | 38.4 ± 2.49 | 20.2 ± 0.51 | <0.0001 |
| omental fat | 30.4 ± 1.42 | 23 ± 1.71 | 0.004 |

To evaluate the clinical relevance of intermittent glucocorticoid treatment in humans, data and samples from DMD patients were analyzed. In DMD, most patients receive daily steroids, but pulsatile weekend high-dose treatment (two consecutive days per week) has been proposed as alternative to improve ambulation and limit side effects (Connolly et al., 2002). Clinical data and serum biomarkers were compared from DMD boys receiving daily (1-2.5 mg/kg) or weekend (1-4 mg/kg) steroids (n=12 patients/group; 7/12 on prednisone and 5/12 on deflazacort in each group), matching age, treatment duration and body mass index (Table 3). As shown by dual-energy X-ray absorptiometry (DEXA) scans, weekend steroid treatment was associated with decreased fat mass ratio by approximately 30% and increased lean mass by approximately 30% (FIG. 4B). Weekend dosing was linked to lower levels of circulating BCAA, glucose, insulin, free fatty acids and ketone bodies (FIG. 4B). Importantly, daily and weekend regimens had comparable effects on ambulation, serum creatinine kinase levels, and cardiac function (Table 3). Pulsatile glucocorticoid treatment promotes BCAA disposal and lean mass improvement in DMD, curtailing the dysmetabolism caused by daily glucocorticoid intake.

TABLE 3
In DMD patients, intermittent glucocorticoids
mitigate biomarkers of obesity and metabolic
syndrome compared to daily glucocorticoids.

| | daily GC regimen | weekend GC regimen | |
|---|---|---|---|
| | mean ± s.e.m | mean ± s.e.m | P value |
| MEASUREMENTS | | | |
| age (years) | 10.92 ± 0.92 | 9.59 ± 0.82 | 0.199 |
| treatment duration (months) | 54.92 ± 6.94 | 47.17 ± 7.74 | 0.417 |
| height (cm) | 129.07 ± 4.33 | 137.09 ± 6.6 | 0.217 |
| weight (kg) | 36.6 ± 3.65 | 43.46 ± 8.63 | 0.377 |
| BMI (kg/m2) | 21.89 ± 1.72 | 21.77 ± 2.59 | 0.963 |
| fat mass TBLH (%) | 50.36 ± 3.07 | 36.28 ± 3.61 | 0.002 |
| lean mass TBLH (%) | 48.19 ± 3.94 | 64.93 ± 4.19 | 0.002 |
| BMD TBLH (Z-score) | −2.92 ± 0.22 | −1.2 ± 0.26 | <0.001 |
| BMD L1-L4 (Z-score) | −2.13 ± 0.28 | −0.34 ± 0.25 | <0.001 |
| SERUM | | | |
| cortisol (ng/ml) | 2.76 ± 0.54 | 15.4 ± 3.65 | 0.001 |
| glucose (mg/dl) | 126.67 ± 7.25 | 105.25 ± 3.69 | 0.005 |
| insulin (ng/ml) | 3.1 ± 0.79 | 0.64 ± 0.16 | 0.003 |
| BCAA (μM) | 633 ± 31.8 | 492 ± 31.3 | 0.005 |
| free fatty acids (μM) | 402.62 ± 9.03 | 361.86 ± 8.87 | 0.004 |
| β-hydroxybutyrate (μM) | 290.65 ± 16.44 | 235.36 ± 5.01 | 0.011 |
| lactate (mM) | 2.18 ± 0.17 | 2.81 ± 0.14 | 0.003 |
| MOBILITY and MUSCLE | | | |
| DAMAGE | | | |
| Brooke's score (AU) | 6.00 ± 1.10 | 5.25 ± 1.14 | 0.571 |
| 10 m run test (sec) | (n = 5) | (n = 7) | 0.785 |
| | 7.5 ± 0.57 | 6.7 ± 1.07 | |
| creatine kinase (U/L) | 12626 ± 3448 | 17770 ± 4850 | 0.312 |
| HEART FUNCTION | | | |
| PR interval (msec) | 113.45 ± 2.87 | 118.55 ± 5.32 | 0.339 |
| QRS interval (msec) | 83.09 ± 3.11 | 77.64 ± 3.01 | 0.202 |
| QT interval (msec) | 336.36 ± 9.20 | 337.09 ± 10.09 | 0.951 |
| LV septum thickness (cm) | 0.70 ± 0.04 | 0.69 ± 0.06 | 0.917 |
| LV PW thickness (cm) | 0.73 ± 0.04 | 0.70 ± 0.05 | 0.592 |
| fractional shortening (%) | 31.49 ± 1.35 | 32.05 ± 0.90 | 0.786 |

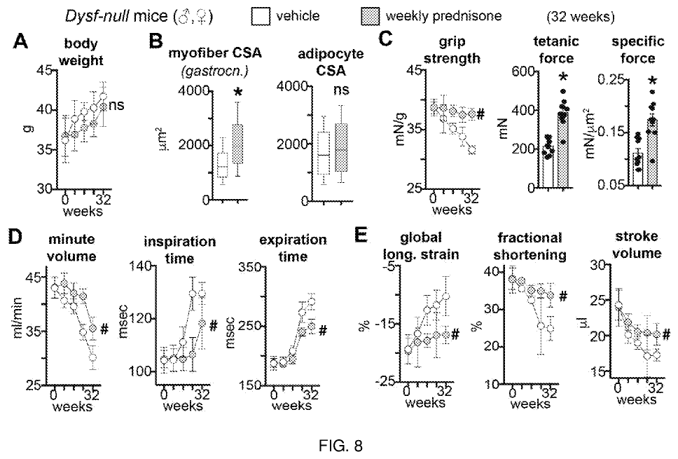

To explore whether pulsatile glucocorticoids may be useful in other forms of muscular dystrophy, the metabolic effects in a mouse model of limb girdle muscular dystrophy was interrogated. A form of muscular dystrophy for which clinical data suggested a deleterious effects from daily prednisone in patients (Walter et al., 2013) was specifically selected. Dysferlin deficient (Dysf-null) mice, a genetic model of this disease, received long-term treatment with weekly prednisone for 32 weeks from the age of disease onset (approximately 9 months; n=10 mice/group; randomized males/females). Consistent with observations in mdx mice and DMD patients, intermittent prednisone improved BCAA utilization in muscle (FIG. 4C). Circulating free fatty acids and ketone bodies were also decreased after treatment (Table 4). Furthermore, endpoint levels of ATP, NAD+, and glycogen were increased in muscle and heart (FIG. 4C). Pulsatile prednisone increased muscle mass and improved performance of limb muscles, respiratory muscles, and heart (FIG. 8), expanding this favorable metabolic reprogramming regimen to a pathologically distinct form of muscular dystrophy.

TABLE 4
Weekly steroid dosing promotes favorable remodeling of glucose,
fatty acid and ketone metabolism in Dysf-null mice.

| | vehicle | weekly prednisone | |
|---|---|---|---|
| | mean ± s.e.m | mean ± s.e.m | P value |
| BLOOD and SERUM | | | |
| fasting glycemia (mg/dl) | 114 ± 3.51 | 109 ± 3.17 | 0.228 |
| resting lactate (mM) | 2.02 ± 0.11 | 3.36 ± 0.21 | <0.0001 |
| insulin (ng/ml) | 1.26 ± 0.1 | 1.22 ± 0.11 | 0.815 |
| BCAA (μM) | 519 ± 12.7 | 419 ± 9.95 | <0.0001 |
| free fatty acids (μM) | 543 ± 11.7 | 475 ± 9.18 | 0.001 |
| β-hydroxybutyrate (μM) | 386 ± 8.16 | 323 ± 9.61 | <0.0001 |
| corticosterone (ng/ml) | 149 ± 6.08 | 143 ± 5.2 | 0.446 |
| creatine kinase (U/ml) | 2.42 ± 0.08 | 1.31 ± 0.04 | <0.0001 |
| TISSUE BCAA (nmol/mg) | | | |
| quadriceps | 107 ± 3.46 | 90.9 ± 2.58 | 0.002 |
| diaphragm | 51.3 ± 1.82 | 45.4 ± 1.06 | 0.014 |
| heart | 1.89 ± 0.04 | 0.91 ± 0.03 | <0.0001 |
| omental fat | 35.2 ± 0.81 | 24.5 ± 0.8 | <0.0001 |
| TISSUE FREE FATTY | | | |
| ACIDS (nmol/mg) | | | |
| quadriceps | 2.98 ± 0.24 | 1.05 ± 0.06 | <0.0001 |
| diaphragm | 1.7 ± 0.26 | 0.753 ± 0.07 | 0.005 |
| heart | 0.69 ± 0.04 | 0.438 ± 0.05 | 0.001 |
| omental fat | 6.69 ± 0.31 | 3.49 ± 0.38 | <0.0001 |
| TISSUE β- | | | |
| HYDROXYBUTYRATE | | | |
| (nmol/mg) | | | |
| quadriceps | 110 ± 3.78 | 43.8 ± 2.23 | <0.0001 |
| diaphragm | 81.6 ± 6.48 | 26.5 ± 2.11 | <0.0001 |
| heart | 19.6 ± 1.69 | 11.9 ± 0.51 | 0.001 |
| omental fat | 37.8 ± 1.59 | 30.2 ± 0.85 | 0.001 |

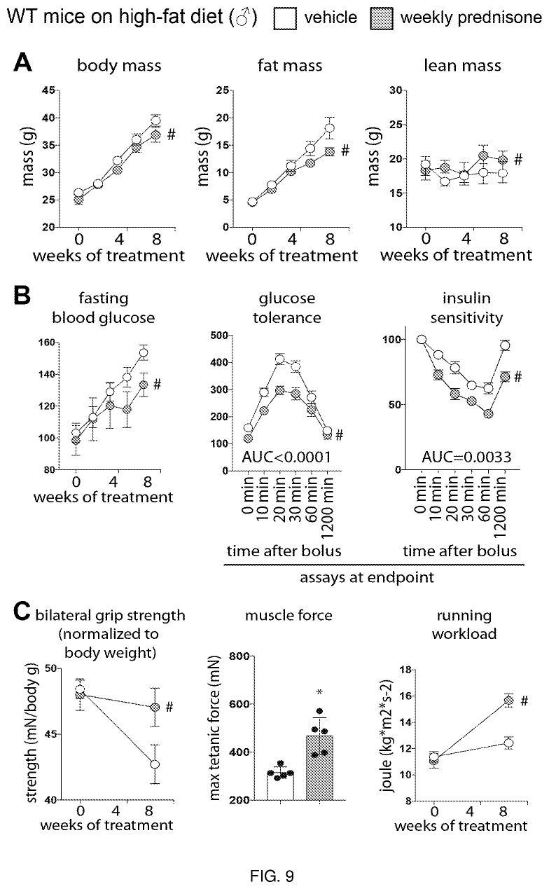

To investigate the impact of pulsatile glucocorticoids in conditions of metabolic stress, the effects of this drug regimen were monitored in experimental conditions of obesity (FIG. 9). Wildtype (WT) mice were fed high-fat chow and treated with either vehicle or weekly (pulsatile) 1 mg/kg intraperitoneal prednisone administration for 8 weeks. (FIG. 9A) As compared to vehicle treatment, weekly prednisone slightly but significantly reduced gain of body weight and fat mass, while improved lean mass retention. (FIG. 9B) Weekly prednisone reduced the gain of hyperglycemia, as shown by fasting blood glucose levels over time. At diet exposure endpoint, obese mice treated with weekly prednisone showed improved body-wide glucose homeostasis, as shown by glucose and insulin tolerance tests. (FIG. 9C) Weekly prednisone improved grip strength (forelimbs, bilateral), tetanic force production (tibialis anterior, in situ) and aerobic exercise capacity (run-to-exhaustion, treadmill) at the end of high-fat diet regimen.

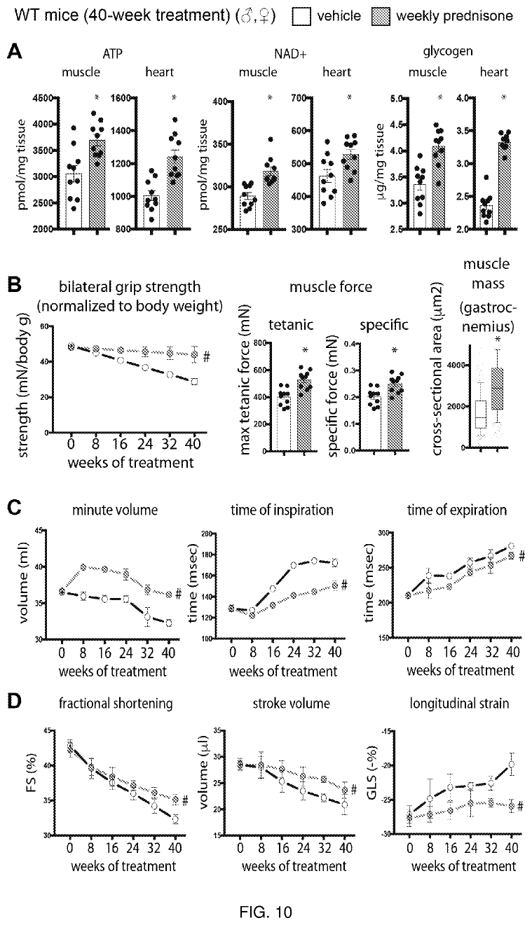

Whether pulsatile glucocorticoid treatment improved energy production and muscle function in aging mice was investigated next (FIG. 10). Wildtype (WT) mice were treated with either vehicle or weekly (pulsatile) 1 mg/kg intraperitoneal prednisone administration for 40 weeks from the age of 6 weeks. (FIG. 10A) As compared to vehicle treatment, weekly prednisone increased levels of ATP, NAD+ and glycogen in muscle and heart tissues. (FIG. 10B) In aged mice, weekly prednisone improved grip strength, tetanic and specific force, and muscle mass, seen as myofiber cross-sectional area (CSA). (FIG. 10C) Weekly prednisone improved parameters of respiratory function over time, as measured by whole-body plethysmography. (FIG. 10D) Weekly prednisone improved parameters of cardiac contractile function over time, as measured by echocardiography.

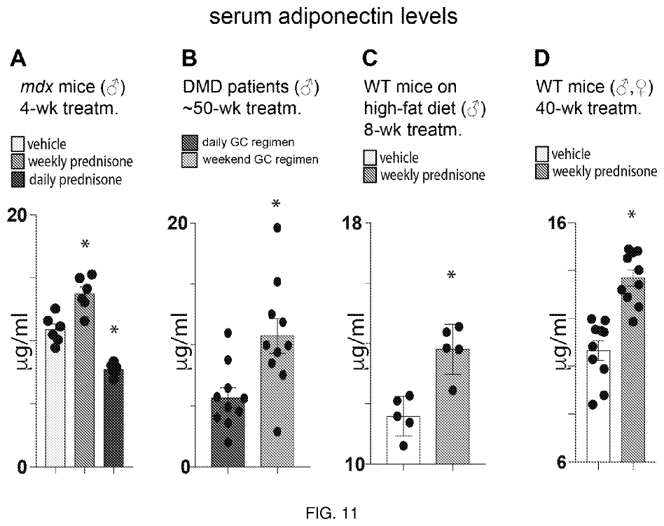

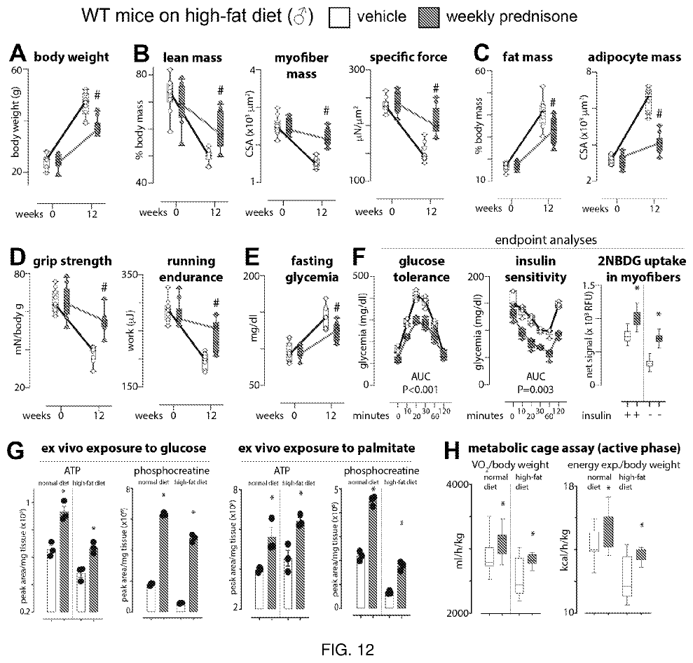

Considering the beneficial metabolic remodeling, the effects of pulsatile steroid administration on adiponectin levels were tested (FIG. 11). The analyses showed that pulsatile glucocorticoid treatment increased circulating adiponectin levels in mice and humans, including dystrophic mdx mice (FIG. 11A), in dystrophic DMD patients (FIG. 11B), in mice under diet-induced obesity (FIG. 11C), and in aging mice (FIG. 11D). Experiments were also performed to evaluate longer term outcomes from weekly steroids in a mouse model of obesity. Wildtype (WT) mice were fed high-fat chow and treated with either vehicle or once weekly (pulsatile) 1 mg/kg intraperitoneal prednisone administration for 12 weeks. FIG. 12 shows that 12-week-long pulsatile glucocorticoid exposure curbed obesity, insulin resistance, and metabolic dysfunction in wildtype mice with high fat diet-induced obesity.

### Discussion

Glucocorticoids are among the most highly prescribed drugs worldwide and are part of the standard of care to promote ambulation in DMD patients despite adverse side effects (McDonald et al., 2018). Studies of glucocorticoid effects in muscle are dominated by atrophic remodeling, which is especially prominent in mouse models (Schakman et al., 2009). Distinct from human muscle, mouse muscle has a higher ratio of type IIb myofibers, defined by fast myosin isoforms and a high reliance on glycolysis (Schiaffino and Reggiani, 2011). Fast myofibers are more susceptible than slow myofibers to FOXO3 activation and, hence, to glucocorticoid-driven atrophy (Sandri et al., 2006). Pulsatile glucocorticoids were discovered to induce a pro-ergogenic program supported by BCAA-mediated mitochondrial respiration and aerobic energy production, directed by a distinct epigenomic and transcriptional program linking GR to KLF15 and the muscle factor MEF2C. KLF15 is a circadian factor controlling amino acid metabolism that has been implicated in pro-ergogenic glucocorticoid cascades (Morrison-Nozik et al., 2015; Sun et al., 2016). The combination of KLF15 and MEF2C advances those findings to define a molecular regulatory combination effective for promoting muscle performance in dystrophic muscle.

Muscle catabolism of BCAA influences muscle function and whole-body metabolic homeostasis (Li et al., 2017; White et al., 2018), whereas disruption of BCAA disposal and utilization, including its accumulation in circulation and tissues, is associated with metabolic dysfunction and obesity (Lynch and Adams, 2014). The data presented here support that pulsatile glucocorticoids couple higher BCAA-mediated mitochondrial respiration to increased glycolysis, resulting in improved energy production and insulin sensitivity. Moreover, pulsatile steroid dosing increased NAD biogenesis pathway expression and NAD⁺ levels, further stabilizing favorable reprogramming of dystrophic muscle metabolism (Zhang et al., 2016). The combination of BCAA-mediated respiration, glycolysis and NAD repletion boosts energy production and muscle function in dystrophic muscle.

Strikingly, metabolic programming by pulsatile glucocorticoids was not limited to dystrophin-linked muscular dystrophy but was also seen in a genetic model of limb-girdle muscular dystrophy linked to a completely distinct cellular defect. There are currently no indications for glucocorticoids in muscular dystrophies beyond DMD, and efficacy has been questioned in small studies of daily steroid dosing (Godfrey et al., 2006; Walter et al., 2013). Intriguingly, it was recently reported that a glucocorticoid-KLF15-BCAA axis benefits a mouse model of spinal muscular atrophy, a genetic disorder with a significant neuronal component (Walter et al., 2018). It is therefore possible that favorable metabolic reprogramming by pulsed glucocorticoid regimens is applicable beyond muscle.

The findings disclosed herein demonstrate that pulsatile glucocorticoids enable a GR-KLF15-MEF2C axis in dystrophic muscle to support BCAA utilization and energy production, providing useful signatures to monitor these effects in other conditions of diseased, normal or aging muscle.

### REFERENCES

Ahn, B., Soundarapandian, M. M., Sessions, H., Peddibhotia, S., Roth, G. P., Li, J. L., Sugarman, E., Koo, A., Malany, S., Wang, M., et al. (2016). MondoA coordinately regulates skeletal myocyte lipid homeostasis and insulin signaling. J Clin Invest 126, 3567-3579.
Bentzinger, C. F., Romanino, K., Cloetta, D., Lin, S., Mascarenhas, J. B., Oliveri, F., Xia, J., Casanova, E., Costa, C. F., Brink, M., et al. (2008). Skeletal muscle-specific ablation of raptor, but not of rictor, causes metabolic changes and results in muscle dystrophy. Cell Metab 8, 411-424.
Bodine, S. C., Latres, E., Baumhueter, S., Lai, V. K., Nunez, L., Clarke, B. A., Poueymirou, W. T., Panaro, F. J., Na, E., Dharmarajan, K., et al. (2001). Identification of ubiquitin ligases required for skeletal muscle atrophy. Science 294, 1704-1708.
Bullard, S. A., Seo, S., Schilling, B., Dyle, M. C., Dierdorff, J. M., Ebert, S. M., DeLau, A. D., Gibson, B. W., and Adams, C. M. (2016). Gadd45a Protein Promotes Skeletal Muscle Atrophy by Forming a Complex with the Protein Kinase MEKK4. J Biol Chem 291, 17496-17509.
Connolly, A. M., Schierbecker, J., Renna, R., and Florence, J. (2002). High dose weekly oral prednisone improves strength in boys with Duchenne muscular dystrophy. Neuromuscul Disord 12, 917-925.
D'Antona, G., Ragni, M., Cardile, A., Tedesco, L., Dossena, M., Bruttini, F., Caliaro, F., Corsetti, G., Bottinelli, R., Carruba, M. O., et al. (2010). Branched-chain amino acid supplementation promotes survival and supports cardiac and skeletal muscle mitochondrial biogenesis in middle-aged mice. Cell Metab 12, 362-372.
Godfrey, C., Escolar, D., Brockington, M., Clement, E. M., Mein, R., Jimenez-Mallebrera, C., Torelli, S., Feng, L., Brown, S. C., Sewry, C. A., et al. (2006). Fukutin gene mutations in steroid-responsive limb girdle muscular dystrophy. Ann Neurol 60, 603-610.
Li, T., Zhang, Z., Kolwicz, S. C., Jr., Abell, L., Roe, N. D., Kim, M., Zhou, B., Cao, Y., Ritterhoff, J., Gu, H., et al. (2017). Defective Branched-Chain Amino Acid Catabolism Disrupts Glucose Metabolism and Sensitizes the Heart to Ischemia-Reperfusion Injury. Cell Metab 25, 374-385.
Lin, Q., Schwarz, J., Bucana, C., and Olson, E. N. (1997). Control of mouse cardiac morphogenesis and myogenesis by transcription factor MEF2C. Science 276, 1404-1407.
Lynch, C. J., and Adams, S. H. (2014). Branched-chain amino acids in metabolic signalling and insulin resistance. Nat Rev Endocrinol 10, 723-736.
McDonald, C. M., Henricson, E. K., Abresch, R. T., Duong, T., Joyce, N. C., Hu, F., Clemens, P. R., Hoffman, E. P., Cnaan, A., Gordish-Dressman, H., et al. (2018). Long-term effects of glucocorticoids on function, quality of life, and survival in patients with Duchenne muscular dystrophy: a prospective cohort study. Lancet 391, 451-461.
Morrison-Nozik, A., Anand, P., Zhu, H., Duan, Q., Sabeh, M., Prosdocimo, D. A., Lemieux, M. E., Nordsborg, N., Russell, A. P., MacRae, C. A., et al. (2015). Glucocorticoids enhance muscle endurance and ameliorate Duchenne muscular dystrophy through a defined metabolic program. Proc Natl Acad Sci USA 112, E6780-6789.
Nadal, A., Quesada, I., Tuduri, E., Nogueiras, R., and Alonso-Magdalena, P. (2017). Endocrine-disrupting chemicals and the regulation of energy balance. Nat Rev Endocrinol 13, 536-546.
Quattrocelli, M., Barefield, D. Y., Warner, J. L., Vo, A. H., Hadhazy, M., Earley, J. U., Demonbreun, A. R., and McNally, E. M. (2017a). Intermittent glucocorticoid steroid dosing enhances muscle repair without eliciting muscle atrophy. J Clin Invest 127, 2418-2432.
Quattrocelli, M., Salamone, I. M., Page, P. G., Warner, J. L., Demonbreun, A. R., and McNally, E. M. (2017b). Intermittent Glucocorticoid Dosing Improves Muscle Repair and Function in Mice with Limb-Girdle Muscular Dystrophy. Am J Pathol 187, 2520-2535.
Rivera, C. M., and Ren, B. (2013). Mapping human epigenomes. Cell 155, 39-55.
Ryu, D., Zhang, H., Ropelle, E. R., Sorrentino, V., Mazala, D. A., Mouchiroud, L., Marshall, P. L., Campbell, M. D., Ali, A. S., Knowels, G. M., et al. (2016). NAD+ repletion improves muscle function in muscular dystrophy and counters global PARylation. Sci Transl Med 8, 361ra139.
Sancak, Y., Peterson, T. R., Shaul, Y. D., Lindquist, R. A., Thoreen, C. C., Bar-Peled, L., and Sabatini, D. M. (2008). The Rag GTPases bind raptor and mediate amino acid signaling to mTORC1. Science 320, 1496-1501.
Sandri, M., Lin, J., Handschin, C., Yang, W., Arany, Z. P., Lecker, S. H., Goldberg, A. L., and Spiegelman, B. M. (2006). PGC-1alpha protects skeletal muscle from atrophy by suppressing FoxO3 action and atrophy-specific gene transcription. Proc Natl Acad Sci USA 103, 16260-16265.
Sandri, M., Sandri, C., Gilbert, A., Skurk, C., Calabria, E., Picard, A., Walsh, K., Schiaffino, S., Lecker, S. H., and Goldberg, A. L. (2004). Foxo transcription factors induce the atrophy-related ubiquitin ligase atrogin-1 and cause skeletal muscle atrophy. Cell 117, 399-412.
Schakman, O., Gilson, H., Kalista, S., and Thissen, J. P. (2009). Mechanisms of muscle atrophy induced by glucocorticoids. Horm Res 72 Suppl 1, 36-41.
Schiaffino, S., and Reggiani, C. (2011). Fiber types in mammalian skeletal muscles. Physiol Rev 91, 1447-1531.
Shintaku, J., Peterson, J. M., Talbert, E. E., Gu, J. M., Ladner, K. J., Williams, D. R., Mousavi, K., Wang, R., Sartorelli, V., and Guttridge, D. C. (2016). MyoD Regulates Skeletal Muscle Oxidative Metabolism Cooperatively with Alternative NF-kappaB. Cell Rep 17, 514-526.
Sun, H., Olson, K. C., Gao, C., Prosdocimo, D. A., Zhou, M., Wang, Z., Jeyaraj, D., Youn, J. Y., Ren, S., Liu, Y., et al. (2016). Catabolic Defect of Branched-Chain Amino Acids Promotes Heart Failure. Circulation 133, 2038-2049.
Vockley, C. M., D'Ippolito, A. M., McDowell, I. C., Majoros, W. H., Safi, A., Song, L., Crawford, G. E., and Reddy, T. E. (2016). Direct GR Binding Sites Potentiate Clusters of TF Binding across the Human Genome. Cell 166, 1269-1281 e1219.
Walter, L. M., Deguise, M. O., Meijboom, K. E., Betts, C. A., Ahlskog, N., van Westering, T. L. E., Hazell, G., McFall, E., Kordala, A., Hammond, S. M., et al. (2018). Interventions Targeting Glucocorticoid-Kruppel-like Factor 15-Branched-Chain Amino Acid Signaling Improve Disease Phenotypes in Spinal Muscular Atrophy Mice. EBioMedicine 31, 226-242.
Walter, M. C., Reilich, P., Thiele, S., Schessl, J., Schreiber, H., Reiners, K., Kress, W., Muller-Reible, C., Vorgerd, M., Urban, P., et al. (2013). Treatment of dysferlinopathy with deflazacort: a double-blind, placebo-controlled clinical trial. Orphanet J Rare Dis 8, 26.
White, P. J., McGarrah, R. W., Grimsrud, P. A., Tso, S. C., Yang, W. H., Haldeman, J. M., Grenier-Larouche, T., An, J., Lapworth, A. L., Astapova, I., et al. (2018). The BCKDH Kinase and Phosphatase Integrate BCAA and Lipid Metabolism via Regulation of ATP-Citrate Lyase. Cell Metab 27, 1281-1293 e1287.
Zhang, H., Ryu, D., Wu, Y., Gariani, K., Wang, X., Luan, P., D'Amico, D., Ropelle, E. R., Lutolf, M. P., Aebersold, R., et al. (2016). NAD(+) repletion improves mitochondrial and stem cell function and enhances life span in mice. Science 352, 1436-1443.
Anders, S., Pyl, P. T., and Huber, W. (2015). HTSeq—a Python framework to work with high-throughput sequencing data. Bioinformatics 31, 166-169.
Ashburner, M., Ball, C. A., Blake, J. A., Botstein, D., Butler, H., Cherry, J. M., Davis, A. P., Dolinski, K., Dwight, S. S., Eppig, J. T., et al. (2000). Gene ontology: tool for the unification of biology. The Gene Ontology Consortium. Nat Genet 25, 25-29.
Bruno, C., Patin, F., Bocca, C., Nadal-Desbarats, L., Bonnier, F., Reynier, P., Emond, P., Vourc'h, P., Joseph-Delafont, K., Corcia, P., et al. (2018). The combination of four analytical methods to explore skeletal muscle metabolomics: Better coverage of metabolic pathways or a marketing argument? J Pharm Biomed Anal 148, 273-279.
Carey, M. F., Peterson, C. L., and Smale, S. T. (2009). Chromatin immunoprecipitation (ChIP). Cold Spring Harb Protoc 2009, pdb prot5279.
Choi, Y. K., and Park, K. G. (2018). Targeting Glutamine Metabolism for Cancer Treatment. Biomol Ther (Seoul) 26, 19-28.
Chong, J., Soufan, O., Li, C., Caraus, I., Li, S., Bourque, G., Wishart, D. S., and Xia, J. (2018). MetaboAnalyst 4.0: towards more transparent and integrative metabolomics analysis. Nucleic Acids Res.
Demonbreun, A. R., Fahrenbach, J. P., Deveaux, K., Earley, J. U., Pytel, P., and McNally, E. M. (2011). Impaired muscle growth and response to insulin-like growth factor 1 in dysferlin-mediated muscular dystrophy. Hum Mol Genet 20, 779-789.
Demonbreun, A. R., and McNally, E. M. (2015). DNA Electroporation, Isolation and Imaging of Myofibers. J Vis Exp, e53551.
Demonbreun, A. R., Rossi, A. E., Alvarez, M. G., Swanson, K. E., Deveaux, H. K., Earley, J. U., Hadhazy, M., Vohra, R., Walter, G. A., Pytel, P., et al. (2014). Dysferlin and myoferlin regulate transverse tubule formation and glycerol sensitivity. Am J Pathol 184, 248-259.
DiFranco, M., Quinonez, M., Capote, J., and Vergara, J. (2009). DNA transfection of mammalian skeletal muscles using in vivo electroporation. J Vis Exp.
Heinz, S., Benner, C., Spann, N., Bertolino, E., Lin, Y. C., Laslo, P., Cheng, J. X., Murre, C., Singh, H., and Glass, C. K. (2010). Simple combinations of lineage-determining transcription factors prime cis-regulatory elements required for macrophage and B cell identities. Mol Cell 38, 576-589.
Hill, A. M., LaForgia, J., Coates, A. M., Buckley, J. D., and Howe, P. R. (2007). Estimating abdominal adipose tissue with DXA and anthropometry. Obesity (Silver Spring) 15, 504-510.
Kambo, A., Sharma, V. S., Casteel, D. E., Woods, V. L., Jr., Pilz, R. B., and Boss, G. R. (2005). Nitric oxide inhibits mammalian methylmalonyl-CoA mutase. J Biol Chem 280, 10073-10082.
Kerr, J. P., Ziman, A. P., Mueller, A. L., Muriel, J. M., Kleinhans-Welte, E., Gumerson, J. D., Vogel, S. S., Ward, C. W., Roche, J. A., and Bloch, R. J. (2013). Dysferlin stabilizes stress-induced Ca2+ signaling in the transverse tubule membrane. Proc Natl Acad Sci USA 110, 20831-20836.
Kozhemyakina, E., Cohen, T., Yao, T. P., and Lassar, A. B. (2009). Parathyroid hormone-related peptide represses chondrocyte hypertrophy through a protein phosphatase 2A/histone deacetylase 4/MEF2 pathway. Mol Cell Biol 29, 5751-5762.
Langmead, B., and Salzberg, S. L. (2012). Fast gapped-read alignment with Bowtie 2. Nat Methods 9, 357-359.
Metsalu, T., and Vilo, J. (2015). ClustVis: a web tool for visualizing clustering of multivariate data using Principal Component Analysis and heatmap. Nucleic Acids Res 43, W566-570.
Perez-Llamas, C., and Lopez-Bigas, N. (2011). Gitools: analysis and visualisation of genomic data using interactive heat-maps. PLoS One 6, e19541.
Quattrocelli, M., Swinnen, M., Giacomazzi, G., Camps, J., Barthelemy, I., Ceccarelli, G., Caluwe, E., Grosemans, H., Thorrez, L., Pelizzo, G., et al. (2015). Mesodermal iPSC-derived progenitor cells functionally regenerate cardiac and skeletal muscle. J Clin Invest 125, 4463-4482.
Ramsey, K. M., Yoshino, J., Brace, C. S., Abrassart, D., Kobayashi, Y., Marcheva, B., Hong, H. K., Chong, J. L., Buhr, E. D., Lee, C., et al. (2009). Circadian clock feedback cycle through NAMPT-mediated NAD+ biosynthesis. Science 324, 651-654.
Robinson, M. D., McCarthy, D. J., and Smyth, G. K. (2010). edgeR: a Bioconductor package for differential expression analysis of digital gene expression data. Bioinformatics 26, 139-140.
Sali, A., Guerron, A. D., Gordish-Dressman, H., Spurney, C. F., Iantorno, M., Hoffman, E. P., and Nagaraju, K. (2012). Glucocorticoid-treated mice are an inappropriate positive control for long-term preclinical studies in the mdx mouse. PLoS One 7, e34204.
Schneider, C. A., Rasband, W. S., and Eliceiri, K. W. (2012). NIH Image to ImageJ: 25 years of image analysis. Nat Methods 9, 671-675.
Shintaku, J., and Guttridge, D. C. (2016). Analysis of Aerobic Respiration in Intact Skeletal Muscle Tissue by Microplate-Based Respirometry. Methods Mol Biol 1460, 337-343.
Trapnell, C., Pachter, L., and Salzberg, S. L. (2009). TopHat: discovering splice junctions with RNA-Seq. Bioinformatics 25, 1105-1111.
Xian, Z. Y., Liu, J. M., Chen, Q. K., Chen, H. Z., Ye, C. J., Xue, J., Yang, H. Q., Li, J. L., Liu, X. F., and Kuang, S. J. (2015). Inhibition of LDHA suppresses tumor progression in prostate cancer. Tumour Biol 36, 8093-8100.
Zou, C., Wang, Y., and Shen, Z. (2005). 2-NBDG as a fluorescent indicator for direct glucose uptake measurement. J Biochem Biophys Methods 64, 207-215.
Gerke, V., C. E. Creutz, and S. E. Moss. 2005. Annexins: linking Ca2+ signalling to membrane dynamics. Nat Rev Mol Cell Biol. 6:449-461.
de Laat, B., R. H. Derksen, I. J. Mackie, M. Roest, S. Schoormans, B. J. Woodhams, P. G. de Groot, and W. L. van Heerde. 2006. Annexin A5 polymorphism (−1C-->T) and the presence of anti-annexin A5 antibodies in the antiphospholipid syndrome. Annals of the rheumatic diseases. 65:1468-1472.
Gerke, V., and S. E. Moss. 2002. Annexins: from structure to function. Physiol Rev. 82:331-371. Blackwood, R. A., and J. D. Ernst. 1990. Characterization of Ca2(+)-dependent phospholipid binding, vesicle aggregation and membrane fusion by annexins. The Biochemical journal. 266:195-200.
Goulet, F., K. G. Moore, and A. C. Sartorelli. 1992. Glycosylation of annexin I and annexin II. Biochemical and biophysical research communications. 188:554-558.
Kaetzel, M. A., Y. D. Mo, T. R. Mealy, B. Campos, W. Bergsma-Schutter, A. Brisson, J. R. Dedman, and B. A. Seaton. 2001. Phosphorylation mutants elucidate the mechanism of annexin IV-mediated membrane aggregation. Biochemistry. 40:4192-4199.
Zaks, W. J., and C. E. Creutz. 1991. Ca(2+)-dependent annexin self-association on membrane surfaces. Biochemistry. 30:9607-9615.
Christmas, P., J. Callaway, J. Fallon, J. Jones, and H. T. Haigler. 1991. Selective secretion of annexin 1, a protein without a signal sequence, by the human prostate gland. The Journal of biological chemistry. 266:2499-2507.
Deora, A. B., G. Kreitzer, A. T. Jacovina, and K. A. Hajjar. 2004. An annexin 2 phosphorylation switch mediates p11-dependent translocation of annexin 2 to the cell surface. The Journal of biological chemistry. 279:43411-43418.
Wallner, B. P., R. J. Mattaliano, C. Hession, R. L. Cate, R. Tizard, L. K. Sinclair, C. Foeller, E. P. Chow, J. L. Browing, K. L. Ramachandran, and et al. 1986. Cloning and expression of human lipocortin, a phospholipase A2 inhibitor with potential anti-inflammatory activity. Nature. 320:77-81.
Hannon, R., J. D. Croxtall, S. J. Getting, F. Roviezzo, S. Yona, M. J. Paul-Clark, F. N. Gavins, M. Perretti, J. F. Morris, J. C. Buckingham, and R. J. Flower. 2003. Aberrant inflammation and resistance to glucocorticoids in annexin 1−/− mouse. FASEB J. 17:253-255.
Ling, Q., A. T. Jacovina, A. Deora, M. Febbraio, R. Simantov, R. L. Silverstein, B. Hempstead, W. H. Mark, and K. A. Hajjar. 2004. Annexin II regulates fibrin homeostasis and neoangiogenesis in vivo. The Journal of clinical investigation. 113:38-48.
Cagliani, R., F. Magri, A. Toscano, L. Merlini, F. Fortunato, C. Lamperti, C. Rodolico, A. Prelle, M. Sironi, M. Aguennouz, P. Ciscato, A. Uncini, M. Moggio, N. Bresolin, and G. P. Comi. 2005. Mutation finding in patients with dysferlin deficiency and role of the dysferlin interacting proteins annexin A1 and A2 in muscular dystrophies. Human mutation. 26:283.

What is claimed is:

1. A method of administering a glucocorticoid steroid to a patient, wherein the patient has a serum or plasma level of one or more of the following biomarkers that is:
(a) less than about 18 μg/dL morning fasting cortisol;
(b) at least about 90 mg/dL fasting morning glucose;
(c) at least about 160 pmol/L insulin;
(d) at least about 40 μmol/L isoleucine;
(e) at least about 100 μmol/L leucine;
(f) at least about 120 μmol/L valine;
(g) at least about 700 μmol/L combined branched chain amino acids;
(h) at least about 110 mg/dL triglycerides;
(i) at least about 300 μmol/L non-esterified fatty acids; and
(j) at least about 100 μmol/L combined ketones;
wherein the administering of the glucocorticoid steroid comprises once-weekly administration of the glucocorticoid steroid.

2. The method of claim 1, wherein the patient suffers from muscle wasting, obesity, a metabolic disorder, sarcopenia, an inflammatory disorder, a muscle injury, or a combination thereof.

3. The method of claim 1 or claim 2, wherein the once-weekly administration of glucocorticoid steroid comprises a single dose of about 0.1 to about 5 mg/kg.

4. The method of any one of claims 1-3, wherein the once-weekly administration of glucocorticoid steroid comprises a single dose of about 1 mg/kg.

5. The method of any one of claims 1-3, wherein the once-weekly administration of glucocorticoid steroid comprises a single dose of about 0.75 mg/kg.

6. The method of any one of claims 2-5, wherein the muscle wasting is aging-related muscle wasting, disease-related muscle wasting, diabetes-associated muscle wasting, muscle atrophy, sarcopenia, cardiomyopathy, a chronic myopathy, an inflammatory myopathy, a muscular dystrophy, or a combination thereof.

7. The method of any one of claims 1-6, wherein the metabolic disorder is metabolic syndrome, insulin resistance, a nutrition disorder, exercise intolerance, or a combination thereof.

8. The method of claim 6, wherein the cardiomyopathy is hypertrophic, dilated, congenital, arrhythmogenic, restrictive, ischemic, or heart failure.

9. The method of claim 8, wherein the heart failure includes reduced ejection fraction.

10. The method of claim 8, wherein the heart failure includes preserved ejection fraction.

11. The method of any one of claims 1-10, wherein the administering results in one or more of decreased insulin resistance, increased glucose tolerance, increased muscle mass, decreased hyperinsulinemia, increased lean mass, increased force, increased systolic function, increased diastolic function, decreased muscle fibrosis, increased exercise tolerance, increased nutrient catabolism, increased energy production, increased serum adiponectin, decreased serum branched chain amino acids (BCAA), decreased serum lipid level, decreased serum ketone level, decreased hyperglycemia, increased serum cortisol level, increased serum corticosterone, and decreased adipocyte size compared to administering the glucocorticoid steroid in a dosing regimen that is not once-weekly or to not administering the glucocorticoid steroid.

12. The method of any one of claims 1-11, further comprising administering an effective amount of (i) an agent that increases the activity of an annexin protein, (ii) mitsugumin 53 (MG53), (iii) a modulator of latent TGF-β binding protein 4 (LTBP4), (iv) a modulator of transforming growth factor β (TGF-β) activity, (v) a modulator of androgen response, (vi) a modulator of an inflammatory response, (vii) a promoter of muscle growth, (viii) a chemotherapeutic agent, (ix) a modulator of fibrosis, (x) a modulator of glucose homeostasis, (xi) a modulator of metabolic function, or a combination thereof.

13. The method of claim 12, wherein the agent that increases the activity of an annexin protein is selected from the group consisting of a recombinant protein, a steroid, and a polynucleotide capable of expressing an annexin protein.

14. The method of claim 13, wherein the polynucleotide is associated with a nanoparticle.

15. The method of claim 13, wherein the polynucleotide is contained in a vector.

16. The method of claim 15, wherein the vector is within a chloroplast.

17. The method of claim 15 wherein the vector is a viral vector.

18. The method of claim 17 wherein the viral vector is selected from the group consisting of a herpes virus vector, an adeno-associated virus (AAV) vector, an adeno virus vector, and a lentiviral vector.

19. The method of claim 18 wherein the AAV vector is recombinant AAV5, AAV6, AAV8, AAV9, or AAV74.

20. The method of claim 19, wherein the AAV74 vector is AAVrh74.

21. The method of any one of claims 12-20, wherein the agent increases the activity of annexin A1 (SEQ ID NO: 1), annexin A2 (SEQ ID NO: 2 or SEQ ID NO: 3), annexin A3 (SEQ ID NO: 4), annexin A4 (SEQ ID NO: 5), annexin A5 (SEQ ID NO: 6), annexin A6 (SEQ ID NO: 7, SEQ ID NO: 8, SEQ ID NO: 44, or a combination thereof), annexin A7 (SEQ ID NO: 9 or SEQ ID NO: 10), annexin A8 (SEQ ID NO: 11 or SEQ ID NO: 12), annexin A9 (SEQ ID NO: 13), annexin A10 (SEQ ID NO: 14), annexin A11 (SEQ ID NO: 15 or SEQ ID NO: 16), annexin A13 (SEQ ID NO: 17 or SEQ ID NO: 18), or a combination thereof.

22. The method of claim 21, wherein the agent increases the activity of annexin A1 (SEQ ID NO: 1), annexin A2 (SEQ ID NO: 2 or SEQ ID NO: 3), and annexin A6 (SEQ ID NO: 7, SEQ ID NO: 8, SEQ ID NO: 44, or a combination thereof).

23. The method of claim 21, wherein the agent increases the activity of annexin A2 (SEQ ID NO: 2 or SEQ ID NO: 3) and annexin A6 (SEQ ID NO: 7, SEQ ID NO: 8, SEQ ID NO:
44, or a combination thereof).

24. The method of claim 21, wherein the agent increases the activity of annexin A1 (SEQ ID NO: 1) and annexin A6 (SEQ ID NO: 7, SEQ ID NO: 8, SEQ ID NO: 44, or a combination thereof).

25. The method of claim 21, wherein the agent increases the activity of annexin A6 (SEQ ID NO: 7, SEQ ID NO: 8, SEQ ID NO: 44, or a combination thereof).
